(19) United States
(12) Patent Application Publication
(10) Pub. No.: US 2019/0365913 A1
(43) Pub. Date: Dec. 5, 2019

(54) STAPLED PEPTIDE CONJUGATES AND PARTICLES

(71) Applicant: TARVEDA THERAPEUTICS, INC., Watertown, MA (US)

(72) Inventors: Richard Wooster, Natick, MA (US); Sudhakar Kadiyala, Newton, MA (US); Mark T. Bilodeau, Waltham, MA (US)

(21) Appl. No.: 16/075,292

(22) PCT Filed: Feb. 3, 2017
(86) PCT No.: PCT/US2017/016396

§ 371 Date: Aug. 3, 2018

Related U.S. Application Data
(60) Provisional application No. 62/291,212, filed on Feb. 4, 2016.

(51) Int. Cl. A61K 47/64; A61K 38/17; A61K 47/69

(52) U.S. Cl. CPC A61K 47/6425; B82Y 5/00; A61K 47/6937; A61K 38/177

(57) ABSTRACT

Conjugates of an active agent such as a therapeutic, prophylactic, or diagnostic agent attached to a targeting moiety via a linker, and particles comprising such conjugates have been designed which can provide improved temporospatial delivery of the active agent and/or improved biodistribution. Methods of making the conjugates, the particles, and the formulations thereof are provided. Methods of administering the formulations to a subject in need thereof are provided, for example, to treat or prevent cancer or infectious diseases.

### CROSS REFERENCE TO RELATED APPLICATIONS

This application claims priority to U.S. Provisional Application No. 62/291,212 filed Feb. 4, 2016.

### FIELD OF THE INVENTION

This invention is generally in the field of conjugates and particles for drug delivery.

### BACKGROUND OF THE INVENTION

Developments in nanomedicine are generally directed towards improving the pharmaceutical properties of the drugs and, in some cases, enhancing the targeted delivery in a more cell-specific manner. Several cell-specific drugs have been described, and include monoclonal antibodies, aptamers, peptides, and small molecules. Despite some of the potential advantages of such drugs, a number of problems have limited their clinical application, including size, stability, manufacturing cost, immunogenicity, poor pharmacokinetics and other factors.

Nanoparticulate drug delivery systems are attractive for systemic drug delivery because they may be able to prolong the half-life of a drug in circulation, reduce nonspecific uptake of a drug, and improve accumulation of a drug at tumors, e.g., through an enhanced permeation and retention (EPR) effect. There are limited examples of therapeutics formulated for delivery as nanoparticles, which include DOXIL® (liposomal encapsulated doxyrubicin) and ABRAXANE® (albumin bound paclitaxel nanoparticles).

The development of nanotechnologies for effective delivery of drugs or drug candidates to specific diseased cells and tissues, e.g., to cancer cells, in specific organs or tissues, in a temporospatially regulated manner potentially can overcome or ameliorate therapeutic challenges, such as systemic toxicity. However, while targeting of the delivery system may preferentially deliver drug to a site where therapy is needed, the drug released from the nanoparticle may not for example, remain in the region of the targeted cells in efficacious amounts or may not remain in the circulation in a relatively non-toxic state for a sufficient amount of time to decrease the frequency of treatment or permit a lower amount of drug to be administered while still achieving a therapeutic effect. Accordingly, there is a need in the art for improved drug targeting and delivery, including identification of targeting molecules that can be incorporated into particles and whose presence does not substantially interfere with efficacy of the drug.

### SUMMARY OF THE INVENTION

Applicants have created molecules that are conjugates of a targeting moiety and an active agent, e.g., a cancer therapeutic agent such as a platinum-containing agent. Furthermore, particles comprising the conjugates are provided. The conjugates can be encapsulated into particles, included in the particle/medium interface, or deposited on the surface of particles. The conjugates and particles are useful for improving the delivery of active agents such as tumor cytotoxic agents to tumor tissue and tumor cells via both passive and active targeting mechanism.

Applicants have developed novel conjugates and particles comprising these conjugates, including polymeric nanoparticles, self-assembling particles, conjugate/surfactant and conjugate/block co-polymers mixed micelles, composite nanoparticles formed by conjugates, surfactants and phospholipids or block co-polymers, or polyaminoacids, or proteins, inorganic nanoparticles, and pharmaceutical formulations thereof. The conjugates of an active agent such as a therapeutic, prophylactic, or diagnostic agent are attached via a linker to a targeting moiety. The conjugates and particles can provide improved temporospatial delivery of the active agent and/or improved biodistribution compared to delivery of the active agent alone. In some cases, the targeting moiety can also act as a therapeutic agent. In some embodiments, the targeting moiety does not substantially interefere with efficacy of the therapeutic agent in vivo. Methods of making conjugates, particles, and formulations comprising such particles are described herein. Such particles are useful for treating or preventing diseases that are susceptible to the active agent, for example, treating or preventing cancer or infectious diseases.

The conjugates include a targeting ligand and an active agent connected by a linker, wherein the conjugate in some embodiments has the formula:

(X—Y—Z)

wherein X is a targeting moiety; Y is a linker; and Z is an active agent.

One ligand can be conjugated to two or more active agents where the conjugate has the formula: X—(Y—Z)ₙ. In other embodiments, one active agent molecule can be linked to two or more ligands wherein the conjugate has the formula: (X—Y)ₙ—Z. n is an integer equal to or greater than 1.

Methods of making the conjugates and particles containing the conjugates are provided. Methods are also provided for treating a disease or condition, the method comprising administering a therapeutically effective amount of the particles containing a conjugate to a subject in need thereof. In some embodiments, the conjugates are targeted to a cancer or hyperproliferative disease, for example, lymphoma (e.g., non-Hodgkin's lymphoma), renal cell carcinoma, prostate cancer, ovarian cancer, breast cancer, colorectal cancer, neuroendodrine cancer, endometrial cancer, pancreatic cancer leukemia, lung cancer, glioblastoma multiforme, stomach cancer, liver cancer, sarcoma, bladder cancer, testicular cancer, esophageal cancer, head and neck cancer, and leptomeningeal carcinomatosis.

### DETAILED DESCRIPTION OF THE INVENTION

Applicants have created novel conjugates and/or particles comprising such conjugates to improve targeting a conjugate comprising an active agent to a diseased tissue such as tumor tissues. Such targeting can, for example, improve the amount of active agent delivered at an action site and decrease the active agent's systemic toxicity. As used herein, “toxicity” refers to the capacity of a substance or composition to hit off targets and/or be harmful or poisonous to a cell, tissue, organ tissue, vasculature, or cellular environment. Low toxicity refers to a reduced capacity of a substance or composition to be harmful or poisonous to a cell, tissue, organ tissue or cellular environment. Such reduced or low toxicity may be relative to a standard measure, relative to a treatment or relative to the absence of a treatment.

Toxicity may further be measured relative to a subject's weight loss where weight loss over 15%, over 20% or over 30% of the body weight is indicative of toxicity. Other metrics of toxicity may also be measured such as patient presentation metrics including lethargy and general malaiase. Neutropenia or thrombopenia may also be metrics of toxicity.

Biomarkers of toxicity include elevated AST/ALT levels, neurotoxicity, kidney damage, GI damage and the like.

The conjugates described herein that are formulated with particles are released after administration of the particles. The targeted drug conjugates utilize active molecular targeting in combination with enhanced permeability and retention effect (EPR) and improved overall biodistribution of the nanoparticles to provide greater efficacy and improved tolerability as compared to the administration of targeted particles, encapsulated untargeted drug, or unencapsuted drug.

In addition, the toxicity of a conjugate containing a targeting moiety linked to an active agent for cells that do not express the target of the targeting moiety is predicted to be decreased compared to the toxicity of the active agent alone. Without committing to any particular theory, applicants believe that this feature is because of the ability of the conjugated active agent to enter a cell is decreased compared the ability to enter a cell of the active agent alone. Accordingly, the conjugates comprising an active agent and particles containing the conjugates as described herein generally have reduced toxicity for cells that do not express the target of the targeting moiety and at least the same or increased toxicity for cells that express the target of the targeting moiety compared to the active agent alone.

Furthermore, a conjugate comprising an active agent may be degraded and/or compromised before it reaches a target site. For example, there may be specific enzymes in the plasma that may degrade the conjugate. The particles of the present invention may shield the conjugate from degradation and/or compromise before the conjugate reaches the target site.

It is an object of the invention to provide improved compounds, compositions, and formulations for temporospatial drug delivery.

It is further an object of the invention to provide methods of making improved compounds, compositions, and formulations for temporospatial drug delivery.

It is also an object of the invention to provide methods of administering the improved compounds, compositions, and formulations to individuals in need thereof.

### I. Conjugates

Conjugates include an active agent or prodrug thereof attached to a targeting moiety by a linker. The conjugates can be a conjugate between a single active agent and a single targeting moiety, e.g. a conjugate having the structure X—Y—Z where X is the targeting moiety, Y is the linker, and Z is the active agent.

In some embodiments the conjugate contains more than one targeting moiety, more than one linker, more than one active agent, or any combination thereof. The conjugate can have any number of targeting moieties, linkers, and active agents. The conjugate can have the structure X—Y—Z—Y—X, (X—Y)ₙ—Z, X—(Y—Z)ₙ, X—Y—Zₙ, (X—Y—Z)ₙ, (X—Y—Z—Y)ₙ—Z where X is a targeting moiety, Y is a linker, Z is an active agent, and n is an integer between 1 and 50, between 2 and 20, for example, between 1 and 5. Each occurrence of X, Y, and Z can be the same or different, e.g. the conjugate can contain more than one type of targeting moiety, more than one type of linker, and/or more than one type of active agent.

The conjugate can contain more than one targeting moiety attached to a single active agent. For example, the conjugate can include an active agent with multiple targeting moieties each attached via a different linker. The conjugate can have the structure X—Y—Z—Y—X where each X is a targeting moiety that may be the same or different, each Y is a linker that may be the same or different, and Z is the active agent.

The conjugate can contain more than one active agent attached to a single targeting moiety. For example the conjugate can include a targeting moiety with multiple active agents each attached via a different linker. The conjugate can have the structure Z—Y—X—Y—Z where X is the targeting moiety, each Y is a linker that may be the same or different, and each Z is an active agent that may be the same or different.

The conjugate may comprise pendent or terminal functional groups that allow further modification or conjugation. The pendent or terminal functional groups may be protected with any suitable protecting groups.

A. Active Agents

The conjugate contains at least one active agent or payload. The conjugate can contain more than one active agent, that can be the same or different. The active agent can be a therapeutic, prophylactic, diagnostic, or nutritional agent. A variety of active agents are known in the art and may be used in the conjugates described herein. The active agent can be a protein or peptide, small molecule, nucleic acid or nucleic acid molecule, lipid, sugar, glycolipid, glycoprotein, lipoprotein, or combination thereof. In some embodiments, the active agent is an antigen or adjuvant, radioactive or imaging agent (e.g., a fluorescent moiety) or polynucleotide. In some embodiments the active agent is an organometallic compound.

In some embodiments, the active agent is an anti-inflammatory agent. For example, the active agent may be a nonsteroidal anti-inflammatory drug (NSAID). In some embodiments, the active agent is a folate-targeting agent. In some embodiments, the active agent is a non-steroidal anti-inflammatory drug selected from: indomethacin, diclofenac, flurbiprofen, ketorolac, or suprofen.

In some embodiments, the active agent is an anti-inflammatory agent. For example, the active agent may be a nonsteroidal anti-inflammatory drug (NSAID). In some embodiments, the active agent is a folate-targeting agent. In some embodiments, the active agent is a non-steroidal anti-inflammatory drug selected from: indomethacin, diclofenac, flurbiprofen, ketorolac, or suprofen.

In some embodiments, the active agent is an antibiotic agent. In some embodiments the active agent is selected from levofloxacin, moxifloxacin, gatifloxacin, gemifloxacin, trovafloxacin, ofloxacin, ciprofloxacin, sparfloxacin, grepafloxacin, norfoxacin, enoxacin, lomefloxacin, fleroxacin, tosufloxacin, prulifloxacin, pazufloxacin, clinafloxacin, garenoxacin, sitafloxacin, loracarbef, cephalexin, cefuroxime, ceftriaxone, ceftaxime, ceftizoxime, ceftibuten, ceftazidime, cefprozil, cefpodoxime, cefoxitin, cefotetan, cefotaxime, cefoperazone, cefixime, cefepime, cefditoren, cefdinir, cefoperaxone, moxalactam, cefazolin, cefamandole, cefadroxil, cefaclor, cephalothin, cephradine, cephacetrile, cephalothin, chloramphenicol, tobramycin, streptomycin, gentamicin, kanamycin, amikacin, netilmicin, penicillin g, ticarcillin, methicillin, phenthicillin, cloxacillin, dicloxacillin, nafcillin and oxacillin.

In some embodiments, the active agent is an anti-cancer agent.

Anti-Cancer Agents

In certain embodiments, the active agent is a small molecule having a molecular weight preferably <about 5 kDa, more preferably <about 4 kDa, more preferably about 3 kDa, most preferably <about 1.5 kDa or <about 1 kDa.

The small molecule active agents used in this invention (e.g. antiproliferative (cytotoxic and cytostatic) agents capable of being linked to a polymer carrier) include cytotoxic compounds (e.g., broad spectrum), angiogenesis inhibitors, cell cycle progression inhibitors, PBK/m-TOR/AKT pathway inhibitors, MAPK signaling pathway inhibitors, kinase inhibitors, protein chaperones inhibitors, HDAC inhibitors, PARP inhibitors, Wnt/Hedgehog signaling pathway inhibitors, RNA polymerase inhibitors and proteasome inhibitors. The small molecule active agents in some embodiments the active agent is an analog, derivative, prodrug, or pharmaceutically acceptable salt thereof.

Broad spectrum cytotoxins include, but are not limited to, DNA-binding or alkylating drugs, microtubule stabilizing and destabilizing agents, platinum compounds, and topoisomerase I inhibitors.

Exemplary DNA-binding or alkylating drugs include, CC-1065 and its analogs, anthracyclines (doxorubicin, epirubicin, idarubicin, daunorubicin) and its analogs, alkylating agents, such as calicheamicins, dactinomycines, mitromycines, pyrrolobenzodiazepines, and the like.

Exemplary doxorubicin analogs include nemorubicin metabolite or analog drug moiety disclosed in US 20140227299 to Cohen et al., the contents of which are incorporated herein by reference in their entirety.

Exemplary CC-1065 analogs include duocarmycin SA, duocarmycin CI, duocarmycin C2, duocarmycin B2, DU-86, KW-2189, bizelesin, seco-adozelesin, and those described in U.S. Pat. Nos. 5,475,092; 5,595,499; 5,846,545; 6,534,660; 6,586,618; 6,756,397 and 7,049,316. Doxorubicin and its analogs include PNU-159682 and those described in U.S. Pat. No. 6,630,579 and nemorubicin metabolite or analog drugs disclosed in US 20140227299 to Cohen et al., the contents of which are incorporated herein by reference in their entirety.

Calicheamicins include those described in U.S. Pat. Nos. 5,714,586 and 5,739,116. Duocarmycins include those described in U.S. Pat. Nos. 5,070,092; 5,101,038; 5,187,186; 6,548,530; 6,660,742; and 7,553,816 B2; and Li et al., Tet Letts., 50:2932-2935 (2009). Pyrrolobenzodiazepines include SG2057 and those described in Denny, Exp. Opin. Ther. Patents., 10(4):459-474 (2000), Anti-Cancer Agents in Medicinal Chemistry, 2009, 9, 1-31; WO 2011/130613 A1; EP 2 789 622 A1; Blood 2013, 122, 1455; J. Antimicrob. Chemother. 2012, 67, 1683-1696; Cancer Res. 2004, 64, 6693-6699; WO 2013041606; U.S. Pat. No. 8,481,042; WO 2013177481; WO 2011130613; WO2011130598

Exemplary microtubule stabilizing and destabilizing agents include taxane compounds, such as paclitaxel, docetaxel, cabazitaxel; maytansinoids, auristatins and analogs thereof, tubulysin A and B derivatives, vinca alkaloid derivatives, epothilones, PM060184 and cryptophycins.

Exemplary maytansinoids or maytansinoid analogs include maytansinol and maytansinol analogs, maytansine or DM-1 and DM-4 are those described in U.S. Pat. Nos. 5,208,020; 5,416,064; 6,333.410; 6,441,163; 6,716,821; RE39,151 and 7,276,497. In certain embodiments, the cytotoxic agent is a maytansinoid, another group of anti-tubulin agents (ImmunoGen, Inc.; see also Chari et al., 1992, Cancer Res. 52: 127-131), maytansinoids or maytansinoid analogs. Examples of suitable maytansinoids include maytansinol and maytansinol analogs. Suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Exemplary auristatins include auristatin E (also known as a derivative of dolastatin-10), auristatin EB (AEB), auristatin EFP (AEFP), monomethyl auristatin E (MMAE), monomethyl auristatin F (MMAF), auristatin F and dolastatin. Suitable auristatins are also described in U.S. Publication Nos. 2003/0083263, 2011/0020343, and 2011/0070248; PCT Application Publication Nos. WO 09/117531, WO 2005/081711, WO 04/010957; WO02/088172 and WO01/24763, and U.S. Pat. Nos. 7,498,298; 6,884,869; 6,323,315; 6,239,104; 6,124,431; 6,034,065; 5,780,588; 5,767,237; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, the disclosures of which are incorporated herein by reference in their entirety.

Exemplary tubulysin compounds include compounds described in U.S. Pat. Nos. 7,816,377; 7,776,814; 7,754,885; U.S. Publication Nos. 2011/0021568; 2010/004784; 2010/0048490; 2010/00240701; 2008/0176958; and PCT Application Nos. WO 98/13375; WO 2004/005269; WO 2008/138561; WO 2009/002993; WO 2009/055562; WO 2009/012958; WO 2009/026177; WO 2009/134279; WO 2010/033733; WO 2010/034724; WO 2011/017249; WO 2011/057805; the disclosures of which are incorporated by reference herein in their entirety.

Exemplary vinca alkaloids include vincristine, vinblastine, vindesine, and navelbine (vinorelbine). Suitable Vinca alkaloids that can be used in the present invention are also disclosed in U.S. Publication Nos. 2002/0103136 and 2010/0305149, and in U.S. Pat. No. 7,303,749 B1, the disclosures of which are incorporated herein by reference in their entirety.

Exemplary epothilone compounds include epothilone A, B, C, D, E and F, and derivatives thereof. Suitable epothilone compounds and derivatives thereof are described, for example, in U.S. Pat. Nos. 6,956,036; 6,989,450; 6,121,029; 6,117,659; 6,096,757; 6,043,372; 5,969,145; and 5,886,026; and WO 97/19086; WO 98/08849; WO 98/22461; WO 98/25929; WO 98/38192; WO 99/01124; WO 99/02514; WO 99/03848; WO 99/07692; WO 99/27890; and WO 99/28324; the disclosures of which are incorporated herein by reference in their entirety.

Exemplary cryptophycin compounds are described in U.S. Pat. Nos. 6,680,311 and 6,747,021.

Exemplary platinum compounds include cisplatin (PLATINOL®), carboplatin (PARAPLATIN®), oxaliplatin (ELOX ATINE®), iproplatin, ormaplatin, and tetraplatin.

Exemplary topoisomerase I inhibitors include camptothecin, camptothecin, derivatives, camptothecin analogs and non-natural camptothecins, such as, for example, CPT-11 (irinotecan), SN-38, topotecan, 9-aminocamptothecin, rubitecan, gimatecan, karenitecin, silatecan, lurtotecan, exatecan, diflomotecan, belotecan, lurtotecan and S39625. Other camptothecin compounds that can be used in the present invention include those described in, for example, J. Med. Chem., 29:2358-2363 (1986); J. Med. Chem., 23:554 (1980); J. Med. Chem., 30: 1774 (1987).

Additional agents acting on DNA include Lurbinectedin (PM01183), Trabectedin (also known as ecteinascidin 743 or ET-743) and analogs as described in WO 200107711, WO 2003014127.

Angiogenesis inhibitors include, but are not limited, MetAP2 inhibitors.

Exemplary MetAP2 inhibitors include fumagillol analogs, meaning any compound that includes the fumagillin core structure, including fumagillamine, that inhibits the ability of MetAP-2 to remove NH₂-terminal methionines from proteins as described in Rodeschini et al., /. Org. Chem., 69, 357-373, 2004 and Liu, et al., Science 282, 1324-1327, 1998. Non limiting examples of “fumagillol analogs” are disclosed in /. Org. Chem., 69, 357, 2004; J. Org. Chem., 70, 6870, 2005; European Patent Application 0 354 787; /. Med. Chem., 49, 5645, 2006; Bioorg. Med. Chem., 11, 5051, 2003; Bioorg. Med. Chem., 14, 91, 2004; Tet. Lett. 40, 4797, 1999; WO99/61432; U.S. Pat. Nos. 6,603,812; 5,789,405; 5,767,293; 6,566,541; and 6,207,704.

Exemplary cell cycle progression inhibitors include CDK inhibitors such as, for example, BMS-387032 and PD0332991; Rho-kinase inhibitors such as, for example GSK429286; checkpoint kinase inhibitors such as, for example, AZD7762; aurora kinase inhibitors such as, for example, AZD1152, MLN8054 and MLN8237; PLK inhibitors such as, for example, BI 2536, BI6727 (Volasertib), GSK461364, ON-01910 (Estybon); and KSP inhibitors such as, for example, SB 743921, SB 715992 (ispinesib), MK-0731, AZD8477, AZ3146 and ARRY-520.

Exemplary PI3K/m-TOR/AKT signaling pathway inhibitors include phosphoinositide 3-kinase (PI3K) inhibitors, GSK-3 inhibitors, ATM inhibitors, DNA-PK inhibitors and PDK-1 inhibitors.

Exemplary PI3 kinases are disclosed in U.S. Pat. No. 6,608,053, and include BEZ235, BGT226, BKM120, CAL101, CAL263, demethoxyviridin, GDC-0941, GSK615, IC87114, LY294002, Palomid 529, perifosine, PF-04691502, PX-866, SAR245408, SAR245409, SF1126, Wortmannin, XL147 and XL765.

Exemplary AKT inhibitors include, but are not limited to AT7867.

Exemplary MAPK signaling pathway inhibitors include MEK, Ras, JNK, B-Raf and p38 MAPK inhibitors.

Exemplary MEK inhibitors are disclosed in U.S. Pat. No. 7,517,994 and include GDC-0973, GSK1120212, MSC1936369B, AS703026, R05126766 and R04987655, PD0325901, AZD6244, AZD 8330 and GDC-0973.

Exemplary B-raf inhibitors include CDC-0879, PLX-4032, and SB590885.

Exemplary B p38 MAPK inhibitors include BIRB 796, LY2228820 and SB202190

Receptor tyrosine kinases (RTK) are cell surface receptors which are often associated with signaling pathways stimulating uncontrolled proliferation of cancer cells and neoangiogenesis. Many RTKs, which over express or have mutations leading to constitutive activation of the receptor, have been identified, including, but not limited to, VEGFR, EGFR, FGFR, PDGFR, EphR and RET receptor family receptors. Exemplary RTK specific targets include ErbB2, FLT-3, c-Kit, c-Met, HIF.

Exemplary inhibitors of ErbB2 receptor (EGFR family) include but not limited to AEE788 (NVP-AEE 788), BIBW2992, (Afatinib), Lapatinib, Erlotinib (Tarceva), and Gefitinib (Iressa).

Exemplary RTK inhibitors targeting more then one signaling pathway (multitargeted kinase inhibitors) include AP24534 (Ponatinib) that targets FGFR, FLT-3, VEGFR-PDGFR and Bcr-Abl receptors; ABT-869 (Linifanib) that targets FLT-3 and VEGFR-PDGFR receptors; AZD2171 that targets VEGFR-PDGFR, Flt-1 and VEGF receptors; CHR-258 (Dovitinib) that targets VEGFR-PDGFR, FGFR, Flt-3, and c-Kit receptors.

Exemplary protein chaperon inhibitors include HSP90 inhibitors. Exemplary HSP90 inhibitors include 17AAG derivatives, BIIB021, BIIB028, SNX-5422, NVP-AUY-922 and KW-2478.

Exemplary HDAC inhibitors include Belinostat (PXD101), CUDC-101, Doxinostat, ITF2357 (Givinostat, Gavinostat), JNJ-26481585, LAQ824 (NVP-LAQ824, Dacinostat), LBH-589 (Panobinostat), MC1568, MGCD0103 (Mocetinostat), MS-275 (Entinostat), PCI-24781, Pyroxamide (NSC 696085), SB939, Trichostatin A and Vorinostat (SAHA).

Exemplary PARP inhibitors include iniparib (BSI 201), olaparib (AZD-2281), ABT-888 (Veliparib), AG014699, CEP 9722, MK 4827, KU-0059436 (AZD2281), LT-673, 3-aminobenzamide, A-966492, and AZD2461

Exemplary Wnt/Hedgehog signaling pathway inhibitors include vismodegib (RG3616/GDC-0449), cyclopamine (11-deoxojervine) (Hedgehog pathway inhibitors) and XAV-939 (Wnt pathway inhibitor)

Exemplary RNA polymerase inhibitors include amatoxins. Exemplary amatoxins include a-amanitins, β-amanitins, γ-amanitins, ε-amanitins, amanullin, amanullic acid, amaninamide, amanin, and proamanullin.

Exemplary proteasome inhibitors include bortezomib, carfilzomib, ONX 0912, CEP-18770, and MLN9708.

In one embodiment the drug of the invention is a non-natural camptothecin compound, vinca alkaloid, kinase inhibitor (e.g., PI3 kinase inhibitor (GDC-0941 and PI-103)), MEK inhibitor, KSP inhibitor, RNA polymerse inhibitor, PARP inhibitor, docetaxel, paclitaxel, doxorubicin, duocarmycin, tubulysin, auristatin or a platinum compound. In specific embodiments, the drug is a derivative of SN-38, vindesine, vinblastine, PI-103, AZD 8330, auristatin E, auristatin F, a duocarmycin compound, tubulysin compound, or ARRY-520.

In another embodiment, the drug used in the invention is a combination of two or more drugs, such as, for example, PI3 kinases and MEK inhibitors; broad spectrum cytotoxic compounds and platinum compounds; PARP inhibitors and platinum compounds; broad spectrum cytotoxic compounds and PARP inhibitors.

The active agent can be a cancer therapeutic. The cancer therapeutics may include death receptor agonists such as the TNF-related apoptosis-inducing ligand (TRAIL) or Fas ligand or any ligand or antibody that binds or activates a death receptor or otherwise induces apoptosis. Suitable death receptors include, but are not limited to, TNFR1, Fas, DR3, DR4, DR5, DR6, LTβR and combinations thereof.

In some embodiments, the active agent can be 20-epi-1,25 dihydroxyvitamin D3, 4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, auristatin, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cabazitaxel, cactinomycin, calcipotriol, calphostin C, calusterone, camptothecin, camptothecin derivatives, canarypox IL-2, capecitabine, caracemide, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castano spermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, larotaxel, lanreotide acetate, lapatinib, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, maytansinoid, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum(IV) complexes, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxy ethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, siRNA, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, taxane, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

In certain embodiments, the active agent of the conjugate comprises a predetermined molar weight percentage from about 1% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the molar weight percentages of the components of the conjugate is 100%. The amount of active agent(s) of the conjugate may also be expressed in terms of proportion to the targeting ligand(s). For example, the present teachings provide a ratio of active agent to ligand of about 10:1, 9:1, 8:1, 7:1, 6:1, 5:1, 4:1, 3:1, 2:1, 1:1, 1:2, 1:3, 1:4; 1:5, 1:6, 1:7, 1:8, 1:9, or 1:10.

B. Targeting Moieties

The conjugates contain one or more targeting moieties and/or targeting ligands. Targeting ligands or moieties can be peptides, antibody mimetics, nucleic acids (e.g., aptamers), polypeptides (e.g., antibodies), glycoproteins, small molecules, carbohydrates, or lipids. The targeting moiety, X, can be a peptide such as somatostatin, octreotide, LHRH, an EGFR-binding peptide, RGD-containing peptides, a protein scaffold such as a fibronectin domain, an aptide or bipodal peptide, a single domain antibody, a stable scFv, or a bispecific T-cell engagers, nucleic acid (e.g., aptamer), polypeptide (e.g., antibody or its fragment), glycoprotein, small molecule, carbohydrate, or lipid. The targeting moiety, X can be an aptamer being either RNA or DNA or an artificial nucleic acid; small molecules; carbohydrates such as mannose, galactose and arabinose; vitamins such as ascorbic acid, niacin, pantothenic acid, carnitine, inositol, pyridoxal, lipoic acid, folic acid (folate), riboflavin, biotin, vitamin B12, vitamin A, E, and K; a protein or peptide that binds to a cell-surface receptor such as a receptor for thrombospondin, tumor necrosis factors (TNF), annexin V, interferons, cytokines, transferrin, GM-CSF (granulocyte-macrophage colony-stimulating factor), or growth factors such as vascular endothelial growth factor (VEGF), hepatocyte growth factor (HGF), (platelet-derived growth factor (PDGF), basic fibroblast growth factor (bFGF), and epidermal growth factor (EGF).

The targeting moieties may target cell surface receptors and enable intracellular delivery. In addition, the targeting moiety may target an intracellular receptor.

In some embodiments, the targeting moiety is a stabilized peptide. Intramolecular crosslinkers are used to maintain the peptide in the desired configuration, for example using disulfide bonds, amide bonds, or carbon-carbon bonds to link amino acid side chains. Such peptides which are conformationally stabilized by means of intramolecular cross-linkers are sometimes referred to as “stapled” peptides. The cross-linkers connect at least two amino acids of the peptide. The cross-linkers may comprise at least 5, 6, 7, 8, 9, 10. 11, or 12 consecutive carbon-carbon bonds. The cross-linkers may comprise at least 5, 6, 7, 8, 9, 10, 11, or 12 carbon atoms. Stapled peptides may penetrate cell membranes and bind to an intracellular receptor.

In one non-limiting example, the stapled peptide is a cross-linked alpha-helical polypeptide comprising a crosslinker wherein a hydrogen atom attached to an α-carbon atom of an amino acid of the peptide is replaced with a substituent of formula R—, wherein R— is alkyl, alkenyl, alkynyl, arylalkyl, cycloalkylalkyl, heteroalkyl, or heterocycloalkyl, unsubstituted or substituted with halo-, as disclosed in US 20140323701 to Nash et al., the contents of which are incorporated herein by reference in their entirety. In another example, the stapled peptides have improved in vivo half life such as any stapled peptide disclosed in US 20100298201 to Nash et al., the contents of which are incorporated herein by reference in their entirety. In another example, the targeting moiety may be any stapled peptide disclosed in U.S. Pat. No. 9,175,045 to Nash et al., the contents of which are incorporated herein by reference in their entirety, wherein the stapled peptide possesses reduced affinity to serum proteins while still remaining sufficient affinity to cell membranes. In another example, the cross-linker of the stapled peptide links the a-positions of at least two amino acids, such as any stapled peptide disclosed in U.S. Pat. No. 9,175,047 to Nash et al., the contents of which are incorporated herein by reference in their entirety. In another example, the targeting moiety comprise any stapled peptide disclosed in U.S. Pat. No. 8,927,500 to Guerlavais et al., the contents of which are incorporated herein by reference in their entirety, wherein the stapled peptide has homology to p53 protein and can bind to the MDM2 and/or MDMX proteins. In another example, the stapled peptide generates a reduced antibody response. Any stapled peptide disclosed in U.S. Pat. No. 8,808,694 to Nash et al., the contents of which are incorporated herein by reference in their entirety, may be used as a targeting moiety. In another example, the staped peptide may be any polypeptide with optimized protease stability disclosed in US 20110223149 to Nash et al., the contents of which are incorporated herein by reference in their entirety.

In some embodiments, the targeting moiety is a protein scaffold. The protein scaffold may be an antibody-derived protein scaffold. Non-limiting examples include single domain antibody (dAbs), nanobody, single-chain variable fragment (scFv), antigen-binding fragment (Fab), Avibody, minibody, CH2D domain, Fcab, and bispecific T-cell engager (BiTE) molecules. In some embodiments, scFv is a stable scFv, wherein the scFv has hyperstable properties. In some embodiments, the nanobody may be derived from the single variable domain (VHH) of camelidae antibody.

In some embodiments, the protein scaffold may be a nonantibody-derived protein scaffold, wherein the protein scaffold is based on nonantibody binding proteins. The protein scaffold may be based on enginnered Kunitz domains of human serine protease inhibitors (e.g., LAC1-D1), DARPins (designed ankyrin repeat domains), avimers created from multimerized low-density lipoprotein receptor class A (LDLR-A), anticalins derived from lipocalins, knottins constructed from cysteine-rich knottin peptides, affibodies that are based on the Z-domain of staphylococcal protein A, adnectins or monobodies and pronectins based on the 10^{th} or 14^{th} extracellular domain of human fibronectin III, Fynomers derived from SH3 domains of human Fyn tyrosine kinase, or nanofitins (formerly Affitins) derived from the DNA bindig protein Sac7d.

In some embodiments, the protein scaffold may be any protein scaffold disclosed in Mintz and Crea, BioProcess, vol. 11(2):40-48 (2013), the contents of which are incorporated herein by reference in their entirety. Any of the protein scaffolds disclosed in Tables 2-4 of Mintz and Crea may be used as a targeting moiety of the conjugate of the invention.

In some embodiments, the protein scaffold may be based on a fibronectin domain. In some embodiments, the proten scaffold may be based on fibronectin type III (FN3) repeat protein. In some embodiments, the protein scaffold may be based on a consensus sequence of multiple FN3 domains from human Tenascin-C (hereinafter “Tenascin”). Any protein scaffold based on a fibronectin domain disclosed in U.S. Pat. No. 8,569,227 to Jacobs et al., the contents of which are incorporated herein by reference in their entirety, may be used as a targeting moiety of the conjugate of the invention.

In some embodiments, the targeting moiety or targeting ligand may be any moledule that can bind to luteinizing-hormone-releasing hormone receptor (LHRHR). Such targeting ligands can be peptides, antibody mimetics, nucleic acids (e.g., aptamers), polypeptides (e.g., antibodies), glycoproteins, small molecules, carbohydrates, or lipids. In some embodiments, the targeting moiety is LHRH or a LHRH analog.

Luteinizing-hormone-releasing hormone (LHRH), also known as gonadotropin-releasing hormone (GnRH) controls the pituitary release of gonadotropins (LH and FSH) that stimulate the synthesis of sex steroids in the gonads. LHRH is a 10-amino acid peptide that belongs to the gonadotropin-releasing hormone class. Signaling by LHRH is involved in the first step of the hypothalamic-pituitary-gonadal axis. An approach in the treatment of hormone-sensitive tumors directed to the use of agonists and antagonists of LHRH (A. V. Schally and A. M. Comaru-Schally. Sem. Endocrinol., 5 389-398, 1987) has been reported. Some LHRH agonists, when substituted in position 6, 10, or both are much more active than LHRH and also possess prolonged activity. Some LHRH agonists are approved for clinical use, e.g., Leuprolide, triptorelin, nafarelin and goserelin.

Some human tumors are hormone dependent or hormone-responsive and contain hormone receptors. Certain of these tumors are dependent on or responsive to sex hormones or growth factors, or have components that are dependent or responsive to such hormones. Mammary carcinomas contain estrogen, progesterone, glucocorticoid, LHRH, EGF IGF-I and somatostatin receptors. Peptide hormone receptors have been detected in acute leukaemia, prostate-, breast-, pancreatic, ovarian-, endometri cancer, colon cancer and brain tumors (M. N. Pollak, et al., Cancer Lett. 38 223-230 1987; F. Pekonen, et al., Cancer Res., 48 1343-1347, 1988; M. Fekete, et al., J Clin. Lab. Anal. 3 137-147, 1989; G. Emons, et al., Eur. J. Cancer Oncol., 25215-221 1989). It has been found (M. Fekete, et al., Endocrinology. 124 946-955. 1989; M Fekete, et al. Pancreas 4521-528, 1989) that both agonistic and antagonistic analog of LHRH bind to human breast cancer cell membranes, and also to the cell membranes of pancreatic cancer. It has been demonstrated that biologically active peptides such a melanotropin (MSH), epidermal growth factor, insulin and agonistic and antagonisti analogs of LHRH (L Jennes, et. al., Peptides 5 215-220, 1984) are internalized b their target cells by endocytosis.

The conjugates of the invention can employ any of the large number of known molecules that recognize the LHRH receptor, such as known LHRH receptor agonists and antagonists. In some embodiments, the LHRH analog portion of the conjugate contains between 8 and 18 amino acids.

Examples of LHRH binding molecules useful in the present invention are described herein. Further non-limiting examples are analogs of pyroGlu-His-Trp-Ser-Tyr-Gly-Leu-Arg-Pro-Gly-NH₂, leuprolide, triptorelin, nafarelin, buserelin, goserelin, cetrorelix, ganirelix, azaline-B, degarelix and abarelix.

Methods for synthesizing LHRH peptides and analogs are well documented and are within the ability of a person of ordinary skill in the art as exemplified in the references listed supra. Further synthetic procedures are provided in the following examples. The following examples also illustrate methods for synthesizing the targeted cytotoxic compounds of the present invention. Specific targeting of therapeutic or cytotoxic agents allows selective destruction of a tumor expressing a receptor specific for a biologically active peptide. For example, a tumor expressing a LHRH receptor includes a neoplasm of the lung, breast, prostate, colon, brain, gastrointestinal tract, neuroendocrine axis, liver, or kidney (see Schaer et al., Int. J. Cancer, 70:530-537, 1997; Chave et al., Br. J. Cancer 82(1):124-130, 2000; Evans et al., Br. J. Cancer 75(6):798-803, 1997).

In some embodiments, the targeting moiety, e.g., LHRH analog, used in the invention is hydrophilic, and is therefore water soluble. In some embodiments, such conjugates and particles containing such conjugates are used in treatment paradigms in which this feature is useful, e.g., compared to conjugates comprising hydrophobic analogs. Hydrophilic analogs described herein can be soluble in blood, cerebrospinal fluid, and other bodily fluids, as well as in urine, which may facilitate excretion by the kidneys. This feature can be useful, e.g., in the case of a composition that would otherwise exhibit undesirable liver toxicity. The invention also discloses specific hydrophilic elements (e.g., incorporation of a PEG linker, and other examples in the art) for incorporation into peptide analogs, allowing modulation of the analog's hydrophilicity to adjust for the chemical and structural nature of the various conjugated cytotoxic agents.

In some embodiments, the targeting moiety is an antibody mimetic such as a monobody, e.g., an ADNECTIN™ (Bristol-Myers Squibb, New York, N.Y.), an Affibody® (Affibody AB, Stockholm, Sweden), Affilin, nanofitin (affitin, such as those described in WO 2012/085861, an Anticalin™, an avimers (avidity multimers), a DARPin™, a Fynomer™, Centyrin™, and a Kunitz domain peptide. In certain cases, such mimetics are artificial peptides or proteins with a molar mass of about 3 to 20 kDa. Nucleic acids and small molecules may be antibody mimetic.

In another example, a targeting moiety can be an aptamer, which is generally an oligonucleotide (e.g., DNA, RNA, or an analog or derivative thereof) that binds to a particular target, such as a polypeptide. In some embodiments, the targeting moiety is a polypeptide (e.g., an antibody that can specifically bind a tumor marker). In certain embodiments, the targeting moiety is an antibody or a fragment thereof. In certain embodiments, the targeting moiety is an Fc fragment of an antibody.

In another example, a targeting moiety may be a non-immunoreactive ligand. For example, the non-immunoreactive ligand may be insulin, insulin-like growth factors I and II, lectins, apoprotein from low density lipoprotein, etc. as disclosed in US 20140031535 to Jeffrey, the contents of which are incorporated herein by reference in their entirety. Any protein or peptide comprising a lectin disclosed in WO2013181454 to Radin, the contents of which are incorporated herein by reference in their entirety, may be used as a targeting moiety.

In another example, the conjugate of the invention may target a hepatocyte intracellularly and a hepatic ligand may be used as a targeting moiety. Any hepatic ligand disclosed in US 20030119724 to Ts'o et al., the contents of which are incorporated herein by reference in their entirety, such as the ligands in FIG. 1, may be used. The hepatic ligand specifically binds to a hepatic receptor, thereby directing the conjugate into cells having the hepatic receptor.

In another example, a targeting moiety may interact with a protein that is overexpressed in tumor cells compared to normal cells. The targeting moiety may bind to a chaperonin protein, such as Hsp90, as disclosed in US 20140079636 to Chimmanamada et al., the contents of which are incorporated herein by reference in their entirety. The targeting moiety may be an Hsp90 inhibitor, such as geldanamycins, macbecins, tripterins, tanespimycins, and radicicols.

In another example, the conjugate may have a terminal half-life of longer than about 72 hours and a targeting moiety may be selected from Table 1 or 2 of US 20130165389 to Schellenberger et al., the contents of which are incorporated herein by reference in their entirety. The targeting moiety may be an antibody targeting delta-like protein 3 (DLL3) in disease tissues such as lung cancer, pancreatic cancer, skin cancer, etc., as disclosed in WO2014125273 to Hudson, the contents of which are incorporated herein by reference in their entirety. The targeting moiety may also any targeting moiety in WO2007137170 to Smith, the contents of which are incorporated herein by reference in their entirety. The targeting moiety binds to glypican-3 (GPC-3) and directs the conjugate to cells expressing GPC-3, such as hepatocellular carcinoma cells.

In some embodiments, a target of the targeting moiety may be a marker that is exclusively or primarily associated with a target cell, or one or more tissue types, with one or more cell types, with one or more diseases, and/or with one or more developmental stages. In some embodiments, a target can comprise a protein (e.g., a cell surface receptor, transmembrane protein, glycoprotein, etc.), a carbohydrate (e.g., a glycan moiety, glycocalyx, etc.), a lipid (e.g., steroid, phospholipid, etc.), and/or a nucleic acid (e.g., a DNA, RNA, etc.).

In another embodiment, targeting moieties may be peptides for regulating cellular activity. For example, the targeting moiety may bind to Toll Like Receptor (TLR). It may be a peptide derived from vaccinia virus A52R protein such as a peptide comprising SEQ ID No. 13 as disclosed in U.S. Pat. No. 7,557,086, a peptide comprising SEQ ID No. 7 as disclosed in U.S. Pat. No. 8,071,553 to Hefeneider, et al., or any TLR binding peptide disclosed in WO 2010141845 to McCoy, et al, the contents of each of which are incorporated herein by reference in their entirety. The A52R derived synthetic peptide may significantly inhibit cytokine production in response to both bacterial and viral pathogen associated molecular patterns, and may have application in the treatment of inflammatory conditions that result from ongoing toll-like receptor activation,

In another embodiment, targeting moieties many be amino acid sequences or single domain antibody fragments for the treatment of cancers and/or tumors. For example, targeting moieties may be an amino acid sequence that binds to Epidermal Growth Factor Receptor 2 (HER2). Targeting moieties may be any HER2-binding amino acid sequence described in US 20110059090, U.S. Pat. Nos. 8,217,140, and 8,975,382 to Revets, et al., the contents of each of which are incorporated herein by reference in their entirety. The targeting moiety may be a domain antibody, a single domain antibody, a VHH, a humanized VHH or a camelized VH.

In another embodiment, targeting moieties may be peptidomimetic macrocycles for the treatment of disease. For example, targeting moieties may be peptidomimetic macrocycles that bind to the growth hormone-realising hormone (GHRH) receptor, such as a peptidomimetic macrocycle comprising an amino acid sequence which is at least about 60% identical to GHRE 1-29 and at least two macrocycle-forming linkers as described in US20130123169 to Kawahata et al., the contents of which are incorporated herein by reference in their entirety. In another embodiment, the peptidomimetic macrocycle targeting moiety may be prepared by introducing a cross-linker between two amino acid residues of a polypeptide as described in US 20120149648 and US 20130072439 to Nash et al., the contents of each of which are incorporated herein by reference in their entirety. Nash et al. teaches that the peptidomimetic macrocycle may comprise a peptide sequence that is derived from the BCL-2 family of proteins such as a BH3 domain. The peptidomimetic macrocycle may comprise a BID, BAD, BIM, BIK, NOXA, PUMA peptides.

In another embodiment, targeting moieties may be polypeptide analogues for transport to cells. For example, the polypeptide may be an Angiopep-2 polypeptide analog. It may comprising a polypeptide comprising an amino acid sequence at least 80% identical to SEQ ID No.97 as described in US 20120122798 to Castaigne et al., the contents of which are incorporated herein by reference in their entirety. Additionally, polypeptides may transport to cells, such as liver, lung, kidney, spleen, and muscle, such as Angiopep-4b, Angiopep-5, Angiopep-6, and Angiopep-7 polypeptide as described in EP 2789628 to Beliveau et al., the contents of each of which are incorporated herein by reference in their entirety.

In another embodiment, targeting moieties may be homing peptides to target liver cells in vivo. For example, the melittin delivery peptides that are administered with RNAi polynucleotides as described in U.S. Pat. No. 8,501,930 Rozema, et al., the contents of which are incorporated herein by reference in their entirety, may be used as targeting moieties. In addition, delivery polymers provide membrane penetration function for movement of the RNAi polynucleotides from the outside the cell to inside the cell as described in U.S. Pat. No. 8,313,772 to Rozema et al., the contents of each of which are incorporated herein by reference in their entirety. Any delivery peptide disclosed by Rozema et al. may be used as targeting moeities.

In another embodiment, targeting moieties may be structured polypeptides to target and bind proteins. For example, polypeptides with sarcosine polymer linkers that increase the solubility of structured polypeptides, as described in WO 2013050617 to Tite, et al., the contents of which are incorporated herein by reference in their entirety, may be used as targeting moieties. Additionally, polypeptide with variable binding activity produced by the methods described in WO 2014140342 to Stace, et al., the contents of which are incorporated herein by reference in their entirety. The polypeptides may be evaluated for the desired binding activity.

In another embodiment, modifications of the targeting moieties affect a compound's ability to distribute into tissues. For example, a structure activity relationship analysis was completed on a low orally bioavailable cyclic peptide and the permeability and clearance was determined as described in Rand, A C., et al., Medchemcomm. 2012, 3(10): 1282-1289, the contents of which are incorporated herein by reference in their entirety. Any of the cyclic peptide disclosed by Rand et al., such as N-methylated cyclic hexapeptides, may be used as targeting moieties.

In another embodiment, targeting moieties may be a polypeptide which is capable of internalization into a cell. For example, targeting moieties may be an Alphabody capable of internalization into a cell and specifically binding to an intracellular target molecule as described in US 20140363434 to Lasters, et al., the contents of which are incorporated herein by reference in their entirety. As taught by Lasters et al., an ‘Alphabody’ or an ‘Alphabody structure’ is a self-folded, single-chain, triple-stranded, predominantly alpha-helical, coiled coil amino acid sequence, polypeptide or protein. The Alphabody may be a parallel Alphabody or an antiparallel Alphabody. Moreover, targeting moieties may be any Alphabody in the single-chain Alphabody library used for the screening for and/or selection of one or more Alphabodies that specifically bind to a target molecule of interest as described in WO 2012092970 to Desmet et al., the contents of which are incorporated herein by reference in their entirety.

In another embodiment, targeting moieties may consist of an affinity-matured heavy chain-only antibody. For example, targeting moieties may be any V_{H} heavy chain-only antibodies produced in a transgenic non-human mammal as described in US 20090307787 to Grosveld et al., the contents of which are incorporated herein by reference in their entirety.

In another embodiment, targeting moieties may bind to the hepatocyte growth factor receptor “HGFr” or “cMet”. For example, targeting moieties may be a polypeptide moiety that is conjugated to a detectable label for diagnostic detection of cMet as described in U.S. Pat. No. 9,000,124 to Dransfield et al., the contents of which are incorporated herein by reference in their entirety. Additionally, targeting moieties may bind to human plasma kallikrein and may comprise BPTI-homologous Kunitz domains, especially LACI homologues, to bind to one or more plasma (and/or tissue) kallikreins as described in WO 1995021601 to Markland et al., the contents of which are incorporated herein by reference in their entirety.

In another embodiment, targeting moieties are evolved from weak binders and anchor-scaffold conjugates having improved target binding and other desired pharmaceutical properties through control of both synthetic input and selection criteria. Any target binding element identified in US 20090163371 to Stern et al., the contents of which are incorporated herein by reference in their entirety, may be used as a targeting moiety. Moreover, targeting moieties may be macrocyclic compounds that bind to inhibitors of apoptosis as described in WO 2014074665 to Borzilleri et al., the contents of which are incorporated herein by reference in their entirety.

In another embodiment, targeting moieties may comprise pre-peptides that encode a chimeric or mutant lantibiotic. For example, targeting moieties may be pre-tide that encode a chimera that was accurately and efficiently converted to the mature lantibiotic, as demonstrated by a variety of physical and biological activity assays as described in U.S. Pat. No. 5,861,275 to Hansen, the contents of which are incorporated herein by reference in their entirety. The mixture did contain an active minor component with a biological activity.

In another embodiment, targeting moieties may comprise a leader peptide of a recombinant manganese superoxide dismutase (rMnSOD-Lp). For example, rMnSOD-Lp which delivers cisplatin directly into tumor cells as described in Borrelli, A., et al., Chem Biol Drug Des. 2012, 80(1):9-16, the contents of which are incorporated herein by reference in their entirety, may be used a targeting moiety.

In another embodiment, the targeting moiety may be an antibody for the treatment of glioma. For example, an antibody or antigen binding fragment which specifically binds to JAMM-B or JAM-C as described in U.S. Pat. No. 8,007,797 to Dietrich et al., the contents of which are incorporated herein by reference in their entirety, may be used as a targeting moiety, JAMs are a family of proteins belonging to a class of adhesion molecules generally localized at sites of cell-cell contacts in tight junctions, the specialized cellular structures that keep cell polarity and serve as barriers to prevent the diffusion of molecules across intercellular spaces and along the basolateral-apical regions of the plasma membrane.

In another embodiment, the targeting moiety may be a target interacting modulator. For example, nucleic acid molecules capable of interacting with proteins associated with the Human Hepatitis C virus or corresponding peptides or mimetics capable of interfering with the interaction of the native protein with the HIV accessory protein as described in WO 2011015379 and U.S. Pat. No. 8,685,652, the contents of each of which are incorporated herein by reference in their entirety, may be used as a targeting moiety.

In another embodiment, the targeting moiety may bind with biomolecules. For example, any cystine-knot family small molecule polycyclic molecular scaffolds were designed as peptidomimetics of FSH and used as peptide-vaccine as described in U.S. Pat. No. 7,863,239 to Timmerman, the contents the contents of which are incorporated herein by reference in their entirety, may be used as targeting moieties.

In another embodiment, the targeting moiety may bind to integrin and thereby block or inhibit integrin binding. For example, any highly selective disulfide-rich dimer molecules which inhibit binding of α4β7 to the mucosal addressin cell adhesion molecule (MAdCAM) as described in WO 2014059213 to Bhandari, the contents of which are incorporated herein by reference in their entirety, may be used as a targeting moiety. Any inhibitor of specific integrins-ligand interactions may be used as a targeting moiety. The conjugates comprising such target moieties may be effective as anti-inflammatory agents for the treatment of various autoimmune diseases.

In another embodiment, the targeting moiety may comprise novel peptides. For example, any cyclic peptide or mimetic that is a serine protease inhibitor as described in WO 2013172954 to Wang et al., the contents of which are incorporated herein by reference in their entirety, may be used as a targeting moiety. Additionally, targeting moieties may comprise a targeting peptide that is used in the reduction of cell proliferation and the treatment of cancer. For example, a peptide composition inhibiting the trpv6 calcium channel as described in US 20120316119 to Stewart, the contents of which are incorporated herein by reference in their entirety, may be used as a targeting moiety.

In another embodiment, the targeting moiety may comprise a cyclic peptide. For example, any cyclic peptides exhibit various types of action in vivo, as described in US20100168380 and WO 2008117833 to Suga et al., and WO 2012074129 to Higuchi et al., the contents of each of which are incorporated herein by reference, may be used as targeting moieties. Such cyclic peptide targeting moieties have a stabilized secondary structure and may inhibit biological molecule interactions, increase cell membrane permeability and the peptide's half-life in blood serum.

In another embodiment, the targeting moiety may consist of a therapeutic peptide. For example, peptide targeting moieties may be an AP-1 signaling inhibitor, such as a peptide analog comprising SEQ ID No. 104 of U.S. Pat. No. 8,946,381B2 to Fear that is used for the treatment of wounds, a peptide comprising SEQ ID No. 108 in U.S. Pat. No. 8,822,409B2 to Milech, et al. that is used to treat acute respiratory distress syndrome (ARDS), or a neuroprotective AP-1 signaling inhibitory peptide that is a fusion peptide comprising a protein transduction domain having the amino acid sequence of SEQ ID NO: 1 and a peptide having the sequence of SEQ ID NO:54 as described in U.S. Pat. No. 8,063,012 to Watt, the contents of each of which are incorporated herein by reference in their entirety. In another example, the targeting moiety may be any biological modulator isolated from biodiverse gene fragment libraries as described in U.S. Pat. No. 7,803,765 and EP1754052 to Watt, any inhibitor of c-Jun dimerization as described in EP1601766 and EP1793841 to Watt, any peptide inhibitors of CD40L signaling as described in U.S. Pat. No. 8,802,634 and US20130266605 to Watt, or any peptide modulators of cellular phenotype as described in US20110218118 to Watt, the contents of each of which are incorporated herein by reference in their entirety.

In another embodiment, the targeting moiety may consist of a characterized peptide. For example, any member of the screening libraries created from bioinformatic source data to theoretically predict the secondary structure of a peptide as described in EP1987178 to Watt et al., any peptide identified from peptide libraries that are screened for antagonism or inhibition of other biological interactions by a reverse hybrid screening method as described by EP1268842 to Hopkins, et al., the contents of each of which are incorporated herein by reference in their entirety, may be used as a targeting moiety. Additionally, targeting moieties may be cell-penetrating peptides. For example, any cell-penetrating peptides linked to a cargo that are capable of passing through the blood brain barrier as described by US20140141452A1 to Watt, et al., the contents of which are incorporated herein by reference, may be used a targeting moiety.

In another embodiment, the targeting moiety may comprise a LHRH antagonist, agonist, or analog. For example, the targeting moiety may be Cetrorelix, a decapeptide with a terminal acid amide group (AC-D-Nal(2)-D-pC1-Phe-D-Pal(3)-Ser-Tyr-D-Cit-Leu-Arg-Pro-D-Ala-NH₂) as described in U.S. Pat. Nos. 4,800,191, 6,716,817, 6,828,415, 6,867,191, 7,605,121, 7,718,599, 7,696,149 (Zentaris Ag), or pharmaceutically active decapeptides such as SB-030, SB-075 (cetrorelix) and SB-088 disclosed in EP 0 299 402 (Asta Pharma), the contents of each of which are incorporated herein by reference in their entirety. In another example, the targeting moiety may be LHRH analogues such as D-/L-MeI (4-[bis(2-chloroethyl)amino]-D/L-phenylalanine), cyclopropanealkanoyl, aziridine-2-carbonyl, epoxyalkyl, 1,4-naphthoquinone-5-oxycarbonyl-ethyl, doxorubicinyl (Doxorubicin, DOX), mitomicinyl (Mitomycin C), esperamycinyl or methotrexoyl, as disclosed in U.S. Pat. No. 6,214,969 to Janaky et al., the contents of which are incorporated herein by reference in their entirety.

In another embodiment, the targeting moiety may be any cell-binding molecule disclosed in U.S. Pat. No. 7,741,277 or 7,741,277 to Guenther et al. (Aeterna Zentaris), the contents of which are incorporated herein by reference in their entirety, such as octamer peptide, nonamer peptide, decamer peptide, luteinizing hormone releasing hormone (LHRH), [D-Lys6]-LHRH, LHRH analogue, LHRH agonist, Triptorelin ([D-Trp6]-LHRH), LHRH antagonist, bombesin, bombesin analogue, bombesin antagonist, somatostatin, somatostatin analogue, serum albumin, human serum albumin (HSA). These cell-binding molecules may be conjugated with disorazoles.

In another embodiment, targeting moieties may bind to growth hormone secretagogue (GHS) receptors, including ghrelin analogue ligands of GHS receptors. For example, targeting moieties may be any triazole derivatives with improved receptor activity and bioavailability properties as ghrelin analogue ligands of growth hormone secretagogue receptors as describe by U.S. Pat. No. 8,546,435 to Aicher, at al. (Aeterna Zentaris), the contents of which are incorporated herein by reference in their entirety.

In some embodiments, the targeting moiety X is an aptide or bipodal peptide. X may be any D-Aptamer-Like Peptide (D-Aptide) or retro-inverso Aptide which specifically binds to a target comprising: (a) a structure stabilizing region comprising parallel, antiparallel or parallel and antiparallel D-amino acid strands with interstrand noncovalent bonds; and (b) a target binding region I and a target binding region II comprising randomly selected n and m D-amino acids, respectively, and coupled to both ends of the structure stabilizing region, as disclosed in US Pat. Application No. 20140296479 to Jon et al., the contents of which are incorporated herein by reference in their entirety. X may be any bipodal peptide binder (BPB) comprising a structure stabilizing region of parallel or antiparallel amino acid strands or a combination of these strands to induce interstrand non-covalent bonds, and target binding regions I and II, each binding to each of both termini of the structure stabilizing region, as disclosed in US Pat. Application No. 20120321697 to Jon et al., the contents of which are incorporated herein by reference in their entirety. X may be an intracellular targeting bipodal-peptide binder specifically binding to an intracellular target molecule, comprising: (a) a structure-stabilizing region comprising a parallel amino acid strand, an antiparallel amino acid strand or parallel and antiparallel amino acid strands to induce interstrand non-covalent bonds; (b) target binding regions I and II each binding to each of both termini of the structure-stabilizing region, wherein the number of amino acid residues of the target binding region I is n and the number of amino acid residues of the target binding region II is m; and (c) a cell-penetrating peptide (CPP) linked to the structure-stabilizing region, the target binding region I or the target binding region II, as disclosed in U.S. Pat. Application No. 20120309934 to Jon et al., the contents of which are incorporated herein by reference in their entirety. X may be any bipodal peptide binder comprising a (3-hairpin motif or a leucine-zipper motif as a structure stabilizing region comprising two parallel amino acid strands or two antiparallel amino acid strands, and a target binding region I linked to one terminus of the first of the strands of the structure stabilizing region, and a target binding region II linked to the terminus of the second of the strands of the structure stabilizing region, as disclosed in US Pat. Application No. 20110152500 to Jon et al., the contents of which are incorporated herein by reference in their entirety. X may be any bipodal peptide binder targeting KPI as disclosed in WO2014017743 to Jon et al, any bipodal peptide binder targeting cytokine as disclosed in WO2011132939 to Jon et al., any bipodal peptide binder targeting transcription factor as disclosed in WO201132941 to Jon et al., any bipodal peptide binder targeting G protein-coupled receptor as disclosed in WO2011132938 to Jon et al., any bipodal peptide binder targeting receptor tyrosine kinase as disclosed in WO2011132940 to Jon et al., the contents of each of which are incorporated herein by reference in their entireties. X may also be bipodal peptide binders targeting cluster differentiation (CD7) or an ion channel.

In some embodiments, the target, target cell or marker is a molecule that is present exclusively or predominantly on the surface of malignant cells, e.g., a tumor antigen. In some embodiments, a marker is a prostate cancer marker. In some embodiments the target can be an intra-cellular protein.

In some embodiments, a marker is a breast cancer marker, a colon cancer marker, a rectal cancer marker, a lung cancer marker, a pancreatic cancer marker, a ovarian cancer marker, a bone cancer marker, a renal cancer marker, a liver cancer marker, a neurological cancer marker, a gastric cancer marker, a testicular cancer marker, a head and neck cancer marker, an esophageal cancer marker, or a cervical cancer marker.

The targeting moiety directs the conjugates to specific tissues, cells, or locations in a cell. The target can direct the conjugate in culture or in a whole organism, or both. In each case, the targeting moiety binds to a receptor that is present on the surface of or within the targeted cell(s), wherein the targeting moiety binds to the receptor with an effective specificity, affinity and avidity. In other embodiments the targeting moiety targets the conjugate to a specific tissue such as the liver, kidney, lung or pancreas. The targeting moiety can target the conjugate to a target cell such as a cancer cell, such as a receptor expressed on a cell such as a cancer cell, a matrix tissue, or a protein associated with cancer such as tumor antigen. Alternatively, cells comprising the tumor vasculature may be targeted. Targeting moieties can direct the conjugate to specific types of cells such as specific targeting to hepatocytes in the liver as opposed to Kupffer cells. In other cases, targeting moieties can direct the conjugate to cells of the reticular endothelial or lymphatic system, or to professional phagocytic cells such as macrophages or eosinophils.

In some embodiments the target is member of a class of proteins such as receptor tyrosine kinases (RTK) including the following RTK classes: RTK class I (EGF receptor family) (ErbB family), RTK class II (Insulin receptor family), RTK class III (PDGF receptor family), RTK class IV (FGF receptor family), RTK class V (VEGF receptors family), RTK class VI (HGF receptor family), RTK class VII (Trk receptor family), RTK class VIII (Eph receptor family), RTK class IX (AXL receptor family), RTK class X (LTK receptor family), RTK class XI (TIE receptor family), RTK class XII (ROR receptor family), RTK class XIII (DDR receptor family), RTK class XIV (RET receptor family), RTK class XV (KLG receptor family), RTK class XVI (RYK receptor family) and RTK class XVII (MuSK receptor family).

In some embodiments the target is a serine or threonine kinase, G-protein coupled receptor, methyl CpG binding protein, cell surface glycoprotein, cancer stem cell antigen or marker, carbonic anhydrase, cytolytic T lymphocyte antigen, DNA methyltransferase, an ectoenzyme, a glycosylphosphatidylinositol-anchored co-receptor, a glypican-related integral membrane proteoglycan, a heat shock protein, a hypoxia induced protein, a multi drug resistant transporter, a Tumor-associated macrophage marker, a tumor associated carbohydrate antigen, a TNF receptor family member, a transmembrane protein, a tumor necrosis factor receptor superfamily member, a tumour differentiation antigen, a zinc dependent metallo-exopeptidase, a zinc transporter, a sodium-dependent transmembrane transport protein, a member of the SIGLEC family of lectins, or a matrix metalloproteinase.

Other cell surface markers are useful as potential targets for tumor-homing therapeutics, including, for example HER-2, HER-3, EGFR, and the folate receptor.

In other embodiments, the targeting moiety binds a target such as CD19, CD70, CD56, PSMA, alpha integrin, CD22, CD138, EphA2, AGS-5, Nectin-4, HER2, GPMNB, CD74 and Le.

In some embodiments the target is a protein listed in Category A.

Category A. Non-limiting examples of proteins that may be targeted

| | | | |
|---|---|---|---|
| 5T4 | CD64 | GPIIb/IIIa receptors | PDGFRbeta |
| A20/TNFAIP3 | CD68 | GPR161/RE2 | P-glycoprotein |
| ABCB5 | CD70 | Guanylyl cyclase receptor C | Podoplanin |
| ABCG2 | CD80 | HA-CD44v3 | PON1 |
| AFP | CD86 | HER2/ERBB2 | PRAME |
| ALCAM/CD166 | CD90 | HIF1alpha | PSAM |
| ALDH1A1 | CD96 | HIF-2 | PTEN |
| Apelin J Receptor | CEACAM-5/cd66e | HLA-DR | RAAG12 |
| APN/CD13 | CEACAM-6 | Hsp90 | RON |
| AXL | c-KIT | IGE receptor | sialyl-Le(x) |
| B7H4 | c-Maf | IGF-1R | sialyl-Le(x) |
| BCMA | c-Met | IL-1 alpha | sialyl-Tn |
| BCRP/ABCG2 | Cripto/TDGF-1 | IL-11R | Sigma |
| | | | Receptor/Pgrmc1 |
| BMI-1 | CSFR | IL-1R | SLC34A2 |
| CA9 | CXCR1 | IL-23R | SLC44A4 |
| CAIX | CXCR1 | IL-2R | SLITRK6 |
| mmp | CXCR4 | IL-3 R | SOX2 |
| CanAg | disialylgalactosylgloboside | IL-4R | STAT-3 |
| CD117 | DLL4 | IL-6 R | STEAP-1 |
| CD11a | DNMT1 | Indegrin alpha 6 | STRO-1 |
| CD11b | DNMT3A | iNOS | Tenasin-C |
| CD136 | DNMT3B | Insulin receptor | TF antigen |
| CD138 | DNMT3L | L1CAM | TIM-3 |
| CD14 | EDB (Fibronectin extra | LGR5 | Tissue Factor |
| | domain B) | | (CD142) |
| CD15 | EGFR VIII | LIV-1 (SLC39A6), Zip6 | Tn antigen |
| CD152 (CTLA-4) | E-NPP3/CD203c | LRP | TNFR |
| CD172A | Epcam/TROP1 | MAGE-A3 | TRAIL-R1 |
| CD19 | EphA1 | MBD1 | TRAIL-R2 |
| CD20 | EphA2 | MBD2 | Transferrin receptor |
| CD204 | ERBB3 | MBD4 | TRK-A |
| CD206 | FAP | Mesothelin | TRK-B |
| CD22 | FGFR1 | Metadherin/MTDH/AEG-1 | Trop-2/EGP-1 |
| CD24 | FGFR2 | MICL | UHRF1 |
| CD25 | FGFR3 | MMP-2 | UHRF2 |
| CD26 | FGFR4 | MMP-9 | VEGFR1 |
| CD27 (CD70L) | Fibronectin | MRP1 | VEGFR2 |
| CD28 | Folate receptor | Muc-1 | VEGFR3 |
| CD3 | FRb | MUC16/CA-125 | ZBTB33 |
| CD30 | Galbg4 | Mushai-1 | ZBTB4 |
| CD33 | GD2 ganglioside | NaPi2b | EphA3 |
| CD34 | GD3 ganglioside | Nectin-4 | EphA4 |
| CD38 | GLI-1 | Nestin | EphA5 |
| CD40 | GLI-2 | Neurotensin receptor 1 | EphA6 |
| CD41 | globo-H | NF2 | EphA7 |
| CD44 | GLUT1 | Notch1 | EphA8 |
| CD45 | Glycoprotein NMB | Notch2 | EphB1 |
| CD45.1 | glycosphingolipid P₁ | Notch3 | EphB2 |
| CD45.2 | GM2 ganglioside | Notch4 | EphB3 |
| CD47/IAP | GP130 | Ovastacin | EphB4 |
| CD52 | GPC3 Glypican-3 | PDGFRalpha | EphB5 |
| EphB6 | GRP78 | | |

In some embodiments, the targeting moiety may bind to any human protein below. As a non-limiting example, the protein may be any protein of Category B including: 15 kDa selenoprotein; 1-acylglycerol-3-phosphate O-acyltransferase 1 to 6; 1-acylglycerol-3-phosphate O-acyltransferase 9; 2,3-bisphosphoglycerate mutase; 2′, 3′-cyclic nucleotide 3′ phosphodiesterase; 2,4-dienoyl CoA reductase 1, mitochondrial; 2,4-dienoyl CoA reductase 2, peroxisomal; 24-dehydrocholesterol reductase; 2′-5′-oligoadenylate synthetase 1 to 3; 2′-5′-oligoadenylate synthetase-like; 28S ribosomal protein S17, mitochondrial; 2-aminoethanethiol (cysteamine) dioxygenase; 2-hydroxyacyl-CoA lyase 1; 3′(2′), 5′-bisphosphate nucleotidase 1; 39S ribosomal protein L46, mitochondrial; 3-hydroxy-3-methylglutaryl-CoA reductase, synthase 1 and synthase 2; 3-hydroxyanthranilate 3,4-dioxygenase; 3-hydroxybutyrate dehydrogenase type 1 and type 2; 3-hydroxyisobutyrate dehydrogenase; 3-hydroxyisobutyryl-CoA hydrolase; 3-hydroxymethyl-3-methylglutaryl-CoA lyase and lyase-like 1; 3-ketodihydrosphingosine reductase; 3-oxoacid CoA transferase 1 and 2; 3-oxoacyl-ACP synthase, mitochondrial; 3′-phosphoadenosine 5′-phosphosulfate synthase 1 to 2; 3-phosphoinositide dependent protein kinase-1; 4-aminobutyrate aminotransferase; 4-hydroxy-2-oxoglutarate aldolase 1; 4-hydroxyphenylpyruvate dioxygenase and dioxygenase-like; 5′, 3′-nucleotidase, cytosolic; 5,10-methenyltetrahydrofolate synthetase (5-formyltetrahydrofolate cyclo-ligase); 5′, 3′-nucleotidase, mitochondrial; 5′-3′ exoribonuclease 1 and 2; 5-aminoimidazole-4-carboxamide ribonucleotide formyltransferase/IMP cyclohydrolase; 5-azacytidine induced 1 and 2; 5-hydroxytryptamine (serotonin) receptor 1A-1F, 2A-2C, 3A-3B, 4, 5A, 6 and 7; 5-hydroxytryptamine (serotonin) receptor 3 family member C-E; 5-methyltetrahydrofolate-homocysteine methyltransferase and methyltransferase reductase; 5′-nucleotidase, cytosolic IA, IB, II, III, III-like; 5′-nucleotidase, ecto (CD73); 6-phosphofructo-2-kinase/fructose-2,6-biphosphatase 1 to 4; 6-phosphogluconolactonase; 6-pyruvoyltetrahydropterin synthase; 7-dehydrocholesterol reductase; 8-oxoguanine DNA glycosylase; A kinase (PRKA) anchor protein 1 to 14, 17A, 8-like; A kinase (PRKA) interacting protein 1; Abelson helper integration site 1; ABI family, member 3; ABI family, member 3 (NESH) binding protein; abl-interactor 1 and 2; absent in melanoma 1 and 1-like, 2; acetoacetyl-CoA synthetase; acetylcholinesterase; acetyl-CoA acetyltransferase 1 and 2; acetyl-CoA acyltransferase 1 and 2; acetyl-CoA carboxylase alpha and beta; acetylserotonin O-methyltransferase and O-methyltransferase-like; achalasia, adrenocortical insufficiency, alacrimia; acid phosphatase 1, soluble; acid phosphatase 2 (lysosomal), 2-like, 5 (tartrate resistant), 6 (lysophosphatidic), prostate, testicular; acidic (leucine-rich) nuclear phosphoprotein 32 family, member A, B, D and E; acidic repeat containing; acireductone dioxygenase 1; aconitase 1 (soluble) and 2 (mitochondrial); acrosin and acrosin binding protein; acrosomal vesicle protein 1; actin binding LIM protein 1; actin binding LIM protein family, member 2 and 3; actin filament associated protein 1, 1-like 1 and 1-like 2; actin related protein 2/3 complex, subunit 1A, 1B, 2, -5 and 5-like; actin alpha 1 (skeletal muscle), alpha 2 (smooth muscle, aorta), alpha (cardiac muscle 1), beta, beta-like 2, gamma 1, and gamma 2 (smooth muscle, enteric); actin-binding Rho activating protein; actinin alpha 1, alpha 2 and alpha 4; actin-like 6A, 6B, 7A, 7B, 8 and 9; actin-related protein M1, T1 and T2; activated leukocyte cell adhesion molecule; activating signal cointegrator 1 complex subunit 1; activating signal cointegrator 1 complex subunit 2; activating signal cointegrator 1 complex subunit 3; activating transcription factor 1 to 7, 7 interacting protein and 7 interacting protein 2; activation-induced cytidine deaminase; activator of basal transcription 1; active BCR-related gene; activin A receptor type I, IB, IC, IIA, IIB, II-like1; activity-dependent neuroprotector homeobox; activity-regulated cytoskeleton-associated protein; acyl-CoA dehydrogenase family, member 8 to 11; acyl-CoA dehydrogenase C-2 to C-3 short chain, C-4 to C-12 straight chain, long chain, short/branched chain, very long chain; acyl-CoA oxidase 1 (palmitoyl), 2 (branched chain), 3 (pristanoyl) and oxidase-like; acyl-CoA synthetase bubblegum family member 1 and 2, long-chain family member 1, 3 4, 5 and 6, medium chain family member 1, 2A, 2B, 3 and 5, short-chain family member 1, 2, and 3; acyl-CoA thioesterase 1, 2, 4, 6-9, and 11-13; acyl-CoA wax alcohol acyltransferase 1 and 2; acylglycerol kinase; acyloxyacyl hydrolase (neutrophil); acylphosphatase 1 (erythrocyte (common) type) and 2 (muscle type); ADAM metallopeptidase domain 2, 7-12, 15, 17-23, 28-30, 32 and 33; ADAM metallopeptidase with thrombospondin type 1 motif, 1-10 and 12-20; ADAM-like, decysin 1; ADAMTS-like 1-5; adaptor-related protein complex 1-4 and the corresponding associated regulatory protein, alpha 1 subunit, alpha 2 subunit, beta 1 subunit, beta 2 subunit, gamma 1 subunit, gamma 2 subunit, mu 1 subunit, mu 2 subunit, 1 subunit, sigma 2 subunit, sigma 3 subunit, delta 1 subunit, epsilon 1 subunit; adducin 1 (alpha), 2 (beta), 3 (gamma); adenine phosphoribosyltransferase; adenomatosis polyposis coli 2, down-regulated 1, down-regulated 1-like; adenomatous polyposis coli; adenosine A1, A2a, A2b and A3 receptor; adenosine deaminase; adenosine deaminase, RNA-specific, B1 and B2; adenosine deaminase, tRNA-specific 1-3; adenosine deaminase-like; adenosine kinase; adenosine monophosphate deaminase 1-3; adenosylhomocysteinase; adenosylhomocysteinase-like 1 and like 2; adenosylmethionine decarboxylase 1; adenylate cyclase 1-10; adenylate cyclase activating polypeptide 1 pituitary and pituitary receptor type I; adenylate kinase 1-8; adenylosuccinate lyase; adenylosuccinate synthase and synthase like 1; adherens junctions associated protein 1; adhesion molecule with Ig-like domain 1-3; adhesion molecule, interacts with CXADR antigen 1; adhesion regulating molecule 1; adipogenin; adiponectin receptor 1 and 2; adiponectin C1Q and collagen domain containing; ADNP homeobox 2; ADP-dependent glucokinase; ADP-ribosylarginine hydrolase; ADP-ribosylation factor 1 and 3-6; ADP-ribosylation factor GTPase activating protein 1-3; ADP-ribosylation factor guanine nucleotide-exchange factor (brefeldin A-inhibited) 1 and 2; ADP-ribosylation factor interacting protein 1 and 2; ADP-ribosylation factor related protein 1; ADP-ribosylation factor-like 1-3, 4A, 4C, 4D, 5A, 5B, 5C, 6, 6 interacting protein 1, 8A, 8B, 9-11, 13A, 13B, 14-16, 17A, 17B and 17-like isoform A; ADP-ribosylation-like factor 6 interacting protein 4-6; ADP-ribosylhydrolase like 1 and 2; ADP-ribosyltransferase 1 and 3-5; adrenergic receptor alpha-1A-, alpha-1B-, alpha-1D-, alpha-2A-, alpha-2B-, alpha-2C-, beta 1, beta 2 and beta 3; adrenergic, beta, receptor kinase 1 and 2; adrenomedullin; adrenomedullin 2; advanced glycosylation end product-specific receptor; advillin; AE binding protein 1 and 2; AF4/FMR2 family, member 1-4; afamin; aftiphilin; age-related maculopathy susceptibility 2; aggrecan; agmatine ureohydrolase (agmatinase); agouti signaling protein; agrin; AHNAK nucleoprotein; AHNAK nucleoprotein 2; AIG2-like domain 1; ajuba LIM protein; akirin 1 and 2; AKT interacting protein; AKT1 substrate 1 (proline-rich); alanine-glyoxylate aminotransferase; alanine-glyoxylate aminotransferase 2; alanine-glyoxylate aminotransferase 2-like 1 and 2; alanyl (membrane) aminopeptidase; alanyl-tRNA synthetase; albumin; alcohol dehydrogenase 1A (class I), alpha polypeptide; alcohol dehydrogenase 1B (class I), beta polypeptide; alcohol dehydrogenase 4 (class II), pi polypeptide; alcohol dehydrogenase 5 (class III), chi polypeptide; alcohol dehydrogenase 6 (class V); alcohol dehydrogenase 7 (class IV), mu and sigma polypeptide; alcohol dehydrogenase, iron containing, 1; aldehyde dehydrogenase 1 family, member A1, A2, A3, B1, L1 and L2; aldehyde dehydrogenase 16 family, member A1; aldehyde dehydrogenase 18 family, member A1; aldehyde dehydrogenase 2 family (mitochondrial); aldehyde dehydrogenase 3 family, member A1, A2, B2; aldehyde dehydrogenase 4 family, member A1; aldehyde dehydrogenase 5 family, member A1; aldehyde dehydrogenase 6 family, member A1; aldehyde dehydrogenase 7 family, member A1; aldehyde dehydrogenase 8 family, member A1; aldehyde dehydrogenase 9 family, member A1; aldehyde oxidase 1; aldo-keto reductase family 1, member A1 (aldehyde reductase), B1 (aldose reductase), B10 (aldose reductase), B15, C1 (dihydrodiol dehydrogenase 1; 20-alpha (3-alpha)-hydroxysteroid dehydrogenase), C2 (dihydrodiol dehydrogenase 2; bile acid binding protein; 3-alpha hydroxysteroid dehydrogenase, type III), C3 (3-alpha hydroxysteroid dehydrogenase, type II), C4 (chlordecone reductase; 3-alpha hydroxysteroid dehydrogenase, type I; dihydrodiol dehydrogenase 4), C-like 1, D1 (delta 4-3-ketosteroid-5-beta-reductase), E2; aldo-keto reductase family 7 member A2 (aflatoxin aldehyde reductase) and A3 (aflatoxin aldehyde reductase); aldo-keto reductase family 7-like; aldolase A, fructose-bisphosphate; aldolase B, fructose-bisphosphate; aldolase C, fructose-bisphosphate; alkaline ceramidase 1-3; alkaline phosphatase, intestinal, liver/bone/kidney, placental, and placental-like 2; alkylglycerol monooxygenase; alkylglycerone phosphate synthase; allantoicase; allograft inflammatory factor 1; allograft inflammatory factor 1-like; alpha 1,4-galactosyltransferase; alpha- and gamma-adaptin binding protein; alpha hemoglobin stabilizing protein; alpha thalassemia/mental retardation syndrome X-linked; alpha tubulin acetyltransferase 1; Alpha-1,3-mannosyl-glycoprotein 4-beta-N-acetylglucosaminyltransferase-like protein LOC641515; alpha-1,4-N-acetylglucosaminyltransferase; alpha-1-B glycoprotein; alpha-1-microglobulin/bikunin precursor; alpha-2-glycoprotein 1, zinc-binding; alpha-2-HS-glycoprotein; alpha-2-macroglobulin; alpha-2-macroglobulin-like 1; alpha-fetoprotein; alpha-kinase 1-3; alpha-methylacyl-CoA racemase; Alport syndrome, mental retardation, midface hypoplasia and elliptocytosis chromosomal region gene 1; ALS2 C-terminal like; Alstrom syndrome 1; alveolar soft part sarcoma chromosome region, candidate 1; ALX homeobox 1, 3 and 4; Aly/REF export factor; amelotin; amiloride binding protein 1 (amine oxidase (copper-containing)); amiloride-sensitive cation channel 1 (neuronal), 2 (neuronal), 3, 4 (pituitary), and 5 (intestinal); aminoacyl tRNA synthetase complex-interacting multifunctional protein 1; aminoacyl tRNA synthetase complex-interacting multifunctional protein 2; aminoacylase 1; aminoadipate aminotransferase; aminoadipate-semialdehyde dehydrogenase; aminoadipate-semialdehyde dehydrogenase-phosphopantetheinyl transferase; aminoadipate-semialdehyde synthase; aminocarboxymuconate semialdehyde decarboxylase; aminolevulinate dehydratase; aminolevulinate, delta-, synthase 1 and synthase 2; aminomethyltransferase; aminopeptidase puromycin sensitive; Aminopeptidase Q; aminopeptidase-like 1; amino-terminal enhancer of split; AMME chromosomal region gene 1-like; amphiphysin; amphiregulin; amphiregulin B; amylase, alpha 1A (salivary), alpha 1B (salivary), alpha 1C (salivary), alpha 2A (pancreatic), and alpha 2B (pancreatic); amylo-alpha-1, 6-glucosidase, 4-alpha-glucanotransferase; amyloid beta (A4) precursor protein; amyloid beta (A4) precursor protein-binding, family A, member 1, 2 and 3; amyloid beta (A4) precursor protein-binding, family B, member 1 (Fe65), member 1 interacting protein, member 2 and member 3; amyloid beta (A4) precursor-like protein 1 and 2; amyloid beta precursor protein (cytoplasmic tail) binding protein 2; amyloid P component, serum; amyotrophic lateral sclerosis 2 (juvenile); amyotrophic lateral sclerosis 2 (juvenile) chromosome region, candidate 8, 11 and 12; anaphase promoting complex subunit 1, 2, 4, 5, 7, 10, 11, 13 and 16; anaplastic lymphoma receptor tyrosine kinase; ancient ubiquitous protein 1; androgen receptor; androgen-induced 1; angio-associated, migratory cell protein; angiogenic factor with G patch and FHA domains 1; angiogenin, ribonuclease, RNase A family, 5; angiomotin; angiomotin like 1 and 2; angiopoietin 1, 2 and 4; angiopoietin-like 1-7; angiotensin I converting enzyme (peptidyl-dipeptidase A) 1 and 2; angiotensin II receptor, type 1 and 2; angiotensin II receptor-associated protein; angiotensinogen (serpin peptidase inhibitor, clade A, member 8); anillin, actin binding protein; ANKHD1-EIF4EBP3 readthrough; ankyrin 1 (erythrocytic), ankyrin 2 (neuronal) and 3 (node of Ranvier (ankyrin G)); ankyrin and armadillo repeat containing; ankyrin repeat and GTPase domain Arf GTPase activating protein 11; ankyrin repeat domain 1, 2, 5, 6, 7, 9-12, 13A-13D, 16, 17, 18A-18B, 20 family member A1-A4, 22-24, 26-29, 30A-30B, 31-33, 34A-34C, 35-36, 36C, 37, 39-40, 42-46, 49-50, 52-58, 60, 62, 63 and 65; ankyrin repeat, family A (RFXANK-like), 2; annexin A1-A11, A13, A8-like 1 and A8-like 2; anoctamin 1-10 and 7-like 1; anthrax toxin receptor 1 and 2; antigen identified by monoclonal antibody Ki-67; antigen p97 (melanoma associated) identified by monoclonal antibodies 133.2 and 96.5; anti-Mullerian hormone; anti-Mullerian hormone receptor, type II; antizyme inhibitor 1; AP2 associated kinase 1; APAF1 interacting protein; apelin; apelin receptor; APEX nuclease (apurinic/apyrimidinic endonuclease) 2; APEX nuclease (multifunctional DNA repair enzyme) 1; APITD1-CORT readthrough; APOBEC1 complementation factor; apolipoprotein A-I; apolipoprotein A-I binding protein; apolipoprotein A-II, A-IV, A-V, B, C-I, C-II, C-IV, D, E, F, H, L1, L2, L3, L4, L5, L6, M, O and O-like; apolipoprotein B mRNA editing enzyme, catalytic polypeptide 1; apolipoprotein B mRNA editing enzyme, catalytic polypeptide-like 2, 3A-3D, and 3F-3H; apolipoprotein B receptor; apoptogenic 1; Apoptogenic protein 1, mitochondrial; apoptosis antagonizing transcription factor; apoptosis enhancing nuclease; apoptosis inhibitor 5; apoptosis, caspase activation inhibitor; apoptosis-associated tyrosine kinase; apoptosis-inducing factor, mitochondrion-associated, 1-3; apoptosis-inducing, TAF9-like domain 1; apoptotic chromatin condensation inducer 1; apoptotic peptidase activating factor 1; aprataxin; aprataxin and PNKP like factor; aquaporin 1-11, 12A and 12B; arachidonate 12-lipoxygenase; arachidonate 12-lipoxygenase, 12R type; arachidonate 15-lipoxygenase; arachidonate 15-lipoxygenase, type B; arachidonate 5-lipoxygenase; arachidonate 5-lipoxygenase-activating protein; arachidonate lipoxygenase 3; aralkylamine N-acetyltransferase; archaelysin family metallopeptidase 1 and 2; archain 1; ArfGAP with coiled-coil, ankyrin repeat and PH domains 1-3; ArfGAP with dual PH domains 1-2; ArfGAP with FG repeats 1-2; ArfGAP with GTPase domain, ankyrin repeat and PH domain 1-10; ArfGAP with RhoGAP domain, ankyrin repeat and PH domain 1-3; ArfGAP with SH3 domain, ankyrin repeat and PH domain 1-3; arginase, liver; arginase, type II; arginine and glutamate rich 1; arginine decarboxylase; arginine vasopressin; arginine vasopressin receptor 1A, 1B and 2; arginine vasopressin-induced 1; arginine/serine-rich coiled-coil 1; arginine/serine-rich coiled-coil 2; arginine-fifty homeobox; arginine-glutamic acid dipeptide (RE) repeats; argininosuccinate lyase; argininosuccinate synthase 1; arginyl aminopeptidase (aminopeptidase B); arginyl aminopeptidase (aminopeptidase B)-like 1; arginyltransferase 1; arginyl-tRNA synthetase; arginyl-tRNA synthetase 2, mitochondrial; aristaless related homeobox; ARL17 protein; armadillo repeat containing, X-linked 1-6; armadillo repeat gene deleted in velocardiofacial syndrome; arrestin 3, retinal (X-arrestin); arrestin, beta 1; arrestin, beta 2; arsenic (+3 oxidation state) methyltransferase; artemin; aryl hydrocarbon receptor; aryl hydrocarbon receptor interacting protein; aryl hydrocarbon receptor interacting protein-like 1; aryl hydrocarbon receptor nuclear translocator; aryl hydrocarbon receptor nuclear translocator-like; aryl hydrocarbon receptor nuclear translocator-like 2; arylacetamide deacetylase (esterase); arylacetamide deacetylase-like 2; arylacetamide deacetylase-like 3; arylacetamide deacetylase-like 4; arylformamidase; aryl-hydrocarbon receptor nuclear translocator 2; aryl-hydrocarbon receptor repressor; arylsulfatase A, B, D, E, F and G; arylsulfatase family, member H-K; arylsulfatase family, member I; arylsulfatase family, member J; arylsulfatase family, member K; arylsulfatase G; asialoglycoprotein receptor 1 and 2; asparaginase like 1; asparagine synthetase (glutamine-hydrolyzing); asparagine-linked glycosylation 1-like; asparaginyl-tRNA synthetase; aspartate beta-hydroxylase; aspartate dehydrogenase domain containing; aspartic peptidase, retroviral-like 1; aspartoacylase; aspartoacylase (aminocyclase) 3; aspartyl aminopeptidase; aspartylglucosaminidase; aspartyl-tRNA synthetase; aspartyl-tRNA synthetase 2, mitochondrial; asporin; astacin-like metallo-endopeptidase (M12 family); astrotactin 1; astrotactin 2; AT rich interactive domain 1A, 1B, 2, 3A-3C, 4A-4B and 5A-5B; ataxia telangiectasia and Rad3 related; ataxia telangiectasia mutated; ataxia, cerebellar, Cayman type; ataxin 1-3, 7, 10 1-like to 3-like, and 7-like 1 to 7-like 3; AT-hook transcription factor; atlastin GTPase 1; atlastin GTPase 2; atlastin GTPase 3; ATM interactor; ATP binding domain 4; ATP citrate lyase; ATP synthase mitochondrial F1 complex assembly factor 1; ATP synthase mitochondrial F1 complex assembly factor 2; ATP synthase protein 8; ATP synthase, H+ transporting, mitochondrial F1 complex, alpha subunit 1 (cardiac muscle), beta polypeptide, delta subunit, epsilon subunit, gamma polypeptide 1 and 0 subunit; ATP synthase, H+ transporting, mitochondrial Fo complex, subunit B1, C1, C2, C3, D, E, F2, F6, G, G2 and S; ATP/GTP binding protein 1; ATP/GTP binding protein-like 1-5; ATP1A1 opposite strand; ATP5S-like; ATPase inhibitory factor 1; ATPase type 13A1-13A5; ATPase, aminophospholipid transporter (APLT), class I, type 8A, member 1; ATPase, aminophospholipid transporter, class I, type 8A, member 2; ATPase, aminophospholipid transporter, class I, type 8B, member 1; ATPase, aminophospholipid transporter, class I, type 8B, member 3; ATPase, Ca++ transporting, cardiac muscle, fast twitch 1; ATPase, Ca++ transporting, cardiac muscle, slow twitch 2; ATPase, Ca++ transporting, plasma membrane 1-4; ATPase, Ca++ transporting, type 2C, member 1 and member 2; ATPase, Ca++ transporting, ubiquitous; ATPase, class I, type 8B, member 2 and member 4; ATPase, class II, type 9A; ATPase, class II, type 9B; ATPase, class V, type 10A, 10B and 10D; ATPase, class VI, type 11A, 11B and 11C; ATPase, Cu++ transporting, alpha polypeptide; ATPase, Cu++ transporting, beta polypeptide; ATPase, H+ transporting V0 subunit e2; ATPase, H+ transporting, lysosomal 13 kDa, V1 subunit G1; ATPase, H+ transporting, lysosomal 13 kDa, V1 subunit G2; ATPase, H+ transporting, lysosomal 13 kDa, V1 subunit G3; ATPase, H+ transporting, lysosomal 14 kDa, V1 subunit F; ATPase, H+ transporting, lysosomal 16 kDa, V0 subunit c; ATPase, H+ transporting, lysosomal 21 kDa, V0 subunit b; ATPase, H+ transporting, lysosomal 31 kDa, V1 subunit E1; ATPase, H+ transporting, lysosomal 31 kDa, V1 subunit E2; ATPase, H+ transporting, lysosomal 34 kDa, V1 subunit D; ATPase, H+ transporting, lysosomal 38 kDa, V0 subunit d1; ATPase, H+ transporting, lysosomal 38 kDa, V0 subunit d2; ATPase, H+ transporting, lysosomal 42 kDa, V1 subunit C1; ATPase, H+ transporting, lysosomal 42 kDa, V1 subunit C2; ATPase, H+ transporting, lysosomal 50/57 kDa, V1 subunit H; ATPase, H+ transporting, lysosomal 56/58 kDa, V1 subunit B1; ATPase, H+ transporting, lysosomal 56/58 kDa, V1 subunit B2; ATPase, H+ transporting, lysosomal 70 kDa, V1 subunit A; ATPase, H+ transporting, lysosomal 9 kDa, V0 subunit e1; ATPase, H+ transporting, lysosomal accessory protein 1; ATPase, H+ transporting, lysosomal accessory protein 1-like; ATPase, H+ transporting, lysosomal accessory protein 2; ATPase, H+ transporting, lysosomal V0 subunit al and subunit a2; ATPase, H+ transporting, lysosomal V0 subunit a4; ATPase, H+/K+ exchanging, alpha polypeptide and beta polypeptide; ATPase, H+/K+ transporting, nongastric, alpha polypeptide; ATPase, Na+/K+ transporting, alpha 1 polypeptide, alpha 2 polypeptide, alpha 3 polypeptide, alpha 4 polypeptide, beta 1 polypeptide, beta 2 polypeptide, beta 3 polypeptide, and beta 4 polypeptide; ATP-binding cassette, sub-family A (ABC1), member 1-member 13; ATP-binding cassette, sub-family B (MDR/TAP), member 1 and member 4-11; ATP-binding cassette, sub-family C (CFTR/MRP), member 1-6 and member 8-12; ATP-binding cassette, sub-family D (ALD), member 1-member 4; ATP-binding cassette, sub-family E (OABP), member 1; ATP-binding cassette, sub-family F (GCN20), member 1-member 3; ATP-binding cassette, sub-family G (WHITE), member 1, member 2, member 4, member 5 and member 8; ATR interacting protein; atrophin 1; attractin; attractin-like 1; AU RNA binding protein/enoyl-CoA hydratase; aurora kinase A; aurora kinase A interacting protein 1; aurora kinase B; aurora kinase C; autism susceptibility candidate 2; autocrine motility factor receptor; autoimmune regulator; autophagy/beclin-1 regulator 1; axin 1; axin 2; axin interactor, dorsalization associated; AXL receptor tyrosine kinase; azurocidin 1; B and T lymphocyte associated; B double prime 1, subunit of RNA polymerase III transcription initiation factor IIIB; B lymphoid tyrosine kinase; B melanoma antigen family, member 4; B9 protein domain 1; B9 protein domain 2; bactericidal/permeability-increasing protein; BAI1-associated protein 2; BAI1-associated protein 2-like 2; BAI1-associated protein 3; Bardet-Biedl syndrome 1, 2, 4, 5, 7, 9, 10 and 12; BarH-like homeobox 1 and 2; barrier to autointegration factor 1 and 2; Bartter syndrome, infantile, with sensorineural deafness (Barttin); BARX homeobox 1 and 2; basal cell adhesion molecule (Lutheran blood group); basic charge, Y-linked, 2; basic charge, Y-linked, 2B; basic charge, Y-linked, 2C; basic helix-loop-helix domain containing, class B, 9; basic helix-loop-helix family, member a15, a9, e22, e23, e40 and e41; basic leucine zipper and W2 domains 1 and 2; basic leucine zipper nuclear factor 1; basic leucine zipper transcription factor, ATF-like; basic leucine zipper transcription factor, ATF-like 2; basic leucine zipper transcription factor, ATF-like 3; basic transcription factor 3; basic transcription factor 3-like 4; basic, immunoglobulin-like variable motif containing; basigin (Ok blood group); basonuclin 1 and 2; bassoon (presynaptic cytomatrix protein); B-box and SPRY domain containing; BBSome interacting protein 1; BCDIN3 domain containing; B-cell CLL/lymphoma 2, 3, 6, 6 member B, 7A, 7B, 7C, 9, 9-like, 10, 11A and 11B; B-cell linker; B-cell receptor-associated protein 29; B-cell receptor-associated protein 31; B-cell scaffold protein with ankyrin repeats 1; B-cell translocation gene 1, anti-proliferative; B-cell translocation gene 4; BCL2 binding component 3; Bc12 modifying factor; BCL2/adenovirus E1B 19 kD interacting protein like; BCL2/adenovirus E1B 19 kDa interacting protein 1, 2, 3 and 3-like; BCL2-antagonist/killer 1; BCL2-associated agonist of cell death; BCL2-associated athanogene; BCL2-associated athanogene 2-6; BCL2-associated transcription factor 1; BCL2-associated X protein; BCL2-interacting killer (apoptosis-inducing); BCL2-like 1, like 2, like 10, like 11, like 12, like 13, like 14, like 15; BCL2-related ovarian killer; BCL2-related protein A1; BCL6 corepressor; BCL6 corepressor-like 1; beaded filament structural protein 2, phakinin; beclin 1, autophagy related; benzodiazapine receptor (peripheral) associated protein 1; Berardinelli-Seip congenital lipodystrophy 2 (seipin); bestrophin 1-4; beta 1,3-galactosyltransferase-like; beta-1,3-glucuronyltransferase 1 (glucuronosyltransferase P); beta-1,3-glucuronyltransferase 2 (glucuronosyltransferase S); beta-1,3-glucuronyltransferase 3 (glucuronosyltransferase I); beta-1,3-N-acetylgalactosaminyltransferase 1 (globoside blood group); beta-1,3-N-acetylgalactosaminyltransferase 2; beta-1,4-N-acetyl-galactosaminyl transferase 1; beta-1,4-N-acetyl-galactosaminyl transferase 2; beta-1,4-N-acetyl-galactosaminyl transferase 3; beta-1,4-N-acetyl-galactosaminyl transferase 4; beta-2-microglobulin; beta-carotene 15,15′-monooxygenase 1; beta-carotene oxygenase 2; betacellulin; Beta-defensin 130; betaine-homocysteine S-methyltransferase; betaine-homocysteine S-methyltransferase 2; beta-site APP-cleaving enzyme 1; beta-site APP-cleaving enzyme 2; beta-transducin repeat containing; BH3 interacting domain death agonist; BH3-like motif containing, cell death inducer; bifunctional apoptosis regulator; biglycan; bile acid CoA: amino acid N-acyltransferase (glycine N-choloyltransferase); biliverdin reductase A; biliverdin reductase B (flavin reductase (NADPH)); biogenesis of lysosomal organelles complex-1 subunit 1, subunit 2 and subunit 3; biogenesis of lysosomal organelles complex-1, subunit 3; biorientation of chromosomes in cell division 1; biorientation of chromosomes in cell division 1-like; biotinidase; biphenyl hydrolase-like (serine hydrolase); bladder cancer associated protein; bleomycin hydrolase; block of proliferation 1; blood vessel epicardial substance; Bloom syndrome, RecQ helicase-like; BMI1 polycomb ring finger oncogene; BMP binding endothelial regulator; BMP2 inducible kinase; BMX non-receptor tyrosine kinase; bombesin-like receptor 3; bone gamma-carboxyglutamate (gla) protein; bone marrow stromal cell antigen 1 and 2; bone morphogenetic protein 1-7, 8A, 8B, 10 and 15; bone morphogenetic protein receptor, type IA and IB; bone morphogenetic protein receptor, type II (serine/threonine kinase); bora, aurora kinase A activator; BR serine/threonine kinase 1 and 2; bradykinin receptor B1 and B2; brain abundant, membrane attached signal protein 1; brain and acute leukemia, cytoplasmic; brain and reproductive organ-expressed (TNFRSF1A modulator); brain expressed X-linked 2; brain expressed, associated with NEDD4, 1; brain expressed, X-linked 1, 4 and 5; brain protein 44; brain protein 44-like; brain protein 13; brain-derived neurotrophic factor; brain-specific angiogenesis inhibitor 1, 2 and 3; Brain-specific angiogenesis inhibitor 1-associated protein 2-like protein 1; brain-specific homeobox; branched chain amino-acid transaminase 1, cytosolic; branched chain amino-acid transaminase 2, mitochondrial; branched chain keto acid dehydrogenase E1, alpha polypeptide; branched chain keto acid dehydrogenase E1, beta polypeptide; branched chain ketoacid dehydrogenase kinase; BRCA1 associated protein; BRCA1 associated protein-1 (ubiquitin carboxy-terminal hydrolase); BRCA1 associated RING domain 1; BRCA1 interacting protein C-terminal helicase 1; BRCA1-associated ATM activator 1; BRCA2 and CDKN1A interacting protein; breakpoint cluster region; breast cancer 1, early onset; breast cancer 2, early onset; breast cancer anti-estrogen resistance 1 and 3; breast cancer metastasis suppressor 1; breast cancer metastasis-suppressor 1-like; breast carcinoma amplified sequence 1, 2, 3 and 4; brevican; BRF2, subunit of RNA polymerase III transcription initiation factor, BRF1-like; BRI3 binding protein; BRICK1, SCAR/WAVE actin-nucleating complex subunit; bridging integrator 1-3; BRISC and BRCA1 A complex member 1; BRO1 domain and CAAX motif containing; bromodomain adjacent to zinc finger domain 1A, 1B, 2A and 2B; bromodomain and PHD finger containing 1 and 3; bromodomain PHD finger transcription factor; bromodomain, testis-specific; Bruton agammaglobulinemia tyrosine kinase; BTG family, member 2 and 3; BTG3 associated nuclear protein; butyrobetaine (gamma), 2-oxoglutarate dioxygenase (gamma-butyrobetaine hydroxylase) 1; butyrophilin, subfamily 1, member A1; butyrophilin, subfamily 2, member A1 and member A2; butyrophilin, subfamily 3, member A1, member A2 and member A3; butyrophilin, subfamily 3, member A2; butyrophilin, subfamily 3, member A3; butyrophilin-like 2 (MHC class II associated); butyrophilin-like 3; butyrophilin-like 8; butyrophilin-like 9; butyrylcholinesterase; bystin-like; C15orf37 protein; C1D nuclear receptor corepressor; C1GALT1-specific chaperone 1; C1q and tumor necrosis factor related protein 1-9 and 9B; C2CD2-like; Ca++-dependent secretion activator; Ca++-dependent secretion activator 2; c-abl oncogene 1, non-receptor tyrosine kinase; cadherin 1, type 1, E-cadherin (epithelial); cadherin 10, type 2 (T2-cadherin); cadherin 11, type 2, OB-cadherin (osteoblast); cadherin 12, type 2 (N-cadherin 2); cadherin 13, H-cadherin (heart); cadherin 15, type 1, M-cadherin (myotubule); cadherin 16, KSP-cadherin; cadherin 17, LI cadherin (liver-intestine); cadherin 18, type 2; cadherin 19, type 2; cadherin 2, type 1, N-cadherin (neuronal); cadherin 20, type 2; cadherin 22, type 2; cadherin 24, type 2; cadherin 26; cadherin 3, type 1, P-cadherin (placental); cadherin 4, type 1, R-cadherin (retinal); cadherin 5, type 2 (vascular endothelium); cadherin 6, type 2, K-cadherin (fetal kidney); cadherin 7, type 2; cadherin 8, type 2; cadherin 9, type 2 (T1-cadherin); cadherin-related 23; cadherin-related family member 1-5; calbindin 1, 28 kDa; calbindin 2; calcineurin binding protein 1; calcitonin receptor; calcitonin receptor-like; calcitonin-related polypeptide alpha; calcitonin-related polypeptide beta; calcium activated nucleotidase 1; calcium and integrin binding 1 (calmyrin); calcium and integrin binding family member 2, 3 and 4; calcium binding and coiled-coil domain 1 and 2; calcium binding protein 1, 2, 4, 5, 7, 39, and 39-like; calcium binding tyrosine-(Y)-phosphorylation regulated; calcium channel, voltage-dependent, alpha 2/delta subunit 1-4; calcium channel, voltage-dependent, beta 1 subunit-beta 4 subunit; calcium channel, voltage-dependent, gamma subunit 1-8; calcium channel, voltage-dependent, L type, alpha 1C subunit; calcium channel, voltage-dependent, L type, alpha 1D subunit; calcium channel, voltage-dependent, L type, alpha 1F subunit; calcium channel, voltage-dependent, L type, alpha 1S subunit; calcium channel, voltage-dependent, N type, alpha 1B subunit; calcium channel, voltage-dependent, P/Q type, alpha 1A subunit; calcium channel, voltage-dependent, R type, alpha 1E subunit; calcium channel, voltage-dependent, T type, alpha 1G subunit; calcium channel, voltage-dependent, T type, alpha 1H subunit; calcium channel, voltage-dependent, T type, alpha 1I subunit; calcium homeostasis endoplasmic reticulum protein; calcium homeostasis modulator 1-3; calcium modulating ligand; calcium regulated heat stable protein 1, 24 kDa; calcium/calmodulin-dependent protein kinase I, ID and IG; calcium/calmodulin-dependent protein kinase II alpha, beta, delta, gamma, inhibitor 1 and inhibitor 2; calcium/calmodulin-dependent protein kinase IV; calcium/calmodulin-dependent protein kinase kinase 1, alpha; calcium/calmodulin-dependent protein kinase kinase 2, beta; calcium/calmodulin-dependent serine protein kinase (MAGUK family); Calcium-binding protein p22; calcium-binding protein, spermatid-specific 1; calcium-sensing receptor; calcyclin binding protein; calcyon neuron-specific vesicular protein; calcyphosine; calcyphosine 2; calcyphosine-like; caldesmon 1; calicin; calmegin; calmin (calponin-like, transmembrane); calmodulin 1 (phosphorylase kinase, delta); calmodulin 2 (phosphorylase kinase, delta); calmodulin 3 (phosphorylase kinase, delta); calmodulin binding transcription activator 1; calmodulin binding transcription activator 2; calmodulin regulated spectrin-associated protein 1; calmodulin regulated spectrin-associated protein family, member 2; calmodulin regulated spectrin-associated protein family, member 3; calmodulin-like 3, -like 4, -like 5 and -like 6; calmodulin-lysine N-methyltransferase; calneuron 1; calnexin; calpain 1-3, and 5-14; calpain, small subunit 1; calpain, small subunit 2; calpastatin; calponin 1, basic, smooth muscle; calponin 2; calponin 3, acidic; calreticulin; calreticulin 3; calsequestrin 1 (fast-twitch, skeletal muscle); calsequestrin 2 (cardiac muscle); calsyntenin 1-3; calumenin; CaM kinase-like vesicle-associated; cAMP responsive element binding protein 1, 3, 3-like 1, 3-like 2, 3-like 3, 3-like 4, and 5; cAMP responsive element binding protein-like 2; cAMP responsive element modulator; cAMP-regulated phosphoprotein 19 kDa and 21 kDa; cancer antigen 1; cancer susceptibility candidate 1 and 3-5; cancer/testis antigen 1A, 1B, 2 and 62; cancer/testis antigen family 45, member A1-A6; cancer/testis antigen family 47, member A1-A12 and B1; cannabinoid receptor 1 (brain); cannabinoid receptor 2 (macrophage); cannabinoid receptor interacting protein 1; CAP, adenylate cyclase-associated protein 1 (yeast); CAP, adenylate cyclase-associated protein, 2 (yeast); capping protein (actin filament) muscle Z-line, alpha 1, alpha 2, alpha 3 and beta; capping protein (actin filament), gelsolin-like; caprin family member 2; carbamoyl-phosphate synthase 1, mitochondrial; carbamoyl-phosphate synthetase 2, aspartate transcarbamylase, and dihydroorotase; carbohydrate (chondroitin 4) sulfotransferase 11, 12 and 13; carbohydrate (chondroitin 6) sulfotransferase 3; carbohydrate (keratan sulfate Gal-6) sulfotransferase 1; carbohydrate (N-acetylgalactosamine 4-O) sulfotransferase 8, 9 and 14; carbohydrate (N-acetylgalactosamine 4-sulfate 6-O) sulfotransferase 15; carbohydrate (N-acetylglucosamine 6-O) sulfotransferase 2 and 4-7; carbohydrate kinase domain containing; carbohydrate sulfotransferase 10; carbonic anhydrase I, II, III, IV, VA, VB, VI, VII, VIII, IX, X, XI, XII, XIII and XIV; carbonyl reductase 1, 3 and 4; carboxyl ester lipase (bile salt-stimulated lipase); carboxylesterase 1-3, 4A, and 5A; carboxypeptidase A1 (pancreatic), A2 (pancreatic), A3 (mast cell), A4, A5, A6, B1 (tissue), B2 (plasma), D, E, M, N polypeptide 1, N polypeptide 2, O, X (M14 family) member 1, X (M14 family) member 2, Z and vitellogenic-like; carcinoembryonic antigen-related cell adhesion molecule 1 (biliary glycoprotein), 3-8, 16, 18, 19 and 21; cardiolipin synthase 1; cardiomyopathy associated 5; cardiotrophin 1; cardiotrophin-like cytokine factor 1; carnitine O-acetyltransferase; carnitine O-octanoyltransferase; carnitine palmitoyltransferase 1A (liver), 1B (muscle), 1C and 2; carnosine dipeptidase 1 (metallopeptidase M20 family); carnosine synthase 1; CART prepropeptide; cartilage acidic protein 1; cartilage associated protein; cartilage intermediate layer protein 2; cartilage intermediate layer protein, nucleotide pyrophosphohydrolase; cartilage oligomeric matrix protein; Cas scaffolding protein family member 4; Cas-Br-M (murine) ecotropic retroviral transforming sequence; Cas-Br-M (murine) ecotropic retroviral transforming sequence b; Cas-Br-M (murine) ecotropic retroviral transforming sequence c; Cas-Br-M (murine) ecotropic retroviral transforming sequence-like 1; casein alpha sl; casein beta; casein kappa; casein kinase 1 alpha 1, alpha 1-like, delta, epsilon, gamma 1, gamma 2, and gamma 3; casein kinase 2, alpha 1 polypeptide; casein kinase 2, alpha prime polypeptide; casein kinase 2, beta polypeptide; CASK interacting protein 1 and 2; CASP8 and FADD-like apoptosis regulator; caspase 1-10 and 14; caspase recruitment domain family, member 6-8, 10, 11, 14 and 16-18; castor zinc finger 1; cat eye syndrome chromosome region, candidate 1, canidate 2, canidate 5 and canidate 6; catalase; catechol-O-methyltransferase; catenin (cadherin-associated protein), alpha 1, 102 kDa; catenin (cadherin-associated protein), alpha 2; catenin (cadherin-associated protein), alpha 3; catenin (cadherin-associated protein), alpha-like 1; catenin (cadherin-associated protein), beta 1, 88 kDa; catenin (cadherin-associated protein), delta 1; catenin (cadherin-associated protein), delta 2 (neural plakophilin-related arm-repeat protein); catenin, beta interacting protein 1; catenin, beta like 1; cathelicidin antimicrobial peptide; cathepsin A, B, C, D, E, F, G, H, K, L1, L2, O, S, W and Z; cation channel sperm associated 1, 2, 3 and 4; cation channel, sperm-associated, beta; cation channel, sperm-associated, gamma; caudal type homeobox 1, 2 and 4; caveolin 1, caveolae protein, 22 kDa; caveolin 2; caveolin 3; Cbp/p300-interacting transactivator, with Glu/Asp-rich carboxy-terminal domain 1, 2 and 4; CBP80/20-dependent translation initiation factor; CCAAT/enhancer binding protein (C/EBP) alpha, beta, delta, epsilon, gamma and zeta; CCCTC-binding factor (zinc finger protein); CCCTC-binding factor (zinc finger protein)-like; CCDC169-SOHLH2 readthrough; CCHC-type zinc finger, nucleic acid binding protein; CCR4-NOT transcription complex, subunit 1, 2, 3, 4, 6, 6-like, 7, 8 and 10; CD101 molecule; CD109 molecule; CD14 molecule; CD151 molecule (Raph blood group); CD160 molecule; CD163 molecule; CD163 molecule-like 1; CD164 molecule, sialomucin; CD164 sialomucin-like 2; CD177 molecule; CD180 molecule; CD19 molecule; CD1a molecule; CD1b molecule; CD1c molecule; CD1d molecule; CD1e molecule; CD2 (cytoplasmic tail) binding protein 2; CD2 molecule; CD200 molecule; CD200 receptor 1; CD200 receptor 1-like; CD207 molecule, langerin; CD209 molecule; CD22 molecule; CD226 molecule; CD244 molecule, natural killer cell receptor 2B4; CD247 molecule; CD248 molecule, endosialin; CD27 molecule; CD274 molecule; CD276 molecule; CD28 molecule; CD2-associated protein; CD300 molecule-like family member b, d, f and g; CD300a molecule; CD300c molecule; CD300e molecule; CD302 molecule; CD320 molecule; CD33 molecule; CD34 molecule; CD36 molecule (thrombospondin receptor); CD37 molecule; CD38 molecule; CD3d molecule, delta (CD3-TCR complex); CD3e molecule, epsilon (CD3-TCR complex); CD3e molecule, epsilon associated protein; CD3g molecule, gamma (CD3-TCR complex); CD4 molecule; CD40 ligand; CD40 molecule, TNF receptor superfamily member 5; CD44 molecule (Indian blood group); CD46 molecule, complement regulatory protein; CD47 molecule; CD48 molecule; CD5 molecule; CD5 molecule-like; CD52 molecule; CD53 molecule; CD55 molecule, decay accelerating factor for complement (Cromer blood group); CD58 molecule; CD59 molecule, complement regulatory protein; CD6 molecule; CD63 molecule; CD68 molecule; CD69 molecule; CD7 molecule; CD70 molecule; CD72 molecule; CD74 molecule, major histocompatibility complex, class II invariant chain; CD79a molecule, immunoglobulin-associated alpha; CD79b molecule, immunoglobulin-associated beta; CD80 molecule; CD81 molecule; CD82 molecule; CD83 molecule; CD84 molecule; CD86 molecule; CD8a molecule; CD8b molecule; CD9 molecule; CD93 molecule; CD96 molecule; CD97 molecule; CD99 molecule; CD99 molecule-like 2; CDC28 protein kinase regulatory subunit 1B; CDC28 protein kinase regulatory subunit 2; CDC42 binding protein kinase alpha (DMPK-like); CDC42 binding protein kinase beta (DMPK-like); CDC42 binding protein kinase gamma (DMPK-like); CDC42 effector protein (Rho GTPase binding) 1-5; Cdc42 guanine nucleotide exchange factor (GEF) 9; CDC42 small effector 1 and 2; CDC-like kinase 1-4; CDGSH iron sulfur domain 1-3; Cdk5 and Abl enzyme substrate 1; Cdk5 and Abl enzyme substrate 2; CDK5 regulatory subunit associated protein 1, 1-like 1, 2 and 3; CDKN1A interacting zinc finger protein 1; CDKN2A interacting protein; CDKN2A interacting protein N-terminal like; CDP-diacylglycerol synthase (phosphatidate cytidylyltransferase) 1 and 2; CDP-diacylglycerol-inositol 3-phosphatidyltransferase; cell adhesion molecule 1-4; cell cycle associated protein 1; cell cycle exit and neuronal differentiation 1; cell cycle progression 1; cell death-inducing DFFA-like effector a-c; cell division cycle 42 (GTP binding protein, 25 kDa); cell division cycle and apoptosis regulator 1; cell division cycle associated 2-8 and 7-like; cell growth regulator with EF-hand domain 1; cell growth regulator with ring finger domain 1; cellular repressor of E1A-stimulated genes 1 and 2; cellular retinoic acid binding protein 1 and 2; cementum protein 1; centlein, centrosomal protein; centrin, EF-hand protein, 1-3; centriolar coiled coil protein 110 kDa; centriolin; centrobin, centrosomal BRCA2 interacting protein; centromere protein A, B, c, E, F, H, I, J, K, L, M, N, O, P, Q, T, V and W; centrosomal protein 104 kDa, 112 kDa, 120 kDa, 128 kDa, 135 kDa, 152 kDa, 164 kDa, 170 kDa, 192 kDa, 19 kDa, 250 kDa, 290 kDa, 350 kDa, 41 kDa, 44 kDa, 55 kDa, 57 kDa, 57 kDa-like 1, 63 kDa, 68 kDa, 70 kDa, 72 kDa, 76 kDa, 78 kDa, 85 kDa, 85 kDa-like, 89 kDa, 95 kDa, and 97 kDa; centrosome and spindle pole associated protein 1; ceramide kinase; ceramide kinase-like; ceramide synthase 1-6; cerebellar degeneration-related protein 1, 34 kDa; cerebellar degeneration-related protein 2, 62 kDa; cerebellar degeneration-related protein 2-like; cerebellin 1 precursor; cerebellin 2 precursor; cerebellin 3 precursor; cerebellin 4 precursor; cereblon; cerebral cavernous malformation 2; cerebral dopamine neurotrophic factor; cerebral endothelial cell adhesion molecule; ceroid-lipofuscinosis, neuronal 3, 5, 6 and 8; ceruloplasmin (ferroxidase); c-fos induced growth factor (vascular endothelial growth factor D); CGG triplet repeat binding protein 1; CGRP receptor component; Charcot-Leyden crystal protein; charged multivesicular body protein 1A, 1B, 2A, 2B, 3, 4A, 4B, 4C, 5, 6 and 7; checkpoint kinase 1 and 2; checkpoint with forkhead and ring finger domains; chemokine (C motif) ligand 1 and 2; chemokine (C motif) receptor 1; chemokine (C—C motif) ligand 1, 2, 3, 3-like 1, 3-like 3, 4, 4-like 1, 4-like 2, 5, 7, 8, 11, 13, 14, 15, 16, 17, 18 (pulmonary and activation-regulated), 19, 2, 20, 21, 22, 23, 24, 25, 26, 27 and 28; chemokine (C—C motif) receptor 1-10, like 1 and like 2; chemokine (C—X3-C motif) ligand 1; chemokine (C—X3-C motif) receptor 1; chemokine (C—X—C motif) ligand 1 (melanoma growth stimulating activity, alpha), 2, 3, 5, 6 (granulocyte chemotactic protein 2), 9-14 and 16-17; chemokine (C—X—C motif) receptor 1-7; chemokine binding protein 2; chemokine-like factor; chemokine-like receptor 1; chimerin (chimaerin) 1 and 2; chitinase 1 (chitotriosidase); chitinase 3-like 1 (cartilage glycoprotein-39); chitinase 3-like 2; chitinase, acidic; chitobiase, di-N-acetyl-; chloride channel 1 (skeletal muscle) and 2-7; chloride channel accessory 1, 2 and 4; chloride channel accessory 4; chloride channel CLIC-like 1; chloride channel Ka and Kb; chloride channel, nucleotide-sensitive, 1A; chloride intracellular channel 1-6; cholecystokinin; cholecystokinin A receptor; cholecystokinin B receptor; cholesterol 25-hydroxylase; cholesteryl ester transfer protein, plasma; choline dehydrogenase; choline kinase alpha; choline kinase beta; choline 0-acetyltransferase; choline phosphotransferase 1; choline/ethanolamine phosphotransferase 1; cholinergic receptor, muscarinic 1-muscarinic 5; cholinergic receptor, nicotinic, alpha 1 (muscle), alpha 2 (neuronal), alpha 3-7, and alpha 9-10; cholinergic receptor, nicotinic, beta 1 (muscle), beta 2 (neuronal), beta 3 and beta 4; cholinergic receptor, nicotinic, delta; cholinergic receptor, nicotinic, epsilon; cholinergic receptor, nicotinic, gamma; chondroadherin; chondroadherin-like; chondroitin polymerizing factor; chondroitin polymerizing factor 2; chondroitin sulfate N-acetylgalactosaminyltransferase 1 and 2; chondroitin sulfate proteoglycan 4; chondroitin sulfate proteoglycan 5 (neuroglycan C); chondroitin sulfate synthase 1 and 3; chondrolectin; chondrosarcoma associated gene 1; chordin; chordin-like 1; chordin-like 2; chorionic gonadotropin, beta polypeptide; chorionic gonadotropin, beta polypeptide 1, 2, 5 and 7; chorionic somatomammotropin hormone 1 (placental lactogen); chorionic somatomammotropin hormone 2; chorionic somatomammotropin hormone-like 1; choroideremia (Rab escort protein 1); choroideremia-like (Rab escort protein 2); CHRNA7 (cholinergic receptor, nicotinic, alpha 7, exons 5-10) and FAM7A (family with sequence similarity 7A, exons A-E) fusion; chromatin accessibility complex 1; chromatin assembly factor 1, subunit A (p150); chromatin assembly factor 1, subunit B (p60); chromatin complexes subunit BAP18 isoform 2; chromatin licensing and DNA replication factor 1; chromatin target of PRMT1; chromodomain helicase DNA binding protein 1, 1-like, and 2-9; chromodomain protein, Y-like; chromodomain protein, Y-like 2; chromodomain protein, Y-linked, 1; chromodomain protein, Y-linked, 1B; chromodomain protein, Y-linked, 2A; chromodomain protein, Y-linked, 2B; chromogranin A (parathyroid secretory protein 1); chromogranin B (secretogranin 1); chronic lymphocytic leukemia up-regulated 1 opposite strand; Chronic lymphocytic leukemia up-regulated protein 1; CHURC1-FNTB readthrough; chymase 1, mast cell; chymotrypsin C (caldecrin); chymotrypsin-like; chymotrypsin-like elastase family, member 1, 2A, 2B, 3A and 3B; chymotrypsinogen B1, B2; ciliary neurotrophic factor; ciliary neurotrophic factor receptor; ciliary rootlet coiled-coil, rootletin; cingulin; cingulin-like 1; cirrhosis, autosomal recessive 1A (cirhin); citrate lyase beta like; citrate synthase; citron (rho-interacting, serine/threonine kinase 21); clarin 1-3; claspin; class II, major histocompatibility complex, transactivator; clathrin interactor 1; clathrin, heavy chain (Hc); clathrin, heavy chain-like 1; clathrin, light chain A; clathrin, light chain B; claudin 1-12, 14-20, and 22-25; clavesin 1; clavesin 2; cleavage and polyadenylation specific factor 1, 160 kDa; cleavage and polyadenylation specific factor 2, 100 kDa; cleavage and polyadenylation specific factor 3, 73 kDa; cleavage and polyadenylation specific factor 3-like; cleavage and polyadenylation specific factor 4, 30 kDa; cleavage and polyadenylation specific factor 4-like; cleavage and polyadenylation specific factor 6, 68 kDa; cleavage and polyadenylation specific factor 7, 59 kDa; cleavage stimulation factor, 3′ pre-RNA, subunit 1, 50 kDa; cleavage stimulation factor, 3′ pre-RNA, subunit 2, 64 kDa; cleavage stimulation factor, 3′ pre-RNA, subunit 2, 64 kDa, tau variant; cleavage stimulation factor, 3′ pre-RNA, subunit 3, 77 kDa; cleft lip and palate associated transmembrane protein 1; CLK4-associating serine/arginine rich protein; CLPTM1-like; clusterin; clusterin associated protein 1; clusterin-like 1 (retinal); c-Maf inducing protein; c-mer proto-oncogene tyrosine kinase; CMT1A duplicated region transcript 1; CMT1A duplicated region transcript 1 protein; CMT1A duplicated region transcript 15; CMT1A duplicated region transcript 15-like 2; CMT1A duplicated region transcript 4; c-myc binding protein; CNDP dipeptidase 2 (metallopeptidase M20 family); CNKSR family member 3; CoA synthase; coactivator-associated arginine methyltransferase 1; coactosin-like 1 (Dictyostelium); coagulation factor II (thrombin); coagulation factor II (thrombin) receptor; coagulation factor II (thrombin) receptor-like 1; coagulation factor II (thrombin) receptor-like 2; coagulation factor II (thrombin) receptor-like 3; coagulation factor III (thromboplastin, tissue factor); coagulation factor IX; coagulation factor V (proaccelerin, labile factor); coagulation factor VII (serum prothrombin conversion accelerator); coagulation factor VIII, procoagulant component; coagulation factor VIII-associated 1; coagulation factor VIII-associated 2; coagulation factor VIII-associated 3; coagulation factor X; coagulation factor XI; coagulation factor XII (Hageman factor); coagulation factor XIII, A1 polypeptide; coagulation factor XIII, B polypeptide; coatomer protein complex, subunit alpha; coatomer protein complex, subunit beta 1; coatomer protein complex, subunit beta 2 (beta prime); coatomer protein complex, subunit epsilon; coatomer protein complex, subunit gamma; coatomer protein complex, subunit gamma 2; coatomer protein complex, subunit zeta 1; coatomer protein complex, subunit zeta 2; COBL-like 1; cofactor of BRCA1; cofilin 1 (non-muscle); cofilin 2 (muscle); coiled-coil alpha-helical rod protein 1; coilin; cold inducible RNA binding protein; cold shock domain protein A; colipase, pancreatic; collagen and calcium binding EGF domains 1; collagen, type I, alpha 1 and alpha 2; collagen, type II, alpha 1; collagen, type III, alpha 1; collagen, type IV, alpha 1, alpha 2, alpha 3 (goodpasture antigen), alpha 4, alpha 5 and alpha 6; collagen, type IV, alpha 3 (Goodpasture antigen) binding protein; collagen, type IX, alpha 1-alpha 3; collagen, type V, alpha 1-alpha 3; collagen, type VI, alpha 1-alpha 3, and alpha 5-alpha 6; collagen, type VII, alpha 1; collagen, type VIII, alpha 1 and alpha 2; collagen, type X, alpha 1; collagen, type XI, alpha 1 and alpha 2; collagen, type XII, alpha 1; collagen, type XIII, alpha 1; collagen, type XIV, alpha 1; collagen, type XIX, alpha 1; collagen, type XV, alpha 1; collagen, type XVI, alpha 1; collagen, type XVII, alpha 1; collagen, type XVIII, alpha 1; collagen, type XX, alpha 1; collagen, type XXI, alpha 1; collagen, type XXII, alpha 1; collagen, type XXIII, alpha 1; collagen, type XXIV, alpha 1; collagen, type XXV, alpha 1; collagen, type XXVII, alpha 1; collagen, type XXVIII, alpha 1; collagen-like tail subunit (single strand of homotrimer) of asymmetric acetylcholinesterase; collapsin response mediator protein 1; collectin sub-family member 10 (C-type lectin); collectin sub-family member 11 and 12; colony stimulating factor 1 (macrophage); colony stimulating factor 1 receptor; colony stimulating factor 2 (granulocyte-macrophage); colony stimulating factor 2 receptor, alpha, low-affinity (granulocyte-macrophage); colony stimulating factor 2 receptor, beta, low-affinity (granulocyte-macrophage); colony stimulating factor 3 (granulocyte); colony stimulating factor 3 receptor (granulocyte); complement C4-B-like preproprotein; complement component (3b/4b) receptor 1 (Knops blood group); complement component (3b/4b) receptor 1-like; complement component (3d/Epstein Barr virus) receptor 2; complement component 1, q subcomponent binding protein; complement component 1, q subcomponent, A chain; complement component 1, q subcomponent, B chain; complement component 1, q subcomponent, C chain; complement component 1, q subcomponent-like 1; complement component 1, q subcomponent-like 2; complement component 1, q subcomponent-like 3; complement component 1, q subcomponent-like 4; complement component 1, r subcomponent; complement component 1, r subcomponent-like; complement component 1, s subcomponent; complement component 2; complement component 3; complement component 3a receptor 1; complement component 4 binding protein, alpha; complement component 4 binding protein, beta; complement component 4A (Rodgers blood group); complement component 4B (Chido blood group); complement component 5; complement component 5a receptor 1; complement component 6; complement component 7; complement component 8, alpha polypeptide, beta polypeptide and gamma polypeptide; complement component 9; complement factor B; complement factor D (adipsin); complement factor H; complement factor H-related 1-5; complement factor I; complement factor properdin; complexin 1-4; component of oligomeric golgi complex 1-8; cone-rod homeobox; congenital dyserythropoietic anemia, type I; connective tissue growth factor; connector enhancer of kinase suppressor of Ras 1 and 2; conserved helix-loop-helix ubiquitous kinase; consortin, connexin sorting protein; contactin 1-6; contactin associated protein 1; contactin associated protein-like 2; contactin associated protein-like 3; contactin associated protein-like 3B; contactin associated protein-like 4; contactin associated protein-like 5; copine family member IX; copine I, II, III, IV, V, VI, VII and VIII; copper chaperone for superoxide dismutase; coproporphyrinogen oxidase; core 1 synthase, glycoprotein-N-acetylgalactosamine 3-beta-galactosyltransferase, 1; core-binding factor, beta subunit; core-binding factor, runt domain, alpha subunit 2; translocated to, 2; core-binding factor, runt domain, alpha subunit 2; translocated to, 3; corepressor interacting with RBPJ, 1; corin, serine peptidase; corneodesmosin; cornifelin; cornulin; coronin 6; coronin 7; coronin, actin binding protein, 1A-1C and 2A-2B; cortactin; cortactin binding protein 2; cortexin 1-3; corticotropin releasing hormone; corticotropin releasing hormone binding protein; corticotropin releasing hormone receptor 1; corticotropin releasing hormone receptor 2; corticotropin-releasing factor receptor 1 isoform 1 precursor; cortistatin; COX4 neighbor; coxsackie virus and adenovirus receptor; CPX chromosome region, candidate 1; craniofacial development protein 1; C-reactive protein, pentraxin-related; creatine kinase, brain; creatine kinase, mitochondrial 1A; creatine kinase, mitochondrial 1B; creatine kinase, mitochondrial 2 (sarcomeric); creatine kinase, muscle; CREB binding protein; CREB regulated transcription coactivator 1-3; CREB/ATF bZIP transcription factor; cripto, FRL-1, cryptic family 1; cripto, FRL-1, cryptic family 1B; c-ros oncogene 1, receptor tyrosine kinase; cryptochrome 1 (photolyase-like); cryptochrome 2 (photolyase-like); crystallin, alpha A, alpha B, beta A1, beta A2, beta A4, beta B1, beta B2, beta B3, gamma A, gamma B, gamma C, gamma D, gamma N, gamma S, lambda 1, mu, zeta (quinone reductase), and zeta (quinone reductase)-like 1; CSE1 chromosome segregation 1-like (yeast); c-src tyrosine kinase; CSRP2 binding protein; CTAGE family, member 4, 5, 8 and 9; CTD (carboxy-terminal domain, RNA polymerase II, polypeptide A) phosphatase, subunit 1; CTD (carboxy-terminal domain, RNA polymerase II, polypeptide A) small phosphatase 1 and 2; CTD (carboxy-terminal domain, RNA polymerase II, polypeptide A) small phosphatase like 2; CTD (carboxy-terminal domain, RNA polymerase II, polypeptide A) small phosphatase-like; CTD nuclear envelope phosphatase 1; CTD-Protein Coding; C-terminal binding protein 1 and 2; CTP synthase; CTP synthase II; CTS telomere maintenance complex component 1; CTTNBP2 N-terminal like; C-type lectin domain family 1, member A and member B; C-type lectin domain family 10, member A; C-type lectin domain family 11, member A; C-type lectin domain family 12, member A and member B; C-type lectin domain family 14, member A; C-type lectin domain family 16, member A; C-type lectin domain family 17, member A; C-type lectin domain family 18, member A-member C; C-type lectin domain family 2, member A, member B, member D and member L; C-type lectin domain family 3, member A and member B; C-type lectin domain family 4, member A, member C-member G, and member M; C-type lectin domain family 5, member A; C-type lectin domain family 6, member A; C-type lectin domain family 7, member A; C-type lectin domain family 9, member A; C-type lectin-like 1; CUB and Sushi multiple domains 1-3; CUB and zona pellucida-like domains 1; cubilin (intrinsic factor-cobalamin receptor); CUGBP, Elav-like family member 1-member 6; cullin 1-3, 4A, 4B, 5, 7 and 9; cullin-associated and neddylation-dissociated 1; cutaneous T-cell lymphoma-associated antigen 1; cut-like homeobox 1 and 2; CXADR-like membrane protein; CXXC finger protein 1, 4, 5 and 11; cyclic nucleotide gated channel alpha 1-4, beta 1 and beta 3; cyclin A1, A2, B1, B2, B3, C, D1, D2, D3, E1, E2, F, G1, G2, H, I, J, J-like, K, L1, L2, M1-M4, 0, T1, T2, Y, Y-like 1, Y-like 2 and Y-like 3; cyclin B1 interacting protein 1, E3 ubiquitin protein ligase; cyclin D binding myb-like transcription factor 1; cyclin G associated kinase; cyclin-dependent kinase 1-10, 11A, 12-20, 2 associated protein 1, 2 associated protein 2, 2 interacting protein, 5 regulatory subunit 1 (p35), 5 regulatory subunit 2 (p39); cyclin-dependent kinase inhibitor 1A (p21, Cipl), 1B (p2′7, Kipl), 1C (p57, Kip2), 2A (melanoma, p16, inhibits CDK4), 2B (p15, inhibits CDK4), 2C (p18, inhibits CDK4), 2D (p19, inhibits CDK4), and 3; cyclin-dependent kinase-like 1 (CDC2-related kinase), like 2 (CDC2-related kinase), like 3, like 4 and like 5; cylicin, basic protein of sperm head cytoskeleton 1 and 2; cylindromatosis (turban tumor syndrome); CYP2B proteinCytochrome P450 2B7 short isoform; cystathionase (cystathionine gamma-lyase); cystathionine-beta-synthase; cystatin 11; cystatin 8 (cystatin-related epididymal specific); cystatin 9 (testatin); cystatin 9-like; cystatin A (stefin A); cystatin B (stefin B); cystatin C; cystatin D; cystatin E/M; cystatin F (leukocystatin); cystatin S; cystatin SA; cystatin SN; cystatin-like 1; cysteine and glycine-rich protein 1-3; cysteine conjugate-beta lyase 2; cysteine conjugate-beta lyase, cytoplasmic; cysteine dioxygenase, type I; cysteine rich transmembrane BMP regulator 1 (chordin-like); cysteine sulfinic acid decarboxylase; cysteine/histidine-rich 1; cysteine/tyrosine-rich 1; cysteine-rich C-terminal 1; cysteine-rich hydrophobic domain 1 and 2; cysteine-rich PAK1 inhibitor; cysteine-rich PDZ-binding protein; cysteine-rich protein 1 (intestinal); cysteine-rich protein 2; cysteine-rich protein 3; cysteine-rich secretory protein 1-3; cysteine-rich with EGF-like domains 1-2; cysteine-rich, angiogenic inducer, 61; cysteine-serine-rich nuclear protein 1-3; cysteinyl leukotriene receptor 1-2; cysteinyl-tRNA synthetase; cystic fibrosis transmembrane conductance regulator (ATP-binding cassette sub-family C, member 7); cystin 1; cystinosin, lysosomal cystine transporter; cytidine deaminase; cytidine monophosphate (UMP-CMP) kinase 1, cytosolic; cytidine monophosphate (UMP-CMP) kinase 2, mitochondrial; cytidine monophosphate N-acetylneuraminic acid synthetase; cytochrome b reductase 1; cytochrome b, ascorbate dependent 3; cytochrome b-245, alpha polypeptide; cytochrome b-245, beta polypeptide; cytochrome b5 reductase 1-4; cytochrome b5 reductase-like; cytochrome b5 type A (microsomal); cytochrome b5 type B (outer mitochondrial membrane); cytochrome b-561; cytochrome C oxidase assembly factor 5; cytochrome c oxidase assembly factor-like precursor; cytochrome c oxidase subunit IV isoform 1, subunit IV isoform 2 (lung), subunit Va, subunit Vb, subunit VIa polypeptide 1, subunit VIa polypeptide 2, subunit VIb polypeptide 1 (ubiquitous), subunit VIb polypeptide 2 (testis), subunit Vic, subunit VIIa polypeptide 1 (muscle), subunit VIIa polypeptide 2 (liver), subunit VIIa polypeptide 2 like, subunit VIIb, subunit VIIb2, subunit VIIc, subunit VIIIA (ubiquitous) and subunit VIIIC; cytochrome c, somatic; cytochrome c-1; cytochrome P450, family 1, subfamily A, polypeptide 1 and polypeptide 2; cytochrome P450, family 1, subfamily B, polypeptide 1; cytochrome P450, family 11, subfamily A, polypeptide 1; cytochrome P450, family 11, subfamily B, polypeptide 1 and polypeptide 2; cytochrome P450, family 17, subfamily A, polypeptide 1; cytochrome P450, family 19, subfamily A, polypeptide 1; cytochrome P450, family 2, subfamily A, polypeptide 6, 7 and 13; cytochrome P450, family 2, subfamily B, polypeptide 6; cytochrome P450, family 2, subfamily C, polypeptide 8, 9, 18 and 19; cytochrome P450, family 2, subfamily C, polypeptide 8; cytochrome P450, family 2, subfamily C, polypeptide 9; cytochrome P450, family 2, subfamily D, polypeptide 6; cytochrome P450, family 2, subfamily E, polypeptide 1; cytochrome P450, family 2, subfamily F, polypeptide 1; cytochrome P450, family 2, subfamily J, polypeptide 2; cytochrome P450, family 2, subfamily R, polypeptide 1; cytochrome P450, family 2, subfamily S, polypeptide 1; cytochrome P450, family 2, subfamily U, polypeptide 1; cytochrome P450, family 2, subfamily W, polypeptide 1; cytochrome P450, family 20, subfamily A, polypeptide 1; cytochrome P450, family 21, subfamily A, polypeptide 2; cytochrome P450, family 24, subfamily A, polypeptide 1; cytochrome P450, family 26, subfamily A, polypeptide 1; cytochrome P450, family 26, subfamily B, polypeptide 1; cytochrome P450, family 26, subfamily C, polypeptide 1; cytochrome P450, family 27, subfamily A, polypeptide 1; cytochrome P450, family 27, subfamily B, polypeptide 1; cytochrome P450, family 27, subfamily C, polypeptide 1; cytochrome P450, family 3, subfamily A, polypeptide 4, 5, 7 and 43; cytochrome P450, family 39, subfamily A, polypeptide 1; cytochrome P450, family 4, subfamily A, polypeptide 11; cytochrome P450, family 4, subfamily A, polypeptide 22; cytochrome P450, family 4, subfamily B, polypeptide 1; cytochrome P450, family 4, subfamily F, polypeptide 2, 3, 8, 11, 12 and 22; cytochrome P450, family 4, subfamily V, polypeptide 2; cytochrome P450, family 4, subfamily X, polypeptide 1; cytochrome P450, family 4, subfamily Z, polypeptide 1; cytochrome P450, family 46, subfamily A, polypeptide 1; cytochrome P450, family 51, subfamily A, polypeptide 1; cytochrome P450, family 7, subfamily A, polypeptide 1; cytochrome P450, family 7, subfamily B, polypeptide 1; cytochrome P450, family 8, subfamily B, polypeptide 1; cytoglobin; cytohesin 1; cytohesin 1 interacting protein; cytohesin 2; cytohesin 3; cytohesin 4; cytokine induced apoptosis inhibitor 1; cytokine receptor-like factor 1; cytokine receptor-like factor 2; cytokine receptor-like factor 3; cytokine-dependent hematopoietic cell linker; cytokine-like 1; cytoplasmic FMR1 interacting protein 1 and 2; cytoplasmic linker associated protein 1 and 2; cytoplasmic polyadenylation element binding protein 1-4; cytoskeleton associated protein 2; cytoskeleton associated protein 2-like; cytoskeleton associated protein 5; cytoskeleton-associated protein 4; cytosolic iron-sulfur protein assembly 1; cytotoxic and regulatory T cell molecule; cytotoxic T-lymphocyte-associated protein 4; D site of albumin promoter (albumin D-box) binding protein; D-2-hydroxyglutarate dehydrogenase; D4, zinc and double PHD fingers family 1-3; DAB2 interacting protein; damage-specific DNA binding protein 1, 127 kDa; damage-specific DNA binding protein 2, 48 kDa; D-amino acid oxidase activator; D-amino-acid oxidase; DAN domain family, member 5; D-aspartate oxidase; DAZ associated protein 1 and 2; DAZ interacting protein 1; DAZ interacting protein 1-like; DAZ interacting protein 3, zinc finger; dCMP deaminase; dCTP pyrophosphatase 1; DDB1 and CUL4 associated factor 4-8, 10-13, 15-17, 4-like 1, 4-like 2, 8-like 1, 12-like 1 and 12-like 2; D-dopachrome tautomerase; D-dopachrome tautomerase-like; DEAD (Asp-Glu-Ala-As) box polypeptide 19A and 19B; DEAD (Asp-Glu-Ala-Asp) box polypeptide 1, 3 (X-linked), 3 (Y-linked), 4, 5, 6, 10, 17, 18, 20, 21, 23, 24, 25, 27, 28, 31, 39A, 39B, 41, 42, 43, 46, 47, 49-56, 58-60 and 60-like; DEAD/H (Asp-Glu-Ala-Asp/His) box polypeptide 11; DEAD/H (Asp-Glu-Ala-Asp/His) box polypeptide 26B; deafness, autosomal dominant 5; deafness, autosomal recessive 31; deafness, autosomal recessive 59; DEAH (Asp-Glu-Ala-Asp/His) box polypeptide 57; DEAH (Asp-Glu-Ala-His) box polypeptide 8, 9, 15, 16, 29, 30, 32, 33, 34, 35, 36, 37, 38, and 40; DEAQ box RNA-dependent ATPase 1; death associated protein 3; death associated protein-like 1; death effector domain containing; death inducer-obliterator 1; death-associated protein; death-associated protein kinase 1-3; death-domain associated protein; decapping enzyme, scavenger; decorin; dedicator of cytokinesis 1-11; defender against cell death 1; defensin, alpha 1; defensin, alpha 1B; defensin, alpha 3, neutrophil-specific; defensin, alpha 4, corticostatin; defensin, alpha 5, Paneth cell-specific; defensin, alpha 6, Paneth cell-specific; defensin, beta 1, 4A, 4B, 103A, 103B, 104A, 104B, 105A, 105B, 106A, 106B, 107A, 107B, 108B, 110 locus, 112-119, 121, and 123-136; dehydrodolichyl diphosphate synthase; dehydrogenase/reductase (SDR family) member 1-4, 4 like 2, 7, 7B, 7C, 9, and 11-13; dehydrogenase/reductase (SDR family) X-linked; deiodinase, iodothyronine, type MIL DEK oncogene; deleted in azoospermia 1-4; deleted in azoospermia-like; deleted in bladder cancer 1; deleted in colorectal carcinoma; deleted in esophageal cancer 1; deleted in liver cancer 1; deleted in lung and esophageal cancer 1; deleted in lymphocytic leukemia 2-like; deleted in lymphocytic leukemia, 7; deleted in malignant brain tumors 1; delta(4)-desaturase, sphingolipid 1; delta(4)-desaturase, sphingolipid 2; delta/notch-like EGF repeat containing; dendrin; density-regulated protein; dentin matrix acidic phosphoprotein 1; dentin sialophosphoprotein; deoxycytidine kinase; deoxyguanosine kinase; deoxyhypusine hydroxylase/monooxygenase; deoxyhypusine synthase; deoxynucleotidyltransferase, terminal; deoxynucleotidyltransferase, terminal, interacting protein 1 and 2; deoxyribonuclease I; deoxyribonuclease II beta; deoxyribonuclease II, lysosomal; deoxyribonuclease I-like 1; deoxyribonuclease I-like 2; deoxyribonuclease I-like 3; deoxythymidylate kinase (thymidylate kinase); deoxyuridine triphosphatase; dephospho-CoA kinase domain containing; Derl-like domain family, member 1; Derl-like domain family, member 2; Derl-like domain family, member 3; dermatan sulfate epimerase; dermatan sulfate epimerase-like; dermatopontin; dermcidin; dermokine; desert hedgehog; desmin; desmocollin 1-3; desmoglein 1-4; desmoplakin; destrin (actin depolymerizing factor); DET1 and DDB1 associated 1; developing brain homeobox 1; developing brain homeobox 2; developmental pluripotency associated 2-5; developmentally regulated GTP binding protein 1; developmentally regulated GTP binding protein 2; DEXH (Asp-Glu-X-His) box polypeptide 58; diablo, IAP-binding mitochondrial protein; diacylglycerol kinase, alpha 80 kDa, beta 90 kDa, delta 130 kDa, epsilon 64 kDa, eta, gamma 90 kDa, iota, theta 110 kDa and zeta; diacylglycerol lipase, alpha and beta; diacylglycerol O-acyltransferase 1, 2 and 2-like 6; diazepam binding inhibitor (GABA receptor modulator, acyl-CoA binding protein); dicarbonyl/L-xylulose reductase; dicer 1, ribonuclease type III; dickkopf-like 1; diencephalon/mesencephalon homeobox 1; differential display clone 8 isoform 2; diffuse panbronchiolitis critical region 1; DiGeorge syndrome critical region gene 2, 6, 6-like, 8 and 14; dihydrodiol dehydrogenase (dimeric); dihydrofolate reductase; dihydrofolate reductase-like 1; dihydrolipoamide branched chain transacylase E2; dihydrolipoamide dehydrogenase; dihydrolipoamide S-acetyltransferase; dihydrolipoamide S-succinyltransferase (E2 component of 2-oxo-glutarate complex); dihydroorotate dehydrogenase (quinone); dihydropyrimidinase; dihydropyrimidinase-like 2-like 5; dihydropyrimidine dehydrogenase; dimethylarginine dimethylaminohydrolase 1; dimethylarginine dimethylaminohydrolase 2; dimethylglycine dehydrogenase; dipeptidase 1 (renal); dipeptidase 2; dipeptidase 3; dipeptidyl-peptidase 10 (non-functional); dipeptidyl-peptidase 3, 4, and 6-9; diphosphoinositol pentakisphosphate kinase 1; diphosphoinositol pentakisphosphate kinase 2; DIRAS family, GTP-binding RAS-like 1-3; discoidin domain receptor tyrosine kinase 1 and 2; dishevelled associated activator of morphogenesis 1 and 2; disrupted in renal carcinoma 1 and 2; disrupted in schizophrenia 1; distal-less homeobox 1-6; divergent-paired related homeobox; DMRT-like family A1; DMRT-like family A2; DMRT-like family B with proline-rich C-terminal, 1; DMRT-like family C1; DMRT-like family C1B; DMRT-like family C2; Dmx-like 1; Dmx-like 2; DNA (cytosine-5-)-methyltransferase 1; DNA (cytosine-5-)-methyltransferase 3 alpha; DNA (cytosine-5-)-methyltransferase 3 beta; DNA (cytosine-5-)-methyltransferase 3-like; DNA cross-link repair 1A, 1B and 1C; DNA fragmentation factor, 40 kDa, beta polypeptide (caspase-activated DNase); DNA fragmentation factor, 45 kDa, alpha polypeptide; DNA methyltransferase 1 associated protein 1; DNA-damage regulated autophagy modulator 1 and 2; DNA-damage-inducible transcript 3 and 4; DNA-damage-inducible transcript 4-like; DNAJC25-GNG10 readthrough; DNL-type zinc finger; docking protein 1 (62 kDa (downstream of tyrosine kinase 1)), 2 (56 kDa), 3, 4, 5, 6 and 7; dolichol kinase; dolichyl pyrophosphate phosphatase 1; dolichyl-diphosphooligosaccharide-protein glycosyltransferase; dolichyl-phosphate (UDP-N-acetylglucosamine)N-acetylglucosaminephosphotransferase 1 (GlcNAc-1-P transferase); dolichyl-phosphate mannosyltransferase polypeptide 1, catalytic subunit; dolichyl-phosphate mannosyltransferase polypeptide 2, regulatory subunit; dolichyl-phosphate mannosyltransferase polypeptide 3; dopa decarboxylase (aromatic L-amino acid decarboxylase); dopachrome tautomerase (dopachrome delta-isomerase, tyrosine-related protein 2); dopamine beta-hydroxylase (dopamine beta-monooxygenase); dopamine receptor D1-D5; dopey family member 1 and 2; dorsal root ganglia homeobox; double C2-like domains, alpha and beta; double homeobox 4; double homeobox 4 like 2-like 7; double homeobox A; double zinc ribbon and ankyrin repeat domains 1; doublecortin; doublecortin-like kinase 1-3; doublesex and mab-3 related transcription factor 1-3; Down syndrome cell adhesion molecule; Down syndrome cell adhesion molecule like 1; Down syndrome critical region gene 3, 4, 6 and 8; down-regulator of transcription 1, TBP-binding (negative cofactor 2); DR1-associated protein 1 (negative cofactor 2 alpha); drebrin 1; drebrin-like; drosha, ribonuclease type III; dual adaptor of phosphotyrosine and 3-phosphoinositides; dual oxidase 1; dual oxidase 2; dual oxidase maturation factor 1 and 2; dual serine/threonine and tyrosine protein kinase; dual specificity phosphatase 1-16, 18-19, 21-23 and 28; dual-specificity tyrosine-(Y)-phosphorylation regulated kinase 1A, 1B, 2, 3, and 4; Duffy blood group, chemokine receptor; dymeclin; dynactin 1, 2 (p50), 3 (p22), 4 (p62), 5 (p25) and 6; dynamin 1, 1-like, 2 and 3; dynamin binding protein; dynein heavy chain domain 1; dynein, axonemal, assembly factor 1 and 2; dynein, axonemal, heavy chain 1-3, 5-12, 14 and 17; dynein, axonemal, intermediate chain 1 and 2; dynein, axonemal, light chain 1 and 4; dynein, axonemal, light intermediate chain 1; dynein, cytoplasmic 1, heavy chain 1; dynein, cytoplasmic 1, intermediate chain 1 and 2; dynein, cytoplasmic 1, light intermediate chain 1 and 2; dynein, cytoplasmic 2, heavy chain 1; dynein, cytoplasmic 2, light intermediate chain 1; dynein, light chain, LC8-type 1 and 2; dynein, light chain, roadblock-type 1 and 2; dynein, light chain, Tctex-type 1 and 3; dysferlin, limb girdle muscular dystrophy 2B (autosomal recessive); dyskeratosis congenita 1, dyskerin; dystonin; dystrobrevin binding protein 1; dystrobrevin, alpha; dystrobrevin, beta; dystroglycan 1 (dystrophin-associated glycoprotein 1); dystrophia myotonica, WD repeat containing; dystrophia myotonica-protein kinase; dystrophin; dystrophin related protein 2; dystrotelin; E1A binding protein p300; E1A binding protein p400; E2F transcription factor 1-8; E2F-associated phosphoprotein; E4F transcription factor 1; E74-like factor 1 (ets domain transcription factor); E74-like factor 2 (ets domain transcription factor); E74-like factor 3 (ets domain transcription factor, epithelial-specific); E74-like factor 4 (ets domain transcription factor); E74-like factor 5 (ets domain transcription factor); early B-cell factor 1-4; early endosome antigen 1; early growth response 1-4; EBNA1 binding protein 2; echinoderm microtubule associated protein like 1-6; ecotropic viral integration site 2A, 2B, 5 and 5-like; ectodermal-neural cortex 1 (with BTB-like domain); ectodysplasin A; ectodysplasin A receptor; ectodysplasin A2 receptor; ecto-NOX disulfide-thiol exchanger 1; ecto-NOX disulfide-thiol exchanger 2; ectonucleoside triphosphate diphosphohydrolase 1-8; ectonucleotide pyrophosphatase/phosphodiesterase 1-3, 6 and 7; EDAR-associated death domain; EF-hand calcium binding domain 1, 2, 3, 4A, 4B, 5, 6, 7, 9, 10, and 11; EF-hand domain family, member A1, A2, B, D1 and D2; egf-like module containing, mucin-like, hormone receptor-like 1; egf-like module containing, mucin-like, hormone receptor-like 2; egf-like module containing, mucin-like, hormone receptor-like 3; EGF-like repeats and discoidin I-like domains 3; EGF-like, fibronectin type III and laminin G domains; EGF-like-domain, multiple 6-8; EH domain binding protein 1; EH domain binding protein 1-like 1; elastase, neutrophil expressed; elastin; elastin microfibril interfacer 1-3; electron-transfer-flavoprotein, alpha polypeptide; electron-transfer-flavoprotein, beta polypeptide; electron-transferring-flavoprotein dehydrogenase; ELK1, member of ETS oncogene family; ELK3, ETS-domain protein (SRF accessory protein 2); ELK4, ETS-domain protein (SRF accessory protein 1); ELKS/RAB6-interacting/CAST family member 1; ELKS/RAB6-interacting/CAST family member 2; ELL associated factor 1 and 2; Ellis van Creveld syndrome; Ellis van Creveld syndrome 2; elongation factor RNA polymerase II; elongation factor RNA polymerase II-like 3; elongation factor, RNA polymerase II, 2; ELOVL fatty acid elongase 1-7; embigin; embryonal Fyn-associated substrate; embryonic ectoderm development; emerin; emopamil binding protein (sterol isomerase); emopamil binding protein-like; empty spiracles homeobox 1; empty spiracles homeobox 2; Enah/Vasp-like; enamelin; endo/exonuclease (5′-3′), endonuclease G-like; endo-beta-N-acetylglucosaminidase; endogenous Bornavirus-like nucleoprotein 2; endogenous retrovirus group 3, member 1; endogenous retrovirus group FRD, member 1; endogenous retrovirus group W, member 1; endoglin; endomucin; endonuclease G; endonuclease V; endonuclease, polyU-specific; endoplasmic reticulum aminopeptidase 1; endoplasmic reticulum aminopeptidase 2; endoplasmic reticulum lectin 1; endoplasmic reticulum metallopeptidase 1; endoplasmic reticulum protein 27; endoplasmic reticulum protein 29; endoplasmic reticulum protein 44; endoplasmic reticulum to nucleus signaling 1; endoplasmic reticulum to nucleus signaling 2; endoplasmic reticulum-golgi intermediate compartment (ERGIC) 1; endosulfine alpha; endothelial cell adhesion molecule; endothelial cell-specific chemotaxis regulator; endothelial cell-specific molecule 1; endothelial differentiation-related factor 1; endothelial PAS domain protein 1; endothelin 1; endothelin 2; endothelin 3; endothelin converting enzyme 1 and 2; endothelin converting enzyme-like 1; endothelin receptor type A; endothelin receptor type B; energy homeostasis associated; engrailed homeobox 1; engrailed homeobox 2; engulfment and cell motility 1-3; enhancer of mRNA decapping 4; enkurin, TRPC channel interacting protein; enolase 1, (alpha); enolase 2 (gamma, neuronal); enolase 3 (beta, muscle); enolase family member 4; enolase superfamily member 1; enolase-phosphatase 1; enoyl CoA hydratase 1, peroxisomal; enoyl CoA hydratase, short chain, 1, mitochondrial; enoyl-CoA delta isomerase 1; enoyl-CoA delta isomerase 2; enoyl-CoA, hydratase/3-hydroxyacyl CoA dehydrogenase; envoplakin; envoplakin-like; eomesodermin; eosinophil peroxidase; EP300 interacting inhibitor of differentiation 1, 2, 2B and 3; EP400 N-terminal like; ependymin related protein 1 (zebrafish); EPH receptor A1-A8, A10, B1-B4 and B6; ephrin-A1, A2, A3, A4, A5, B1, B2 and B3; epidermal growth factor; epidermal growth factor receptor; epidermal growth factor receptor pathway substrate 15; epidermal growth factor receptor pathway substrate 15-like 1; epidermal growth factor receptor pathway substrate 8; epididymal protein 3A and 3B; epididymal secretory glutathione peroxidase isoform 1 precursor; epididymal sperm binding protein 1; epilepsy, progressive myoclonus type 2A, Lafora disease (laforin); epiphycan; epiplakin 1; epiregulin; epithelial cell adhesion molecule; epithelial cell transforming sequence 2 oncogene; epithelial cell transforming sequence 2 oncogene-like; epithelial membrane protein 1-3; epithelial splicing regulatory protein 1 and 2; epithelial stromal interaction 1 (breast); EPM2A (laforin) interacting protein 1; epoxide hydrolase 1, microsomal (xenobiotic); epoxide hydrolase 2, cytoplasmic; epoxide hydrolase 3; epoxide hydrolase 4; EPS8-like 1; EPS8-like 2; EPS8-like 3; epsin 1; epsin 2; epsin 3; Epstein-Barr virus induced 3; ER degradation enhancer, mannosidase alpha-like 1-like 3; ER lipid raft associated 1; ER lipid raft associated 2; Erb-b2 receptor tyrosine kinase 2; ERBB receptor feedback inhibitor 1; erbb2 interacting protein; ERGIC and golgi 2; ERGIC and golgi 3; ERI1 exoribonuclease family member 2; ERI1 exoribonuclease family member 3; ermin, ERM-like protein; erythroblast membrane-associated protein (Scianna blood group); erythrocyte membrane protein band 4.1 (elliptocytosis 1, RH-linked); erythrocyte membrane protein band 4.1 like 4A, like 4B, like 5, like 1, like 2, and like 3; erythrocyte membrane protein band 4.2; erythrocyte membrane protein band 4.9 (dematin); erythropoietin; erythropoietin receptor; ES cell expressed Ras; espin; espin-like; esterase D; estrogen receptor 1; estrogen receptor 2 (ER beta); estrogen receptor binding site associated, antigen, 9; estrogen-related receptor alpha; estrogen-related receptor beta; estrogen-related receptor gamma; ESX homeobox 1; ethanolamine kinase 1; ethanolamine kinase 2; ethanolaminephosphotransferase 1 (CDP-ethanolamine-specific); ethylmalonic encephalopathy 1; etoposide induced 2.4 mRNA; ets variant 1, 2, 3, 3-like, 4, 5, 6 and 7; Ets2 repressor factor; euchromatic histone-lysine N-methyltransferase 1; euchromatic histone-lysine N-methyltransferase 2; eukaryotic elongation factor, selenocysteine-tRNA-specific; eukaryotic elongation factor-2 kinase; eukaryotic translation elongation factor 1 alpha 1, alpha 2, beta 2, delta (guanine nucleotide exchange protein), epsilon 1, and gamma; eukaryotic translation elongation factor 2; eukaryotic translation initiation factor 1; eukaryotic translation initiation factor 1A domain containing; eukaryotic translation initiation factor 1A, X-linked and Y-linked; eukaryotic translation initiation factor 1B; eukaryotic translation initiation factor 2 alpha kinase 4; eukaryotic translation initiation factor 2, subunit 1 alpha (35 kDa), subunit 2 beta (38 kDa) and subunit 3 gamma (52 kDa); eukaryotic translation initiation factor 2A, 65 kDa; eukaryotic translation initiation factor 2-alpha kinase 1-alpha kinase 3; eukaryotic translation initiation factor 2B, subunit 1 alpha (26 kDa), subunit 2 beta (29 kDa), 3 gamma (58 kDa), 4 delta (67 kDa), and subunit 5 epsilon (82 kDa); eukaryotic translation initiation factor 2C, 1-4; eukaryotic translation initiation factor 2D; eukaryotic translation initiation factor 3, subunit A-subunit M and subunit C-like; eukaryotic translation initiation factor 4 gamma, 1-3; eukaryotic translation initiation factor 4A1-4A3; eukaryotic translation initiation factor 4B; eukaryotic translation initiation factor 4E; eukaryotic translation initiation factor 4E binding protein 1-3; eukaryotic translation initiation factor 4E family member 1B, 2 and 3; eukaryotic translation initiation factor 4E nuclear import factor 1; eukaryotic translation initiation factor 4H; eukaryotic translation initiation factor 5; eukaryotic translation initiation factor 5A; eukaryotic translation initiation factor 5A2; eukaryotic translation initiation factor 5A-like 1; eukaryotic translation initiation factor 5B; eukaryotic translation initiation factor 6; eukaryotic translation termination factor 1; even-skipped homeobox 1; even-skipped homeobox 2; Ewing sarcoma breakpoint region 1; Ewing tumor-associated antigen 1; excision repair cross-complementing rodent repair deficiency, complementation group 1 (includes overlapping antisense sequence); excision repair cross-complementing rodent repair deficiency, complementation group 2; excision repair cross-complementing rodent repair deficiency, complementation group 3 (xeroderma pigmentosum group B complementing); excision repair cross-complementing rodent repair deficiency, complementation group 4; excision repair cross-complementing rodent repair deficiency, complementation group 5; excision repair cross-complementing rodent repair deficiency, complementation group 6; excision repair cross-complementing rodent repair deficiency, complementation group 6-like; excision repair cross-complementing rodent repair deficiency, complementation group 8; exocyst complex component 1-8, 3-like 1, 3-like 2, 3-like 3, and 6B; exonuclease 1; exophilin 5; exoribonuclease 1; exosome component 1-10; exostoses (multiple)-like 1; exostoses (multiple)-like 2; exostoses (multiple)-like 3; exostosin 1 and 2; exportin 4-7; exportin, tRNA (nuclear export receptor for tRNAs); extended synaptotagmin-like protein 1; extended synaptotagmin-like protein 2; extended synaptotagmin-like protein 3; extracellular matrix protein 1; extracellular matrix protein 2, female organ and adipocyte specific; ezrin; F11 receptor; FAM18B2-CDRT4 readthrough; family with sequence similarity 102, member A; family with sequence similarity 107, member A; family with sequence similarity 108, member A1 and member B1; family with sequence similarity 109, member A and member B; family with sequence similarity 118, member A; family with sequence similarity 120A-120C; family with sequence similarity 123B; family with sequence similarity 125, member A-member B; family with sequence similarity 126, member A; family with sequence similarity 129, member A-member B; family with sequence similarity 13, member A; family with sequence similarity 131, member A; family with sequence similarity 132, member A; family with sequence similarity 132, member B; family with sequence similarity 134, member A-member C; family with sequence similarity 135, member A-member B; family with sequence similarity 135, member B; family with sequence similarity 150, member A-member B; family with sequence similarity 151, member A; family with sequence similarity 155, member A-member B; family with sequence similarity 156, member A-member B; family with sequence similarity 159, member A; family with sequence similarity 161, member A; family with sequence similarity 162, member A-member B; family with sequence similarity 163, member A-member B; family with sequence similarity 165, member A-member B; family with sequence similarity 167, member A; family with sequence similarity 171, member A1; family with sequence similarity 171, member A2; family with sequence similarity 171, member B; family with sequence similarity 172, member A; family with sequence similarity 173, member A; family with sequence similarity 173, member B; family with sequence similarity 174, member A; family with sequence similarity 174, member B; family with sequence similarity 175, member A; family with sequence similarity 175, member B; family with sequence similarity 176, member A; family with sequence similarity 176, member B; family with sequence similarity 178, member A; family with sequence similarity 18, member A; family with sequence similarity 18, member B1; family with sequence similarity 18, member B2; family with sequence similarity 180, member A; family with sequence similarity 180, member B; family with sequence similarity 187, member B; family with sequence similarity 188, member A; family with sequence similarity 189, member A1; family with sequence similarity 189, member A2; family with sequence similarity 189, member B; family with sequence similarity 19 (chemokine (C—C motif)-like) member A1, member A3, member A4 and member A5; family with sequence similarity 192, member A; family with sequence similarity 198, member A; family with sequence similarity 198, member B; family with sequence similarity 20, member A-member C; family with sequence similarity 200, member A; family with sequence similarity 205, member A; family with sequence similarity 209, member A-member B; family with sequence similarity 21, member A-member C; family with sequence similarity 210, member A-member B; family with sequence similarity 213, member A; family with sequence similarity 214, member B; family with sequence similarity 24, member B; family with sequence similarity 26, member D; family with sequence similarity 26, member E; family with sequence similarity 26, member F; family with sequence similarity 3, member A; family with sequence similarity 3, member B; family with sequence similarity 3, member C; family with sequence similarity 3, member D; family with sequence similarity 32, member A; family with sequence similarity 36, member A; family with sequence similarity 40, member A; family with sequence similarity 46, member C; family with sequence similarity 48, member A; family with sequence similarity 5, member B; family with sequence similarity 5, member C; family with sequence similarity 50, member A; family with sequence similarity 50, member B; family with sequence similarity 53, member A; family with sequence similarity 55, member A-member D; family with sequence similarity 57, member A; family with sequence similarity 57, member B; family with sequence similarity 58, member A; family with sequence similarity 64, member A; family with sequence similarity 65, member B; family with sequence similarity 69, member A; family with sequence similarity 69, member B; family with sequence similarity 69, member C; family with sequence similarity 70, member A; family with sequence similarity 70, member B; family with sequence similarity 71, member B; family with sequence similarity 73, member A; family with sequence similarity 73, member B; family with sequence similarity 74, member A1; family with sequence similarity 74, member A2; family with sequence similarity 75, member A1-member A7 and member D1; family with sequence similarity 8, member A1; family with sequence similarity 82, member A1; family with sequence similarity 82, member A2; family with sequence similarity 82, member B; family with sequence similarity 83, member H; family with sequence similarity 84, member B; family with sequence similarity 9, member A; family with sequence similarity 9, member B; family with sequence similarity 9, member C; family with sequence similarity 90, member A1; FANCD2/FANCI-associated nuclease 1; Fanconi anemia, complementation group A-group C, group D, group E-group G, group I, group L and group M; far upstream element (FUSE) binding protein 1 and protein 3; far upstream element (FUSE) binding protein 3; farnesyl diphosphate synthase; farnesyl-diphosphate farnesyltransferase 1; farnesyltransferase, CAAX box, alpha; farnesyltransferase, CAAX box, beta; Fas (TNF receptor superfamily, member 6); Fas (TNFRSF6) associated factor 1; Fas (TNFRSF6) binding factor 1; Fas (TNFRSF6)-associated via death domain; Fas apoptotic inhibitory molecule; Fas apoptotic inhibitory molecule 2; Fas apoptotic inhibitory molecule 3; Fas associated factor family member 2; Fas ligand (TNF superfamily, member 6); Fas-activated serine/threonine kinase; fasciculation and elongation protein zeta 1 (zygin I); fasciculation and elongation protein zeta 2 (zygin II); FAST kinase domains 1-domains 3 and domains 5; FAST kinase domains 2; FAST kinase domains 3; FAST kinase domains 5; fat mass and obesity associated; fat storage-inducing transmembrane protein 1; fat storage-inducing transmembrane protein 2; fatty acid 2-hydroxylase; fatty acid amide hydrolase; fatty acid amide hydrolase 2; fatty acid binding protein 1, liver; fatty acid binding protein 12; fatty acid binding protein 2, intestinal; fatty acid binding protein 3, muscle and heart (mammary-derived growth inhibitor); fatty acid binding protein 4, adipocyte; fatty acid binding protein 5 (psoriasis-associated); fatty acid binding protein 6, ileal; fatty acid binding protein 7, brain; fatty acid binding protein 9, testis; fatty acid desaturase 1; fatty acid desaturase 2; fatty acid desaturase 3; fatty acid desaturase domain family, member 6; fatty acid synthase; fatty acyl CoA reductase 1; fatty acyl CoA reductase 2; F-box and leucine-rich repeat protein 2-8, 12-20 and 22; F-box protein 2-11, 15-16, 21, 22, 24, 25, 27, 28, 30-34, 36, and 38-48; F-box protein, helicase, 18; F-box/LRR-repeat protein 22; Fc fragment of IgA, receptor for; Fc fragment of IgE, high affinity I, receptor for; alpha polypeptide; Fc fragment of IgE, high affinity I, receptor for; gamma polypeptide; Fc fragment of IgE, low affinity II, receptor for (CD23); Fc fragment of IgG binding protein; Fc fragment of IgG, high affinity Ia, receptor (CD64); Fc fragment of IgG, high affinity Ib, receptor (CD64); Fc fragment of IgG, low affinity IIa, receptor (CD32); Fc fragment of IgG, low affinity IIb, receptor (CD32); Fc fragment of IgG, low affinity Ma, receptor (CD16a); Fc fragment of IgG, low affinity Mb, receptor (CD16b); Fc fragment of IgG, receptor, transporter, alpha; Fc receptor, IgA, IgM, high affinity; Fc receptor-like 1-6, A and B; FCH and double SH3 domains 1; FCH and double SH3 domains 2; FCH domain only 1; FCH domain only 2; feline leukemia virus subgroup C cellular receptor 1; Feline leukemia virus subgroup C receptor-related protein 2; feline sarcoma oncogene; FERM, RhoGEF (ARHGEF) and pleckstrin domain protein 1 (chondrocyte-derived); FERM, RhoGEF and pleckstrin domain protein 2; fermitin family member 1; fermitin family member 2; fermitin family member 3; ferredoxin 1; ferredoxin 1-like; ferredoxin reductase; ferric-chelate reductase 1; ferritin mitochondrial; ferritin, heavy polypeptide 1; ferritin, heavy polypeptide-like 17; ferritin, light polypeptide; ferrochelatase; fetal and adult testis expressed 1; fetuin B; FEV (ETS oncogene family); FEZ family zinc finger 1; FEZ family zinc finger 2; FGFR1 oncogene partner; FGFR1 oncogene partner 2; FGFR1OP N-terminal like; FGGY carbohydrate kinase domain containing; fibrillarin; fibrillin 1; fibrillin 2; fibrillin 3; fibrinogen alpha chain; fibrinogen beta chain; fibrinogen gamma chain; fibrinogen-like 1; fibrinogen-like 2; fibroblast activation protein, alpha; fibroblast growth factor (acidic) intracellular binding protein; fibroblast growth factor 1 (acidic), 2 (basic), 3-7, 8 (androgen-induced), 9 (glia-activating factor), and 10-23; fibroblast growth factor binding protein 1-3; fibroblast growth factor receptor 1; fibroblast growth factor receptor 2; fibroblast growth factor receptor 3; fibroblast growth factor receptor 4; fibroblast growth factor receptor substrate 2; fibroblast growth factor receptor substrate 3; fibroblast growth factor receptor-like 1; fibromodulin; fibronectin 1; fibronectin leucine rich transmembrane protein 1; fibronectin leucine rich transmembrane protein 2; fibronectin leucine rich transmembrane protein 3; fibronectin type III and ankyrin repeat domains 1; fibrosin; fibrosin-like 1; fibrous sheath CABYR binding protein; fibrous sheath interacting protein 1; fibrous sheath interacting protein 2; fibulin 1, 2, 5 and 7; FIC domain containing; ficolin (collagen/fibrinogen domain containing) 1; ficolin (collagen/fibrinogen domain containing) 3 (Hakata antigen); fidgetin; fidgetin-like 1; filaggrin; filaggrin family member 2; filamin A interacting protein 1; filamin A interacting protein 1-like; filamin A, alpha; filamin B, beta; filamin binding LIM protein 1; filamin C, gamma; Finkel-Biskis-Reilly murine sarcoma virus (FBR-MuSV) ubiquitously expressed; FK506 binding protein 10, 65 kDa; FK506 binding protein 11, 19 kDa; FK506 binding protein 14, 22 kDa; FK506 binding protein 15, 133 kDa; FK506 binding protein 1A, 12 kDa; FK506 binding protein 1B, 12.6 kDa; FK506 binding protein 1C; FK506 binding protein 2, 13 kDa; FK506 binding protein 3, 25 kDa; FK506 binding protein 4, 59 kDa; FK506 binding protein 5; FK506 binding protein 6, 36 kDa; FK506 binding protein 7; FK506 binding protein 8, 38 kDa; FK506 binding protein 9, 63 kDa; FK506 binding protein like; flap structure-specific endonuclease 1; flotillin 1; flotillin 2; FLT3-interacting zinc finger 1; FLYWCH family member 2; FLYWCH-type zinc finger 1; fms-related tyrosine kinase 1 (vascular endothelial growth factor/vascular permeability factor receptor); fms-related tyrosine kinase 3; fms-related tyrosine kinase 3 ligand; fms-related tyrosine kinase 4; folate hydrolase (prostate-specific membrane antigen) 1; folate receptor 1 (adult); folate receptor 2 (fetal); folate receptor 3 (gamma); follicle stimulating hormone receptor; follicle stimulating hormone, beta polypeptide; follicular dendritic cell secreted protein; folliculin; folliculin interacting protein 1; folliculin interacting protein 2; folliculogenesis specific basic helix-loop-helix; follistatin; follistatin-like 1; follistatin-like 3 (secreted glycoprotein); follistatin-like 4; follistatin-like 5; folylpolyglutamate synthase; forkhead box A1, A2, A3, B1, B2, C1, C2, D2, D3, D4, D4-like 1, D4-like 2, D4-like 3, D4-like 4, D1-like 5, D4-like 6, E1, E3, F1, F2, G1, H1, I1, I2, J1, J2, J3, K1, K2, L1, L2, Ml, N1, N2, N3, N4, O1, O3, O4, O6, P1, P2, P3, P4, Q1, R1, R2 and S1; forkhead-associated (FHA) phosphopeptide binding domain 1; formiminotransferase cyclodeaminase; formin 1; formin 2; formin binding protein 1; formin binding protein 1-like; formin binding protein 4; formin-like 1; formin-like 2; formin-like 3; formyl peptide receptor 1; formyl peptide receptor 2; formyl peptide receptor 3; FOS-like antigen 1; FOS-like antigen 2; four and a half LIM domains 1; four and a half LIM domains 2; four and a half LIM domains 3; four and a half LIM domains 5; FPGT-TNNI3K fusion protein isoform a; FPGT-TNNI3K readthrough; fragile histidine triad gene; fragile site, folic acid type, rare, fra(10)(q23.3) and fra(10)(q24.2) candidate 1; fragile X mental retardation 1; fragile X mental retardation 1 neighbor; FRAS1 related extracellular matrix 1; FRAS1 related extracellular matrix protein 2; Fraser syndrome 1; frataxin; free fatty acid receptor 1; free fatty acid receptor 2; free fatty acid receptor 3; frequently rearranged in advanced T-cell lymphomas; frequently rearranged in advanced T-cell lymphomas 2; Friend leukemia virus integration 1; frizzled family receptor 1-10; frizzled-related protein; fructosamine 3 kinase; fructosamine 3 kinase related protein; fructose-1,6-bisphosphatase 1; fructose-1,6-bisphosphatase 2; FRY-like; FSHD region gene 1; FSHD region gene 1 family, member B; FSHD region gene 2; FSHD region gene 2 family, member B; FSHD region gene 2 family, member C; fucokinase; fucose-1-phosphate guanylyltransferase; fucosidase, alpha-L-1, tissue; fucosidase, alpha-L-2, plasma; fucosyltransferase 1 (galactoside 2-alpha-L-fucosyltransferase, H blood group); fucosyltransferase 10 (alpha (1,3) fucosyltransferase); fucosyltransferase 11 (alpha (1,3) fucosyltransferase); fucosyltransferase 2 (secretor status included); fucosyltransferase 3 (galactoside 3(4)-L-fucosyltransferase, Lewis blood group); fucosyltransferase 4 (alpha (1,3) fucosyltransferase, myeloid-specific); fucosyltransferase 5 (alpha (1,3) fucosyltransferase); fucosyltransferase 6 (alpha (1,3) fucosyltransferase); fucosyltransferase 7 (alpha (1,3) fucosyltransferase); fucosyltransferase 8 (alpha (1,6) fucosyltransferase); fucosyltransferase 9 (alpha (1,3) fucosyltransferase); fukutin; fukutin related protein; Full-length cDNA 5-PRIME end of clone CS0CAP004Y005 of Thymus of Homo sapiens (human); Full-length cDNA clone CSODK012YO09 of HeLa cells of Homo sapiens (human); fumarate hydratase; fumarylacetoacetate hydrolase (fumarylacetoacetase); furin (paired basic amino acid cleaving enzyme); fused in sarcoma; FYN binding protein; FYN oncogene related to SRC, FGR, YES; fyn-related kinase; G antigen 1, 2A, 2B, 2C, 2D, 2E, 4, 10, 12B, 12C, 12D, 12E, 12F, 12G, 12H, 12I, 12J, and 13; G elongation factor, mitochondrial 1; G elongation factor, mitochondrial 2; G kinase anchoring protein 1; G patch domain and ankyrin repeats 1; G patch domain and KOW motifs; G protein pathway suppressor 1; G protein pathway suppressor 2; G protein regulated inducer of neurite outgrowth 1; G protein regulated inducer of neurite outgrowth 2; G protein-coupled bile acid receptor 1; G protein-coupled estrogen receptor 1; G protein-coupled receptor 1, 3, 4, 6, 12, 15, 17, 18, 19, 20, 21, 22, 25, 26, 27, 31, 32, 34, 35, 37 (endothelin receptor type B-like), 37 like 1, 39, 45, 50, 52, 55, 56, 61, 62, 63, 64, 65, 68, 75, 77, 78, 82, 83, 84, 85, 87, 88, 89A, 89B, 89C, 97, 98, 101, 107, 108, 110, 111, 112, 113, 114, 115, 116, 119, 123, 124, 125, 126, 128, 132, 133, 135, 137, 139, 141, 142, 143, 144, 146, 148, 149, 150, 151, 152, 153, 155, 156, 157, 158, 160, 161, 162, 171, 173, 174, 176, 179, 180, 182, 183, 137B, 137C, 172A, and 172B; G protein-coupled receptor associated sorting protein 1 and 2; G protein-coupled receptor associated sorting protein 2; G protein-coupled receptor kinase 1; G protein-coupled receptor kinase 4; G protein-coupled receptor kinase 5; G protein-coupled receptor kinase 6; G protein-coupled receptor kinase 7; G protein-coupled receptor kinase interacting ArfGAP 1; G protein-coupled receptor kinase interacting ArfGAP 2; G protein-coupled receptor, family C, group 5, member A; G protein-coupled receptor, family C, group 5, member B; G protein-coupled receptor, family C, group 5, member C; G protein-coupled receptor, family C, group 5, member D; G protein-coupled receptor, family C, group 6, member A; G0/G1 switch 2; G1 to S phase transition 1; G1 to S phase transition 2; G-2 and S-phase expressed 1; G2/M-phase specific E3 ubiquitin protein ligase; GA binding protein transcription factor, alpha subunit 60 kDa; GA binding protein transcription factor, beta subunit 1; GA binding protein transcription factor, beta subunit 2; GABA(A) receptor-associated protein; GABA(A) receptor-associated protein like 1; GABA(A) receptor-associated protein-like 2; galactokinase 1; galactokinase 2; galactosamine (N-acetyl)-6-sulfate sulfatase; galactose mutarotase (aldose 1-epimerase); galactose-1-phosphate uridylyltransferase; galactose-3-O-sulfotransferase 1; galactose-3-O-sulfotransferase 2; galactose-3-O-sulfotransferase 3; galactose-3-O-sulfotransferase 4; galactosidase, alpha; galactosidase, beta 1; galactosidase, beta 1-like; galactosidase, beta 1-like 2; galactosidase, beta 1-like 3; galactosylceramidase; galanin prepropeptide; galanin receptor 1; galanin receptor 2; galanin receptor 3; galanin-like peptide; gametocyte specific factor 1; gametocyte specific factor 1-like; gametogenetin; gametogenetin binding protein 1; gametogenetin binding protein 2; gamma-aminobutyric acid (GABA) A receptor, alpha 1-alpha 6, beta 1-beta 3, delta, epsilon, gamma 1-gamma 3 and pi; gamma-aminobutyric acid (GABA) B receptor, 1; gamma-aminobutyric acid (GABA) B receptor, 2; gamma-aminobutyric acid (GABA) receptor, rho 1; gamma-aminobutyric acid (GABA) receptor, rho 2; gamma-aminobutyric acid (GABA) receptor, theta; gamma-glutamyl carboxylase; gamma-glutamyl hydrolase (conjugase, folylpolygammaglutamyl hydrolase); gamma-glutamylcyclotransferase; gamma-glutamyltransferase 1; gamma-glutamyltransferase 2; gamma-glutamyltransferase 5; gamma-glutamyltransferase 6; gamma-glutamyltransferase 7; gamma-glutamyltransferase light chain 1; gamma-glutamyltransferase light chain 2; gamma-glutamyltransferase light chain 3; ganglioside induced differentiation associated protein 2; ganglioside-induced differentiation-associated protein 1; ganglioside-induced differentiation-associated protein 1-like 1; gap junction protein, alpha 1, 43 kDa; gap junction protein, alpha 10, 62 kDa; gap junction protein, alpha 3, 46 kDa; gap junction protein, alpha 4, 37 kDa; gap junction protein, alpha 5, 40 kDa; gap junction protein, alpha 8, 50 kDa; gap junction protein, alpha 9, 59 kDa; gap junction protein, beta 1, 32 kDa; gap junction protein, beta 2, 26 kDa; gap junction protein, beta 3, 31 kDa; gap junction protein, beta 4, 30.3 kDa; gap junction protein, beta 5, 31.1 kDa; gap junction protein, beta 6, 30 kDa; gap junction protein, beta 7, 25 kDa; gap junction protein, delta 2, 36 kDa; gap junction protein, delta 3, 31.9 kDa; gap junction protein, delta 4, 40.1 kDa; gap junction protein, gamma 1, 45 kDa; gap junction protein, gamma 2, 47 kDa; gap junction protein, gamma 3, 30.2 kDa; gasdermin A-D; gastric inhibitory polypeptide; gastric inhibitory polypeptide receptor; gastric intrinsic factor (vitamin B synthesis); gastrin; gastrin-releasing peptide; gastrin-releasing peptide receptor; gastrokine 1; gastrokine 2; gastrulation brain homeobox 1; gastrulation brain homeobox 2; GATA binding protein 1 (globin transcription factor 1); GATA binding protein 2; GATA binding protein 3; GATA binding protein 4; GATA binding protein 5; GATA binding protein 6; GATS protein-like 1; GATS protein-like 2; GATS protein-like 3; GATS, stromal antigen 3 opposite strand; GCN1 general control of amino-acid synthesis 1-like 1 (yeast); GC-rich promoter binding protein 1; GC-rich promoter binding protein 1-like 1; GC-rich sequence DNA-binding factor 1; GC-rich sequence DNA-binding factor 2; GDNF family receptor alpha 1; GDNF family receptor alpha 2; GDNF family receptor alpha 3; GDNF family receptor alpha 4; GDNF family receptor alpha like; GDNF-inducible zinc finger protein 1; GDP dissociation inhibitor 1; GDP dissociation inhibitor 2; GDP-mannose 4,6-dehydratase; GDP-mannose pyrophosphorylase A; GDP-mannose pyrophosphorylase B; gelsolin; gem (nuclear organelle) associated protein 2, 4, 5, 6, 7 and 8; GEM interacting protein; geminin coiled-coil domain containing; geminin, DNA replication inhibitor; general transcription factor IIA, 1, 19/37 kDa; general transcription factor IIA, 1-like; general transcription factor IIA, 2, 12 kDa; general transcription factor IIB; general transcription factor IIE, polypeptide 1, alpha 56 kDa; general transcription factor IIE, polypeptide 2, beta 34 kDa; general transcription factor IIF, polypeptide 1, 74 kDa; general transcription factor IIF, polypeptide 2, 30 kDa; general transcription factor IIH, polypeptide 1, 62 kDa; general transcription factor IIH, polypeptide 2, 44 kDa; general transcription factor IIH, polypeptide 2C; general transcription factor IIH, polypeptide 3, 34 kDa; general transcription factor IIH, polypeptide 4, 52 kDa; general transcription factor IIH, polypeptide 5; general transcription factor IIi; general transcription factor IIIA; general transcription factor IIIC, polypeptide 1, alpha 220 kDa; general transcription factor IIIC, polypeptide 2, beta 110 kDa; general transcription factor IIIC, polypeptide 3, 102 kDa; general transcription factor IIIC, polypeptide 4, 90 kDa; general transcription factor IIIC, polypeptide 5, 63 kDa; general transcription factor IIIC, polypeptide 6, alpha 35 kDa; gephyrin; geranylgeranyl diphosphate synthase 1; germ cell associated 1; germ cell associated 2 (haspin); germinal center expressed transcript 2; GH3 domain containing; ghrelin/obestatin prepropeptide; gigaxonin; GLI family zinc finger 1; GLI family zinc finger 2; GLI family zinc finger 3; GLI family zinc finger 4; GLI pathogenesis-related 1; GLI pathogenesis-related 1 like 1; GLI pathogenesis-related 1 like 2; GLI pathogenesis-related 2; glia maturation factor, beta; glia maturation factor, gamma; glial cell derived neurotrophic factor; glial fibrillary acidic protein; glioblastoma amplified sequence; glioma tumor suppressor candidate region gene 1; glioma tumor suppressor candidate region gene 2; gliomedin; GLIS family zinc finger 1; GLIS family zinc finger 2; GLIS family zinc finger 3; globoside alpha-1,3-N-acetylgalactosaminyltransferase 1; glomulin, FKBP associated protein; glucagon; glucagon receptor; glucagon-like peptide 1 receptor; glucagon-like peptide 2 receptor; glucan (1,4-alpha-), branching enzyme 1; glucocorticoid induced transcript 1; glucocorticoid modulatory element binding protein 1; glucocorticoid modulatory element binding protein 2; glucokinase (hexokinase 4); glucokinase (hexokinase 4) regulator; glucosamine (N-acetyl)-6-sulfatase; glucosamine (UDP-N-acetyl)-2-epimerase/N-acetylmannosamine kinase; glucosamine-6-phosphate deaminase 1; glucosamine-6-phosphate deaminase 2; glucosamine-phosphate N-acetyltransferase 1; glucosaminyl (N-acetyl) transferase 1, core 2; glucosaminyl (N-acetyl) transferase 2, I-branching enzyme (I blood group); glucosaminyl (N-acetyl) transferase 3, mucin type; glucosaminyl (N-acetyl) transferase 4, core 2; glucosaminyl (N-acetyl) transferase 6; glucosaminyl (N-acetyl) transferase family member 7; glucose 6 phosphatase, catalytic, 3; glucose-6-phosphatase, catalytic subunit; glucose-6-phosphatase, catalytic, 2; glucose-6-phosphate dehydrogenase; glucose-6-phosphate isomerase; glucosidase, alpha; acid; glucosidase, alpha; neutral AB; glucosidase, alpha; neutral C; glucosidase, beta (bile acid) 2; glucosidase, beta, acid; glucoside xylosyltransferase 1; glucoside xylosyltransferase 2; glucuronic acid epimerase; glucuronidase, beta; glutamate decarboxylase 1 (brain, 67 kDa); glutamate decarboxylase 2 (pancreatic islets and brain, 65 kDa); glutamate decarboxylase-like 1; glutamate dehydrogenase 1; glutamate dehydrogenase 2; glutamate receptor interacting protein 1; glutamate receptor, ionotrophic, AMPA 3; glutamate receptor, ionotrophic, AMPA 4; glutamate receptor, ionotropic, AMPA 1; glutamate receptor, ionotropic, AMPA 2; glutamate receptor, ionotropic, delta 1; glutamate receptor, ionotropic, delta 2; glutamate receptor, ionotropic, delta 2 (Grid2) interacting protein; glutamate receptor, ionotropic, kainate 1; glutamate receptor, ionotropic, kainate 2; glutamate receptor, ionotropic, kainate 3; glutamate receptor, ionotropic, kainate 4; glutamate receptor, ionotropic, kainate 5; glutamate receptor, ionotropic, N-methyl D-aspartate 1; glutamate receptor, ionotropic, N-methyl D-aspartate 2A; glutamate receptor, ionotropic, N-methyl D-aspartate 2B; glutamate receptor, ionotropic, N-methyl D-aspartate 2C; glutamate receptor, ionotropic, N-methyl D-aspartate 2D; glutamate receptor, ionotropic, N-methyl D-aspartate-associated protein 1 (glutamate binding); glutamate receptor, ionotropic, N-methyl-D-aspartate 3A; glutamate receptor, ionotropic, N-methyl-D-aspartate 3B; glutamate receptor, metabotropic 1-8; glutamate-ammonia ligase; glutamate-cysteine ligase, catalytic subunit; glutamate-cysteine ligase, modifier subunit; glutamate-rich 1; glutamic pyruvate transaminase (alanine aminotransferase) 2; glutamic-oxaloacetic transaminase 1, soluble (aspartate aminotransferase 1); glutamic-oxaloacetic transaminase 1-like 1; glutamic-oxaloacetic transaminase 2, mitochondrial (aspartate aminotransferase 2); glutamic-pyruvate transaminase (alanine aminotransferase); glutaminase; glutaminase 2 (liver, mitochondrial); glutamine and serine rich 1; glutamine rich 2; glutamine-fructose-6-phosphate transaminase 1; glutamine-fructose-6-phosphate transaminase 2; glutamine-rich 1; glutaminyl-peptide cyclotransferase; glutaminyl-peptide cyclotransferase-like; glutaminyl-tRNA synthase (glutamine-hydrolyzing)-like 1; glutaminyl-tRNA synthetase; glutamyl aminopeptidase (aminopeptidase A); glutamyl-prolyl-tRNA synthetase; Glutamyl-tRNA(Gln) amidotransferase subunit C, mitochondrial; glutaredoxin (thioltransferase); glutaredoxin 2; glutaredoxin 3; glutaredoxin 5; glutaredoxin, cysteine rich 1; glutaredoxin, cysteine rich 2; glutaryl-CoA dehydrogenase; glutathione peroxidase 1; glutathione peroxidase 2 (gastrointestinal); glutathione peroxidase 3 (plasma); glutathione peroxidase 4 (phospholipid hydroperoxidase); glutathione peroxidase 5 (epididymal androgen-related protein); glutathione peroxidase 6 (olfactory); glutathione peroxidase 7; glutathione reductase; glutathione S-transferase alpha 1; glutathione S-transferase alpha 2; glutathione S-transferase alpha 3; glutathione S-transferase alpha 4; glutathione S-transferase alpha 5; glutathione S-transferase kappa 1; glutathione S-transferase mu 1; glutathione S-transferase mu 2 (muscle); glutathione S-transferase mu 3 (brain); glutathione S-transferase mu 4; glutathione S-transferase mu 5; glutathione S-transferase omega 1; glutathione S-transferase omega 2; glutathione S-transferase pi 1; glutathione S-transferase theta 1; glutathione S-transferase theta 2; glutathione S-transferase, C-terminal domain containing; glutathione synthetase; glutathione transferase zeta 1; glyceraldehyde-3-phosphate dehydrogenase; glyceraldehyde-3-phosphate dehydrogenase, spermatogenic; glycerate kinase; glycerol kinase; glycerol kinase 2; glycerol-3-phosphate acyltransferase 2, mitochondrial; glycerol-3-phosphate acyltransferase, mitochondrial; glycerol-3-phosphate dehydrogenase 1 (soluble); glycerol-3-phosphate dehydrogenase 1-like; glycerol-3-phosphate dehydrogenase 2 (mitochondrial); glyceronephosphate O-acyltransferase; glycerophosphodiester phosphodiesterase 1; glycine amidinotransferase (L-arginine: glycine amidinotransferase); glycine C-acetyltransferase; glycine cleavage system protein H (aminomethyl carrier); glycine dehydrogenase (decarboxylating); glycine N-methyltransferase; glycine receptor, alpha 1; glycine receptor, alpha 2; glycine receptor, alpha 3; glycine receptor, alpha 4; glycine receptor, beta; glycine-N-acyltransferase; glycine-N-acyltransferase-like 1; glycine-N-acyltransferase-like 2; glycine-N-acyltransferase-like 3; glycogen synthase 1 (muscle); glycogen synthase 2 (liver); glycogen synthase kinase 3 alpha; glycogen synthase kinase 3 beta; glycogenin 1; glycogenin 2; glycolipid transfer protein; glycophorin A (MNS blood group); glycophorin B (MNS blood group); glycophorin C (Gerbich blood group); glycophorin E (MNS blood group); glycoprotein (transmembrane) nmb; glycoprotein 2 (zymogen granule membrane); glycoprotein A33 (transmembrane); glycoprotein hormone alpha 2; glycoprotein hormones, alpha polypeptide; glycoprotein Ib (platelet), alpha polypeptide; glycoprotein Ib (platelet), beta polypeptide; glycoprotein IX (platelet); glycoprotein M6A; glycoprotein M6B; glycoprotein V (platelet); glycoprotein VI (platelet); glycosylphosphatidylinositol anchored high density lipoprotein binding protein 1; glycosylphosphatidylinositol anchored molecule like protein; glycosylphosphatidylinositol specific phospholipase D1; glycosyltransferase-like 1B; glycyl-tRNA synthetase; glyoxalase I; glyoxylate reductase/hydroxypyruvate reductase; glypican 1; glypican 2; glypican 3; glypican 4; glypican 5; glypican 6; GM2 ganglioside activator; GNAS complex locus; golgi brefeldin A resistant guanine nucleotide exchange factor 1; golgi glycoprotein 1; golgi integral membrane protein 4; golgi membrane protein 1; golgi phosphoprotein 3 (coat-protein); golgi phosphoprotein 3-like; golgi reassembly stacking protein 1, 65 kDa; golgi reassembly stacking protein 2, 55 kDa; golgi SNAP receptor complex member 1; golgi SNAP receptor complex member 2; golgi transport 1A; golgi transport 1B; golgi-associated PDZ and coiled-coil motif containing; golgi-associated, gamma adaptin ear containing, ARF binding protein 1-3; golgin A1-A5, A7 and B1; golgin A6 family, member A-member D; golgin A6 family-like 1, 2, 4, 6, 9 and 10; golgin A7 family, member B; golgin A8 family, member A; golgin A8 family, member B; golgin A8 family, member J; Golgin subfamily A member 6-like protein 9; Golgin subfamily A member 8-like protein 2; gonadotropin-releasing hormone (type 2) receptor 2; gonadotropin-releasing hormone 1 (luteinizing-releasing hormone); gonadotropin-releasing hormone 2; gonadotropin-releasing hormone receptor; goosecoid homeobox; goosecoid homeobox 2; GPN-loop GTPase 1; GPN-loop GTPase 2; GPN-loop GTPase 3; GPRIN family member 3; G-protein signaling modulator 1; G-protein signaling modulator 2; G-protein signaling modulator 3; grancalcin, EF-hand calcium binding protein; granulin; granulysin; granzyme A (granzyme 1, cytotoxic T-lymphocyte-associated serine esterase 3); granzyme B (granzyme 2, cytotoxic T-lymphocyte-associated serine esterase 1); granzyme H (cathepsin G-like 2, protein h-CCPX); granzyme K (granzyme 3; tryptase II); granzyme M (lymphocyte met-ase 1); GRB10 interacting GYF protein 1; GRB10 interacting GYF protein 2; GRB2-associated binding protein 1; GRB2-associated binding protein 2; GRB2-associated binding protein 3; GRB2-associated binding protein family, member 4; GRB2-binding adaptor protein, transmembrane; GRB2-related adapter protein-like; GRB2-related adaptor protein; GRB2-related adaptor protein 2; gremlin 1; gremlin 2; G-rich RNA sequence binding factor 1; GRINL1A complex locus 1; GRIP1 associated protein 1; group-specific component (vitamin D binding protein); growth arrest and DNA-damage-inducible, alpha; growth arrest and DNA-damage-inducible, beta; growth arrest and DNA-damage-inducible, gamma; growth arrest and DNA-damage-inducible, gamma interacting protein 1; growth arrest-specific 1; growth arrest-specific 2; growth arrest-specific 2 like 1; growth arrest-specific 2 like 2; growth arrest-specific 2 like 3; growth arrest-specific 6; growth arrest-specific 7; growth arrest-specific 8; growth associated protein 43; growth differentiation factor 1, 2, 3, 5, 5 opposite strand, 6, 7, 9, 10, 11 and 15; growth factor independent 1 transcription repressor; growth factor independent 1B transcription repressor; growth factor receptor-bound protein 10; growth factor receptor-bound protein 14; growth factor receptor-bound protein 2; growth factor receptor-bound protein 7; growth factor, augmenter of liver regeneration; growth hormone 1; growth hormone 2; growth hormone inducible transmembrane protein; growth hormone receptor; growth hormone regulated TBC protein 1; growth hormone releasing hormone; growth hormone releasing hormone receptor; growth hormone secretagogue receptor; growth regulation by estrogen in breast cancer 1; growth regulation by estrogen in breast cancer-like; GRP1 (general receptor for phosphoinositides 1)-associated scaffold protein; GS homeobox 1; GS homeobox 2; GSG1-like; GTP binding protein 1; GTP binding protein 2; GTP binding protein 3 (mitochondrial); GTP binding protein 4; GTP binding protein overexpressed in skeletal muscle; GTP cyclohydrolase 1; GTP cyclohydrolase I feedback regulator; GTPase activating protein (SH3 domain) binding protein 1; GTPase activating protein (SH3 domain) binding protein 2; GTPase activating protein and VPS9 domains 1; GTPase activating Rap/RanGAP domain-like 3; GTPase, IMAP family member 1, member 2, and member 4-member 8; guanidinoacetate N-methyltransferase; guanine deaminase; guanine monphosphate synthetase; guanine nucleotide binding protein (G protein) alpha 12; guanine nucleotide binding protein (G protein), alpha 11 (Gq class); guanine nucleotide binding protein (G protein), alpha 13; guanine nucleotide binding protein (G protein), alpha 14; guanine nucleotide binding protein (G protein), alpha 15 (Gq class); guanine nucleotide binding protein (G protein), alpha activating activity polypeptide 0; guanine nucleotide binding protein (G protein), alpha activating activity polypeptide, olfactory type; guanine nucleotide binding protein (G protein), alpha inhibiting activity polypeptide 1; guanine nucleotide binding protein (G protein), alpha inhibiting activity polypeptide 2; guanine nucleotide binding protein (G protein), alpha inhibiting activity polypeptide 3; guanine nucleotide binding protein (G protein), alpha transducing activity polypeptide 1; guanine nucleotide binding protein (G protein), alpha transducing activity polypeptide 2; guanine nucleotide binding protein (G protein), alpha z polypeptide; guanine nucleotide binding protein (G protein), beta 5; guanine nucleotide binding protein (G protein), beta polypeptide 1; guanine nucleotide binding protein (G protein), beta polypeptide 1-like; guanine nucleotide binding protein (G protein), beta polypeptide 2; guanine nucleotide binding protein (G protein), beta polypeptide 2-like 1; guanine nucleotide binding protein (G protein), beta polypeptide 3; guanine nucleotide binding protein (G protein), beta polypeptide 4; guanine nucleotide binding protein (G protein), gamma 10; guanine nucleotide binding protein (G protein), gamma 11; guanine nucleotide binding protein (G protein), gamma 12; guanine nucleotide binding protein (G protein), gamma 13; guanine nucleotide binding protein (G protein), gamma 2; guanine nucleotide binding protein (G protein), gamma 3; guanine nucleotide binding protein (G protein), gamma 4; guanine nucleotide binding protein (G protein), gamma 5; guanine nucleotide binding protein (G protein), gamma 7; guanine nucleotide binding protein (G protein), gamma 8; guanine nucleotide binding protein (G protein), gamma transducing activity polypeptide 1; guanine nucleotide binding protein (G protein), gamma transducing activity polypeptide 2; guanine nucleotide binding protein (G protein), q polypeptide; guanine nucleotide binding protein, alpha transducing 3; guanine nucleotide binding protein-like 1; guanine nucleotide binding protein-like 2 (nucleolar); guanine nucleotide binding protein-like 3 (nucleolar); guanine nucleotide binding protein-like 3 (nucleolar)-like; guanosine monophosphate reductase; guanosine monophosphate reductase 2; guanylate binding protein 1, interferon-inducible; guanylate binding protein 2, interferon-inducible; guanylate binding protein 3; guanylate binding protein 4; guanylate binding protein 5; guanylate binding protein 7; guanylate binding protein family, member 6; guanylate cyclase 1, soluble, alpha 2; guanylate cyclase 1, soluble, alpha 3; guanylate cyclase 1, soluble, beta 3; guanylate cyclase 2C (heat stable enterotoxin receptor); guanylate cyclase 2D, membrane (retina-specific); guanylate cyclase 2F, retinal; guanylate cyclase activator 1A (retina); guanylate cyclase activator 1B (retina); guanylate cyclase activator 1C; guanylate cyclase activator 2A (guanylin); guanylate cyclase activator 2B (uroguanylin); guanylate kinase 1; gypsy retrotransposon integrase 1; H1 histone family, member 0; H1 histone family, member N, testis-specific; H1 histone family, member 0, oocyte-specific; H1 histone family, member X; H2.0-like homeobox; H2A histone family, member B1-member B3, member J, member V, member X, member Y, member Y2 and member Z; H2B histone family, member M; H2B histone family, member W, testis-specific; H3 histone, family 3A; H3 histone, family 3B (H3.3B); H3 histone, family 3C; H6 family homeobox 1; H6 family homeobox 2; H6 family homeobox 3; hairy/enhancer-of-split related with YRPW motif 1; hairy/enhancer-of-split related with YRPW motif 2; hairy/enhancer-of-split related with YRPW motif-like; haptoglobin; haptoglobin-related protein; harakiri, BCL2 interacting protein (contains only BH3 domain); harbinger transposase derived 1; HAUS augmin-like complex, subunit 1-subunit 8; HCLS1 associated protein X-1; HCLS1 binding protein 3; heart and neural crest derivatives expressed 1; heart and neural crest derivatives expressed 2; HEAT repeat family member 7B2; heat shock 105 kDa/110 kDa protein 1; heat shock 10 kDa protein 1 (chaperonin 10); heat shock 22 kDa protein 8; heat shock 27 kDa protein 1; heat shock 27 kDa protein 3; heat shock 27 kDa protein family, member 7 (cardiovascular); heat shock 60 kDa protein 1 (chaperonin); heat shock 70 kD protein 12B; heat shock 70 kDa protein 12A; heat shock 70 kDa protein 14; heat shock 70 kDa protein 1A; heat shock 70 kDa protein 1B; heat shock 70 kDa protein 1-like; heat shock 70 kDa protein 2; heat shock 70 kDa protein 4; heat shock 70 kDa protein 4-like; heat shock 70 kDa protein 5 (glucose-regulated protein, 78 kDa); heat shock 70 kDa protein 6 (HSP70B′); heat shock 70 kDa protein 8; heat shock 70 kDa protein 9 (mortalin); heat shock factor binding protein 1; heat shock factor binding protein 1-like 1; heat shock protein 70 kDa family, member 13; heat shock protein 90 kDa alpha (cytosolic), class A member 1; heat shock protein 90 kDa alpha (cytosolic), class B member 1; heat shock protein 90 kDa beta (Grp94), member 1; Heat shock protein beta-2; heat shock protein family B (small), member 11; heat shock protein, alpha-crystallin-related, B6; heat shock protein, alpha-crystallin-related, B9; heat shock transcription factor 1; heat shock transcription factor 2; heat shock transcription factor 2 binding protein; heat shock transcription factor 4; heat shock transcription factor family member 5; heat shock transcription factor family, X linked 1; heat shock transcription factor family, X linked 2; heat shock transcription factor, Y linked 2; heat shock transcription factor, Y-linked 1; heat-responsive protein 12; HECT domain and ankyrin repeat containing, E3 ubiquitin protein ligase 1; hect domain and RLD 2-6; hedgehog acyltransferase; hedgehog acyltransferase-like; hedgehog interacting protein; helicase (DNA) B; helicase with zinc finger; helicase, lymphoid-specific; helicase, POLQ-like; helicase-like transcription factor; helt bHLH transcription factor; hematological and neurological expressed 1; hematological and neurological expressed 1-like; hematopoietic cell signal transducer; hematopoietic cell-specific Lyn substrate 1; hematopoietic prostaglandin D synthase; hematopoietic SH2 domain containing; hematopoietically expressed homeobox; heme binding protein 1; heme binding protein 2; heme oxygenase (decycling) 1; heme oxygenase (decycling) 2; hemicentin 1; hemicentin 2; Hemicentin-2; HemK methyltransferase family member 1; hemochromatosis; hemochromatosis type 2 (juvenile); hemogen; hemoglobin, alpha 1; hemoglobin, alpha 2; hemoglobin, beta; hemoglobin, delta; hemoglobin, epsilon 1; hemoglobin, gamma A; hemoglobin, gamma G; hemoglobin, mu; hemoglobin, theta 1; hemoglobin, zeta; hemopexin; hemopoietic cell kinase; HEPACAM family member 2; heparan sulfate (glucosamine) 3-O-sulfotransferase 1, 2, 3A1, 3B1, 4, 5 and 6; heparan sulfate 2-O-sulfotransferase 1; heparan sulfate 6-O-sulfotransferase 1, 2 and 3; heparan sulfate proteoglycan 2; heparan-alpha-glucosaminide N-acetyltransferase; heparanase; heparanase 2; heparin-binding EGF-like growth factor; hepatic and glial cell adhesion molecule; hepatic leukemia factor; hepatitis A virus cellular receptor 1; hepatitis A virus cellular receptor 2; hepatitis B virus x interacting protein; hepatocellular carcinoma, down-regulated 1; Hepatocellular carcinoma-associated antigen HCA25a; hepatocyte growth factor (hepapoietin A; scatter factor); hepatocyte growth factor-regulated tyrosine kinase substrate; hepatocyte nuclear factor 4, alpha; hepatocyte nuclear factor 4, gamma; hepatoma derived growth factor-like 1; hepatoma-derived growth factor; Hepatoma-derived growth factor-related protein 2; Hepatoma-derived growth factor-related protein 3; hepcidin antimicrobial peptide; hephaestin; hephaestin-like 1; hepsin; Hermansky-Pudlak syndrome 1, 3, 4, 5 and 6; HERPUD family member 2; HERV-H LTR-associating 1; HERV-H LTR-associating 2; HERV-H LTR-associating 3; HESX homeobox 1; heterochromatin protein 1, binding protein 3; heterogeneous nuclear ribonucleoprotein A/B, A0, A1, A1-like 2, A2/B1, A3, C (C1/C2), C-like 1, D (AU-rich element RNA binding protein 1, 37 kDa), D-like, F, H1 (H), H2 (H′), H3 (2H9), K, L, L-like, M, R, U (scaffold attachment factor A), U-like 1, and U-like 2; hexamethylene bis-acetamide inducible 1; hexamethylene bis-acetamide inducible 2; hexokinase 1; hexokinase 2; hexokinase 3 (white cell); hexosaminidase (glycosyl hydrolase family 20, catalytic domain) containing; hexosaminidase A (alpha polypeptide); hexosaminidase B (beta polypeptide); hexose-6-phosphate dehydrogenase (glucose 1-dehydrogenase); HGF activator; HHIP-like 1; HHIP-like 2; HIG1 hypoxia inducible domain family, member 1A-member 1C, member 2A and member 2B; high density lipoprotein binding protein; high mobility group 20A; high mobility group 20B; high mobility group AT-hook 1; high mobility group AT-hook 2; high mobility group box 1; high mobility group box 2; high mobility group box 3; high mobility group box 4; high mobility group nucleosomal binding domain 2; high mobility group nucleosomal binding domain 3; high mobility group nucleosomal binding domain 4; high mobility group nucleosome binding domain 1; high mobility group nucleosome binding domain 5; highly divergent homeobox; hippocalcin; hippocalcin like 4; hippocalcin-like 1; hippocampus abundant transcript 1; hippocampus abundant transcript-like 1; hippocampus abundant transcript-like 2; HIRA interacting protein 3; histamine N-methyltransferase; histamine receptor H1; histamine receptor H2; histamine receptor H3; histamine receptor H4; histatin 1; histatin 3; histidine ammonia-lyase; histidine decarboxylase; histidine rich calcium binding protein; histidine rich carboxyl terminus 1; histidine triad nucleotide binding protein 1; histidine triad nucleotide binding protein 2; histidine triad nucleotide binding protein 3; histidine-rich glycoprotein; histidyl-tRNA synthetase; histo-blood group ABO system transferase; histocompatibility (minor) 13; histocompatibility (minor) HA-1; histocompatibility (minor) HB-1; histocompatibility (minor) serpin domain containing; histone acetyltransferase 1; histone cluster 1 H1a, H1b, H1c, H1d, H1e, H1t, H2aa, H2ab, H2ac, H2ad, H2ae, H2ag, H2ah, H2ai, H2aj, H2ak, H2al, H2am, H2ba, H2bb, H2bc, H2bd, H2be, H2bf, H2bg, H2bh, H2bi, H2bj, H2bk, H2b1, H2bm, H2bn, H2bo, H3a, H3b, H3c, H3d, H3e, H3f, H3g, H3h, H3i, H3j, H4a, H4b, H4c, H4d, H4e, H4f, H4g, H4h, H4i, H4j, H4k and H41; histone cluster 2 H2aa3, H2aa4, H2ab, H2ac, H2be, H2bf, H3a, H3c, H3d, H4a and H4b; histone cluster 3, H2a; histone cluster 3, H2bb; histone cluster 3, H3; histone cluster 4, H4; histone deacetylase 1-11; histone H4 transcription factor; Histone-lysine N-methyltransferase MLL4; HIV-1 Tat interactive protein 2, 30 kDa; HIV-1 Tat specific factor 1; HKR1, GLI-Kruppel zinc finger family member; HMG-box transcription factor 1; HNF1 homeobox A; HNF1 homeobox B; Holliday junction recognition protein; holocarboxylase synthetase (biotin-(proprionyl-CoA-carboxylase (ATP-hydrolysing)) ligase); holocytochrome c synthase; homeobox A1-A7, A9-A11, A13, B1-B9, B13, C4-C6, C8-C13, D1, D3-D4, D8-D13; homeobox and leucine zipper encoding; homeodomain interacting protein kinase 1-4; homocysteine-inducible, endoplasmic reticulum stress-inducible, ubiquitin-like domain member 1; homogentisate 1,2-dioxygenase; HOP homeobox; hormonally up-regulated Neu-associated kinase; hornerin; host cell factor C1 (VP16-accessory protein); host cell factor C1 regulator 1 (XPO1 dependent); host cell factor C2; HRAS-like suppressor; HRAS-like suppressor 2; HRAS-like suppressor family, member 5; HSPA (heat shock 70 kDa) binding protein, cytoplasmic cochaperone 1; HSPB (heat shock 27 kDa) associated protein 1; HtrA serine peptidase 1; HtrA serine peptidase 2; HtrA serine peptidase 3; HtrA serine peptidase 4; human immunodeficiency virus type I enhancer binding protein 1; human immunodeficiency virus type I enhancer binding protein 2; human immunodeficiency virus type I enhancer binding protein 3; huntingtin; huntingtin interacting protein 1; huntingtin interacting protein 1 related; huntingtin-associated protein 1; hyaluronan and proteoglycan link protein 1; hyaluronan and proteoglycan link protein 2; hyaluronan and proteoglycan link protein 3; hyaluronan and proteoglycan link protein 4; hyaluronan binding protein 2; hyaluronan binding protein 4; hyaluronan synthase 1; hyaluronan synthase 2; hyaluronan synthase 3; hyaluronan-mediated motility receptor (RHAMM); hyaluronoglucosaminidase 1; hyaluronoglucosaminidase 2; hyaluronoglucosaminidase 3; hyaluronoglucosaminidase 4; hydrogen voltage-gated channel 1; hydrolethalus syndrome 1; hydroxyacid oxidase (glycolate oxidase) 1; hydroxyacid oxidase 2 (long chain); hydroxyacyl-CoA dehydrogenase; hydroxyacyl-CoA dehydrogenase/3-ketoacyl-CoA thiolase/enoyl-CoA hydratase (trifunctional protein), alpha subunit; hydroxyacyl-CoA dehydrogenase/3-ketoacyl-CoA thiolase/enoyl-CoA hydratase (trifunctional protein), beta subunit; hydroxyacylglutathione hydrolase; hydroxyacylglutathione hydrolase-like; Hydroxyacyl-thioester dehydratase type 2, mitochondrial; hydroxycarboxylic acid receptor 1; hydroxycarboxylic acid receptor 2; hydroxycarboxylic acid receptor 3; hydroxy-delta-5-steroid dehydrogenase, 3 beta- and steroid delta-isomerase 1; hydroxy-delta-5-steroid dehydrogenase, 3 beta- and steroid delta-isomerase 2; hydroxy-delta-5-steroid dehydrogenase, 3 beta- and steroid delta-isomerase 7; hydroxymethylbilane synthase; hydroxyprostaglandin dehydrogenase 15-(NAD); hydroxysteroid (11-beta) dehydrogenase 1, 1-like and 2; hydroxysteroid (17-beta) dehydrogenase 1-4, 7-8 and 10-14; hydroxysteroid dehydrogenase like 1; hydroxysteroid dehydrogenase like 2; hypermethylated in cancer 1; hypermethylated in cancer 2; hyperpolarization activated cyclic nucleotide-gated potassium channel 1-4; hypocretin (orexin) neuropeptide precursor; hypocretin (orexin) receptor 1; hypocretin (orexin) receptor 2; hypoxanthine phosphoribosyltransferase 1; hypoxia inducible factor 1, alpha subunit (basic helix-loop-helix transcription factor); hypoxia inducible factor 1, alpha subunit inhibitor; hypoxia inducible factor 3, alpha subunit; hypoxia inducible lipid droplet-associated; hypoxia up-regulated 1; iduronate 2-sulfatase; iduronidase, alpha-L-; IGF-like family member 1; IGF-like family member 2; IGF-like family member 3; IGF-like family member 4; IGF-like family receptor 1; IgLON family member 5; IK cytokine, down-regulator of HLA II; IKAROS family zinc finger 1 (Ikaros); IKAROS family zinc finger 2 (Helios); IKAROS family zinc finger 3 (Aiolos); IKAROS family zinc finger 4 (Eos); IKAROS family zinc finger 5 (Pegasus); IKBKB interacting protein; IL2-inducible T-cell kinase; ilvB (bacterial acetolactate synthase)-like; immature colon carcinoma transcript 1; immediate early response 2; immediate early response 3; immediate early response 3 interacting protein 1; immediate early response 5; immediate early response 5-like; immunity-related GTPase family, cinema; immunity-related GTPase family, M; immunity-related GTPase family, Q; immunoglobin superfamily, member 21; immunoglobulin (CD79A) binding protein 1; immunoglobulin heavy constant gamma 1 (Glm marker); immunoglobulin heavy constant gamma 2 (G2m marker); immunoglobulin heavy constant gamma 4 (G4m marker); immunoglobulin J polypeptide, linker protein for immunoglobulin alpha and mu polypeptides; immunoglobulin kappa constant; immunoglobulin kappa variable 4-1; immunoglobulin lambda-like polypeptide 1; immunoglobulin lambda-like polypeptide 5; immunoglobulin mu binding protein 2; immunoglobulin superfamily, DCC subclass, member 3; immunoglobulin superfamily, DCC subclass, member 4; immunoglobulin superfamily, member 1, 3, 5, 6, 8, 9, 9B, 10, 11, 22 and 23; IMP (inosine 5′-monophosphate) dehydrogenase 1; IMP (inosine 5′-monophosphate) dehydrogenase 2; importin 4, 5, 7, 8, 9, 11, 13; 11Importin subunit alpha-2-like protein; Indian hedgehog; indoleamine 2,3-dioxygenase 1; indoleamine 2,3-dioxygenase 2; indolethylamine N-methyltransferase; inducible T-cell co-stimulator; inducible T-cell co-stimulator ligand; influenza virus NS1A binding protein; inhibin, alpha; inhibin, beta A; inhibin, beta B; inhibin, beta C; inhibin, beta E; inhibitor of Bruton agammaglobulinemia tyrosine kinase; inhibitor of CDK, cyclin A1 interacting protein 1; inhibitor of DNA binding 1, dominant negative helix-loop-helix protein; inhibitor of DNA binding 2, dominant negative helix-loop-helix protein; inhibitor of DNA binding 3, dominant negative helix-loop-helix protein; inhibitor of DNA binding 4, dominant negative helix-loop-helix protein; inhibitor of growth family, member 1; inhibitor of growth family, member 2; inhibitor of growth family, member 3; inhibitor of growth family, member 4; inhibitor of growth family, member 5; inhibitor of kappa light polypeptide gene enhancer in B-cells, kinase beta; inhibitor of kappa light polypeptide gene enhancer in B-cells, kinase complex-associated protein; inhibitor of kappa light polypeptide gene enhancer in B-cells, kinase epsilon; inhibitor of kappa light polypeptide gene enhancer in B-cells, kinase gamma; inner centromere protein antigens 135/155 kDa; inner membrane protein, mitochondrial; INO80 complex subunit B; INO80 complex subunit C; INO80 complex subunit D; INO80 complex subunit E; inosine triphosphatase (nucleoside triphosphate pyrophosphatase); inositol 1,3,4,5,6-pentakisphosphate 2-kinase; inositol 1,4,5-trisphosphate receptor interacting protein; inositol 1,4,5-trisphosphate receptor interacting protein-like 1; inositol 1,4,5-trisphosphate receptor interacting protein-like 2; inositol 1,4,5-trisphosphate receptor, type 1; inositol 1,4,5-trisphosphate receptor, type 2; inositol 1,4,5-trisphosphate receptor, type 3; inositol hexakisphosphate kinase 1; inositol hexakisphosphate kinase 2; inositol hexakisphosphate kinase 3; inositol polyphosphate multikinase; inositol polyphosphate phosphatase-like 1; inositol polyphosphate-1-phosphatase; inositol polyphosphate-4-phosphatase, type I, 107 kDa; inositol polyphosphate-4-phosphatase, type II, 105 kDa; inositol polyphosphate-5-phosphatase F; inositol polyphosphate-5-phosphatase J; inositol polyphosphate-5-phosphatase K; inositol polyphosphate-5-phosphatase, 145 kDa; inositol polyphosphate-5-phosphatase, 40 kDa; inositol polyphosphate-5-phosphatase, 72 kDa; inositol polyphosphate-5-phosphatase, 75 kDa; inositol(myo)-1(or 4)-monophosphatase 1; inositol(myo)-1(or 4)-monophosphatase 2; inositol-3-phosphate synthase 1; inositol-tetrakisphosphate 1-kinase; inositol-trisphosphate 3-kinase A; inositol-trisphosphate 3-kinase B; inositol-trisphosphate 3-kinase C; insulin; insulin induced gene 1; insulin induced gene 2; insulin receptor; insulin receptor substrate 1; insulin receptor substrate 2; insulin receptor substrate 4; insulin receptor-related receptor; insulin-degrading enzyme; insulin-like 3 (Leydig cell); insulin-like 4 (placenta); insulin-like 5; insulin-like 6; insulin-like growth factor 1 (somatomedin C); insulin-like growth factor 1 receptor; insulin-like growth factor 2 (somatomedin A); insulin-like growth factor 2 mRNA binding protein 1; insulin-like growth factor 2 mRNA binding protein 2; insulin-like growth factor 2 mRNA binding protein 3; insulin-like growth factor 2 receptor; insulin-like growth factor binding protein 1-7; insulin-like growth factor binding protein, acid labile subunit; insulin-like growth factor binding protein-like 1; insulinoma-associated 1; insulinoma-associated 2; integral membrane protein 2A; integral membrane protein 2B; integral membrane protein 2C; integrator complex subunit 1-12; integrin beta 1 binding protein (melusin) 2; integrin beta 1 binding protein 1; integrin beta 1 binding protein 3; integrin beta 3 binding protein (beta3-endonexin); integrin, alpha 1; integrin, alpha 10; integrin, alpha 11; integrin, alpha 2 (CD49B, alpha 2 subunit of VLA-2 receptor); integrin, alpha 2b (platelet glycoprotein IIb of IIb/IIIa complex, antigen CD41); integrin, alpha 3 (antigen CD49C, alpha 3 subunit of VLA-3 receptor); integrin, alpha 4 (antigen CD49D, alpha 4 subunit of VLA-4 receptor); integrin, alpha 5 (fibronectin receptor, alpha polypeptide); integrin, alpha 6; integrin, alpha 7; integrin, alpha 8; integrin, alpha 9; integrin, alpha D; integrin, alpha E (antigen CD103, human mucosal lymphocyte antigen 1; alpha polypeptide); integrin, alpha L (antigen CD11A (p180), lymphocyte function-associated antigen 1; alpha polypeptide); integrin, alpha M (complement component 3 receptor 3 subunit); integrin, alpha V (vitronectin receptor, alpha polypeptide, antigen CD51); integrin, alpha X (complement component 3 receptor 4 subunit); integrin, beta 1 (fibronectin receptor, beta polypeptide, antigen CD29 includes MDF2, MSK12); integrin, beta 2 (complement component 3 receptor 3 and 4 subunit); integrin, beta 3 (platelet glycoprotein Ma, antigen CD61); integrin, beta 4; integrin, beta 5; integrin, beta 6; integrin, beta 7; integrin, beta 8; integrin, beta-like 1 (with EGF-like repeat domains); integrin-binding sialoprotein; integrin-linked kinase; integrin-linked kinase-associated serine/threonine phosphatase; intelectin 1 (galactofuranose binding); intelectin 2; interaction protein for cytohesin exchange factors 1; inter-alpha-trypsin inhibitor heavy chain 1; inter-alpha-trypsin inhibitor heavy chain 2; inter-alpha-trypsin inhibitor heavy chain 3; inter-alpha-trypsin inhibitor heavy chain family, member 4; inter-alpha-trypsin inhibitor heavy chain family, member 5; inter-alpha-trypsin inhibitor heavy chain family, member 6; intercellular adhesion molecule 1; intercellular adhesion molecule 2; intercellular adhesion molecule 3; intercellular adhesion molecule 4 (Landsteiner-Wiener blood group); intercellular adhesion molecule 5, telencephalin; interferon (alpha, beta and omega) receptor 1; interferon (alpha, beta and omega) receptor 2; interferon alpha responsive protein isoform a; interferon gamma receptor 1; interferon gamma receptor 2 (interferon gamma transducer 1); interferon induced transmembrane protein 1 (9-27); interferon induced transmembrane protein 10; interferon induced transmembrane protein 2 (1-8D); interferon induced transmembrane protein 3; interferon induced transmembrane protein 5; interferon induced with helicase C domain 1; interferon regulatory factor 1-9; interferon regulatory factor 2 binding protein 1; interferon regulatory factor 2 binding protein 2; interferon regulatory factor 2 binding protein-like; interferon stimulated exonuclease gene 20 kDa; interferon stimulated exonuclease gene 20 kDa-like 2; interferon, alpha 1, alpha 2, alpha 4-alpha 8, alpha 10, alpha 13, alpha 14, alpha 16, alpha 17 and alpha 21; interferon, alpha-inducible protein 27; interferon, alpha-inducible protein 27-like 1; interferon, alpha-inducible protein 27-like 2; interferon, alpha-inducible protein 6; interferon, beta 1, fibroblast; interferon, epsilon; interferon, gamma; interferon, gamma-inducible protein 16; interferon, gamma-inducible protein 30; interferon, kappa; interferon, omega 1; interferon-induced protein 35; interferon-induced protein 44; interferon-induced protein 44-like; interferon-induced protein with tetratricopeptide repeats 1, 1B, 2, 3 and 5; interferon-related developmental regulator 1; interferon-related developmental regulator 2; interleukin 1 family, member 10 (theta); interleukin 1 receptor accessory protein; interleukin 1 receptor accessory protein-like 1; interleukin 1 receptor accessory protein-like 2; interleukin 1 receptor antagonist; interleukin 1 receptor, type I; interleukin 1 receptor, type II; interleukin 1 receptor-like 1; interleukin 1 receptor-like 2; interleukin 1, alpha; interleukin 1, beta; interleukin 10; interleukin 10 receptor, alpha; interleukin 10 receptor, beta; interleukin 11; interleukin 11 receptor, alpha; interleukin 12 receptor, beta 1; interleukin 12 receptor, beta 2; interleukin 12A (natural killer cell stimulatory factor 1, cytotoxic lymphocyte maturation factor 1, p35); interleukin 12B (natural killer cell stimulatory factor 2, cytotoxic lymphocyte maturation factor 2, p40); interleukin 13; interleukin 13 receptor, alpha 1; interleukin 13 receptor, alpha 2; interleukin 15; interleukin 15 receptor, alpha; interleukin 16; interleukin 17 receptor A; interleukin 17 receptor B; interleukin 17 receptor C; interleukin 17 receptor D; interleukin 17 receptor E; interleukin 17 receptor E-like; interleukin 17A; interleukin 17B; interleukin 17C; interleukin 17D; interleukin 17F; interleukin 18 (interferon-gamma-inducing factor); interleukin 18 binding protein; interleukin 18 receptor 1; interleukin 18 receptor accessory protein; interleukin 19; interleukin 2; interleukin 2 receptor, alpha; interleukin 2 receptor, beta; interleukin 2 receptor, gamma; interleukin 20; interleukin 20 receptor beta; interleukin 20 receptor, alpha; interleukin 21; interleukin 21 receptor; interleukin 22; interleukin 22 receptor, alpha 1; interleukin 22 receptor, alpha 2; interleukin 23 receptor; interleukin 23, alpha subunit p19; interleukin 24; interleukin 25; interleukin 26; interleukin 27; interleukin 27 receptor, alpha; interleukin 28 receptor, alpha (interferon, lambda receptor); interleukin 28A (interferon, lambda 2); interleukin 28B (interferon, lambda 3); interleukin 29 (interferon, lambda 1); interleukin 3 (colony-stimulating factor, multiple); interleukin 3 receptor, alpha (low affinity); interleukin 31; interleukin 31 receptor A; interleukin 32; interleukin 33; interleukin 34; interleukin 36 receptor antagonist; interleukin 36, alpha; interleukin 36, beta; interleukin 36, gamma; interleukin 37; interleukin 4; interleukin 4 induced 1; interleukin 4 receptor; interleukin 5 (colony-stimulating factor, eosinophil); interleukin 5 receptor, alpha; interleukin 6 (interferon, beta 2); interleukin 6 receptor; interleukin 6 signal transducer (gp130, oncostatin M receptor); interleukin 7; interleukin 7 receptor; interleukin 8; interleukin 9; interleukin 9 receptor; interleukin enhancer binding factor 2, 45 kDa; interleukin enhancer binding factor 3, 90 kDa; interleukin-1 receptor-associated kinase 1; interleukin-1 receptor-associated kinase 1 binding protein 1; interleukin-1 receptor-associated kinase 2; interleukin-1 receptor-associated kinase 3; interleukin-1 receptor-associated kinase 4; Interleukin-like; intermediate filament family orphan 1; intermediate filament family orphan 2; internexin neuronal intermediate filament protein, alpha; interphotoreceptor matrix proteoglycan 1; interphotoreceptor matrix proteoglycan 2; intersectin 1 (SH3 domain protein); intersectin 2; intestinal cell (MAK-like) kinase; intestine-specific homeobox; intracisternal A particle-promoted polypeptide; inversin; inverted formin, FH2 and WH2 domain containing; involucrin; iodotyrosine deiodinase; IQ motif and Sec7 domain 1; IQ motif and Sec7 domain 2; IQ motif and Sec7 domain 3; IQ motif and ubiquitin domain containing; IQCJ-SCHIP1 readthrough; Iron/zinc purple acid phosphatase-like protein; iron-responsive element binding protein 2; iroquois homeobox 1-6; ISG15 ubiquitin-like modifier; ISL LIM homeobox 1; ISL LIM homeobox 2; islet amyloid polypeptide; islet cell autoantigen 1, 69 kDa; islet cell autoantigen 1.69 kDa-like; isocitrate dehydrogenase 1 (NADP+), soluble; isocitrate dehydrogenase 2 (NADP+), mitochondrial; isocitrate dehydrogenase 3 (NAD+) alpha; isocitrate dehydrogenase 3 (NAD+) beta; isocitrate dehydrogenase 3 (NAD+) gamma; isoleucyl-tRNA synthetase; isoleucyl-tRNA synthetase 2, mitochondrial; isopentenyl-diphosphate delta isomerase 1; isopentenyl-diphosphate delta isomerase 2; isoprenoid synthase domain containing; isoprenylcysteine carboxyl methyltransferase; isovaleryl-CoA dehydrogenase; IZUMO family member 2; IZUMO family member 3; IZUMO family member 4; izumo sperm-egg fusion 1; jagged 1; jagged 2; Janus kinase 1; Janus kinase 2; Janus kinase 3; j anus kinase and microtubule interacting protein 1; j anus kinase and microtubule interacting protein 2; Janus kinase and microtubule interacting protein 3; JAZF zinc finger 1; JMJD7-PLA2G4B readthrough; JNK1/MAPK8-associated membrane protein; jumonji, AT rich interactive domain 2; jumping translocation breakpoint; jun B proto-oncogene; jun D proto-oncogene; Jun dimerization protein 2; jun proto-oncogene; junction mediating and regulatory protein, p53 cofactor; junction plakoglobin; junctional adhesion molecule 2; junctional adhesion molecule 3; junctional sarcoplasmic reticulum protein 1; junctophilin 1; junctophilin 2; junctophilin 3; junctophilin 4; K(lysine) acetyltransferase 2A; K(lysine) acetyltransferase 2B; K(lysine) acetyltransferase 5; K(lysine) acetyltransferase 6A; K(lysine) acetyltransferase 6B; K(lysine) acetyltransferase 7; K(lysine) acetyltransferase 8; kalirin, RhoGEF kinase; kallikrein 1; kallikrein B, plasma (Fletcher factor) 1; kallikrein-related peptidase 2-15; Kallmann syndrome 1 sequence; kaptin (actin binding protein); karyopherin (importin) beta 1; karyopherin alpha 1 (importin alpha 5); karyopherin alpha 2 (RAG cohort 1, importin alpha 1); karyopherin alpha 3 (importin alpha 4); karyopherin alpha 4 (importin alpha 3); karyopherin alpha 5 (importin alpha 6); karyopherin alpha 6 (importin alpha 7); karyopherin alpha 7 (importin alpha 8); KAT8 regulatory NSL complex subunit 2; KAT8 regulatory NSL complex subunit 3; katanin p60 (ATPase containing) subunit A 1; katanin p60 subunit A-like 1; katanin p60 subunit A-like 2; katanin p80 (WD repeat containing) subunit B 1; Kazal-type serine peptidase inhibitor domain 1; kazrin, periplakin interacting protein; KCNE1-like; KDEL (Lys-Asp-Glu-Leu) endoplasmic reticulum protein retention receptor 1; KDEL (Lys-Asp-Glu-Leu) endoplasmic reticulum protein retention receptor 2; KDEL (Lys-Asp-Glu-Leu) endoplasmic reticulum protein retention receptor 3; kelch-like ECH-associated protein 1; Kell blood group, metallo-endopeptidase; keratin 1, 2, 3, 4, 5, 6A, 6B, 6C, 7, 8, 9, 10, 12, 13, 14, 15, 16, 17, 18, 19, 20, 23 (histone deacetylase inducible), 24, 25, 26, 27, 28, 31, 32, 33A, 33B, 34, 35, 36, 37, 38, 39, 40, 71, 72, 73, 74, 75, 76, 77, 78, 79, 80, 81, 82, 83, 84, 85, 86, and 222; keratin associated protein 1-3, 2-1, 3-1, 3-2, 3-3, 4-1, 4-11, 4-12, 4-2, 4-3, 4-4, 4-5, 4-6, 4-7, 4-8, 4-9, 5-1, 5-10, 5-11, 5-2, 5-3, 5-4, 5-5, 5-6, 5-7, 5-8, 5-9, 6-1, 6-2, 6-3, 8-1, 9-1, 9-2, 9-3, 9-4, 9-6, 9-7, 9-8, 9-9, 10-1, 10-10, 10-11, 10-12, 10-2, 10-3, 10-4, 10-5, 10-6, 10-7, 10-8, 10-9, 1-1, 11-1, 12-1, 12-2, 12-3, 12-4, 13-1, 13-2, 13-3, 13-4, 1-4, 1-5, 15-1, 16-1, 17-1, 19-1, 19-2, 19-3, 19-4, 19-5, 19-6, 19-7, 19-8, 20-1, 20-2, 20-3, 20-4, 21-1, 21-2, 21-3, 2-2, 22-1, 22-2, 2-3, 23-1, 2-4, 24-1, 25-1, 26-1, 27-1, 29-1; Keratin, type II cuticular Hb6; keratinocyte associated protein 2; keratinocyte associated protein 3; keratinocyte differentiation-associated protein; keratinocyte proline-rich protein; keratocan; ketohexokinase (fructokinase); KH and NYN domain containing; KH domain containing, RNA binding, signal transduction associated 1; KH domain containing, RNA binding, signal transduction associated 2; KH domain containing, RNA binding, signal transduction associated 3; KH-type splicing regulatory protein; kidney associated antigen 1; killer cell immunoglobulin-like receptor, three domains, long cytoplasmic tail, 1; killer cell immunoglobulin-like receptor, three domains, long cytoplasmic tail, 2; Killer cell immunoglobulin-like receptor, three domains, long cytoplasmic tail, 2 (NKAT4); killer cell immunoglobulin-like receptor, three domains, long cytoplasmic tail, 3; killer cell immunoglobulin-like receptor, three domains, short cytoplasmic tail, 1; killer cell immunoglobulin-like receptor, three domains, X1; killer cell immunoglobulin-like receptor, two domains, long cytoplasmic tail, 1; killer cell immunoglobulin-like receptor, two domains, long cytoplasmic tail, 3; killer cell immunoglobulin-like receptor, two domains, long cytoplasmic tail, 4; killer cell lectin-like receptor subfamily B, member 1; killer cell lectin-like receptor subfamily C, member 1; killer cell lectin-like receptor subfamily C, member 2; killer cell lectin-like receptor subfamily C, member 3; killer cell lectin-like receptor subfamily C, member 4; killer cell lectin-like receptor subfamily D, member 1; killer cell lectin-like receptor subfamily F, member 1; killer cell lectin-like receptor subfamily F, member 2; killer cell lectin-like receptor subfamily G, member 1; killer cell lectin-like receptor subfamily G, member 2; killer cell lectin-like receptor subfamily K, member 1; killin, p53-regulated DNA replication inhibitor; kinase D-interacting substrate, 220 kDa; kinase insert domain receptor (a type III receptor tyrosine kinase); kinase suppressor of ras 1; kinase suppressor of ras 2; kinectin 1 (kinesin receptor); kinesin family member 1A, 1B, 1C, 2B, 2C, 3A, 3B, 3C, 4A, 4B, 5A, 5B, 5C, 6, 7, 9, 11, 12, 13A, 13B, 14, 15, 16B, 17, 18A, 18B, 19, 20A, 20B, 21A, 21B, 22, 23, 24, 25, 26A, 26B, 27, C1, C2 and C3; kinesin heavy chain member 2A; kinesin light chain 1-4; kinesin-associated protein 3; kinetochore associated 1; kininogen 1; kinocilin; KiSS-1 metastasis-suppressor; KIS S1 receptor; KIT ligand; klotho; klotho beta; KM-PA-2 protein; KN motif and ankyrin repeat domains 1; KN motif and ankyrin repeat domains 2; KN motif and ankyrin repeat domains 3; KN motif and ankyrin repeat domains 4; KRIT1, ankyrin repeat containing; Kruppel-like factor 1 (erythroid), 2 (lung), 3 (basic), 4 (gut), 5 (intestinal), 6, 7 (ubiquitous), 8, 9, 10, 11, 12, 13, 14, 15, 16, and 17; Kv channel interacting protein 1; Kv channel interacting protein 2; Kv channel interacting protein 3, calsenilin; Kv channel interacting protein 4; kynureninase; kynurenine 3-monooxygenase (kynurenine 3-hydroxylase); kyphoscoliosis peptidase; L antigen family, member 3; L1 cell adhesion molecule; L-2-hydroxyglutarate dehydrogenase; La ribonucleoprotein domain family, member 1; La ribonucleoprotein domain family, member 1B; La ribonucleoprotein domain family, member 4; La ribonucleoprotein domain family, member 4B; La ribonucleoprotein domain family, member 6; La ribonucleoprotein domain family, member 7; lacritin; lactalbumin, alpha-; lactamase, beta; lactamase, beta 2; lactase; lactase-like; lactate dehydrogenase A; lactate dehydrogenase A-like 6A; lactate dehydrogenase A-like 6B; lactate dehydrogenase B; lactate dehydrogenase C; lactate dehydrogenase D; lactation elevated 1; lactoperoxidase; lactotransferrin; ladinin 1; ladybird homeobox 1; ladybird homeobox 2; lamin A/C; lamin B receptor; lamin B1; lamin B2; laminin, alpha 1; laminin, alpha 2; laminin, alpha 3; laminin, alpha 4; laminin, alpha 5; laminin, beta 1; laminin, beta 2 (laminin S); laminin, beta 3; laminin, beta 4; laminin, gamma 1 (formerly LAMB2); laminin, gamma 2; laminin, gamma 3; LanC lantibiotic synthetase component C-like 1 (bacterial); LanC lantibiotic synthetase component C-like 2 (bacterial); LanC lantibiotic synthetase component C-like 3 (bacterial); lanosterol synthase (2,3-oxidosqualene-lanosterol cyclase); late cornified envelope 1A, 1B, 1C, 1D, 1E, 1F, 2A, 2B, 2C, 2D, 3A, 3B, 3C, 3D, 3E, 4A, 5A, and 6A; late cornified envelope-like proline-rich 1; late endosomal/lysosomal adaptor, MAPK and MTOR activator 1; late endosomal/lysosomal adaptor, MAPK and MTOR activator 2; late endosomal/lysosomal adaptor, MAPK and MTOR activator 3; latent transforming growth factor beta binding protein 1; latent transforming growth factor beta binding protein 2; latent transforming growth factor beta binding protein 3; latent transforming growth factor beta binding protein 4; latexin; latrophilin 1; latrophilin 2; latrophilin 3; layilin; Lck interacting transmembrane adaptor 1; Leber congenital amaurosis 5; Leber congenital amaurosis 5-like; lecithin retinol acyltransferase (phosphatidylcholine-retinol O-acyltransferase); lecithin-cholesterol acyltransferase; lectin, galactoside-binding, soluble, 1; lectin, galactoside-binding, soluble, 12; lectin, galactoside-binding, soluble, 13; lectin, galactoside-binding, soluble, 14; lectin, galactoside-binding, soluble, 16; lectin, galactoside-binding, soluble, 2; lectin, galactoside-binding, soluble, 3; lectin, galactoside-binding, soluble, 3 binding protein; lectin, galactoside-binding, soluble, 4; lectin, galactoside-binding, soluble, 7; lectin, galactoside-binding, soluble, 7B; lectin, galactoside-binding, soluble, 8; lectin, galactoside-binding, soluble, 9; lectin, galactoside-binding, soluble, 9B; lectin, galactoside-binding, soluble, 9C; lectin, galactoside-binding-like; lectin, mannose-binding 2; lectin, mannose-binding 2-like; lectin, mannose-binding, 1; lectin, mannose-binding, 1 like; left-right determination factor 1; left-right determination factor 2; legumain; leiomodin 1 (smooth muscle); leiomodin 2 (cardiac); leiomodin 3 (fetal); leishmanolysin-like (metallopeptidase M8 family); lemur tyrosine kinase 2; lemur tyrosine kinase 3; lengsin, lens protein with glutamine synthetase domain; lens epithelial protein; lens intrinsic membrane protein 2, 19 kDa; leprecan-like 1; leprecan-like 2; leprecan-like 4; leptin; leptin receptor; leptin receptor overlapping transcript; leptin receptor overlapping transcript-like 1; leucine aminopeptidase 3; leucine carboxyl methyltransferase 1; leucine carboxyl methyltransferase 2; leucine proline-enriched proteoglycan (leprecan) 1; leucine rich repeat (in FLII) interacting protein 1; leucine rich repeat (in FLII) interacting protein 2; leucine rich repeat neuronal 1; leucine rich repeat neuronal 2; leucine rich repeat neuronal 3; leucine rich repeat neuronal 4; leucine rich repeat protein 1; leucine rich repeat transmembrane neuronal 1; leucine rich repeat transmembrane neuronal 2; leucine rich repeat transmembrane neuronal 3; leucine rich repeat transmembrane neuronal 4; leucine rich transmembrane and O-methyltransferase domain containing; leucine twenty homeobox; leucine zipper and CTNNBIP1 domain containing; leucine zipper protein 1; leucine zipper protein 2; leucine zipper protein 4; leucine zipper transcription factor-like 1; leucine zipper, down-regulated in cancer 1; leucine zipper, down-regulated in cancer 1-like; leucine, glutamate and lysine rich 1; leucine-rich alpha-2-glycoprotein 1; leucine-rich PPR-motif containing; leucine-rich repeat kinase 1; leucine-rich repeat kinase 2; leucine-rich repeat LGI family, member 2; leucine-rich repeat LGI family, member 3; leucine-rich repeat LGI family, member 4; leucine-rich repeat, immunoglobulin-like and transmembrane domains 1; leucine-rich repeat, immunoglobulin-like and transmembrane domains 2; leucine-rich repeat, immunoglobulin-like and transmembrane domains 3; leucine-rich repeats and guanylate kinase domain containing; leucine-rich repeats and immunoglobulin-like domains 1; leucine-rich repeats and immunoglobulin-like domains 2; leucine-rich repeats and immunoglobulin-like domains 3; leucine-rich repeats and transmembrane domains 1; leucine-rich repeats and transmembrane domains 2; leucine-rich, glioma inactivated 1; leucine-zipper-like transcription regulator 1; leucyl/cystinyl aminopeptidase; leucyl-tRNA synthetase; leucyl-tRNA synthetase 2, mitochondrial; leukemia inhibitory factor (cholinergic differentiation factor); leukemia inhibitory factor receptor alpha; leukemia NUP98 fusion partner 1; leukocyte cell derived chemotaxin 1; leukocyte cell-derived chemotaxin 2; leukocyte immunoglobulin-like receptor, subfamily A (with TM domain), member 1; leukocyte immunoglobulin-like receptor, subfamily A (with TM domain), member 2; leukocyte immunoglobulin-like receptor, subfamily A (with TM domain), member 4; leukocyte immunoglobulin-like receptor, subfamily A (with TM domain), member 5; leukocyte immunoglobulin-like receptor, subfamily A (with TM domain), member 6; leukocyte immunoglobulin-like receptor, subfamily A (without TM domain), member 3; leukocyte immunoglobulin-like receptor, subfamily B (with TM and ITIM domains), member 1; leukocyte immunoglobulin-like receptor, subfamily B (with TM and ITIM domains), member 2; leukocyte immunoglobulin-like receptor, subfamily B (with TM and ITIM domains), member 3; leukocyte immunoglobulin-like receptor, subfamily B (with TM and ITIM domains), member 4; leukocyte immunoglobulin-like receptor, subfamily B (with TM and ITIM domains), member 5; leukocyte receptor cluster (LRC) member 1; leukocyte receptor cluster (LRC) member 8; leukocyte receptor cluster (LRC) member 9; leukocyte receptor tyrosine kinase; leukocyte specific transcript 1; leukocyte-associated immunoglobulin-like receptor 1; leukocyte-associated immunoglobulin-like receptor 2; leukotriene A4 hydrolase; leukotriene B4 receptor; leukotriene B4 receptor 2; leukotriene C4 synthase; leupaxin; LFNG O-fucosylpeptide 3-beta-N-acetylglucosaminyltransferase; ligand dependent nuclear receptor corepressor; ligand dependent nuclear receptor corepressor-like; ligand dependent nuclear receptor interacting factor 1; ligand of numb-protein X 1; ligand of numb-protein X 2; ligase I, DNA, ATP-dependent; ligase III, DNA, ATP-dependent; ligase IV, DNA, ATP-dependent; like-glycosyltransferase; LIM and cysteine-rich domains 1; LIM and senescent cell antigen-like domains 1; LIM and senescent cell antigen-like domains 2; LIM and senescent cell antigen-like domains 3-like; LIM and SH3 protein 1; LIM domain 7; LIM domain and actin binding 1; LIM domain binding 1; LIM domain binding 2; LIM domain binding 3; LIM domain kinase 1; LIM domain kinase 2; LIM domain only 1 (rhombotin 1); LIM domain only 2 (rhombotin-like 1); LIM domain only 3 (rhombotin-like 2); LIM domain only 4; LIM homeobox 1; LIM homeobox 2; LIM homeobox 3; LIM homeobox 4; LIM homeobox 5; LIM homeobox 6; LIM homeobox 8; LIM homeobox 9; LIM homeobox transcription factor 1, alpha; LIM homeobox transcription factor 1, beta; limbic system-associated membrane protein; linker for activation of T cells; linker for activation of T cells family, member 2; lipase A, lysosomal acid, cholesterol esterase; lipase maturation factor 1; lipase maturation factor 2; lipase, endothelial; lipase, family member J; lipase, family member K; lipase, family member M; lipase, family member N; lipase, gastric; lipase, hepatic; lipase, hormone-sensitive; lipase, member H; lipase, member I; Lipid phosphate phosphatase-related protein type 1; Lipid phosphate phosphatase-related protein type 2; Lipid phosphate phosphatase-related protein type 3; Lipid phosphate phosphatase-related protein type 4; Lipid phosphate phosphatase-related protein type 5; lipin 1; lipin 2; lipin 3; lipocalin 1; lipocalin 10; lipocalin 12; lipocalin 15; lipocalin 2; lipocalin 6; lipocalin 8; lipocalin 9; lipocalin-like 1; lipoic acid synthetase; lipolysis stimulated lipoprotein receptor; lipoma HMGIC fusion partner; lipoma HMGIC fusion partner-like 1; lipoma HMGIC fusion partner-like 2; lipoma HMGIC fusion partner-like 3; lipoma HMGIC fusion partner-like 4; lipoma HMGIC fusion partner-like 5; lipopolysaccharide binding protein; lipopolysaccharide-induced TNF factor; lipoprotein lipase; lipoprotein, Lp(a); lipoyltransferase 1; listerin E3 ubiquitin protein ligase 1; liver expressed antimicrobial peptide 2; lon peptidase 1, mitochondrial; lon peptidase 2, peroxisomal; LON peptidase N-terminal domain and ring finger 1; LON peptidase N-terminal domain and ring finger 2; LON peptidase N-terminal domain and ring finger 3; loricrin; loss of heterozygosity, 12, chromosomal region 1; low density lipoprotein receptor; low density lipoprotein receptor adaptor protein 1; low density lipoprotein receptor-related protein 1; low density lipoprotein receptor-related protein 10; low density lipoprotein receptor-related protein 11; low density lipoprotein receptor-related protein 12; low density lipoprotein receptor-related protein 1B; low density lipoprotein receptor-related protein 2; low density lipoprotein receptor-related protein 3; low density lipoprotein receptor-related protein 4; low density lipoprotein receptor-related protein 5; low density lipoprotein receptor-related protein 5-like; low density lipoprotein receptor-related protein 6; low density lipoprotein receptor-related protein 8, apolipoprotein e receptor; low density lipoprotein receptor-related protein associated protein 1; LPS-responsive vesicle trafficking, beach and anchor containing; LRP2 binding protein; LRRN4 C-terminal like; LSM10, U7 small nuclear RNA associated; LSM11, U7 small nuclear RNA associated; lumican; luteinizing hormone beta polypeptide; luteinizing hormone/choriogonadotropin receptor; Ly6/neurotoxin 1; lymphatic vessel endothelial hyaluronan receptor 1; lymphoblastic leukemia derived sequence 1; lymphocyte antigen 6 complex, locus D; lymphocyte antigen 6 complex, locus E; lymphocyte antigen 6 complex, locus GSB; lymphocyte antigen 6 complex, locus GSC; lymphocyte antigen 6 complex, locus G6C; lymphocyte antigen 6 complex, locus G6D; Lymphocyte antigen 6 complex, locus G6ELymphocyte antigen 6 complex, locus G6E, isoform CRA_a; lymphocyte antigen 6 complex, locus G6F; lymphocyte antigen 6 complex, locus H; lymphocyte antigen 6 complex, locus K; lymphocyte antigen 75; lymphocyte antigen 86; lymphocyte antigen 9; lymphocyte antigen 96; lymphocyte cytosolic protein 1 (L-plastin); lymphocyte transmembrane adaptor 1; lymphocyte-activation gene 3; lymphocyte-specific protein 1; lymphocyte-specific protein tyrosine kinase; lymphoid enhancer-binding factor 1; lymphoid-restricted membrane protein; lymphotoxin alpha (TNF superfamily, member 1); lymphotoxin beta (TNF superfamily, member 3); lymphotoxin beta receptor (TNFR superfamily, member 3); lysine (K)-specific demethylase 1A; lysine (K)-specific demethylase 1B; lysine (K)-specific demethylase 2A; lysine (K)-specific demethylase 2B; lysine (K)-specific demethylase 3A; lysine (K)-specific demethylase 3B; lysine (K)-specific demethylase 4A; lysine (K)-specific demethylase 4B; lysine (K)-specific demethylase 4C; lysine (K)-specific demethylase 4D; lysine (K)-specific demethylase 4D-like; lysine (K)-specific demethylase 5A; lysine (K)-specific demethylase 5B; lysine (K)-specific demethylase 5C; lysine (K)-specific demethylase 5D; lysine (K)-specific demethylase 6A; lysine (K)-specific demethylase 6B; lysine-rich coiled-coil 1; lysocardiolipin acyltransferase 1; lysophosphatidic acid receptor 1; lysophosphatidic acid receptor 2; lysophosphatidic acid receptor 3; lysophosphatidic acid receptor 4; lysophosphatidic acid receptor 5; lysophosphatidic acid receptor 6; lysophosphatidylcholine acyltransferase 1; lysophosphatidylcholine acyltransferase 2; lysophosphatidylcholine acyltransferase 3; lysophosphatidylcholine acyltransferase 4; lysophosphatidylglycerol acyltransferase 1; lysophospholipase I; lysophospholipase II; lysophospholipase-like 1; lysosomal protein transmembrane 4 alpha; lysosomal protein transmembrane 4 beta; lysosomal protein transmembrane 5; lysosomal trafficking regulator; lysosomal-associated membrane protein 1; lysosomal-associated membrane protein 2; lysosomal-associated membrane protein 3; lysosomal-associated membrane protein family, member 5; lysozyme; lysozyme G-like 1; lysozyme G-like 2; lysozyme-like 1; lysozyme-like 2; lysozyme-like 4; lysozyme-like 6; lysyl oxidase; lysyl oxidase-like 1; lysyl oxidase-like 2; lysyl oxidase-like 3; lysyl oxidase-like 4; macrophage erythroblast attacher; macrophage expressed 1; Macrophage mannose receptor 1; macrophage migration inhibitory factor (glycosylation-inhibiting factor); macrophage receptor with collagenous structure; macrophage scavenger receptor 1; macrophage stimulating 1 (hepatocyte growth factor-like); macrophage stimulating 1 receptor (c-met-related tyrosine kinase); MAD1 mitotic arrest deficient-like 1 (yeast); MAD2 mitotic arrest deficient-like 1 (yeast); MAD2 mitotic arrest deficient-like 2 (yeast); MAD2L1 binding protein; maestro; MAFF interacting protein; MAGI family member, X-linked; magnesium transporter 1; magnesium-dependent phosphatase 1; mahogunin, ring finger 1; major histocompatibility complex, class I A-C and E-G; major histocompatibility complex, class II, DM alpha, DM beta, DO alpha, DO beta, DP alpha 1, DP beta 1, DQ alpha 1, DQ alpha 2, DQ beta 1, DQ beta 2, DR alpha, DR beta 1, DR beta 3, DR beta 4 and DR beta 5; major histocompatibility complex, class I-related; major intrinsic protein of lens fiber; major vault protein; makorin ring finger protein 1; makorin ring finger protein 2; makorin ring finger protein 3; mal, T-cell differentiation protein; mal, T-cell differentiation protein-like; malate dehydrogenase 1, NAD (soluble); malate dehydrogenase 1B, NAD (soluble); malate dehydrogenase 2, NAD (mitochondrial); male germ cell-associated kinase; malectin; male-enhanced antigen 1; malic enzyme 1, NADP(+)-dependent, cytosolic; malic enzyme 2, NAD(+)-dependent, mitochondrial; malic enzyme 3, NADP(+)-dependent, mitochondrial; malignant fibrous histiocytoma amplified sequence 1; malignant T cell amplified sequence 1; malonyl CoA:ACP acyltransferase (mitochondrial); malonyl-CoA decarboxylase; maltase-glucoamylase (alpha-glucosidase); mannan-binding lectin serine peptidase 1 (C4/C2 activating component of Ra-reactive factor); mannan-binding lectin serine peptidase 2; mannose phosphate isomerase; mannose receptor, C type 1 and type 2; mannose-6-phosphate receptor (cation dependent); mannose-binding lectin (protein C) 2, soluble; mannose-P-dolichol utilization defect 1; mannosidase, alpha, class 1A, member 1 and member 2; mannosidase, alpha, class 1B, member 1; mannosidase, alpha, class 1C, member 1; mannosidase, alpha, class 2A, member 1 and member 2; mannosidase, alpha, class 2B, member 1 and member 2; mannosidase, alpha, class 2C, member 1; mannosidase, beta A, lysosomal; mannosidase, beta A, lysosomal-like; mannosidase, endo-alpha; mannosidase, endo-alpha-like; mannosyl (alpha-1,3-)-glycoprotein beta-1,2-N-acetylglucosaminyltransferase; mannosyl (alpha-1,3-)-glycoprotein beta-1,4-N-acetylglucosaminyltransferase, isozyme A and isozyme B; mannosyl (alpha-1,6-)-glycoprotein beta-1,2-N-acetylglucosaminyltransferase; mannosyl (alpha-1,6-)-glycoprotein beta-1,6-N-acetyl-glucosaminyltransferase; mannosyl (alpha-1,6-)-glycoprotein beta-1,6-N-acetyl-glucosaminyltransferase, isozyme B; mannosyl (beta-1,4-)-glycoprotein beta-1,4-N-acetylglucosaminyltransferase; mannosyl-oligosaccharide glucosidase; MAP kinase interacting serine/threonine kinase 1 and 2; MAP/microtubule affinity-regulating kinase 1-4; MAP3K12 binding inhibitory protein 1; MAP-kinase activating death domain; MARCKS-like 1; MAS 1 oncogene; MAS 1 oncogene-like; MAS-related GPR, member D-member G and member X1-member X4; maternal embryonic leucine zipper kinase; matrilin 1 (cartilage matrix protein)-4; matrin 3; matrix extracellular phosphoglycoprotein; matrix Gla protein; matrix metallopeptidase 2 (gelatinase A, 72 kDa gelatinase, 72 kDa type IV collagenase), 3 (stromelysin 1, progelatinase), 7 (matrilysin, uterine), 8 (neutrophil collagenase), 9 (gelatinase B, 92 kDa gelatinase, 92 kDa type IV collagenase), 10 (stromelysin 2), 11 (stromelysin 3), 13 (collagenase 3), 14 (membrane-inserted), 15 (membrane-inserted), 16 (membrane-inserted), 17 (membrane-inserted), 19, 20, 21, 23B, 24 (membrane-inserted), 25, 26, 27, and 28; matrix-remodelling associated 5, 7 and 8; mature T-cell proliferation 1; mature T-cell proliferation 1 neighbor; MAX binding protein; MAX dimerization protein 1, 3 and 4; MAX gene associated; MAX interactor 1; MAX-like protein X; MCF.2 cell line derived transforming sequence; MCF.2 cell line derived transforming sequence-like; MCF.2 cell line derived transforming sequence-like 2; McKusick-Kaufman syndrome; Mdm2, transformed 3T3 cell double minute 2, p53 binding protein (mouse) binding protein, 104 kDa; MDS1 and EVI1 complex locus; mechanistic target of rapamycin (serine/threonine kinase); Meckel syndrome, type 1; mediator complex subunit 1, 4, 6, 7, 8, 9, 10, 11, 12, 12-like, 13, 13-like, 14, 15, 16, 17, 18, 19, 20, 21, 22, 23, 24, 25, 26, 27, 28, 29, 30, and 31; mediator of cell motility 1; mediator of DNA-damage checkpoint 1; Mediterranean fever; MEF2B neighbor; megakaryoblastic leukemia (translocation) 1; megakaryocyte-associated tyrosine kinase; megalencephalic leukoencephalopathy with subcortical cysts 1; meiosis inhibitor 1; meiosis-specific nuclear structural 1; Meis homeobox 1-3; melan-A; melanin-concentrating hormone receptor 1; melanin-concentrating hormone receptor 2; melanocortin 1 receptor (alpha melanocyte stimulating hormone receptor); melanocortin 2 receptor (adrenocorticotropic hormone); melanocortin 2 receptor accessory protein; melanocortin 2 receptor accessory protein 2; melanocortin 3 receptor; melanocortin 4 receptor; melanocortin 5 receptor; Melanocyte-stimulating hormone receptor; melanoma antigen family A, 1 (directs expression of antigen MZ2-E), 2-4, 6, 9B and 8-12; melanoma antigen family B, 1-6, 10 and 16-18; melanoma antigen family C, 1-3; melanoma antigen family D, 1, 2, 4 and 4B; melanoma antigen family E, 1; melanoma antigen family E, 2; melanoma antigen family F, 1; melanoma antigen family H, 1; melanoma associated antigen (mutated) 1; melanoma associated antigen (mutated) 1-like 1; melanoma cell adhesion molecule; melanoma inhibitory activity; melanoma inhibitory activity 2; melanoma inhibitory activity family, member 3; melanophilin; melanoregulin; melatonin receptor 1A; melatonin receptor 1B; Membrane frizzled-related protein; membrane magnesium transporter 1; membrane metallo-endopeptidase; membrane metallo-endopeptidase-like 1; membrane protein, palmitoylated 1, 55 kDa; membrane protein, palmitoylated 2 (MAGUK p55 subfamily member 2), 3 (MAGUK p55 subfamily member 3), 4 (MAGUK p55 subfamily member 4), 5 (MAGUK p55 subfamily member 5), 6 (MAGUK p55 subfamily member 6), and palmitoylated 7 (MAGUK p55 subfamily member 7); membrane-associated ring finger (C3HC4) 1-11; membrane-bound transcription factor peptidase, site 1 and site 2; membrane-spanning 4-domains, subfamily A, member 1-member 8, member 4E, member 6A, member 6E, member 8B, member 10, member 12-member 15 and member 18; meningioma (disrupted in balanced translocation) 1; meningioma expressed antigen 5 (hyaluronidase); meprin A, alpha (PABA peptide hydrolase); meprin A, beta; mercaptopyruvate sulfurtransferase; mesencephalic astrocyte-derived neurotrophic factor; mesenchyme homeobox 1; mesenchyme homeobox 2; mesoderm development candidate 1; mesoderm development candidate 2; mesoderm induction early response 1, family member 2; mesoderm induction early response 1, family member 3; mesogenin 1; mesothelin; mesothelin-like; met proto-oncogene (hepatocyte growth factor receptor); metadherin; metal response element binding transcription factor 2; metallophosphoesterase 1; metallothionein 1A, 1B, 1E, 1F, 1G, 1H, 1M, 1X, 2A, 3 and 4; metallothionein-like 5, testis-specific (tesmin); metal-regulatory transcription factor 1; metastasis associated 1; metastasis associated 1 family, member 2; metastasis associated 1 family, member 3; metastasis associated in colon cancer 1; metastasis suppressor 1; metastasis suppressor 1-like; metaxin 1-3; meteorin, glial cell differentiation regulator; meteorin, glial cell differentiation regulator-like; methenyltetrahydrofolate synthetase domain containing; methionine adenosyltransferase I, alpha; methionine adenosyltransferase II, alpha and beta; methionine sulfoxide reductase A, B2 and B3; methionyl aminopeptidase 1 and 2; methionyl aminopeptidase type 1D (mitochondrial); methionyl-tRNA synthetase; methionyl-tRNA synthetase 2, mitochondrial; methyl CpG binding protein 2 (Rett syndrome); methyl-CpG binding domain protein 1-6, 3-like 1, 3-like 2, 3-like 3, 3-like 4, and 3-like 5; methylcrotonoyl-CoA carboxylase 1 (alpha); methylcrotonoyl-CoA carboxylase 2 (beta); methylenetetrahydrofolate dehydrogenase (NADP+ dependent) 1, methenyltetrahydrofolate cyclohydrolase, formyltetrahydrofolate synthetase; methylenetetrahydrofolate dehydrogenase (NADP+ dependent) 1-like; methylenetetrahydrofolate dehydrogenase (NADP+ dependent) 2, methenyltetrahydrofolate cyclohydrolase; methylenetetrahydrofolate dehydrogenase (NADP+ dependent) 2-like; methylenetetrahydrofolate reductase (NAD(P)H); methylmalonic aciduria (cobalamin deficiency) cblA type; methylmalonic aciduria (cobalamin deficiency) cb1B type; methylmalonic aciduria (cobalamin deficiency) cb1C type, with homocystinuria; methylmalonic aciduria (cobalamin deficiency) cb1D type, with homocystinuria; methylmalonyl CoA epimerase; methylmalonyl CoA mutase; methylphosphate capping enzyme; methylsterol monooxygenase 1; methylthioadenosine phosphorylase; methyltransferase like 1, like 2A, like 2B, like 3-like 5, like 7A, like 7B, like 8-like10, like 11A, like 11B, like 12-like 20, like 21A, like 21B, like 21C, like 21D, like 22 and like 23; mevalonate (diphospho) decarboxylase; mevalonate kinase; MFNG O-fucosylpeptide 3-beta-N-acetylglucosaminyltransferase; MGC39584 protein; MGC9913 protein; MEW class I polypeptide-related sequence A; MEW class I polypeptide-related sequence B; MICAL C-terminal like; MICAL-like 1; MICAL-like 2; microcephalin 1; microfibrillar associated protein 1-5 and 3-like; microphthalmia-associated transcription factor; microseminoprotein, beta-; microseminoprotein, prostate associated; microsomal glutathione S-transferase 1-3; microsomal triglyceride transfer protein; microspherule protein 1; microtubule associated serine/threonine kinase 1; microtubule associated serine/threonine kinase 2; microtubule associated serine/threonine kinase 3; microtubule associated serine/threonine kinase family member 4; microtubule associated serine/threonine kinase-like; microtubule associated tumor suppressor 1; microtubule associated tumor suppressor candidate 2; microtubule-actin crosslinking factor 1; microtubule-associated protein 1 light chain 3 alpha, beta, beta 2 and gamma; microtubule-associated protein 1A, 1B, 1S, 2, 4, 6, 7, and 9; microtubule-associated protein tau; microtubule-associated protein tau isoform 8; microtubule-associated protein, RP/EB family, member 1-member 3; midkine (neurite growth-promoting factor 2); midline 1 (Opitz/BBB syndrome); midline 2; midnolin; MIF4G domain containing; migration and invasion enhancer 1; migration and invasion inhibitory protein; milk fat globule-EGF factor 8 protein; minichromosome maintenance complex binding protein; minichromosome maintenance complex component 2-10 and 3 associated protein; mirror-image polydactyly 1; MIS18 binding protein 1; misshapen-like kinase 1; mitochondrial amidoxime reducing component 1 and 2; mitochondrial antiviral signaling protein; mitochondrial calcium uniporter; mitochondrial calcium uptake 1; mitochondrial carrier 1; mitochondrial carrier 2; mitochondrial carrier triple repeat 1; mitochondrial carrier triple repeat 2; mitochondrial carrier triple repeat 6; mitochondrial coiled-coil domain 1; mitochondrial E3 ubiquitin protein ligase 1; mitochondrial fission factor; mitochondrial fission process 1; mitochondrial fission regulator 1; Mitochondrial GTPase 1; mitochondrial inner membrane organizing system 1; mitochondrial intermediate peptidase; mitochondrial methionyl-tRNA formyltransferase; mitochondrial poly(A) polymerase; Mitochondrial ribonuclease P protein 3; mitochondrial ribosomal protein 63, L1, L10, L11, L12, L13, L14, L15, L16, L17, L18, L19, L2, L20, L21, L22, L23, L24, L27, L28, L3, L30, L32, L33, L34, L35, L36, L37, L38, L39, L4, L40, L41, L42, L43, L44, L45, L47, L48, L49, L50, L51, L52, L53, L54, L55, L9, S10, S11, S12, S14, S15, S16, S17, S18A, S18B, S18C, S2, 521, S22, S23, S24, S25, S26, S27, S28, S30, 531, S33, S34, S35, S36, S5, S6, S7, and S9; mitochondrial ribosome recycling factor; mitochondrial trans-2-enoyl-CoA reductase; mitochondrial transcription termination factor; mitochondrial translational initiation factor 2; mitochondrial translational initiation factor 3; mitochondrial translational release factor 1; mitochondrial translational release factor 1-like; mitochondrially encoded ATP synthase 6; mitochondrially encoded cytochrome b; mitochondrially encoded cytochrome c oxidase I-III; mitochondrially encoded NADH dehydrogenase 1; mitochondrially encoded NADH dehydrogenase 2; mitochondrially encoded NADH dehydrogenase 3; mitochondrially encoded NADH dehydrogenase 4; mitochondrially encoded NADH dehydrogenase 4L; mitochondrially encoded NADH dehydrogenase 5; mitochondrially encoded NADH dehydrogenase 6; mitofusin 1; mitofusin 2; mitogen-activated protein kinase 1, 3, 4, 6-15, 1 interacting protein 1-like, 8 interacting protein 1, 8 interacting protein 2 and 8 interacting protein 3; mitogen-activated protein kinase associated protein 1; mitogen-activated protein kinase binding protein 1; mitogen-activated protein kinase kinase 1-7; mitogen-activated protein kinase kinase kinase 1-15; mitogen-activated protein kinase kinase kinase kinase 1-5; Mitogen-activated protein kinase kinase kinase MLK4; Mitogen-activated protein kinase kinase kinase MLT; mitogen-activated protein kinase-activated protein kinase 2-5; mitotic spindle organizing protein 1, 2A and 2B; Mix paired-like homeobox; mixed lineage kinase domain-like; MKI67 (FHA domain) interacting nucleolar phosphoprotein; MKL/myocardin-like 2; MLF1 interacting protein; MLX interacting protein; MLX interacting protein-like; MMS22-like, DNA repair protein; MOB family member 4, phocein; MOB kinase activator 1A, 1B, 3A, 3B and 3C; modulator of apoptosis 1; moesin; mohawk homeobox; MOK protein kinase; molybdenum cofactor sulfurase; molybdenum cofactor synthesis 1-3; monoacylglycerol O-acyltransferase 1-3; monoamine oxidase A; monoamine oxidase B; monocyte to macrophage differentiation-associated; monocyte to macrophage differentiation-associated 2; monoglyceride lipase; monooxygenase, DBH-like 1; MORC family CW-type zinc finger 1-4; Morf4 family associated protein 1; Morf4 family associated protein 1-like 1; mortality factor 4 like 1; mortality factor 4 like 2; motilin; motilin receptor; motor neuron and pancreas homeobox 1; M-phase phosphoprotein 10 (U3 small nucleolar ribonucleoprotein); M-phase phosphoprotein 6; M-phase phosphoprotein 8; M-phase phosphoprotein 9; MPN domain containing; MpV17 mitochondrial inner membrane protein; MPV17 mitochondrial membrane protein-like; MPV17 mitochondrial membrane protein-like 2; msh homeobox 1; msh homeobox 2; MT-RNR2-like 1, like 3-5, like 7, like 8 and like 10; MU-2/AP1M2 domain containing, death-inducing; mucin 1, cell surface associated; mucin 12, cell surface associated; mucin 13, cell surface associated; mucin 15, cell surface associated; mucin 16, cell surface associated; mucin 17, cell surface associated; mucin 19, oligomeric; mucin 2, oligomeric mucus/gel-forming; mucin 20, cell surface associated; mucin 21, cell surface associated; mucin 3A, cell surface associated; mucin 4, cell surface associated; mucin SAC, oligomeric mucus/gel-forming; mucin 5B, oligomeric mucus/gel-forming; mucin 6, oligomeric mucus/gel-forming; mucin 7, secreted; Mucin-21; mucin-like 1; mucolipin 1; mucolipin 2; mucolipin 3; mucosa associated lymphoid tissue lymphoma translocation gene 1; mucosal vascular addressin cell adhesion molecule 1; multimerin 1; multimerin 2; multiple C2 domains, transmembrane 1 and 2; multiple coagulation factor deficiency 2; multiple EGF-like-domains 6, and 8-11; multiple endocrine neoplasia I; multiple inositol-polyphosphate phosphatase 1; multiple PDZ domain protein; muscle, skeletal, receptor tyrosine kinase; muscle-related coiled-coil protein; muscular LMNA-interacting protein; musculin; musculoskeletal, embryonic nuclear protein 1; mutated in colorectal cancers; MYB binding protein (P160) la; Myb-like, SWIRM and MPN domains 1; Myb-related transcription factor, partner of profilin; MYC associated factor X; MYC binding protein 2; MYC induced nuclear antigen; myc target 1; MYC-associated zinc finger protein (purine-binding transcription factor); MYCBP associated protein; myelin associated glycoprotein; myelin basic protein; myelin expression factor 2; myelin oligodendrocyte glycoprotein; myelin protein zero; myelin protein zero-like 1, like 2 and like 3; myelin transcription factor 1; myelin transcription factor 1-like; myelin-associated oligodendrocyte basic protein; myelodysplastic syndrome 2 translocation associated; myeloid cell leukemia sequence 1 (BCL2-related); myeloid cell nuclear differentiation antigen; myeloid differentiation primary response gene (88); myeloid leukemia factor 1; myeloid leukemia factor 2; myeloid zinc finger 1; myeloid/lymphoid and mixed-lineage leukemia 2; myeloid/lymphoid and mixed-lineage leukemia 3; myeloid-associated differentiation marker; myeloid-associated differentiation marker-like 2; myeloma overexpressed (in a subset of t(11;14) positive multiple myelomas); myeloma overexpressed 2; myeloperoxidase; myeloproliferative leukemia virus oncogene; myocardin; myocilin, trabecular meshwork inducible glucocorticoid response; myocyte enhancer factor 2A-2D; MyoD family inhibitor; MyoD family inhibitor domain containing; myoferlin; myogenic differentiation 1; myogenic factor 5; myogenic factor 6 (herculin); myogenin (myogenic factor 4); myoglobin; myo-inositol oxygenase; myomesin (M-protein) 2, 165 kDa; myomesin 1, 185 kDa; myomesin family, member 3; myoneurin; myopalladin; myosin binding protein C, cardiac; myosin binding protein C, fast type; myosin binding protein C, slow type; myosin binding protein H; myosin binding protein H-like; myosin IA-IH, IIIA, IIIB, IXA, IXB, VA, VB, VC, VI, VIIA, VIIA and Rab interacting protein, VIIB, X, XIX, XVA, XVI, XVIIIA, and XVIIIB; myosin light chain kinase; myosin light chain kinase 2; myosin light chain kinase 3; myosin light chain kinase family, member 4; myosin light chain, phosphorylatable, fast skeletal muscle; myosin phosphatase Rho interacting protein; myosin regulatory light chain myosin, heavy chain 1 (skeletal muscle, adult), 2 (skeletal muscle, adult), 3 (skeletal muscle, embryonic), 4 (skeletal muscle), 6 (cardiac muscle, alpha), 7 (cardiac muscle, beta), 7B (cardiac muscle, beta), 8 (skeletal muscle, perinatal), 9 (non-muscle), 10 (non-muscle), 11 (smooth muscle), 13 (skeletal muscle), 14 (non-muscle), and 15; myosin, light chain 1 (alkali, skeletal, fast), 2 (regulatory, cardiac, slow), 3 (alkali, ventricular, skeletal, slow), 4 (alkali atrial, embryonic), 5 (regulatory), 6 (alkali, smooth muscle and non-muscle), 6B (alkali, smooth muscle and non-muscle), 7 (regulatory), 9 (regulatory), 10 (regulatory), 12A (regulatory, non-sarcomeric), and 12B (regulatory); myostatin; myotilin; myotrophin; myotubularin 1; myotubularin related protein 1-12 and 14; myozenin 1-3; myristoylated alanine-rich protein kinase C substrate; MYST/Esa1-associated factor 6; myxovirus (influenza virus) resistance 1, interferon-inducible protein p78 (mouse); myxovirus (influenza virus) resistance 2 (mouse); N(alpha)-acetyltransferase 10, NatA catalytic subunit; N(alpha)-acetyltransferase 11, NatA catalytic subunit; N(alpha)-acetyltransferase 15, NatA auxiliary subunit; N(alpha)-acetyltransferase 16, NatA auxiliary subunit; N(alpha)-acetyltransferase 20, NatB catalytic subunit; N(alpha)-acetyltransferase 25, NatB auxiliary subunit; N(alpha)-acetyltransferase 30, NatC catalytic subunit; N(alpha)-acetyltransferase 35, NatC auxiliary subunit; N(alpha)-acetyltransferase 38, NatC auxiliary subunit; N(alpha)-acetyltransferase 50, NatE catalytic subunit; N(alpha)-acetyltransferase 60, NatF catalytic subunit; Na+/K+ transporting ATPase interacting 1; Na+/K+ transporting ATPase interacting 2; Na+/K+ transporting ATPase interacting 3; Na+/K+ transporting ATPase interacting 4; NAC alpha domain containing; NACC family member 2, BEN and BTB (POZ) domain containing; N-acetylated alpha-linked acidic dipeptidase 2; N-acetylated alpha-linked acidic dipeptidase-like 1; N-acetylated alpha-linked acidic dipeptidase-like 2; N-acetylgalactosaminidase, alpha-; N-acetylglucosamine kinase; N-acetylglucosamine-1-phosphate transferase, alpha and beta subunits; N-acetylglucosamine-1-phosphate transferase, gamma subunit; N-acetylglucosamine-1-phosphodiester alpha-N-acetylglucosaminidase; N-acetylglucosaminidase, alpha; N-acetylglutamate synthase; N-acetylneuraminate pyruvate lyase (dihydrodipicolinate synthase); N-acetylneuraminic acid phosphatase; N-acetylneuraminic acid synthase; N-acetyltransferase 1 (arylamine N-acetyltransferase); N-acetyltransferase 10 (GCNS-related); N-acetyltransferase 2 (arylamine N-acetyltransferase); N-acetyltransferase 6 (GCNS-related); N-acyl phosphatidylethanolamine phospholipase D; N-acylaminoacyl-peptide hydrolase; N-acylethanolamine acid amidase; N-acylsphingosine amidohydrolase (acid ceramidase) 1; N-acylsphingosine amidohydrolase (non-lysosomal ceramidase) 2, 2B and 2C; NAD kinase; NAD synthetase 1; NAD(P) dependent steroid dehydrogenase-like; NAD(P)H dehydrogenase, quinone 1; NAD(P)H dehydrogenase, quinone 2; NADH dehydrogenase (ubiquinone) 1 alpha subcomplex, 1 (7.5 kDa), 2 (8 kDa), 3 (9 kDa), 4 (9 kDa), 4-like 2), 5 (13 kDa), 6 (14 kDa), 7 (14.5 kDa), 8 (19 kDa), 9 (39 kDa), 10 (42 kDa), 11 (14.7 kDa), 12, and 13; NADH dehydrogenase (ubiquinone) 1 alpha subcomplex, assembly factor 1-assembly factor 4; NADH dehydrogenase (ubiquinone) 1 beta subcomplex, 1 (7 kDa), 2 (8 kDa), 3 (12 kDa), 4 (15 kDa), 5 (16 kDa), 6 (17 kDa), 7 (18 kDa), 8 (19 kDa), 9 (22 kDa), 10 (22 kDa), and 11 (17.3 kDa); NADH dehydrogenase (ubiquinone) 1, alpha/beta subcomplex, 1, 8 kDa; NADH dehydrogenase (ubiquinone) 1, subcomplex unknown, 1, 6 kDa; NADH dehydrogenase (ubiquinone) 1, subcomplex unknown, 2, 14.5 kDa; NADH dehydrogenase (ubiquinone) Fe—S protein 1 (75 kDa (NADH-coenzyme Q reductase)), protein 2 (49 kDa (NADH-coenzyme Q reductase)), protein 3 (30 kDa (NADH-coenzyme Q reductase)), protein 4 (18 kDa (NADH-coenzyme Q reductase)), protein 5 (15 kDa (NADH-coenzyme Q reductase)), protein 6 (13 kDa (NADH-coenzyme Q reductase)), protein 7 (20 kDa (NADH-coenzyme Q reductase)), and protein 8 (23 kDa (NADH-coenzyme Q reductase)); NADH dehydrogenase (ubiquinone) flavoprotein 1, 51 kDa; NADH dehydrogenase (ubiquinone) flavoprotein 2, 24 kDa; NADH dehydrogenase (ubiquinone) flavoprotein 3, 10 kDa; NADPH dependent diflavin oxidoreductase 1; NADPH oxidase 1, 3 and 4; NADPH oxidase activator 1; NADPH oxidase organizer 1; NADPH oxidase, EF-hand calcium binding domain 5; Nance-Horan syndrome (congenital cataracts and dental anomalies); Nanog homeobox; NANOG neighbor homeobox; napsin A aspartic peptidase; nardilysin (N-arginine dibasic convertase); nasal embryonic LHRH factor; nascent polypeptide-associated complex alpha subunit; nascent polypeptide-associated complex alpha subunit 2; natriuretic peptide A; natriuretic peptide B; natriuretic peptide C; natriuretic peptide receptor A/guanylate cyclase A (atrionatriuretic peptide receptor A); natriuretic peptide receptor B/guanylate cyclase B (atrionatriuretic peptide receptor B); natriuretic peptide receptor C/guanylate cyclase C (atrionatriuretic peptide receptor C); natural cytotoxicity triggering receptor 1-receptor 3; natural killer cell group 7 sequence; natural killer-tumor recognition sequence; NCK adaptor protein 1; NCK adaptor protein 2; NCK interacting protein with SH3 domain; NCK-associated protein 1; NCK-associated protein 1-like; NCK-associated protein 5; NCK-associated protein 5-like; N-deacetylase/N-sulfotransferase (heparan glucosaminyl) 1-4; NDRG family member 2-member 4; nebulette; nebulin; nebulin-related anchoring protein; NECAP endocytosis associated 1; NECAP endocytosis associated 2; necdin-like 2; NEDD4 binding protein 1; NEDD4 binding protein 2; NEDD4 binding protein 2-like 1; NEDD4 binding protein 2-like 2; NEDD4 binding protein 3; Nedd4 family interacting protein 1; Nedd4 family interacting protein 2; NEDD8 activating enzyme E1 subunit 1; negative regulator of ubiquitin-like proteins 1; neighbor of BRCA1 gene 1; NEL-like 1 (chicken); NEL-like 2 (chicken); nemo-like kinase; neogenin 1; nephroblastoma overexpressed gene; nephronectin; nephronophthisis 1 (juvenile); nephronophthisis 3 (adolescent); nephronophthisis 4; nephrosis 1, congenital, Finnish type (nephrin); nephrosis 2, idiopathic, steroid-resistant (podocin); nerve growth factor (beta polypeptide); nerve growth factor receptor; nerve growth factor receptor (TNFRSF16) associated protein 1; nescient helix loop helix 1; nescient helix loop helix 2; nestin; N-ethylmaleimide-sensitive factor; N-ethylmaleimide-sensitive factor attachment protein, alpha; N-ethylmaleimide-sensitive factor attachment protein, beta; N-ethylmaleimide-sensitive factor attachment protein, gamma; netrin 1, 3-5, G1 and G2; neudesin neurotrophic factor; neugrin, neurite outgrowth associated; neural cell adhesion molecule 1 and 2; neural precursor cell expressed, developmentally down-regulated 1, 4, 4-like, 8 and 9; neural proliferation, differentiation and control, 1; neural retina leucine zipper; neuregulin 1-4; neurensin 1-2; neurexin 1-3; neurexophilin 1-4; neuritin 1; neuritin 1-like; neurobeachin; neurobeachin-like 1 and like 2; neuroblastoma amplified sequence; neuroblastoma breakpoint family, member 3, member 4, member 6, member 9-member 12, member 14-member 16, member 20 and member 24; neuroblastoma, suppression of tumorigenicity 1; neurocalcin delta; neurocan; neurochondrin; neuroepithelial cell transforming 1; neurofascin; neurofibromin 1; neurofibromin 2 (merlin); neurofilament, heavy polypeptide; neurofilament, medium polypeptide; neurogenic differentiation 1, 2, 4 and 6; neurogenin 1-3; neuroglobin; neurogranin (protein kinase C substrate, RC3); neuroguidin, EIF4E binding protein; neuroligin 1-3, 4 (X-linked) and 4 (Y-linked); neurolysin (metallopeptidase M3 family); neuromedin B; neuromedin B receptor; neuromedin S; neuromedin U; neuromedin U receptor 1; neuromedin U receptor 2; neuron navigator 1-3; neuronal calcium sensor 1; neuronal cell adhesion molecule; neuronal growth regulator 1; neuronal guanine nucleotide exchange factor; neuronal PAS domain protein 1-protein 4; neuronal pentraxin I; neuronal pentraxin II; neuronal pentraxin receptor; neuronal tyrosine-phosphorylated phosphoinositide-3-kinase adaptor 1; neuronal tyrosine-phosphorylated phosphoinositide-3-kinase adaptor 2; neuronatin; neuron-derived neurotrophic factor; Neuron-specific protein family member 1; Neuron-specific protein family member 2; neuro-oncological ventral antigen 1; neuro-oncological ventral antigen 2; neuropeptide B; neuropeptide FF receptor 1; neuropeptide FF receptor 2; neuropeptide FF-amide peptide precursor; neuropeptide S; neuropeptide S receptor 1; neuropeptide VF precursor; neuropeptide W; neuropeptide Y; neuropeptide Y receptor Yl; neuropeptide Y receptor Y2; neuropeptide Y receptor Y5; neuropeptides B/W receptor 1; neuropeptides B/W receptor 2; neuropilin (NRP) and tolloid (TLL)-like 1; neuropilin (NRP) and tolloid (TLL)-like 2; neuropilin 1; neuropilin 2; neuroplastin; neurotensin; neurotensin receptor 1 (high affinity); neurotensin receptor 2; neurotrimin; neurotrophic tyrosine kinase, receptor, type 1; neurotrophic tyrosine kinase, receptor, type 2; neurotrophic tyrosine kinase, receptor, type 3; neurotrophin 3; neurotrophin 4; neurturin; neutral cholesterol ester hydrolase 1; neutral sphingomyelinase (N-SMase) activation associated factor; neutrophil cytosolic factor 1; neutrophil cytosolic factor 2; neutrophil cytosolic factor 4, 40 kDa; nexilin (F actin binding protein); NFAT activating protein with ITAM motif 1; NFKB activating protein; NFKB activating protein-like; NFKB inhibitor interacting Ras-like 1; NFKB inhibitor interacting Ras-like 2; NFKB repressing factor; NGFI-A binding protein 1 (EGR1 binding protein 1); NGFI-A binding protein 2 (EGR1 binding protein 2); N-glycanase 1; NHS-like 1; NHS-like 2; nibrin; nicalin; nicastrin; nicolin 1; nicotinamide N-methyltransferase; nicotinamide nucleotide adenylyltransferase 1-3; nicotinamide nucleotide transhydrogenase; nicotinamide phosphoribosyltransferase; nicotinamide phosphoribosyltransferase-like; nidogen 1; nidogen 2 (osteonidogen); Niemann-Pick disease, type C1 and type C2; Nik related kinase; NIMA (never in mitosis gene a)-related kinase 1-11; ninein (GSK3B interacting protein); ninein-like; ninjurin 1; ninjurin 2; nischarin; nitric oxide associated 1; nitric oxide synthase 1 (neuronal); nitric oxide synthase 1 (neuronal) adaptor protein; nitric oxide synthase 2, inducible; nitric oxide synthase 3 (endothelial cell); nitric oxide synthase interacting protein; nitric oxide synthase trafficker; nitrilase 1; nitrilase family, member 2; NK1 homeobox 1 and 2; NK2 homeobox 1-6 and 8; NK3 homeobox 1 and 2; NK6 homeobox 1-3; NLR family member X1; NLR family, apoptosis inhibitory protein; NMDA receptor regulated 2; NME gene family member 9; NME1-NME2 readthrough; N-methylpurine-DNA glycosylase; N-myc (and STAT) interactor; N-myc downstream regulated 1; N-myristoyltransferase 1; N-myristoyltransferase 2; NOBOX oogenesis homeobox; NODAL modulator 1-3; noggin; non imprinted in Prader-Willi/Angelman syndrome 1; non imprinted in Prader-Willi/Angelman syndrome 2; non-metastatic cells 1, protein (NM23A) expressed in; non-metastatic cells 2, protein (NM23B) expressed in; non-metastatic cells 3, protein expressed in; non-metastatic cells 4, protein expressed in; non-metastatic cells 5, protein expressed in (nucleoside-diphosphate kinase); non-metastatic cells 6, protein expressed in (nucleoside-diphosphate kinase); non-metastatic cells 7, protein expressed in (nucleoside-diphosphate kinase); non-POU domain containing, octamer-binding; non-SMC condensin I complex, subunit D2; non-SMC condensin I complex, subunit G; non-SMC condensin I complex, subunit H; non-SMC condensin II complex, subunit D3; non-SMC condensin II complex, subunit G2; non-SMC condensin II complex, subunit H2; NOP2/Sun domain family, member 2-member 7; Norrie disease (pseudoglioma); notch 1; notch 2; notch 2 N-terminal like; notch 3; notch 4; NOTCH-regulated ankyrin repeat protein; notochord homeobox; Novel protein (FLJ40547); Novel protein similar to contactin associated protein-like 3 (CNTNAP3); Novel protein Similar to bA90M5.1 (Novel protein); NPC1 (Niemann-Pick disease, type C1, gene)-like 1; NPIP-like protein 1; NSFL1 (p9′7) cofactor (p4′7); N-sulfoglucosamine sulfohydrolase; N-terminal asparagine amidase; N-terminal EF-hand calcium binding protein 1; N-terminal EF-hand calcium binding protein 2; N-terminal EF-hand calcium binding protein 3; NTF2-like export factor 1; NUAK family, SNF1-like kinase, 1; NUAK family, SNF1-like kinase, 2; nuclear apoptosis inducing factor 1; nuclear autoantigenic sperm protein (histone-binding); nuclear cap binding protein subunit 1, 80 kDa; nuclear cap binding protein subunit 2, 20 kDa; nuclear cap binding protein subunit 2-like; nuclear casein kinase and cyclin-dependent kinase substrate 1; nuclear export mediator factor; nuclear factor (erythroid-derived 2), 45 kDa; nuclear factor (erythroid-derived 2)-like 1; nuclear factor (erythroid-derived 2)-like 2; nuclear factor (erythroid-derived 2)-like 3; nuclear factor UA; nuclear factor IB; nuclear factor I/C (CCAAT-binding transcription factor); nuclear factor I/X (CCAAT-binding transcription factor); nuclear factor of activated T-cells 5, tonicity-responsive; nuclear factor of activated T-cells, cytoplasmic, calcineurin-dependent 1, 2, 2 interacting protein, 3 and 4; nuclear factor of kappa light polypeptide gene enhancer in B-cells 1; nuclear factor of kappa light polypeptide gene enhancer in B-cells 2 (p49/p100); nuclear factor of kappa light polypeptide gene enhancer in B-cells inhibitor, alpha, beta, delta, epsilon, and zeta; nuclear factor of kappa light polypeptide gene enhancer in B-cells inhibitor-like 1; nuclear factor related to kappaB binding protein; nuclear factor, interleukin 3 regulated; nuclear fragile X mental retardation protein interacting protein 1; nuclear fragile X mental retardation protein interacting protein 2; nuclear mitotic apparatus protein 1; nuclear pore complex interacting protein; nuclear pore complex interacting protein-like 1-like 3; nuclear prelamin A recognition factor; nuclear prelamin A recognition factor-like; nuclear protein, ataxia-telangiectasia locus; nuclear protein, transcriptional regulator, 1; nuclear receptor 2C2-associated protein; nuclear receptor binding factor 2; nuclear receptor binding protein 1; nuclear receptor binding protein 2; nuclear receptor binding SET domain protein 1; nuclear receptor coactivator 1-7; nuclear receptor corepressor 1-2; nuclear receptor interacting protein 1; nuclear receptor interacting protein 2; nuclear receptor interacting protein 3; nuclear receptor subfamily 0, group B, member 1; nuclear receptor subfamily 0, group B, member 2; nuclear receptor subfamily 1, group D, member 1; nuclear receptor subfamily 1, group D, member 2; nuclear receptor subfamily 1, group H, member 2; nuclear receptor subfamily 1, group H, member 3; nuclear receptor subfamily 1, group H, member 4; nuclear receptor subfamily 1, group I, member 2; nuclear receptor subfamily 1, group I, member 3; nuclear receptor subfamily 2, group C, member 1; nuclear receptor subfamily 2, group C, member 2; nuclear receptor subfamily 2, group E, member 1; nuclear receptor subfamily 2, group E, member 3; nuclear receptor subfamily 2, group F, member 1; nuclear receptor subfamily 2, group F, member 2; nuclear receptor subfamily 2, group F, member 6; nuclear receptor subfamily 3, group C, member 1 (glucocorticoid receptor); nuclear receptor subfamily 3, group C, member 2; nuclear receptor subfamily 4, group A, member 1; nuclear receptor subfamily 4, group A, member 2; nuclear receptor subfamily 4, group A, member 3; nuclear receptor subfamily 5, group A, member 1; nuclear receptor subfamily 5, group A, member 2; nuclear receptor subfamily 6, group A, member 1; nuclear respiratory factor 1; nuclear RNA export factor 1; nuclear RNA export factor 2; nuclear RNA export factor 2B; nuclear RNA export factor 3; nuclear RNA export factor 5; nuclear speckle splicing regulatory protein 1; nuclear transcription factor Y, alpha; nuclear transcription factor Y, beta; nuclear transcription factor Y, gamma; nuclear transcription factor, X-box binding 1; nuclear transcription factor, X-box binding-like 1; nuclear transport factor 2; nuclear transport factor 2-like export factor 2; nuclear VCP-like; nucleobindin 1; nucleobindin 2; nucleolar and coiled-body phosphoprotein 1; nucleolar and spindle associated protein 1; nucleolar protein 10; nucleolar protein 11; Nucleolar protein 12; nucleolar protein 3 (apoptosis repressor with CARD domain); nucleolar protein 4; nucleolar protein 7, 27 kDa; nucleolar protein 8; nucleolar protein 9; nucleolar protein family 6 (RNA-associated); nucleolar protein with MIF4G domain 1; nucleolin; nucleophosmin (nucleolar phosphoprotein B23, numatrin); nucleophosmin/nucleoplasmin 2; nucleophosmin/nucleoplasmin 3; nucleoporin 107 kDa; nucleoporin 133 kDa; nucleoporin 153 kDa; nucleoporin 155 kDa; nucleoporin 160 kDa; nucleoporin 188 kDa; nucleoporin 205 kDa; nucleoporin 210 kDa; nucleoporin 210 kDa-like; nucleoporin 214 kDa; nucleoporin 35 kDa; nucleoporin 37 kDa; nucleoporin 43 kDa; nucleoporin 50 kDa; nucleoporin 54 kDa; nucleoporin 62 kDa; nucleoporin 62 kDa C-terminal like; nucleoporin 85 kDa; nucleoporin 88 kDa; nucleoporin 93 kDa; nucleoporin 98 kDa; nucleoporin like 1; nucleoporin like 2; nucleoredoxin; nucleoredoxin-like 1; nucleoredoxin-like 2; nucleoside-triphosphatase, cancer-related; nucleosome assembly protein 1-like 1; nucleosome assembly protein 1-like 2; nucleosome assembly protein 1-like 3; nucleosome assembly protein 1-like 4; nucleosome assembly protein 1-like 5; nucleosome assembly protein 1-like 6; nucleotide binding protein 1; nucleotide binding protein 2; nucleotide binding protein-like; nucleus accumbens associated 1, BEN and BTB (POZ) domain containing; nudix (nucleoside diphosphate linked moiety X)-type motif 1, 2, 21, 22, 3, 4, 5, 6, 7, 8, 9, 10, 11, 12, 13, 14, 15, 16, 16-like 1, 17, 18, and 19; nurim (nuclear envelope membrane protein); nyctalopin; NYN domain and retroviral integrase containing; O-6-methylguanine-DNA methyltransferase; Obg-like ATPase 1; obscurin, cytoskeletal calmodulin and titin-interacting RhoGEF; obscurin-like 1; occludin; oculocerebrorenal syndrome of Lowe; oculocutaneous albinism II; odontogenic, ameloblast associated; odorant binding protein 2A; odorant binding protein 2B; oleoyl-ACP hydrolase; olfactomedin 1; olfactomedin 2; olfactomedin 3; olfactomedin 4; olfactomedin-like 1; olfactomedin-like 2A; olfactomedin-like 2B; olfactomedin-like 3; olfactory marker protein; Olfactory receptor 11H12; olfactory receptor, family 1, subfamily A, member 1; olfactory receptor, family 1, subfamily A, member 2; olfactory receptor, family 1, subfamily B, member 1; olfactory receptor, family 1, subfamily C, member 1; olfactory receptor, family 1, subfamily D, member 2; olfactory receptor, family 1, subfamily E, member 1; olfactory receptor, family 1, subfamily E, member 2; olfactory receptor, family 1, subfamily F, member 1; olfactory receptor, family 1, subfamily G, member 1; olfactory receptor, family 1, subfamily I, member 1; olfactory receptor, family 1, subfamily J, member 1; olfactory receptor, family 1, subfamily J, member 2; olfactory receptor, family 1, subfamily J, member 4; olfactory receptor, family 1, subfamily K, member 1; olfactory receptor, family 1, subfamily L, member 1; olfactory receptor, family 1, subfamily L, member 3; olfactory receptor, family 1, subfamily L, member 4; olfactory receptor, family 1, subfamily L, member 6; olfactory receptor, family 1, subfamily L, member 8; olfactory receptor, family 1, subfamily M, member 1; olfactory receptor, family 1, subfamily N, member 1; olfactory receptor, family 1, subfamily N, member 2; olfactory receptor, family 1, subfamily Q, member 1; olfactory receptor, family 1, subfamily S, member 1; olfactory receptor, family 1, subfamily S, member 2; olfactory receptor, family 10, subfamily A, member 2; olfactory receptor, family 10, subfamily A, member 3; olfactory receptor, family 10, subfamily A, member 4; olfactory receptor, family 10, subfamily A, member 5; olfactory receptor, family 10, subfamily A, member 6; olfactory receptor, family 10, subfamily A, member 7; olfactory receptor, family 10, subfamily AD, member 1; olfactory receptor, family 10, subfamily AG, member 1; olfactory receptor, family 10, subfamily C, member 1; olfactory receptor, family 10, subfamily D, member 3 (non-functional); olfactory receptor, family 10, subfamily G, member 2; olfactory receptor, family 10, subfamily G, member 3; olfactory receptor, family 10, subfamily G, member 4; olfactory receptor, family 10, subfamily G, member 7; olfactory receptor, family 10, subfamily G, member 8; olfactory receptor, family 10, subfamily G, member 9; olfactory receptor, family 10, subfamily H, member 1; olfactory receptor, family 10, subfamily H, member 2; olfactory receptor, family 10, subfamily H, member 3; olfactory receptor, family 10, subfamily H, member 4; olfactory receptor, family 10, subfamily H, member 5; olfactory receptor, family 10, subfamily J, member 1; olfactory receptor, family 10, subfamily J, member 3; olfactory receptor, family 10, subfamily J, member 5; olfactory receptor, family 10, subfamily K, member 1; olfactory receptor, family 10, subfamily K, member 2; olfactory receptor, family 10, subfamily P, member 1; olfactory receptor, family 10, subfamily Q, member 1; olfactory receptor, family 10, subfamily R, member 2; olfactory receptor, family 10, subfamily S, member 1; olfactory receptor, family 10, subfamily T, member 2; olfactory receptor, family 10, subfamily V, member 1; olfactory receptor, family 10, subfamily W, member 1; olfactory receptor, family 10, subfamily X, member 1; olfactory receptor, family 10, subfamily Z, member 1; olfactory receptor, family 11, subfamily A, member 1; olfactory receptor, family 11, subfamily G, member 2; olfactory receptor, family 11, subfamily H, member 1; olfactory receptor, family 11, subfamily H, member 4; olfactory receptor, family 11, subfamily H, member 6; olfactory receptor, family 11, subfamily L, member 1; olfactory receptor, family 12, subfamily D, member 2; olfactory receptor, family 12, subfamily D, member 3; olfactory receptor, family 13, subfamily A, member 1; olfactory receptor, family 13, subfamily C, member 2; olfactory receptor, family 13, subfamily C, member 3; olfactory receptor, family 13, subfamily C, member 4; olfactory receptor, family 13, subfamily C, member 5; olfactory receptor, family 13, subfamily C, member 8; olfactory receptor, family 13, subfamily C, member 9; olfactory receptor, family 13, subfamily D, member 1; olfactory receptor, family 13, subfamily F, member 1; olfactory receptor, family 13, subfamily G, member 1; olfactory receptor, family 13, subfamily H, member 1; olfactory receptor, family 13, subfamily J, member 1; olfactory receptor, family 14, subfamily A, member 16; olfactory receptor, family 14, subfamily A, member 2; olfactory receptor, family 14, subfamily C, member 36; olfactory receptor, family 14, subfamily I, member 1; olfactory receptor, family 14, subfamily J, member 1; olfactory receptor, family 14, subfamily K, member 1; olfactory receptor, family 2, subfamily A, member 1; olfactory receptor, family 2, subfamily A, member 12; olfactory receptor, family 2, subfamily A, member 14; olfactory receptor, family 2, subfamily A, member 2; olfactory receptor, family 2, subfamily A, member 25; olfactory receptor, family 2, subfamily A, member 4; olfactory receptor, family 2, subfamily A, member 42; olfactory receptor, family 2, subfamily A, member 5; olfactory receptor, family 2, subfamily A, member 7; olfactory receptor, family 2, subfamily AE, member 1; olfactory receptor, family 2, subfamily AG, member 1; olfactory receptor, family 2, subfamily AJ, member 1; olfactory receptor, family 2, subfamily AK, member 2; olfactory receptor, family 2, subfamily AP, member 1; olfactory receptor, family 2, subfamily AT, member 4; olfactory receptor, family 2, subfamily B, member 11; olfactory receptor, family 2, subfamily B, member 2; olfactory receptor, family 2, subfamily B, member 3; olfactory receptor, family 2, subfamily B, member 6; olfactory receptor, family 2, subfamily C, member 1; olfactory receptor, family 2, subfamily C, member 3; olfactory receptor, family 2, subfamily D, member 2; olfactory receptor, family 2, subfamily D, member 3; olfactory receptor, family 2, subfamily F, member 1; olfactory receptor, family 2, subfamily F, member 2; olfactory receptor, family 2, subfamily G, member 2; olfactory receptor, family 2, subfamily G, member 3; olfactory receptor, family 2, subfamily G, member 6; olfactory receptor, family 2, subfamily H, member 1; olfactory receptor, family 2, subfamily H, member 2; olfactory receptor, family 2, subfamily J, member 2; olfactory receptor, family 2, subfamily J, member 3; olfactory receptor, family 2, subfamily K, member 2; olfactory receptor, family 2, subfamily L, member 13; olfactory receptor, family 2, subfamily L, member 2; olfactory receptor, family 2, subfamily L, member 3; olfactory receptor, family 2, subfamily L, member 5; olfactory receptor, family 2, subfamily L, member 8; olfactory receptor, family 2, subfamily M, member 2; olfactory receptor, family 2, subfamily M, member 3; olfactory receptor, family 2, subfamily M, member 4; olfactory receptor, family 2, subfamily M, member 5; olfactory receptor, family 2, subfamily M, member 7; olfactory receptor, family 2, subfamily S, member 2; olfactory receptor, family 2, subfamily T, member 1; olfactory receptor, family 2, subfamily T, member 10; olfactory receptor, family 2, subfamily T, member 11; olfactory receptor, family 2, subfamily T, member 12; olfactory receptor, family 2, subfamily T, member 2; olfactory receptor, family 2, subfamily T, member 27; olfactory receptor, family 2, subfamily T, member 29; olfactory receptor, family 2, subfamily T, member 3; olfactory receptor, family 2, subfamily T, member 33; olfactory receptor, family 2, subfamily T, member 34; olfactory receptor, family 2, subfamily T, member 35; olfactory receptor, family 2, subfamily T, member 4; olfactory receptor, family 2, subfamily T, member 5; olfactory receptor, family 2, subfamily T, member 6; olfactory receptor, family 2, subfamily T, member 8; olfactory receptor, family 2, subfamily V, member 1; olfactory receptor, family 2, subfamily V, member 2; olfactory receptor, family 2, subfamily W, member 1; olfactory receptor, family 2, subfamily W, member 3; olfactory receptor, family 2, subfamily Y, member 1; olfactory receptor, family 2, subfamily Z, member 1; olfactory receptor, family 3, subfamily A, member 1; olfactory receptor, family 3, subfamily A, member 2; olfactory receptor, family 3, subfamily A, member 3; olfactory receptor, family 4, subfamily A, member 15; olfactory receptor, family 4, subfamily A, member 16; olfactory receptor, family 4, subfamily A, member 47; olfactory receptor, family 4, subfamily A, member 5; olfactory receptor, family 4, subfamily B, member 1; olfactory receptor, family 4, subfamily C, member 11; olfactory receptor, family 4, subfamily C, member 12; olfactory receptor, family 4, subfamily C, member 13; olfactory receptor, family 4, subfamily C, member 15; olfactory receptor, family 4, subfamily C, member 16; olfactory receptor, family 4, subfamily C, member 3; olfactory receptor, family 4, subfamily C, member 46; olfactory receptor, family 4, subfamily C, member 5; olfactory receptor, family 4, subfamily C, member 6; olfactory receptor, family 4, subfamily D, member 1; olfactory receptor, family 4, subfamily D, member 10; olfactory receptor, family 4, subfamily D, member 11; olfactory receptor, family 4, subfamily D, member 2; olfactory receptor, family 4, subfamily D, member 5; olfactory receptor, family 4, subfamily D, member 6; olfactory receptor, family 4, subfamily D, member 9; olfactory receptor, family 4, subfamily E, member 2; olfactory receptor, family 4, subfamily F, member 15; olfactory receptor, family 4, subfamily F, member 16; olfactory receptor, family 4, subfamily F, member 17; olfactory receptor, family 4, subfamily F, member 21; olfactory receptor, family 4, subfamily F, member 29; olfactory receptor, family 4, subfamily F, member 3; olfactory receptor, family 4, subfamily F, member 4; olfactory receptor, family 4, subfamily F, member 5; olfactory receptor, family 4, subfamily F, member 6; olfactory receptor, family 4, subfamily K, member 1; olfactory receptor, family 4, subfamily K, member 13; olfactory receptor, family 4, subfamily K, member 14; olfactory receptor, family 4, subfamily K, member 15; olfactory receptor, family 4, subfamily K, member 17; olfactory receptor, family 4, subfamily K, member 2; olfactory receptor, family 4, subfamily K, member 5; olfactory receptor, family 4, subfamily L, member 1; olfactory receptor, family 4, subfamily M, member 1; olfactory receptor, family 4, subfamily M, member 2; olfactory receptor, family 4, subfamily N, member 2; olfactory receptor, family 4, subfamily N, member 4; olfactory receptor, family 4, subfamily N, member 5; olfactory receptor, family 4, subfamily P, member 4; olfactory receptor, family 4, subfamily Q, member 3; olfactory receptor, family 4, subfamily S, member 1; olfactory receptor, family 4, subfamily S, member 2; olfactory receptor, family 4, subfamily X, member 1; olfactory receptor, family 4, subfamily X, member 2; olfactory receptor, family 5, subfamily A, member 1; olfactory receptor, family 5, subfamily A, member 2; olfactory receptor, family 5, subfamily AC, member 2; olfactory receptor, family 5, subfamily AK, member 2; olfactory receptor, family 5, subfamily AN, member 1; olfactory receptor, family 5, subfamily AP, member 2; olfactory receptor, family 5, subfamily AR, member 1; olfactory receptor, family 5, subfamily AS, member 1; olfactory receptor, family 5, subfamily AU, member 1; olfactory receptor, family 5, subfamily B, member 12; olfactory receptor, family 5, subfamily B, member 17; olfactory receptor, family 5, subfamily B, member 2; olfactory receptor, family 5, subfamily B, member 21; olfactory receptor, family 5, subfamily B, member 3; olfactory receptor, family 5, subfamily C, member 1; olfactory receptor, family 5, subfamily D, member 13; olfactory receptor, family 5, subfamily D, member 14; olfactory receptor, family 5, subfamily D, member 16; olfactory receptor, family 5, subfamily D, member 18; olfactory receptor, family 5, subfamily F, member 1; olfactory receptor, family 5, subfamily H, member 1; olfactory receptor, family 5, subfamily H, member 14; olfactory receptor, family 5, subfamily H, member 15; olfactory receptor, family 5, subfamily H, member 2; olfactory receptor, family 5, subfamily H, member 6; olfactory receptor, family 5, subfamily I, member 1; olfactory receptor, family 5, subfamily J, member 2; olfactory receptor, family 5, subfamily K, member 1; olfactory receptor, family 5, subfamily K, member 2; olfactory receptor, family 5, subfamily K, member 3; olfactory receptor, family 5, subfamily K, member 4; olfactory receptor, family 5, subfamily L, member 1; olfactory receptor, family 5, subfamily L, member 2; olfactory receptor, family 5, subfamily M, member 1; olfactory receptor, family 5, subfamily M, member 10; olfactory receptor, family 5, subfamily M, member 11; olfactory receptor, family 5, subfamily M, member 3; olfactory receptor, family 5, subfamily M, member 8; olfactory receptor, family 5, subfamily M, member 9; olfactory receptor, family 5, subfamily P, member 2; olfactory receptor, family 5, subfamily P, member 3; olfactory receptor, family 5, subfamily R, member 1; olfactory receptor, family 5, subfamily T, member 1; olfactory receptor, family 5, subfamily T, member 2; olfactory receptor, family 5, subfamily T, member 3; olfactory receptor, family 5, subfamily V, member 1; olfactory receptor, family 5, subfamily W, member 2; olfactory receptor, family 51, subfamily A, member 2; olfactory receptor, family 51, subfamily A, member 4; olfactory receptor, family 51, subfamily A, member 7; olfactory receptor, family 51, subfamily B, member 2; olfactory receptor, family 51, subfamily B, member 4; olfactory receptor, family 51, subfamily B, member 5; olfactory receptor, family 51, subfamily B, member 6; olfactory receptor, family 51, subfamily D, member 1; olfactory receptor, family 51, subfamily E, member 1; olfactory receptor, family 51, subfamily E, member 2; olfactory receptor, family 51, subfamily F, member 1; olfactory receptor, family 51, subfamily F, member 2; olfactory receptor, family 51, subfamily G, member 1; olfactory receptor, family 51, subfamily G, member 2; olfactory receptor, family 51, subfamily I, member 1; olfactory receptor, family 51, subfamily I, member 2; olfactory receptor, family 51, subfamily L, member 1; olfactory receptor, family 51, subfamily M, member 1; olfactory receptor, family 51, subfamily Q, member 1; olfactory receptor, family 51, subfamily S, member 1; olfactory receptor, family 51, subfamily T, member 1; olfactory receptor, family 51, subfamily V, member 1; olfactory receptor, family 52, subfamily A, member 1; olfactory receptor, family 52, subfamily A, member 5; olfactory receptor, family 52, subfamily B, member 2; olfactory receptor, family 52, subfamily B, member 4; olfactory receptor, family 52, subfamily B, member 6; olfactory receptor, family 52, subfamily D, member 1; olfactory receptor, family 52, subfamily E, member 2; olfactory receptor, family 52, subfamily E, member 4; olfactory receptor, family 52, subfamily E, member 6; olfactory receptor, family 52, subfamily E, member 8; olfactory receptor, family 52, subfamily H, member 1; olfactory receptor, family 52, subfamily I, member 1; olfactory receptor, family 52, subfamily I, member 2; olfactory receptor, family 52, subfamily J, member 3; olfactory receptor, family 52, subfamily K, member 1; olfactory receptor, family 52, subfamily K, member 2; olfactory receptor, family 52, subfamily L, member 1; olfactory receptor, family 52, subfamily M, member 1; olfactory receptor, family 52, subfamily N, member 1; olfactory receptor, family 52, subfamily N, member 2; olfactory receptor, family 52, subfamily N, member 4; olfactory receptor, family 52, subfamily N, member 5; olfactory receptor, family 52, subfamily R, member 1; olfactory receptor, family 52, subfamily W, member 1; olfactory receptor, family 56, subfamily A, member 1; olfactory receptor, family 56, subfamily A, member 3; olfactory receptor, family 56, subfamily A, member 4; olfactory receptor, family 56, subfamily B, member 1; olfactory receptor, family 56, subfamily B, member 4; olfactory receptor, family 6, subfamily A, member 2; olfactory receptor, family 6, subfamily B, member 1; olfactory receptor, family 6, subfamily B, member 2; olfactory receptor, family 6, subfamily B, member 3; olfactory receptor, family 6, subfamily C, member 1; olfactory receptor, family 6, subfamily C, member 2; olfactory receptor, family 6, subfamily C, member 3; olfactory receptor, family 6, subfamily C, member 4; olfactory receptor, family 6, subfamily C, member 6; olfactory receptor, family 6, subfamily C, member 65; olfactory receptor, family 6, subfamily C, member 68; olfactory receptor, family 6, subfamily C, member 70; olfactory receptor, family 6, subfamily C, member 74; olfactory receptor, family 6, subfamily C, member 75; olfactory receptor, family 6, subfamily C, member 76; olfactory receptor, family 6, subfamily F, member 1; olfactory receptor, family 6, subfamily J, member 1; olfactory receptor, family 6, subfamily K, member 2; olfactory receptor, family 6, subfamily K, member 3; olfactory receptor, family 6, subfamily K, member 6; olfactory receptor, family 6, subfamily M, member 1; olfactory receptor, family 6, subfamily N, member 1; olfactory receptor, family 6, subfamily N, member 2; olfactory receptor, family 6, subfamily P, member 1; olfactory receptor, family 6, subfamily Q, member 1; olfactory receptor, family 6, subfamily S, member 1; olfactory receptor, family 6, subfamily T, member 1; olfactory receptor, family 6, subfamily V, member 1; olfactory receptor, family 6, subfamily X, member 1; olfactory receptor, family 6, subfamily Y, member 1; olfactory receptor, family 7, subfamily A, member 10; olfactory receptor, family 7, subfamily A, member 17; olfactory receptor, family 7, subfamily A, member 5; olfactory receptor, family 7, subfamily C, member 1; olfactory receptor, family 7, subfamily C, member 2; olfactory receptor, family 7, subfamily D, member 2; olfactory receptor, family 7, subfamily D, member 4; olfactory receptor, family 7, subfamily E, member 24; olfactory receptor, family 7, subfamily G, member 1; olfactory receptor, family 7, subfamily G, member 2; olfactory receptor, family 7, subfamily G, member 3; olfactory receptor, family 8, subfamily A, member 1; olfactory receptor, family 8, subfamily B, member 12; olfactory receptor, family 8, subfamily B, member 2; olfactory receptor, family 8, subfamily B, member 3; olfactory receptor, family 8, subfamily B, member 4; olfactory receptor, family 8, subfamily B, member 8; olfactory receptor, family 8, subfamily D, member 1; olfactory receptor, family 8, subfamily D, member 2; olfactory receptor, family 8, subfamily D, member 4; olfactory receptor, family 8, subfamily H, member 1; olfactory receptor, family 8, subfamily H, member 2; olfactory receptor, family 8, subfamily H, member 3; olfactory receptor, family 8, subfamily I, member 2; olfactory receptor, family 8, subfamily J, member 1; olfactory receptor, family 8, subfamily J, member 3; olfactory receptor, family 8, subfamily K, member 1; olfactory receptor, family 8, subfamily K, member 3; olfactory receptor, family 8, subfamily K, member 5; olfactory receptor, family 8, subfamily S, member 1; olfactory receptor, family 8, subfamily U, member 1; olfactory receptor, family 9, subfamily A, member 2; olfactory receptor, family 9, subfamily A, member 4; olfactory receptor, family 9, subfamily G, member 1; olfactory receptor, family 9, subfamily G, member 4; olfactory receptor, family 9, subfamily I, member 1; olfactory receptor, family 9, subfamily K, member 2; olfactory receptor, family 9, subfamily Q, member 1; olfactory receptor, family 9, subfamily Q, member 2; oligodendrocyte lineage transcription factor 2; oligodendrocyte myelin glycoprotein; oligodendrocyte transcription factor 1; oligodendrocyte transcription factor 3; oligodendrocytic myelin paranodal and inner loop protein; oligophrenin 1; oligosaccharyltransferase complex subunit; O-linked N-acetylglucosamine (GlcNAc) transferase (UDP-N-acetylglucosamine:polypeptide-N-acetylglucosaminyl transferase); omega-3 fatty acid receptor 1; oncomodulin; oncomodulin 2; oncoprotein induced transcript 3; oncostatin M; oncostatin M receptor; one cut homeobox 1; one cut homeobox 2; one cut homeobox 3; Opa interacting protein 5; opiate receptor-like 1; opioid binding protein/cell adhesion molecule-like; opioid growth factor receptor; opioid growth factor receptor-like 1; opioid receptor, delta 1; opioid receptor, kappa 1; opioid receptor, mu 1; opsin 1 (cone pigments), long-wave-sensitive; opsin 1 (cone pigments), medium-wave-sensitive; opsin 1 (cone pigments), medium-wave-sensitive 2; opsin 1 (cone pigments), short-wave-sensitive; opsin 3; opsin 4; opsin 5; optic atrophy 1 (autosomal dominant); optic atrophy 3 (autosomal recessive, with chorea and spastic paraplegia); opticin; optineurin; ORAI calcium release-activated calcium modulator 1-3; oral cancer overexpressed 1; oral-facial-digital syndrome 1; organic solute carrier partner 1; Organic solute transporter subunit alpha; Organic solute transporter subunit beta; origin recognition complex, subunit 1; origin recognition complex, subunit 2; origin recognition complex, subunit 3; origin recognition complex, subunit 4; origin recognition complex, subunit 5; origin recognition complex, subunit 6; ornithine aminotransferase; ornithine carbamoyltransferase; ornithine decarboxylase 1; ornithine decarboxylase antizyme 1; ornithine decarboxylase antizyme 3; orofacial cleft 1 candidate 1; orosomucoid 1; orosomucoid 2; orthodenticle homeobox 1; orthodenticle homeobox 2; orthopedia homeobox; O-sialoglycoprotein endopeptidase; 0-sialoglycoprotein endopeptidase-like 1; osteoclast associated, immunoglobulin-like receptor; osteoclast stimulating factor 1; osteocrin; osteoglycin; osteomodulin; osteopetrosis associated transmembrane protein 1; osteosarcoma amplified 9, endoplasmic reticulum lectin; otoancorin; otoconin 90; Otoconin-90; otoferlin; otogelin; otogelin-like; otolin 1; otopetrin 1; otopetrin 2; otopetrin 3; otoraplin; otospiralin; OTU domain, ubiquitin aldehyde binding 1; OTU domain, ubiquitin aldehyde binding 2; outer dense fiber of sperm tails 1; outer dense fiber of sperm tails 2; outer dense fiber of sperm tails 2-like; outer dense fiber of sperm tails 3; outer dense fiber of sperm tails 3B; outer dense fiber of sperm tails 3-like 1; outer dense fiber of sperm tails 3-like 2; outer dense fiber of sperm tails 4; ovarian tumor suppressor candidate 2; oviductal glycoprotein 1, 120 kDa; ovochymase 1; oxidase (cytochrome c) assembly 1-like; oxidation resistance 1; oxidative stress induced growth inhibitor 1; oxidative stress induced growth inhibitor family member 2; oxidative-stress responsive 1; oxidized low density lipoprotein (lectin-like) receptor 1; oxoeicosanoid (OXE) receptor 1; oxoglutarate (alpha-ketoglutarate) dehydrogenase (lipoamide); oxoglutarate (alpha-ketoglutarate) receptor 1; oxoglutarate dehydrogenase-like; oxysterol binding protein; oxysterol binding protein 2; oxysterol binding protein-like 1A, and like 2-like 10; oxytocin receptor; oxytocin, prepropeptide; P antigen family, member 1 (prostate associated); P antigen family, member 2 (prostate associated); P antigen family, member 2B; P antigen family, member 3 (prostate associated); P antigen family, member 4 (prostate associated); P antigen family, member 5 (prostate associated); p21 protein (Cdc42/Rac)-activated kinase 1-kinase 4 and kinase 6; p21 protein (Cdc42/Rac)-activated kinase 7; P450 (cytochrome) oxidoreductase; p53 and DNA-damage regulated 1; p53-induced death domain protein; paired box 1-9; paired immunoglobin-like type 2 receptor alpha; paired immunoglobin-like type 2 receptor beta; paired related homeobox 1; paired related homeobox 2; paired-like homeobox 2a; paired-like homeobox 2b; paired-like homeodomain 1; paired-like homeodomain 2; paired-like homeodomain 3; PAK1 interacting protein 1; palladin, cytoskeletal associated protein; PALM2-AKAP2 readthrough; palmdelphin; palmitoyl-protein thioesterase 1; palmitoyl-protein thioesterase 2; pancreas specific transcription factor, la; pancreatic and duodenal homeobox 1; pancreatic lipase; pancreatic lipase-related protein 1; pancreatic lipase-related protein 2; pancreatic lipase-related protein 3; pancreatic polypeptide; pancreatic polypeptide receptor 1; pannexin 1; pannexin 2; pannexin 3; pantothenate kinase 1; pantothenate kinase 2; pantothenate kinase 3; pantothenate kinase 4; papilin, proteoglycan-like sulfated glycoprotein; papillary renal cell carcinoma (translocation-associated); pappalysin 2; parahox cluster neighbor; paralemmin; paralemmin; paralemmin 2; paralemmin 3; paraneoplastic antigen like 5 and like 6A-6D; paraneoplastic antigen MA1; paraneoplastic antigen MA2; paraneoplastic antigen MA3; paraoxonase 1; paraoxonase 2; paraoxonase 3; paraspeckle component 1; parathymosin; parathyroid hormone; parathyroid hormone 1 receptor; parathyroid hormone 2; parathyroid hormone 2 receptor; parathyroid hormone-like hormone; PARK2 co-regulated; PARK2 co-regulated-like; parkinson protein 2, E3 ubiquitin protein ligase (parkin); parkinson protein 7; paroxysmal nonkinesigenic dyskinesia; partner and localizer of BRCA2; parvalbumin; parvin, alpha; parvin, beta; parvin, gamma; patched 1; patched 2; paternally expressed 10; PAX interacting (with transcription-activation domain) protein 1; paxillin; PBX/knotted 1 homeobox 1; PBX/knotted 1 homeobox 2; PC4 and SFRS1 interacting protein 1; PDGFA associated protein 1; PDLIM1 interacting kinase 1 like; PDX1 C-terminal inhibiting factor 1; PDZ and LIM domain 1; PDZ and LIM domain 2 (mystique); PDZ and LIM domain 3; PDZ and LIM domain 4; PDZ and LIM domain 5; PDZ and LIM domain 7 (enigma); PDZ binding kinase; PDZK1 interacting protein 1; pentatricopeptide repeat domain 1; pentatricopeptide repeat domain 2; Pentatricopeptide repeat domain 3; pentraxin 3, long; pentraxin 4, long; pepsinogen 3, group I (pepsinogen A); pepsinogen 4, group I (pepsinogen A); pepsinogen 5, group I (pepsinogen A); peptidase (mitochondrial processing) alpha; peptidase (mitochondrial processing) beta; peptidase D; peptidase inhibitor 15; peptidase inhibitor 16; peptidase inhibitor 3, skin-derived; peptide deformylase (mitochondrial); peptide YY; peptidoglycan recognition protein 1-4; peptidyl arginine deiminase, type I-type IV; peptidylglycine alpha-amidating monooxygenase; peptidylprolyl cis/trans isomerase, NIMA-interacting 1; peptidylprolyl isomerase (cyclophilin)-like 1-like 4, and like 6; peptidylprolyl isomerase A (cyclophilin A); peptidylprolyl isomerase A (cyclophilin A)-like 4A to like 4D and like 4G; peptidylprolyl isomerase B (cyclophilin B); peptidylprolyl isomerase C (cyclophilin C); peptidylprolyl isomerase D; peptidylprolyl isomerase E (cyclophilin E); peptidylprolyl isomerase F; peptidylprolyl isomerase G (cyclophilin G); peptidylprolyl isomerase H (cyclophilin H); peptidyl-tRNA hydrolase 2; perforin 1 (pore forming protein); periaxin; pericentrin; pericentriolar material 1; perilipin 1-5; periostin, osteoblast specific factor; peripheral myelin protein 2; peripheral myelin protein 22; peripherin; peripherin 2 (retinal degeneration, slow); periphilin 1; periplakin; peroxiredoxin 1-6; peroxisomal biogenesis factor 1, 2, 3, 5, 5-like, 6, 7, 10, 11 alpha, 11 beta, 11 gamma, 12, 13, 14, 16, 19, and 26; peroxisomal membrane protein 2, 22 kDa; peroxisomal membrane protein 4, 24 kDa; Peroxisomal proliferator-activated receptor A-interacting complex 285 kDa protein; peroxisomal trans-2-enoyl-CoA reductase; peroxisomal, testis specific 1; peroxisome proliferator-activated receptor alpha; peroxisome proliferator-activated receptor delta; peroxisome proliferator-activated receptor gamma; peroxisome proliferator-activated receptor gamma, coactivator 1 alpha; peroxisome proliferator-activated receptor gamma, coactivator 1 beta; peroxisome proliferator-activated receptor gamma, coactivator-related 1; PERP, TP53 apoptosis effector; persephin; PH domain and leucine rich repeat protein phosphatase 1; PH domain and leucine rich repeat protein phosphatase 2; PHD and ring finger domains 1, 2, 3, 5A, 6, 7, 8, 10, 11, 12, 13, 14, 15, 16, 17, 19, 20, 20-like 1, 21A, 21B, and 23; phenazine biosynthesis-like protein domain containing; phenylalanine hydroxylase; phenylalanyl-tRNA synthetase 2, mitochondrial; phenylalanyl-tRNA synthetase, alpha subunit; phenylalanyl-tRNA synthetase, beta subunit; phenylethanolamine N-methyltransferase; phorbol-12-myristate-13-acetate-induced protein 1; phosducin; phosducin-like; phosducin-like 2; phosducin-like 3; phosphatase and actin regulator 1-4; phosphatase, orphan 1; phosphatase, orphan 2; phosphate cytidylyltransferase 1, choline, alpha; phosphate cytidylyltransferase 1, choline, beta; phosphate cytidylyltransferase 2, ethanolamine; phosphatidic acid phosphatase type 2A; phosphatidic acid phosphatase type 2B; phosphatidic acid phosphatase type 2C; phosphatidylcholine transfer protein; phosphatidylethanolamine binding protein 1; phosphatidylethanolamine N-methyltransferase; phosphatidylethanolamine-binding protein 4; phosphatidylglycerophosphate synthase 1; phosphatidylinositol 4-kinase type 2 alpha; phosphatidylinositol 4-kinase type 2 beta; phosphatidylinositol 4-kinase, catalytic, alpha; phosphatidylinositol 4-kinase, catalytic, beta; phosphatidylinositol binding clathrin assembly protein; phosphatidylinositol glycan anchor biosynthesis, class A-class C, class F-H, class K-Q, and class S-class Z; Phosphatidylinositol N-acetylglucosaminyltransferase subunit Y; phosphatidylinositol transfer protein, alpha; phosphatidylinositol transfer protein, beta; phosphatidylinositol transfer protein, cytoplasmic 1; phosphatidylinositol transfer protein, membrane-associated 1; phosphatidylinositol transfer protein, membrane-associated 2; phosphatidylinositol-3,4,5-trisphosphate-dependent Rac exchange factor 1 and 2; phosphatidylinositol-4-phosphate 5-kinase, type I, alpha; phosphatidylinositol-4-phosphate 5-kinase, type I, beta; phosphatidylinositol-4-phosphate 5-kinase, type I, gamma; phosphatidylinositol-4-phosphate 5-kinase-like 1; phosphatidylinositol-5-phosphate 4-kinase, type II, alpha; phosphatidylinositol-5-phosphate 4-kinase, type II, beta; phosphatidylinositol-5-phosphate 4-kinase, type II, gamma; phosphatidylserine decarboxylase; phosphatidylserine synthase 1; phosphatidylserine synthase 2; phosphodiesterase 10A; phosphodiesterase 11A; phosphodiesterase 12; phosphodiesterase 1A, calmodulin-dependent; phosphodiesterase 1B, calmodulin-dependent; phosphodiesterase 1C, calmodulin-dependent 70 kDa; phosphodiesterase 2A, cGMP-stimulated; phosphodiesterase 3A, cGMP-inhibited; phosphodiesterase 3B, cGMP-inhibited; phosphodiesterase 4A, cAMP-specific; phosphodiesterase 4B, cAMP-specific; phosphodiesterase 4C, cAMP-specific; phosphodiesterase 4D interacting protein; phosphodiesterase 4D, cAMP-specific; phosphodiesterase 5A, cGMP-specific; phosphodiesterase 6A, cGMP-specific, rod, alpha; phosphodiesterase 6B, cGMP-specific, rod, beta; phosphodiesterase 6C, cGMP-specific, cone, alpha prime; phosphodiesterase 6D, cGMP-specific, rod, delta; phosphodiesterase 6G, cGMP-specific, rod, gamma; phosphodiesterase 6H, cGMP-specific, cone, gamma; phosphodiesterase 7A; phosphodiesterase 7B; phosphodiesterase 8A; phosphodiesterase 8B; phosphodiesterase 9A; phosphoenolpyruvate carboxykinase 1 (soluble); phosphoenolpyruvate carboxykinase 2 (mitochondrial); phosphofructokinase, liver; phosphofructokinase, muscle; phosphofructokinase, platelet; phosphofurin acidic cluster sorting protein 1; phosphofurin acidic cluster sorting protein 2; phosphoglucomutase 1; phosphoglucomutase 2; phosphoglucomutase 2-like 1; phosphoglucomutase 3; phosphoglucomutase 5; phosphogluconate dehydrogenase; phosphoglycerate dehydrogenase; phosphoglycerate kinase 1; phosphoglycerate kinase 2; phosphoglycerate mutase 1 (brain); phosphoglycerate mutase 2 (muscle); phosphoglycerate mutase family member 4; phosphoglycerate mutase family member 5; phosphoglycolate phosphatase; phosphohistidine phosphatase 1; phosphoinositide kinase, FYVE finger containing; phosphoinositide-3-kinase adaptor protein 1; phosphoinositide-3-kinase interacting protein 1; phosphoinositide-3-kinase, catalytic, alpha polypeptide; phosphoinositide-3-kinase, catalytic, beta polypeptide; phosphoinositide-3-kinase, catalytic, delta polypeptide; phosphoinositide-3-kinase, catalytic, gamma polypeptide; phosphoinositide-3-kinase, class 2, alpha polypeptide; phosphoinositide-3-kinase, class 2, beta polypeptide; phosphoinositide-3-kinase, class 2, gamma polypeptide; phosphoinositide-3-kinase, class 3; phosphoinositide-3-kinase, regulatory subunit 1 (alpha); phosphoinositide-3-kinase, regulatory subunit 2 (beta); phosphoinositide-3-kinase, regulatory subunit 3 (gamma); phosphoinositide-3-kinase, regulatory subunit 4; phosphoinositide-3-kinase, regulatory subunit 5; phosphoinositide-interacting regulator of transient receptor potential channels; phospholamban; phospholipase A1 member A; phospholipase A2 receptor 1, 180 kDa; phospholipase A2, group IB (pancreas), group IIA (platelets, synovial fluid), group IIC, group BD, group IIE, group BF, group III, group IVA (cytosolic, calcium-dependent), group IVB (cytosolic), group IVC (cytosolic, calcium-independent), group IVD (cytosolic), group WE, group IVF, group V, group VI (cytosolic, calcium-independent), group VII (platelet-activating factor acetylhydrolase, plasma), group X, group XIIA, group XIIB, group XV, and group XVI; phospholipase A2-activating protein; phospholipase B1; phospholipase C, beta beta 1 (phosphoinositide-specific), beta 2, beta 3 (phosphatidylinositol-specific), beta 4, delta 1, delta 3, delta 4, epsilon 1, eta 1, eta 2, gamma 1, gamma 2 (phosphatidylinositol-specific), and zeta 1; phospholipase C-like 1; phospholipase C-like 2; phospholipase D family, member 3-member 6; phospholipase D1, phosphatidylcholine-specific; phospholipase D2; phospholipid scramblase 1-4; phospholipid scramblase family, member 5; phospholipid transfer protein; phospholysine phosphohistidine inorganic pyrophosphate phosphatase; phosphomannomutase 1; phosphomannomutase 2; phosphomevalonate kinase; phosphopantothenoylcysteine decarboxylase; phosphopantothenoylcysteine synthetase; phosphoprotein associated with glycosphingolipid microdomains 1; phosphoprotein enriched in astrocytes 15; phosphoribosyl pyrophosphate amidotransferase; phosphoribosyl pyrophosphate synthetase 1; phosphoribosyl pyrophosphate synthetase 1-like 1; phosphoribosyl pyrophosphate synthetase 2; phosphoribosyl pyrophosphate synthetase-associated protein 1; phosphoribosyl pyrophosphate synthetase-associated protein 2; phosphoribosylaminoimidazole carboxylase, phosphoribosylaminoimidazole succinocarboxamide synthetase; phosphoribosylformylglycinamidine synthase; phosphoribosylglycinamide formyltransferase, phosphoribosylglycinamide synthetase, phosphoribosylaminoimidazole synthetase; phosphorylase kinase, alpha 1 (muscle); phosphorylase kinase, alpha 2 (liver); phosphorylase kinase, beta; phosphorylase kinase, gamma 1 (muscle); phosphorylase kinase, gamma 2 (testis); phosphorylase, glycogen, liver; phosphorylase, glycogen, muscle; phosphorylase, glycogen; brain; phosphorylated adaptor for RNA export; phosphoserine aminotransferase 1; phosphoserine phosphatase; phosphoseryl-tRNA kinase; phosphotriesterase related; phytanoyl-CoA 2-hydroxylase; phytanoyl-CoA 2-hydroxylase interacting protein; phytanoyl-CoA 2-hydroxylase interacting protein-like; piccolo (presynaptic cytomatrix protein); piezo-type mechanosensitive ion channel component 1; piezo-type mechanosensitive ion channel component 2; piggyBac transposable element derived 1-5; pim-1 oncogene; pim-2 oncogene; pim-3 oncogene; PIN2/TERF1 interacting, telomerase inhibitor 1; pinin, desmosome associated protein; pipecolic acid oxidase; pirin (iron-binding nuclear protein); PITPNM family member 3; pitrilysin metallopeptidase 1; pituitary tumor-transforming 1; pituitary tumor-transforming 1 interacting protein; pituitary tumor-transforming 2; PLAC8-like 1; placental growth factor; placenta-specific 1; placenta-specific 1-like; placenta-specific 8; placenta-specific 9; plakophilin 1 (ectodermal dysplasia/skin fragility syndrome); plakophilin 2; plakophilin 3; plakophilin 4; Plasma cell-induced resident endoplasmic reticulum protein; Plasma glutamate carboxypeptidase; plasmalemma vesicle associated protein; plasminogen; plasminogen activator, tissue; plasminogen activator, urokinase; plasminogen activator, urokinase receptor; plasminogen-like B1; plasminogen-like B2; plasmolipin; plastin 1; plastin 3; platelet derived growth factor C; platelet derived growth factor D; platelet endothelial aggregation receptor 1; platelet factor 4; platelet factor 4 variant 1; platelet-activating factor acetylhydrolase 1b, catalytic subunit 2 (30 kDa); platelet-activating factor acetylhydrolase 1b, catalytic subunit 3 (29 kDa); platelet-activating factor acetylhydrolase 1b, regulatory subunit 1 (45 kDa); platelet-activating factor acetylhydrolase 2, 40 kDa; platelet-activating factor receptor; platelet-derived growth factor alpha polypeptide; platelet-derived growth factor beta polypeptide; platelet-derived growth factor receptor, alpha polypeptide; platelet-derived growth factor receptor, beta polypeptide; platelet-derived growth factor receptor-like; pleckstrin; pleckstrin 2; pleckstrin and Sec7 domain containing; plectin; pleiomorphic adenoma gene 1; pleiomorphic adenoma gene-like 1; pleiomorphic adenoma gene-like 2; pleiotrophin; pleiotropic regulator 1; plexin A1; plexin A2; plexin A3; plexin A4; plexin B1; plexin B2; plexin B3; plexin C1; plexin D1; PMF1-BGLAP protein isoform 1; PML-RARA regulated adaptor molecule 1PNMA-like 1; PNMA-like 2; PNN-interacting serine/arginine-rich protein; POC1B-GALNT4 readthrough; podocalyxin-like; podocalyxin-like 2; podocan; podocan-like 1; podoplanin; pogo transposable element with KRAB domain; pogo transposable element with ZNF domain; poliovirus receptor; poliovirus receptor related immunoglobulin domain containing; poliovirus receptor-related 1 (herpesvirus entry mediator C); poliovirus receptor-related 2 (herpesvirus entry mediator B); poliovirus receptor-related 3; poliovirus receptor-related 4; polo-like kinase 1; polo-like kinase 2; polo-like kinase 3; polo-like kinase 4; poly (ADP-ribose) glycohydrolase; poly (ADP-ribose) polymerase 1; poly (ADP-ribose) polymerase 2; poly (ADP-ribose) polymerase family, member 3-6, 8-12 and 14-16; poly(A) binding protein interacting protein 1; poly(A) binding protein interacting protein 2; poly(A) binding protein interacting protein 2B; poly(A) binding protein, cytoplasmic 1; poly(A) binding protein, cytoplasmic 1-like; poly(A) binding protein, cytoplasmic 1-like 2A; poly(A) binding protein, cytoplasmic 1-like 2B; poly(A) binding protein, cytoplasmic 3; poly(A) binding protein, cytoplasmic 4 (inducible form); poly(A) binding protein, cytoplasmic 5; poly(A) binding protein, nuclear 1; poly(A) binding protein, nuclear 1-like (cytoplasmic); poly(A) polymerase alpha; poly(A) polymerase beta (testis specific); poly(A) polymerase gamma; poly(A)-specific ribonuclease; poly(rC) binding protein 1; poly(rC) binding protein 2; poly(rC) binding protein 3; poly(rC) binding protein 4; polyamine modulated factor 1 binding protein 1; polyamine oxidase (exo-N4-amino); polyamine-modulated factor 1; polybromo 1; polycomb group ring finger 1-3 and 5-6; polycystic kidney and hepatic disease 1 (autosomal recessive); polycystic kidney and hepatic disease 1 (autosomal recessive)-like 1; polycystic kidney disease 1 (autosomal dominant); polycystic kidney disease 1 like 1; polycystic kidney disease 1-like 2; polycystic kidney disease 2 (autosomal dominant); polycystic kidney disease 2-like 1; polycystic kidney disease 2-like 2; polyglutamine binding protein 1; polymerase (DNA directed) alpha 1 (catalytic subunit), alpha 2 (70 kD subunit), beta, delta 1 (catalytic subunit 125 kDa), delta 2 (regulatory subunit 50 kDa), epsilon, epsilon 2 (p59 subunit), epsilon 3 (p17 subunit), eta, gamma, gamma 2 (accessory subunit), lambda, mu, theta, delta 3 (accessory subunit), delta 4, delta interacting protein 3, epsilon 4 (p12 subunit), iota, kappa and nu; polymerase (RNA) I polypeptide A (194 kDa), B (128 kDa), C (30 kDa), D (16 kDa), and E (53 kDa); polymerase (RNA) II (DNA directed) polypeptide A (220 kDa), B (140 kDa), C (33 kDa), D, E (25 kDa), F, G, H, I (14.5 kDa), J (13.3 kDa), J2, J3, K (7.0 kDa), L (7.6 kDa), and M; polymerase (RNA) III (DNA directed) polypeptide A (155 kDa), B, C (62 kD), D (44 kDa), E (80 kD), F (39 kDa), G (32 kD), G (32 kD)-like, H (22.9 kD), and K (12.3 kDa); polymerase (RNA) mitochondrial (DNA directed); polymerase I and transcript release factor; polymeric immunoglobulin receptor; polynucleotide kinase 3′-phosphatase; polypyrimidine tract binding protein 1; polypyrimidine tract binding protein 2; polypyrimidine tract binding protein 3; polyribonucleotide nucleotidyltransferase 1; poly-U binding splicing factor 60KDa; POM121 and ZP3 fusion; POM121 membrane glycoprotein; POM121 membrane glycoprotein C; POM121 membrane glycoprotein-like 12; POM121 membrane glycoprotein-like 2; POM121 membrane glycoprotein-like 7; post-GPI attachment to proteins 1; post-GPI attachment to proteins 2; post-GPI attachment to proteins 3; potassium channel modulatory factor 1; potassium channel regulator; potassium channel, subfamily K, member 1-10 and member 12-13 and member 15-18; potassium channel, subfamily T, member 1 and member 2; potassium channel, subfamily U, member 1; potassium channel, subfamily V, member 1 and member 2; potassium intermediate/small conductance calcium-activated channel, subfamily N, member 1-member 4; potassium inwardly-rectifying channel, subfamily J, member 1-member 6 and member 8-member 16; potassium large conductance calcium-activated channel, subfamily M beta member 3; potassium large conductance calcium-activated channel, subfamily M, alpha member 1; potassium large conductance calcium-activated channel, subfamily M, beta member 1; potassium large conductance calcium-activated channel, subfamily M, beta member 2; potassium large conductance calcium-activated channel, subfamily M, beta member 4; potassium voltage-gated channel, delayed-rectifier, subfamily S, member 1; potassium voltage-gated channel, delayed-rectifier, subfamily S, member 2; potassium voltage-gated channel, delayed-rectifier, subfamily S, member 3; potassium voltage-gated channel, Isk-related family, member 1-member 4; potassium voltage-gated channel, KQT-like subfamily, member 1 member 5; potassium voltage-gated channel, Shab-related subfamily, member 1; potassium voltage-gated channel, Shab-related subfamily, member 2; potassium voltage-gated channel, shaker-related subfamily, beta member 1-member 3; potassium voltage-gated channel, shaker-related subfamily, member 1 (episodic ataxia with myokymia), member 1-member 7 and member 10; potassium voltage-gated channel, Shal-related subfamily, member 1-member 3; potassium voltage-gated channel, Shaw-related subfamily, member 1-member 4; potassium voltage-gated channel, subfamily F, member 1; potassium voltage-gated channel, subfamily G, member 1-member 4; potassium voltage-gated channel, subfamily H (eag-related), member 1-member 8; POTE ankyrin domain family member C-member H and member M; POU class 1 homeobox 1; POU class 2 associating factor 1; POU class 2 homeobox 1-3; POU class 3 homeobox 1-4; POU class 4 homeobox 1-3; POU class 5 homeobox 1 and 1B; POU class 6 homeobox 1 and 2; PPAN-P2RY11 readthrough; PRA1 domain family, member 2; praj a ring finger 1; praj a ring finger 2; PRAME family member 1-22; pre T-cell antigen receptor alpha; pre-B lymphocyte 1; pre-B lymphocyte 3; pre-B-cell leukemia homeobox 1-4; pre-B-cell leukemia homeobox interacting protein 1; preferentially expressed antigen in melanoma; prefoldin subunit 1; prefoldin subunit 2; prefoldin subunit 4; prefoldin subunit 5; prefoldin subunit 6; pregnancy specific beta-1-glycoprotein 1-6, 8-9 and 11; pregnancy up-regulated non-ubiquitously expressed CaM kinase; pregnancy-associated plasma protein A, pappalysin 1; pregnancy-zone protein; premature ovarian failure, 1B; premelanosome protein; Pre-mRNA branch site protein p14; prenyl (decaprenyl) diphosphate synthase, subunit 1; prenyl (decaprenyl) diphosphate synthase, subunit 2; prenylcysteine oxidase 1; prenylcysteine oxidase 1 like; prepronociceptin; presenilin 1; presenilin 2 (Alzheimer disease 4); presenilin associated, rhomboid-like; Primary ciliary dyskinesia protein 1; primase, DNA, polypeptide 1 (49 kDa); prion protein; prion protein (testis specific); prion protein 2 (dublet); PRKC, apoptosis, WT1, regulator; PRKR interacting protein 1 (IL11 inducible); procollagen C-endopeptidase enhancer; procollagen C-endopeptidase enhancer 2; procollagen-lysine, 2-oxoglutarate 5-dioxygenase 1-3; prodynorphin; proenkephalin; profilin 1-3; profilin family, member 4; progastricsin (pepsinogen C); progestagen-associated endometrial protein; progesterone immunomodulatory binding factor 1; progesterone receptor; progesterone receptor membrane component 1; progesterone receptor membrane component 2; progestin and adipoQ receptor family member III-IX; programmed cell death 1; programmed cell death 1 ligand 2; programmed cell death 10; programmed cell death 11; programmed cell death 2; programmed cell death 2-like; programmed cell death 4 (neoplastic transformation inhibitor); programmed cell death 5; programmed cell death 6; programmed cell death 6 interacting protein; programmed cell death 7; progressive rod-cone degeneration; prohibitin; prohibitin 2; prokineticin 1; prokineticin 2; prokineticin receptor 1; prokineticin receptor 2; prolactin; prolactin receptor; prolactin regulatory element binding; prolactin releasing hormone; prolactin releasing hormone receptor; prolactin-induced protein; proliferating cell nuclear antigen; proliferation-associated 2G4, 38 kDa; proline and serine rich 1; proline dehydrogenase (oxidase) 1; proline dehydrogenase (oxidase) 2; proline rich 3, 4 (lacrimal), 5 (renal), 5 like, 7 (synaptic), 9, 11, 12, 13, 14, 14-like, 15, 15-like, 16, 18, 19, 20A, 20B, 20C, 20D, 20E, 21, 22, 23A, 23B, 23C, and 25; proline rich Gla (G-carboxyglutamic acid) 1; proline rich Gla (G-carboxyglutamic acid) 2; proline rich Gla (G-carboxyglutamic acid) 3 (transmembrane); proline rich Gla (G-carboxyglutamic acid) 4 (transmembrane); proline rich membrane anchor 1; proline rich, lacrimal 1; proline/arginine-rich end leucine-rich repeat protein; proline/histidine/glycine-rich 1; proline/serine-rich coiled-coil 1; proline-rich acidic protein 1; proline-rich coiled-coil 1 and 2A-2C; proline-rich nuclear receptor coactivator 1; proline-rich nuclear receptor coactivator 2; proline-rich protein BstNI subfamily 1-4; proline-rich protein HaeIII subfamily 1; proline-rich protein HaeIII subfamily 2; proline-rich transmembrane protein 1-4; proline-serine-threonine phosphatase interacting protein 1; proline-serine-threonine phosphatase interacting protein 2; prolyl 4-hydroxylase, alpha polypeptide I; prolyl 4-hydroxylase, alpha polypeptide II; prolyl 4-hydroxylase, alpha polypeptide III; prolyl 4-hydroxylase, beta polypeptide; prolyl 4-hydroxylase, transmembrane (endoplasmic reticulum); prolyl endopeptidase; prolyl endopeptidase-like; prolylcarboxypeptidase (angiotensinase C); pro-melanin-concentrating hormone; prominin 1; prominin 2; promyelocytic leukemia; proopiomelanocortin; PROP paired-like homeobox 1; propionyl CoA carboxylase, alpha polypeptide; propionyl CoA carboxylase, beta polypeptide; pro-platelet basic protein (chemokine (C—X—C motif) ligand 7); proprotein convertase subtilisin/kexin type 1, 2, 4-7 and 9; proprotein convertase subtilisin/kexin type 1 inhibitor; ProSAP-interacting protein 1; prosaposin; prospero homeobox 1; prospero homeobox 2; prostaglandin D2 receptor (DP); prostaglandin D2 receptor 2; prostaglandin D2 synthase 21 kDa (brain); prostaglandin E receptor 1 (subtype EP1), 42 kDa; prostaglandin E receptor 2 (subtype EP2), 53 kDa; prostaglandin E receptor 3 (subtype EP3); prostaglandin E receptor 4 (subtype EP4); prostaglandin E synthase; prostaglandin E synthase 2; prostaglandin E synthase 3 (cytosolic); prostaglandin F receptor (FP); prostaglandin F2 receptor negative regulator; prostaglandin 12 (prostacyclin) receptor (IP); prostaglandin 12 (prostacyclin) synthase; prostaglandin reductase 1; prostaglandin reductase 2; prostaglandin-endoperoxide synthase 1 (prostaglandin G/H synthase and cyclooxygenase); prostaglandin-endoperoxide synthase 2 (prostaglandin G/H synthase and cyclooxygenase); prostate and breast cancer overexpressed 1; prostate and testis expressed 1-4; prostate androgen-regulated mucin-like protein 1; prostate cancer susceptibility candidate; prostate stem cell antigen; prostate transmembrane protein, androgen induced 1; prostate tumor overexpressed 1; protamine 1-3; protease, serine 1 (trypsin 1), 3, 8, 12 (neurotrypsin, motopsin), 16 (thymus), 21 (testisin), 22, 23, 27, 33, 35, 36, 37, 38, 42, 45, 48, 50, 53, 54, 55, 57, and 58; proteasomal ATPase-associated factor 1; proteasome (prosome, macropain) 26S subunit, ATPase 1, ATPase 2, ATPase 3, ATPase 4, ATPase 5, ATPase 6, non-ATPase 1, non-ATPase 10, non-ATPase 11, non-ATPase 12, non-ATPase 13, non-ATPase 14, non-ATPase 2, non-ATPase 3, non-ATPase 4, non-ATPase 5, non-ATPase 6, non-ATPase 7, non-ATPase 8, non-ATPase 9; proteasome (prosome, macropain) activator subunit 1 (PA28 alpha); proteasome (prosome, macropain) activator subunit 2 (PA28 beta); proteasome (prosome, macropain) activator subunit 3 (PA28 gamma; Ki); proteasome (prosome, macropain) activator subunit 4; proteasome (prosome, macropain) assembly chaperone 1-4; proteasome (prosome, macropain) inhibitor subunit 1 (PI31); proteasome (prosome, macropain) subunit, alpha type 1-8; proteasome (prosome, macropain) subunit, beta type 1-11; proteasome maturation protein; protein (peptidylprolyl cis/trans isomerase) NIMA-interacting, 4 (parvulin); protein arginine methyltransferase 1-3, and 5-8; protein C (inactivator of coagulation factors Va and VIIIa); protein C receptor, endothelial; Protein deltex-2; protein disulfide isomerase family A, member 2-member 6; protein disulfide isomerase-like, testis expressed; Protein FRG2; Protein FRG2-like-2; protein geranylgeranyltransferase type I, beta subunit; Protein Idas; protein inhibitor of activated STAT, 1-4; protein interacting with cyclin A1; protein interacting with PRKCA 1; protein kinase (cAMP-dependent, catalytic) inhibitor alpha; protein kinase (cAMP-dependent, catalytic) inhibitor beta; protein kinase (cAMP-dependent, catalytic) inhibitor gamma; protein kinase C and casein kinase substrate in neurons 1; protein kinase C and casein kinase substrate in neurons 2; protein kinase C and casein kinase substrate in neurons 3; protein kinase C substrate 80K-H; protein kinase C, alpha, beta, delta, delta binding protein, epsilon, eta, gamma, iota, theta and zeta; protein kinase D1-D3 and N1-N3; protein kinase, AMP-activated, alpha 1 catalytic subunit; protein kinase, AMP-activated, alpha 2 catalytic subunit; protein kinase, AMP-activated, beta 1 non-catalytic subunit; protein kinase, AMP-activated, beta 2 non-catalytic subunit; protein kinase, AMP-activated, gamma 1 non-catalytic subunit; protein kinase, AMP-activated, gamma 2 non-catalytic subunit; protein kinase, AMP-activated, gamma 3 non-catalytic subunit; protein kinase, cAMP-dependent, catalytic, alpha; protein kinase, cAMP-dependent, catalytic, beta; protein kinase, cAMP-dependent, catalytic, gamma; protein kinase, cAMP-dependent, regulatory, type I, alpha (tissue specific extinguisher 1); protein kinase, cAMP-dependent, regulatory, type I, beta; protein kinase, cAMP-dependent, regulatory, type II, alpha; protein kinase, cAMP-dependent, regulatory, type II, beta; protein kinase, cGMP-dependent, type I; protein kinase, cGMP-dependent, type II; protein kinase, DNA-activated, catalytic polypeptide; protein kinase, interferon-inducible double stranded RNA dependent activator; protein kinase, membrane associated tyrosine/threonine 1; protein kinase, X-linked; Protein kinase-like protein SgK196; protein MICAL-3 isoform 1; protein O-fucosyltransferase 1; protein O-fucosyltransferase 2; protein O-glucosyltransferase 1; protein O-linked mannose beta1,2-N-acetylglucosaminyltransferase; Protein PCOTH; protein phosphatase 1, catalytic subunit, alpha isozyme; protein phosphatase 1, catalytic subunit, beta isozyme; protein phosphatase 1, catalytic subunit, gamma isozyme; protein phosphatase 1, regulatory (inhibitor) subunit 1A-1C, 2, 11 and 14A-14D; protein phosphatase 1, regulatory subunit 3A-3F, 7, 8, 9A, 10, 12A-12C, 13 like, 13B, 15A-15B, 16A-16B, 17, 18, 21, 26, 27, 32, 35-37 and 42;

protein phosphatase 2, catalytic subunit, alpha isozyme; protein phosphatase 2, catalytic subunit, beta isozyme; protein phosphatase 2, regulatory subunit A, alpha; protein phosphatase 2, regulatory subunit A, beta; protein phosphatase 2, regulatory subunit B, alpha; protein phosphatase 2, regulatory subunit B′, alpha; protein phosphatase 2, regulatory subunit B″, alpha; protein phosphatase 2, regulatory subunit B, beta; protein phosphatase 2, regulatory subunit B′, beta; protein phosphatase 2, regulatory subunit B″, beta; protein phosphatase 2, regulatory subunit B, delta; protein phosphatase 2, regulatory subunit B′, delta; protein phosphatase 2, regulatory subunit B′, epsilon isoform; protein phosphatase 2, regulatory subunit B, gamma; protein phosphatase 2, regulatory subunit B′, gamma; protein phosphatase 2, regulatory subunit B″, gamma; protein phosphatase 2A activator, regulatory subunit 4; protein phosphatase 3, catalytic subunit, alpha isozyme; protein phosphatase 3, catalytic subunit, beta isozyme; protein phosphatase 3, catalytic subunit, gamma isozyme; protein phosphatase 3, regulatory subunit B, alpha; protein phosphatase 3, regulatory subunit B, beta; protein phosphatase 4, catalytic subunit; protein phosphatase 4, regulatory subunit 1; protein phosphatase 4, regulatory subunit 1-like; protein phosphatase 4, regulatory subunit 2; protein phosphatase 4, regulatory subunit 4; protein phosphatase 5, catalytic subunit; protein phosphatase 6, catalytic subunit; protein phosphatase 6, regulatory subunit 1; protein phosphatase 6, regulatory subunit 2; protein phosphatase 6, regulatory subunit 3; protein phosphatase methylesterase 1; protein phosphatase, EF-hand calcium binding domain 1; protein phosphatase, EF-hand calcium binding domain 2; protein phosphatase, Mg2+/Mn2+ dependent, 1A-1B and 1D-M; protein regulator of cytokinesis 1; protein S (alpha); protein serine kinase H1; protein serine kinase H2; Protein transport protein Sec16B; protein tyrosine phosphatase type IVA, member 1-member 3; protein tyrosine phosphatase, mitochondrial 1; protein tyrosine phosphatase, non-receptor type 1, 2, 3, 4 (megakaryocyte), 5 (striatum-enriched), 6, 7, 9, 11, 12, 13 (APO-1/CD95 (Fas)-associated phosphatase), 14, 18 (brain-derived), 20A, 20B, 20C, 21, 22 (lymphoid), and 23; protein tyrosine phosphatase, receptor type, A-H, JK, M-O, Q-U, C-associated protein, N polypeptide 2, Z polypeptide 1, f polypeptide (PTPRF), interacting protein (liprin), alpha 1 and alpha 4; protein tyrosine phosphatase-like (proline instead of catalytic arginine), member A; protein tyrosine phosphatase-like (proline instead of catalytic arginine), member b; protein Z, vitamin K-dependent plasma glycoprotein; proteinase 3; protein-kinase, interferon-inducible double stranded RNA dependent inhibitor, repressor of (P58 repressor); protein-L-isoaspartate (D-aspartate) O-methyltransferase; protein-O-mannosyltransferase 1; protein-O-mannosyltransferase 2; proteoglycan 2, bone marrow (natural killer cell activator, eosinophil granule major basic protein); proteoglycan 3; proteoglycan 4; proteolipid protein 1; proteolipid protein 2 (colonic epithelium-enriched); prothymosin, alpha; protocadherin 1; protocadherin 10; protocadherin 11 X-linked; protocadherin 11 Y-linked; protocadherin 12; protocadherin 17; protocadherin 18; protocadherin 19; protocadherin 20; protocadherin 7; protocadherin 8; protocadherin 9; protocadherin alpha 1-13; protocadherin alpha subfamily C, 1; protocadherin alpha subfamily C, 2; protocadherin beta 1-8 and 10-16; protocadherin gamma subfamily A, 1-3, 5-8, 10-12; protocadherin gamma subfamily B, 1, 2, 4, 6 and 7; protocadherin gamma subfamily C, 3-5; Protocadherin-psi1; protocadherin-related 15; protogenin; protoporphyrinogen oxidase; Pseudopodium-enriched atypical kinase 1; pseudouridylate synthase 1; pseudouridylate synthase 10; pseudouridylate synthase 3; pseudouridylate synthase-like 1; PSMC3 interacting protein; psoriasis susceptibility 1 candidate 1; psoriasis susceptibility 1 candidate 2; pterin-4 alpha-carbinolamine dehydratase/dimerization cofactor of hepatocyte nuclear factor 1 alpha; pterin-4 alpha-carbinolamine dehydratase/dimerization cofactor of hepatocyte nuclear factor 1 alpha (TCF1) 2; PTK2 protein tyrosine kinase 2; PTK2B protein tyrosine kinase 2 beta; PTK6 protein tyrosine kinase 6; PTK7 protein tyrosine kinase 7; PTPN13-like, Y-linked; PTPN13-like, Y-linked 2; PTPRF interacting protein, binding protein 1 (liprin beta 1); PTPRF interacting protein, binding protein 2 (liprin beta 2); purine nucleoside phosphorylase; purinergic receptor P2X, ligand-gated ion channel, 1-7; purinergic receptor P2Y, G-protein coupled, 1, 2, 8, and 10-14; purine-rich element binding protein A, B and G; Purkinje cell protein 2; Purkinje cell protein 4; Purkinje cell protein 4 like 1; Puromycin-sensitive aminopeptidase-like protein; PYD and CARD domain containing; pyridine nucleotide-disulphide oxidoreductase domain 1; pyridine nucleotide-disulphide oxidoreductase domain 2; pyridoxal (pyridoxine, vitamin B6) kinase; pyridoxal (pyridoxine, vitamin B6) phosphatase; pyridoxamine 5′-phosphate oxidase; pyrimidinergic receptor P2Y, G-protein coupled, 4; pyrimidinergic receptor P2Y, G-protein coupled, 6; pyrin and HIN domain family, member 1; pyroglutamylated RFamide peptide; pyroglutamylated RFamide peptide receptor; pyroglutamyl-peptidase I; pyroglutamyl-peptidase I-like; pyrophosphatase (inorganic) 1; pyrophosphatase (inorganic) 2; pyrroline-5-carboxylate reductase 1; pyrroline-5-carboxylate reductase family, member 2; pyrroline-5-carboxylate reductase-like; pyruvate carboxylase; pyruvate dehydrogenase (lipoamide) alpha 1; pyruvate dehydrogenase (lipoamide) alpha 2; pyruvate dehydrogenase (lipoamide) beta; pyruvate dehydrogenase complex, component X; pyruvate dehydrogenase kinase, isozyme 1-4; pyruvate dehydrogenase phosphatase regulatory subunit; pyruvate dehyrogenase phosphatase catalytic subunit 1; pyruvate dehyrogenase phosphatase catalytic subunit 2; pyruvate kinase, liver and RBC; pyruvate kinase, muscle; QKI, KH domain containing, RNA binding; queuine tRNA-ribosyltransferase 1; quiescin Q6 sulfhydryl oxidase 1; quiescin Q6 sulfhydryl oxidase 2; quinoid dihydropteridine reductase; quinolinate phosphoribosyltransferase; R3H domain containing-like; Rab acceptor 1 (prenylated); Rab geranylgeranyltransferase, alpha subunit; Rab geranylgeranyltransferase, beta subunit; RAB GTPase activating protein 1; RAB GTPase activating protein 1-like; RAB guanine nucleotide exchange factor (GEF) 1; RAB interacting factor; Rab interacting lysosomal protein; Rab interacting lysosomal protein-like 1; Rab interacting lysosomal protein-like 2; RAB, member of RAS oncogene family-like 2A; RAB, member of RAS oncogene family-like 2B; RAB, member of RAS oncogene family-like 3; RAB, member RAS oncogene family-like 5; RAB10, member RAS oncogene family; RAB11 family interacting protein 1 (class I); RAB11 family interacting protein 2 (class I); RAB11 family interacting protein 3 (class II); RAB11 family interacting protein 4 (class II); RAB11 family interacting protein 5 (class I); RAB11A, member RAS oncogene family; RAB11B, member RAS oncogene family; RAB12, member RAS oncogene family; RAB13, member RAS oncogene family; RAB14, member RAS oncogene family; RAB15 effector protein; RAB15, member RAS onocogene family; RAB17, member RAS oncogene family; RAB18, member RAS oncogene family; RAB19, member RAS oncogene family; RAB1A, member RAS oncogene family; RAB1B, member RAS oncogene family; RAB20, member RAS oncogene family; RAB21, member RAS oncogene family; RAB22A, member RAS oncogene family; RAB23, member RAS oncogene family; RAB24, member RAS oncogene family; RAB25, member RAS oncogene family; RAB26, member RAS oncogene family; RAB27A, member RAS oncogene family; RAB27B, member RAS oncogene family; RAB28, member RAS oncogene family; RAB2A, member RAS oncogene family; RAB2B, member RAS oncogene family; RAB3 GTPase activating protein subunit 1 (catalytic); RAB3 GTPase activating protein subunit 2 (non-catalytic); RAB30, member RAS oncogene family; RAB31, member RAS oncogene family; RAB32, member RAS oncogene family; RAB33A, member RAS oncogene family; RAB33B, member RAS oncogene family; RAB34, member RAS oncogene family; RAB35, member RAS oncogene family; RAB36, member RAS oncogene family; RAB37, member RAS oncogene family; RAB38, member RAS oncogene family; RAB39A, member RAS oncogene family; RAB39B, member RAS oncogene family; RAB3A interacting protein (rabin3); RAB3A interacting protein (rabin3)-like 1; RAB3A, member RAS oncogene family; RAB3B, member RAS oncogene family; RAB3C, member RAS oncogene family; RAB3D, member RAS oncogene family; RAB40A, member RAS oncogene family; RAB40A, member RAS oncogene family-like; RAB40B, member RAS oncogene family; RAB40C, member RAS oncogene family; RAB41, member RAS oncogene family; RAB42, member RAS oncogene family; RAB43, member RAS oncogene family; RAB44, member RAS oncogene family; RAB4A, member RAS oncogene family; RAB5A, member RAS oncogene family; RAB5B, member RAS oncogene family; RAB5C, member RAS oncogene family; RAB6A, member RAS oncogene family; RAB6B, member RAS oncogene family; RAB6C, member RAS oncogene family; RAB7, member RAS oncogene family-like 1; RAB7A, member RAS oncogene family; RAB8A, member RAS oncogene family; RAB8B, member RAS oncogene family; Rab9 effector protein with kelch motifs; RAB9A, member RAS oncogene family; RAB9B, member RAS oncogene family; rabaptin, RAB GTPase binding effector protein 1; rabaptin, RAB GTPase binding effector protein 2; rabphilin 3A-like (without C2 domains); Rac GTPase activating protein 1; Rac/Cdc42 guanine nucleotide exchange factor (GEF) 6; RAD50 interactor 1; RAD51 associated protein 1; RAD51 associated protein 2; RAD52 motif 1; radixin; raftlin family member 2; raftlin, lipid raft linker 1; Ral GEF with PH domain and SH3 binding motif 1; Ral GEF with PH domain and SH3 binding motif 2; Ral GTPase activating protein, alpha subunit 1 (catalytic); Ral GTPase activating protein, alpha subunit 2 (catalytic); Ral GTPase activating protein, beta subunit (non-catalytic); ral guanine nucleotide dissociation stimulator; ral guanine nucleotide dissociation stimulator-like 1-like 4; ralA binding protein 1; RALY RNA binding protein-like; RAN binding protein 1-3, 3-like, 6, 9, 10 and 17; Ran GTPase activating protein 1; RAN guanine nucleotide release factor; RAN, member RAS oncogene family; Rap guanine nucleotide exchange factor (GEF) 1-6; Rap guanine nucleotide exchange factor (GEF)-like 1; RAP1 GTPase activating protein; RAP1 GTPase activating protein 2; RAP1, GTP-GDP dissociation stimulator 1; RAP1A, member of RAS oncogene family; RAP1B, member of RAS oncogene family; RAP2A, member of RAS oncogene family; RAP2B, member of RAS oncogene family; RAP2C, member of RAS oncogene family; RAR-related orphan receptor A; RAR-related orphan receptor B; RAR-related orphan receptor C; RAS (RAD and GEM)-like GTP binding 2; RAS (RAD and GEM)-like GTP-binding 1; RAS and EF-hand domain containing; Ras and Rab interactor 1; Ras and Rab interactor 2; Ras and Rab interactor 3; Ras and Rab interactor-like; Ras association (RalGDS/AF-6) domain family (N-terminal) member 7-member 9; Ras association (RalGDS/AF-6) domain family member 1-member 6; RAS guanyl releasing protein 1 (calcium and DAG-regulated); RAS guanyl releasing protein 2 (calcium and DAG-regulated); RAS guanyl releasing protein 3 (calcium and DAG-regulated); RAS guanyl releasing protein 4; Ras interacting protein 1; RAS p21 protein activator (GTPase activating protein) 1; RAS p21 protein activator 2; RAS p21 protein activator 3; RAS p21 protein activator 4; RAS p21 protein activator 4B; RAS protein activator like 1 (GAP1 like); RAS protein activator like 2; RAS protein activator like 3; Ras protein-specific guanine nucleotide-releasing factor 1; Ras protein-specific guanine nucleotide-releasing factor 2; ras responsive element binding protein 1; Ras suppressor protein 1; RAS, dexamethasone-induced 1; RASD family, member 2; RasGEF domain family, member 1A; RasGEF domain family, member 1B; RasGEF domain family, member 1C; Ras-like without CAAX 1; Ras-like without CAAX 2; RAS-like, estrogen-regulated, growth inhibitor; RAS-like, family 10, member A; RAS-like, family 10, member B; RAS-like, family 11, member A; RAS-like, family 11, member B; RAS-like, family 12; Ras-related associated with diabetes; ras-related C3 botulinum toxin substrate 1 (rho family, small GTP binding protein Racl); ras-related C3 botulinum toxin substrate 2 (rho family, small GTP binding protein Rac2); ras-related C3 botulinum toxin substrate 3 (rho family, small GTP binding protein Rac3); Ras-related GTP binding A-D; RB1-inducible coiled-coil 1; RB-associated KRAB zinc finger; RBM14-RBM4 readthrough; RCAN family member 3; RD RNA binding protein; RE1-silencing transcription factor; reactive oxygen species modulator 1; rearranged L-myc fusion; receptor (chemosensory) transporter protein 1-4; receptor (G protein-coupled) activity modifying protein 1-3; receptor (TNFRSF)-interacting serine-threonine kinase 1; receptor accessory protein 1-6; receptor tyrosine kinase-like orphan receptor 1; receptor tyrosine kinase-like orphan receptor 2; receptor-associated protein of the synapse; receptor-interacting serine-threonine kinase 2; receptor-interacting serine-threonine kinase 3; receptor-interacting serine-threonine kinase 4; recombination activating gene 1; recombination activating gene 2; recombination signal binding protein for immunoglobulin kappa J region; recombination signal binding protein for immunoglobulin kappa J region-like; recoverin; RecQ protein-like (DNA helicase Q1-like); RecQ protein-like 5; reelin; regenerating islet-derived 1 alpha; regenerating islet-derived 1 beta; regenerating islet-derived 3 alpha; regenerating islet-derived 3 gamma; regenerating islet-derived family, member 4; regucalcin (senescence marker protein-30); regulating synaptic membrane exocytosis 1-4; regulator of calcineurin 1; regulator of calcineurin 2; regulator of chromosome condensation 1; regulator of chromosome condensation 2; regulator of G protein signaling 9 binding protein; regulator of G-protein signaling 1, 2 (24 kDa), 3-22 and 7 binding protein; Regulator of telomere elongation helicase 1; regulatory associated protein of MTOR, complex 1; regulatory factor X, 1 (influences HLA class II expression); regulatory factor X, 2 (influences HLA class II expression); regulatory factor X, 3 (influences HLA class II expression); regulatory factor X, 4 (influences HLA class II expression); regulatory factor X, 5 (influences HLA class II expression); regulatory factor X, 6; regulatory factor X, 7; regulatory factor X, 8; regulatory factor X-associated protein; regulatory solute carrier protein, family 1, member 1; relaxin 1-3; relaxin/insulin-like family peptide receptor 1-4; RELT tumor necrosis factor receptor; RELT-like 1; RELT-like 2; REM2 and RAB-like small GTPase 1; remodeling and spacing factor 1; renalase, FAD-dependent amine oxidase; renin; renin binding protein; repetin; replication factor C (activator 1) 1, 145 kDa; replication factor C (activator 1) 2, 40 kDa; replication factor C (activator 1) 3, 38 kDa; replication factor C (activator 1) 4, 37 kDa; replication factor C (activator 1) 5, 36.5 kDa; replication initiator 1; replication protein A1, 70 kDa; replication protein A2, 32 kDa; replication protein A3, 14 kDa; replication protein A4, 30 kDa; reprimo, TP53 dependent G2 arrest mediator candidate; reprimo-like; RERG/RAS-like; resistin; resistin like beta; REST corepressor 1; REST corepressor 2; REST corepressor 3; ret finger protein-like 1; ret finger protein-like 2; ret finger protein-like 3; ret finger protein-like 4B; ret proto-oncogene; retbindin; reticulocalbin 1, EF-hand calcium binding domain; reticulocalbin 2, EF-hand calcium binding domain; reticulocalbin 3, EF-hand calcium binding domain; reticulon 1; reticulon 2; reticulon 3; reticulon 4; reticulon 4 interacting protein 1; reticulon 4 receptor; reticulon 4 receptor-like 1; reticulon 4 receptor-like 2; retina and anterior neural fold homeobox; retina and anterior neural fold homeobox 2; retinal degeneration 3; retinal G protein coupled receptor; retinal outer segment membrane protein 1; retinal pigment epithelium-specific protein 65 kDa; retinaldehyde binding protein 1; retinitis pigmentosa 1 (autosomal dominant); retinitis pigmentosa 1-like 1; retinitis pigmentosa 2 (X-linked recessive); retinitis pigmentosa 9 (autosomal dominant); retinitis pigmentosa GTPase regulator; retinitis pigmentosa GTPase regulator interacting protein 1; retinoblastoma 1; retinoblastoma binding protein 4-9; retinoblastoma-like 1 (p107); retinoblastoma-like 2 (p130); retinoic acid early transcript 1E; retinoic acid early transcript 1G; retinoic acid early transcript 1L; retinoic acid induced 1; retinoic acid induced 14; retinoic acid induced 2; retinoic acid receptor responder (tazarotene induced) 1-3; retinoic acid receptor, alpha; retinoic acid receptor, beta; retinoic acid receptor, gamma; retinoid X receptor, alpha; retinoid X receptor, beta; retinoid X receptor, gamma; retinol binding protein 1, cellular; retinol binding protein 2, cellular; retinol binding protein 3, interstitial; retinol binding protein 4, plasma; retinol binding protein 5, cellular; retinol binding protein 7, cellular; retinol dehydrogenase 10 (all-trans); retinol dehydrogenase 11 (all-trans/9-cis/11-cis); retinol dehydrogenase 12 (all-trans/9-cis/11-cis); retinol dehydrogenase 13 (all-trans/9-cis); retinol dehydrogenase 14 (all-trans/9-cis/11-cis); retinol dehydrogenase 16 (all-trans); retinol dehydrogenase 5 (11-cis/9-cis); retinol dehydrogenase 8 (all-trans); retinol saturase (all-trans-retinol 13,14-reductase); retinoschisin 1; retrotransposon-like 1; REV3-like, catalytic subunit of DNA polymerase zeta (yeast); reversion-inducing-cysteine-rich protein with kazal motifs; RFNG 0-fucosylpeptide 3-beta-N-acetylglucosaminyltransferase; RGD motif, leucine rich repeats, tropomodulin domain and proline-rich containing; RGM domain family, member A; RGM domain family, member B; Rh blood group, CcEe antigens; Rh blood group, D antigen; Rh family, C glycoprotein; rhabdoid tumor deletion region gene 1; Rh-associated glycoprotein; Rho family GTPase 1; Rho family GTPase 2; Rho family GTPase 3; Rho GDP dissociation inhibitor (GDI) alpha; Rho GDP dissociation inhibitor (GDI) beta; Rho GDP dissociation inhibitor (GDI) gamma; Rho GTPase activating protein 1, 4-6, 8-10, 11A, 11B, 12, 15, 17-36, 39, 40, 42 and 44; rho GTPase-activating protein 27 isoform a; Rho guanine nucleotide exchange factor (GEF) 1, 3, 4, 5, 7, 10, 10-like, 11, 12, 15-17, 19, 25, 26, 33, 35, 37, 38 and 40; Rho/Rac guanine nucleotide exchange factor (GEF) 18; Rho/Rac guanine nucleotide exchange factor (GEF) 2; rhodopsin; Rho-guanine nucleotide exchange factor; rhophilin associated tail protein 1; rhophilin associated tail protein 1B; rhophilin associated tail protein 1-like; rhophilin, Rho GTPase binding protein 1; rhophilin, Rho GTPase binding protein 2; rhotekin; rhotekin 2; Rhox homeobox family, member 1; Rhox homeobox family, member 2; Rhox homeobox family, member 2B; RIB43A domain with coiled-coils 1; RIB43A domain with coiled-coils 2; riboflavin kinase; ribokinase; Ribonuclease 4; ribonuclease H1; ribonuclease H2, subunit A; ribonuclease H2, subunit B; ribonuclease H2, subunit C; ribonuclease L (2′, 5′-oligoisoadenylate synthetase-dependent); ribonuclease P/MRP 14 kDa subunit; ribonuclease P/MRP 21 kDa subunit; ribonuclease P/MRP 25 kDa subunit; ribonuclease P/MRP 30 kDa subunit; ribonuclease P/MRP 38 kDa subunit; ribonuclease P/MRP 40 kDa subunit; ribonuclease T2; ribonuclease, RNase A family, 1 (pancreatic); ribonuclease, RNase A family, 10 (non-active); ribonuclease, RNase A family, 11 (non-active); ribonuclease, RNase A family, 12 (non-active); ribonuclease, RNase A family, 13 (non-active); ribonuclease, RNase A family, 2 (liver, eosinophil-derived neurotoxin); ribonuclease, RNase A family, 3; ribonuclease, RNase A family, 4; ribonuclease, RNase A family, 7; ribonuclease, RNase A family, 8; ribonuclease, RNase A family, 9 (non-active); ribonuclease, RNase A family, k6; ribonuclease, RNase K; ribonuclease/angiogenin inhibitor 1; ribonuclease-like protein 12 precursor; ribonucleoprotein, PTB-binding 1; ribonucleoprotein, PTB-binding 2; ribonucleotide reductase M1; ribonucleotide reductase M2; ribonucleotide reductase M2 B (TP53 inducible); ribophorin I; ribophorin II; ribose 5-phosphate isomerase A; ribosomal modification protein rimK-like family member A; ribosomal modification protein rimK-like family member B; ribosomal protein L10, L10a, L10-like, L11, L12, L13, L13a, L14, L15, L17, L18, L18a, L19, L21, L22, L22-like 1, L23, L23a, L24, L26, L26-like 1, L27, L27a, L28, L29, L3, L30, L31, L32, L34, L35, L35a, L36, L36a, L36a-like, L37, L37a, L38, L39, L39-like, L3-like, L4, L41, L5, L6, L7, L7a, L7-like 1, L8, L9, S10, S11, S12, S13, S14, S15, S15a, S16, S17, S17-like, S18, S19, S19 binding protein 1, S2, S20, S21, S23, S24, S25, S26, S27, S27a, S27-like, S28, S29, S3, S3A, S4 (X-linked), S4 (Y-linked 1), S4 (Y-linked 2), S5, S6, S7, S8, S9, SA; ribosomal protein S6 kinase, 52 kDa, polypeptide 1; ribosomal protein S6 kinase, 70 kDa, polypeptide 1; ribosomal protein S6 kinase, 70 kDa, polypeptide 2; ribosomal protein S6 kinase, 90 kDa, polypeptide 1; ribosomal protein S6 kinase, 90 kDa, polypeptide 2; ribosomal protein S6 kinase, 90 kDa, polypeptide 3; ribosomal protein S6 kinase, 90 kDa, polypeptide 4; ribosomal protein S6 kinase, 90 kDa, polypeptide 5; ribosomal protein S6 kinase, 90 kDa, polypeptide 6; ribosomal protein S6 kinase-like 1; ribosomal protein, large, P0; ribosomal protein, large, P1; ribosomal protein, large, P2; ribulose-5-phosphate-3-epimerase; Rieske (Fe—S) domain containing; RIMS binding protein 2; RIMS binding protein 3; RIMS binding protein 3B; RIMS binding protein 3C; ring finger and CCCH-type domains 1; ring finger and CCCH-type domains 2; ring finger and WD repeat domain 2; ring finger and WD repeat domain 3; ring finger protein (C3H2C3 type) 6; ring finger protein 1, 2, 4, 5, 7, 8 10, 11, 13, 14, 17, 19A, 19B, 20, 24, 25, 26, 31, 32, 34, 38, 39, 40, 41, 43, 44, 103, 111, 113A, 113B, 114, 115, 121, 122, 123, 125, 126, 128, 130, 133, 135, 138, 139, 141, 144A, 144B, 145, 146, 148, 149, 150, 151, 152, 157, 165, 166, 167, 168, 169, 170, 175, 180, 181, 182, 183, 185, 186, 187, 207, 208, 212, 213, 214, 215, 216, 217, 219, 220, 222, 223, and 224; ring finger protein, LIM domain interacting; ring finger protein, transmembrane 1; ring finger protein, transmembrane 2; RING1 and YY1 binding protein; ring-box 1, E3 ubiquitin protein ligase; RIO kinase 1 (yeast); RIO kinase 2 (yeast); RIO kinase 3 (yeast); RNA (guanine-7-) methyltransferase; RNA binding motif (RNP1, RRM) protein 3; RNA binding motif protein 4, 4B, 5, 6, 7, 8A, 10, 11, 12, 12B, 14, 15, 15B, 17, 18, 19, 20, 22, 23, 24, 25, 26, 27, 28, 33, 34, 38, 39, 41, 42, 43, 44, 45, 46, 47, and 48; RNA binding motif protein, X-linked; RNA binding motif protein, X-linked 2; RNA binding motif protein, X-linked-like 1-like 3; RNA binding motif protein, Y-linked, family 1, member A1, B, D, E, F, and J; RNA binding motif, single stranded interacting protein 1; RNA binding motif, single stranded interacting protein 2; RNA binding motif, single stranded interacting protein 3; RNA binding protein S1, serine-rich domain; RNA binding protein with multiple splicing; RNA binding protein with multiple splicing 2; RNA guanylyltransferase and 5′-phosphatase; RNA methyltransferase like 1; RNA polymerase II associated protein 1-3; RNA terminal phosphate cyclase domain 1; RNA terminal phosphate cyclase-like 1; rotatin; round spermatid basic protein 1; round spermatid basic protein 1-like; RPA interacting protein; RPGRIP1-like; RPTOR independent companion of MTOR, complex 2; R-spondin 1-4; runt-related transcription factor 1; runt-related transcription factor 1; translocated to, 1 (cyclin D-related); runt-related transcription factor 2; runt-related transcription factor 3; ryanodine receptor 1 (skeletal); ryanodine receptor 2 (cardiac); ryanodine receptor 3; RYK receptor-like tyrosine kinase; S1 RNA binding domain 1; S100 calcium binding protein A1, A2, A3, A4, A5, A6, A7, A7A, A7-like 2, A8, A9, A10, A11, A12, A13, A14, A16, B, G, P, and Z; S100P binding protein; SAC1 suppressor of actin mutations 1-like (yeast); SAFB-like, transcription modulator; saitohin; salt-inducible kinase 1; salt-inducible kinase 2; SAM domain and HD domain 1; SAM domain, SH3 domain and nuclear localization signals 1; S-antigen; retina and pineal gland (arrestin); SAP30 binding protein; SAP30-like; sarcalumenin; sarcoglycan, alpha (50 kDa dystrophin-associated glycoprotein); sarcoglycan, beta (43 kDa dystrophin-associated glycoprotein); sarcoglycan, delta (35 kDa dystrophin-associated glycoprotein); sarcoglycan, epsilon; sarcoglycan, gamma (35 kDa dystrophin-associated glycoprotein); sarcoglycan, zeta; sarcolemma associated protein; sarcolipin; sarcoma antigen 1; Sarcoma antigen NY-SAR-79; sarcosine dehydrogenase; sarcospan (Kras oncogene-associated gene); SATB homeobox 1; SATB homeobox 2; scaffold attachment factor B; scaffold attachment factor B2; scavenger receptor class A, member 3; scavenger receptor class B, member 1; scavenger receptor class B, member 2; scavenger receptor class F, member 1; scavenger receptor class F, member 2; scavenger receptor cysteine rich domain containing, group B (4 domains); schlafen family member 11; schlafen family member 12; schlafen family member 12-like; schlafen family member 13; schlafen family member 14; schlafen family member 5; schlafen-like 1; schwannomin interacting protein 1; sciellin; scinderin; SCL/TAL1 interrupting locus; sclerostin; Scm-like with four mbt domains 1; Scm-like with four mbt domains 2; SEBOX homeobox; SEC14 and spectrin domains 1; SEC23 interacting protein; Sec61 beta subunit; Sec61 gamma subunit; secernin 1; secernin 2; secernin 3; SECIS binding protein 2; SECIS binding protein 2-like; secretagogin, EF-hand calcium binding protein; secreted and transmembrane 1; secreted frizzled-related protein 1; secreted frizzled-related protein 2; secreted frizzled-related protein 4; secreted frizzled-related protein 5; secreted phosphoprotein 1; secreted phosphoprotein 2, 24 kDa; secreted protein, acidic, cysteine-rich (osteonectin); secretin; secretin receptor; secretion regulating guanine nucleotide exchange factor; secretoglobin, family 1A, member 1 (uteroglobin); secretoglobin, family 1C, member 1; secretoglobin, family 1D, member 1; secretoglobin, family 1D, member 2; secretoglobin, family 1D, member 4; secretoglobin, family 2A, member 1; secretoglobin, family 2A, member 2; secretoglobin, family 2B, member 2; secretoglobin, family 3A, member 1; secretoglobin, family 3A, member 2; secretogranin II; secretogranin III; secretogranin V (7B2 protein); secretory carrier membrane protein 2; secretory carrier membrane protein 3; secretory carrier membrane protein 4; secretory carrier membrane protein 5; secretory leukocyte peptidase inhibitor; selectin E; selectin L; selectin P (granule membrane protein 140 kDa, antigen CD62); selectin P ligand; selenium binding protein 1; selenocysteine lyase; selenophosphate synthetase 1; selenophosphate synthetase 2; Selenoprotein K; Selenoprotein M; selenoprotein N, 1; Selenoprotein O; selenoprotein P, plasma, 1; Selenoprotein S; Selenoprotein T; selenoprotein V; selenoprotein W, 1; selenoprotein X, 1; sema domain, immunoglobulin domain (Ig), short basic domain, secreted, (semaphorin) 3A-3G; sema domain, immunoglobulin domain (Ig), transmembrane domain (TM) and short cytoplasmic domain, (semaphorin) 4A-4D and 4F-4G; sema domain, seven thrombospondin repeats (type 1 and type 1-like), transmembrane domain (TM) and short cytoplasmic domain, (semaphorin) 5A and 5B; sema domain, transmembrane domain (TM), and cytoplasmic domain, (semaphorin) 6A-6D; semaphorin 7A, GPI membrane anchor (John Milton Hagen blood group); semenogelin I; semenogelin II; senataxin; sentan, cilia apical structure protein; Sep (O-phosphoserine) tRNA: Sec (selenocysteine) tRNA synthase; sepiapterin reductase (7,8-dihydrobiopterin:NADP+ oxidoreductase); septin 1-12, 14 and 7-like; sequestosome 1; serglycin; serine carboxypeptidase 1; serine dehydratase; serine dehydratase-like; serine hydrolase-like 2; serine hydroxymethyltransferase 1 (soluble); serine hydroxymethyltransferase 2 (mitochondrial); serine incorporator 1-5; serine palmitoyltransferase, long chain base subunit 1-3; serine palmitoyltransferase, small subunit A and B; serine peptidase inhibitor, Kazal type 1, 2 (acrosin-trypsin inhibitor), 4, 5, 6, and 9; serine peptidase inhibitor, Kunitz type 1-4; serine peptidase inhibitor-like, with Kunitz and WAP domains 1 (eppin); serine racemase; serine threonine kinase 39; serine/arginine repetitive matrix 1-5; serine/arginine-rich splicing factor 1-7 and 9-12; serine/threonine kinase 3, 4, 10-11, 11 interacting protein, 16, 17a, 17b, 18, 24, 25, 31, 32A-32C, 33, 35, 36, 38, 38 like and 40; serine/threonine kinase receptor associated protein; serine/threonine/tyrosine interacting protein; serine/threonine/tyrosine interacting-like 1; serine/threonine/tyrosine kinase 1; Serine/threonine-protein kinase MST4; Serine/threonine-protein kinase NIM1; serologically defined colon cancer antigen 3; serologically defined colon cancer antigen 8; serpin peptidase inhibitor, clade A (alpha-1 antiproteinase, antitrypsin), member 1, 3-7, and 9-12; serpin peptidase inhibitor, clade B (ovalbumin), member 1-13; serpin peptidase inhibitor, clade C (antithrombin), member 1; serpin peptidase inhibitor, clade D (heparin cofactor), member 1; serpin peptidase inhibitor, clade E (nexin, plasminogen activator inhibitor type 1), member 1-3; serpin peptidase inhibitor, clade F (alpha-2 antiplasmin, pigment epithelium derived factor), member 1; serpin peptidase inhibitor, clade F (alpha-2 antiplasmin, pigment epithelium derived factor), member 2; serpin peptidase inhibitor, Glade G (C1 inhibitor), member 1; serpin peptidase inhibitor, clade H (heat shock protein 47), member 1, (collagen binding protein 1); serpin peptidase inhibitor, clade I (neuroserpin), member 1; serpin peptidase inhibitor, clade I (pancpin), member 2; SERPINE1 mRNA binding protein 1; serum amyloid A1; serum amyloid A2; serum amyloid A4, constitutive; serum amyloid A-like 1; serum deprivation response; serum response factor (c-fos serum response element-binding transcription factor); serum response factor binding protein 1; serum/glucocorticoid regulated kinase 1; serum/glucocorticoid regulated kinase 2; serum/glucocorticoid regulated kinase family, member 3; seryl-tRNA synthetase; seryl-tRNA synthetase 2, mitochondrial; sestrin 1; sestrin 2; sestrin 3; SET binding factor 1; SET binding factor 2; SET binding protein 1; SET domain and mariner transposase fusion gene; SET domain, bifurcated 1; SET domain, bifurcated 2; SET nuclear oncogene; Seven transmembrane helix receptor; sex determining region Y; SH2B adaptor protein 1; SH2B adaptor protein 2; SH2B adaptor protein 3; SH3 and cysteine rich domain; SH3 and cysteine rich domain 2; SH3 and cysteine rich domain 3; SH3 and multiple ankyrin repeat domains 1; SH3 and multiple ankyrin repeat domains 2; SH3 and multiple ankyrin repeat domains 3; SH3 and PX domains 2A; SH3 and PX domains 2B; SH3 domain and tetratricopeptide repeats 1; SH3 domain and tetratricopeptide repeats 2; SH3 domain binding glutamic acid-rich protein; SH3 domain binding glutamic acid-rich protein like; SH3 domain binding glutamic acid-rich protein like 2; SH3 domain binding glutamic acid-rich protein like 3; SH3-binding domain kinase 1; SH3-binding domain kinase family, member 2; SH3-binding domain protein 5-like; SH3-domain binding protein 1; SH3-domain binding protein 2; SH3-domain binding protein 4; SH3-domain binding protein 5 (BTK-associated); SH3-domain GRB2-like (endophilin) interacting protein 1; SH3-domain GRB2-like 1; SH3-domain GRB2-like 2; SH3-domain GRB2-like 3; SH3-domain GRB2-like endophilin B1; SH3-domain GRB2-like endophilin B2; SH3-domain kinase binding protein 1; SH3KBP1 binding protein 1; SHANK-associated RH domain interactor; SHC SH2-domain binding protein 1; SHC SH2-domain binding protein 1-like; short chain dehydrogenase/reductase family 16C, member 5; short chain dehydrogenase/reductase family 39U, member 1; short chain dehydrogenase/reductase family 42E, member 1; short chain dehydrogenase/reductase family 42E, member 2; short chain dehydrogenase/reductase family 9C, member 7; short coiled-coil protein; short stature homeobox; short stature homeobox 2; short stature homeobox protein 2 isoform c; shroom family member 1; shroom family member 2; shroom family member 3; Shwachman-Bodian-Diamond syndrome; sialic acid acetylesterase; sialic acid binding Ig-like lectin 1, sialoadhesin; sialic acid binding Ig-like lectin 10; sialic acid binding Ig-like lectin 11; sialic acid binding Ig-like lectin 14; sialic acid binding Ig-like lectin 15; sialic acid binding Ig-like lectin 5; sialic acid binding Ig-like lectin 6; sialic acid binding Ig-like lectin 7; sialic acid binding Ig-like lectin 8; sialic acid binding Ig-like lectin 9; sialidase 1 (lysosomal sialidase); sialidase 2 (cytosolic sialidase); sialidase 3 (membrane sialidase); sialidase 4; sialophorin; SID1 transmembrane family, member 1; SID1 transmembrane family, member 2; sidekick cell adhesion molecule 1; sidekick cell adhesion molecule 2; sideroflexin 1; sideroflexin 2; sideroflexin 3; sideroflexin 4; sideroflexin 5; sigma non-opioid intracellular receptor 1; Signal peptide peptidase-like 2A; Signal peptide peptidase-like 2B; Signal peptide peptidase-like 2C; Signal peptide peptidase-like 3; signal peptide, CUB domain, EGF-like 1; signal peptide, CUB domain, EGF-like 2; signal peptide, CUB domain, EGF-like 3; signal recognition particle 19 kDa; signal recognition particle 54 kDa; signal recognition particle 68 kDa; signal recognition particle 72 kDa; signal recognition particle 9 kDa; signal recognition particle receptor (docking protein); signal recognition particle receptor, B subunit; signal sequence receptor, alpha; signal sequence receptor, beta (translocon-associated protein beta); signal sequence receptor, delta; signal sequence receptor, gamma (translocon-associated protein gamma); signal transducer and activator of transcription 1, 91 kDa; signal transducer and activator of transcription 2, 113 kDa; signal transducer and activator of transcription 3 (acute-phase response factor); signal transducer and activator of transcription 4; signal transducer and activator of transcription 5A; signal transducer and activator of transcription 5B; signal transducer and activator of transcription 6, interleukin-4 induced; signal transducing adaptor family member 1; signal transducing adaptor family member 2; signal transducing adaptor molecule (SH3 domain and ITAM motif) 1; signal transducing adaptor molecule (SH3 domain and ITAM motif) 2; signal-induced proliferation-associated 1; signal-induced proliferation-associated 1 like 1; signal-induced proliferation-associated 1 like 2; signal-induced proliferation-associated 1 like 3; signaling lymphocytic activation molecule family member 1; signaling threshold regulating transmembrane adaptor 1; signal-regulatory protein alpha; signal-regulatory protein beta 1; signal-regulatory protein beta 2; signal-regulatory protein delta; signal-regulatory protein gamma; SIK family kinase 3; Sin3A-associated protein, 130 kDa; Sin3A-associated protein, 18 kDa; Sin3A-associated protein, 25 kDa; Sin3A-associated protein, 30 kDa; single immunoglobulin and toll-interleukin 1 receptor (TIR) domain; single stranded DNA binding protein 3; single stranded DNA binding protein 4; single-stranded DNA binding protein 1; single-stranded DNA binding protein 2; single-strand-selective monofunctional uracil-DNA glycosylase 1; sirtuin 1-7; SIVA1, apoptosis-inducing factor; SIX homeobox 1-6; six transmembrane epithelial antigen of the prostate 1; Sjogren syndrome antigen B (autoantigen La); Sjogren syndrome nuclear autoantigen 1; Sjogren syndrome/scleroderma autoantigen 1; SKI family transcriptional corepressor 1; SKI family transcriptional corepressor 2; SKI-like oncogene; SLAIN motif family, member 1; SLAIN motif family, member 2; SLAM family member 6-9; SLC2A4 regulator; SLIT and NTRK-like family, member 1-6; SLIT-ROBO Rho GTPase activating protein 1-3; SLP adaptor and CSK interacting membrane protein; SMAD family member 1-7 and 9; Smad nuclear interacting protein 1; SMAD specific E3 ubiquitin protein ligase 1; SMAD specific E3 ubiquitin protein ligase 2; small ArfGAP 1; small ArfGAP2; small cell adhesion glycoprotein; small EDRK-rich factor 1A (telomeric); small EDRK-rich factor 1B (centromeric); small EDRK-rich factor 2; small G protein signaling modulator 1; small G protein signaling modulator 2; small G protein signaling modulator 3; small glutamine-rich tetratricopeptide repeat (TPR)-containing, alpha; small glutamine-rich tetratricopeptide repeat (TPR)-containing, beta; small muscle protein, X-linked; small nuclear ribonucleoprotein 200 kDa (U5); small nuclear ribonucleoprotein 25 kDa (U11/U12); small nuclear ribonucleoprotein 27 kDa (U4/U6.U5); small nuclear ribonucleoprotein 35 kDa (U11/U12); small nuclear ribonucleoprotein 40 kDa (U5); small nuclear ribonucleoprotein 48 kDa (U11/U12); small nuclear ribonucleoprotein 70 kDa (U1); small nuclear ribonucleoprotein D1 polypeptide 16 kDa; small nuclear ribonucleoprotein D2 polypeptide 16.5 kDa; small nuclear ribonucleoprotein D3 polypeptide 18 kDa; small nuclear ribonucleoprotein polypeptide A-C, A′, E-G and N; small nuclear ribonucleoprotein polypeptides B and B1; small nuclear RNA activating complex, polypeptide 1, 43 kDa; small nuclear RNA activating complex, polypeptide 2, 45 kDa; small nuclear RNA activating complex, polypeptide 3, 50 kDa; small nuclear RNA activating complex, polypeptide 4, 190 kDa; small nuclear RNA activating complex, polypeptide 5, 19 kDa; small proline-rich protein 1A, 1B, 2A, 2B, 2D-2G, 3 and 4; small VCP/p97-interacting protein; Smith-Magenis syndrome chromosome region, candidate 7; Smith-Magenis syndrome chromosome region, candidate 7-like; Smith-Magenis syndrome chromosome region, candidate 8; smoothelin; smoothelin-like 1; smoothelin-like 2; smoothened, frizzled family receptor; SMYD family member 5; SNAP-associated protein; SNF related kinase; SNF2 histone linker PHD RING helicase; Snf2-related CREBBP activator protein; SNRPN upstream reading frame; SNRPN upstream reading frame-like; snurportin 1; sodium channel and clathrin linker 1; sodium channel modifier 1; sodium channel, nonvoltage-gated 1 alpha; sodium channel, nonvoltage-gated 1, beta; sodium channel, nonvoltage-gated 1, delta; sodium channel, nonvoltage-gated 1, gamma; sodium channel, voltage gated, type VIII, alpha subunit; sodium channel, voltage-gated, type I, alpha subunit; sodium channel, voltage-gated, type I, beta; sodium channel, voltage-gated, type II, alpha subunit; sodium channel, voltage-gated, type II, beta; sodium channel, voltage-gated, type III, alpha subunit; sodium channel, voltage-gated, type III, beta; sodium channel, voltage-gated, type IV, alpha subunit; sodium channel, voltage-gated, type IV, beta; sodium channel, voltage-gated, type IX, alpha subunit; sodium channel, voltage-gated, type V, alpha subunit; sodium channel, voltage-gated, type VII, alpha; sodium channel, voltage-gated, type X, alpha subunit; sodium channel, voltage-gated, type XI, alpha subunit; sodium leak channel, non-selective; solute carrier family 1 (glial high affinity glutamate transporter), member 2; solute carrier family 1 (glial high affinity glutamate transporter), member 3; solute carrier family 1 (glutamate transporter), member 7; solute carrier family 1 (glutamate/neutral amino acid transporter), member 4; solute carrier family 1 (high affinity aspartate/glutamate transporter), member 6; solute carrier family 1 (neuronal/epithelial high affinity glutamate transporter, system Xag), member 1; solute carrier family 1 (neutral amino acid transporter), member 5; solute carrier family 10 (sodium/bile acid cotransporter family), member 1-7; solute carrier family 11 (proton-coupled divalent metal ion transporters), member 1; solute carrier family 11 (proton-coupled divalent metal ion transporters), member 2; solute carrier family 12 (potassium/chloride transporter), member 4-9; solute carrier family 12 (sodium/chloride transporters), member 3; solute carrier family 12 (sodium/potassium/chloride transporters), member 1; solute carrier family 12 (sodium/potassium/chloride transporters), member 2; solute carrier family 13 (sodium/sulfate symporters), member 1; solute carrier family 13 (sodium/sulfate symporters), member 4; solute carrier family 13 (sodium-dependent citrate transporter), member 5; solute carrier family 13 (sodium-dependent dicarboxylate transporter), member 2; solute carrier family 13 (sodium-dependent dicarboxylate transporter), member 3; solute carrier family 14 (urea transporter), member 1 (Kidd blood group); solute carrier family 14 (urea transporter), member 2; solute carrier family 15 (H+/peptide transporter), member 2; solute carrier family 15 (oligopeptide transporter), member 1; solute carrier family 15, member 3; solute carrier family 15, member 4; solute carrier family 16, member 1 (monocarboxylic acid transporter 1); solute carrier family 16, member 10 (aromatic amino acid transporter); solute carrier family 16, member 11 (monocarboxylic acid transporter 11); solute carrier family 16, member 12 (monocarboxylic acid transporter 12); solute carrier family 16, member 13 (monocarboxylic acid transporter 13); solute carrier family 16, member 14 (monocarboxylic acid transporter 14); solute carrier family 16, member 2 (monocarboxylic acid transporter 8); solute carrier family 16, member 3 (monocarboxylic acid transporter 4); solute carrier family 16, member 4 (monocarboxylic acid transporter 5); solute carrier family 16, member 5 (monocarboxylic acid transporter 6); solute carrier family 16, member 6 (monocarboxylic acid transporter 7); solute carrier family 16, member 7 (monocarboxylic acid transporter 2); solute carrier family 16, member 8 (monocarboxylic acid transporter 3); solute carrier family 16, member 9 (monocarboxylic acid transporter 9); solute carrier family 17 (anion/sugar transporter), member 5; solute carrier family 17 (sodium phosphate), member 1; solute carrier family 17 (sodium phosphate), member 2; solute carrier family 17 (sodium phosphate), member 3; solute carrier family 17 (sodium phosphate), member 4; solute carrier family 17 (sodium-dependent inorganic phosphate cotransporter), member 6; solute carrier family 17 (sodium-dependent inorganic phosphate cotransporter), member 7; solute carrier family 17 (sodium-dependent inorganic phosphate cotransporter), member 8; solute carrier family 17, member 9; solute carrier family 18 (vesicular acetylcholine), member 3; solute carrier family 18 (vesicular monoamine), member 1; solute carrier family 18 (vesicular monoamine), member 2; solute carrier family 19 (folate transporter), member 1; solute carrier family 19 (thiamine transporter), member 2; solute carrier family 19, member 3; solute carrier family 2 (facilitated glucose transporter), member 1-4 and 6-14; solute carrier family 2 (facilitated glucose/fructose transporter), member 5; solute carrier family 20 (phosphate transporter), member 1; solute carrier family 20 (phosphate transporter), member 2; solute carrier family 22 (extraneuronal monoamine transporter), member 3; solute carrier family 22 (organic anion transporter), member 13; solute carrier family 22 (organic anion transporter), member 6; solute carrier family 22 (organic anion transporter), member 7; solute carrier family 22 (organic anion transporter), member 8; solute carrier family 22 (organic anion transporter), member 9; solute carrier family 22 (organic anion/urate transporter), member 11; solute carrier family 22 (organic anion/urate transporter), member 12; solute carrier family 22 (organic cation transporter), member 1; solute carrier family 22 (organic cation transporter), member 18 antisense; solute carrier family 22 (organic cation transporter), member 2; solute carrier family 22 (organic cation/carnitine transporter), member 16; solute carrier family 22 (organic cation/carnitine transporter), member 5; solute carrier family 22 (organic cation/ergothioneine transporter), member 4; solute carrier family 22, member 10; solute carrier family 22, member 14; solute carrier family 22, member 15; solute carrier family 22, member 17; solute carrier family 22, member 18; solute carrier family 22, member 23; solute carrier family 22, member 24; solute carrier family 22, member 25; solute carrier family 23 (nucleobase transporters), member 1; solute carrier family 23 (nucleobase transporters), member 2; solute carrier family 23 (nucleobase transporters), member 3; solute carrier family 24 (sodium/potassium/calcium exchanger), member 1; solute carrier family 24 (sodium/potassium/calcium exchanger), member 2; solute carrier family 24 (sodium/potassium/calcium exchanger), member 3; solute carrier family 24 (sodium/potassium/calcium exchanger), member 4; solute carrier family 24 (sodium/potassium/calcium exchanger), member 6; solute carrier family 24, member 5; solute carrier family 25 (carnitine/acylcarnitine translocase), member 20; solute carrier family 25 (mitochondrial carrier), member 18; solute carrier family 25 (mitochondrial carrier, Aralar), member 12; solute carrier family 25 (mitochondrial carrier, brain), member 14; solute carrier family 25 (mitochondrial carrier: glutamate), member 22; solute carrier family 25 (mitochondrial carrier; adenine nucleotide translocator), member 31; solute carrier family 25 (mitochondrial carrier; adenine nucleotide translocator), member 4; solute carrier family 25 (mitochondrial carrier; adenine nucleotide translocator), member 5; solute carrier family 25 (mitochondrial carrier; adenine nucleotide translocator), member 6; solute carrier family 25 (mitochondrial carrier; citrate transporter), member 1; solute carrier family 25 (mitochondrial carrier; Graves disease autoantigen), member 16; solute carrier family 25 (mitochondrial carrier; ornithine transporter) member 15; solute carrier family 25 (mitochondrial carrier; ornithine transporter) member 2; solute carrier family 25 (mitochondrial carrier; oxoglutarate carrier), member 11; solute carrier family 25 (mitochondrial carrier; peroxisomal membrane protein, 34 kDa), member 17; solute carrier family 25 (mitochondrial carrier; phosphate carrier), member 23; solute carrier family 25 (mitochondrial carrier; phosphate carrier), member 24; solute carrier family 25 (mitochondrial carrier; phosphate carrier), member 25; solute carrier family 25 (mitochondrial carrier; phosphate carrier), member 3; solute carrier family 25 (mitochondrial oxodicarboxylate carrier), member 21; solute carrier family 25 (mitochondrial thiamine pyrophosphate carrier), member 19; solute carrier family 25, member 13 (citrin), 26-30, and 32-48; solute carrier family 26 (sulfate transporter), member 1; solute carrier family 26 (sulfate transporter), member 2; solute carrier family 26, member 10; solute carrier family 26, member 11; solute carrier family 26, member 3; solute carrier family 26, member 4; solute carrier family 26, member 5 (prestin); solute carrier family 26, member 6; solute carrier family 26, member 7; solute carrier family 26, member 8; solute carrier family 26, member 9; solute carrier family 27 (fatty acid transporter), member 1-6; solute carrier family 28 (sodium-coupled nucleoside transporter), member 1-3; solute carrier family 29 (nucleoside transporters), member 1-4; solute carrier family 3 (activators of dibasic and neutral amino acid transport), member 2; solute carrier family 3 (cystine, dibasic and neutral amino acid transporters, activator of cystine, dibasic and neutral amino acid transport), member 1; solute carrier family 30 (zinc transporter), member 1-9; solute carrier family 30, member 10; solute carrier family 31 (copper transporters), member 1; solute carrier family 31 (copper transporters), member 2; solute carrier family 32 (GABA vesicular transporter), member 1; solute carrier family 33 (acetyl-CoA transporter), member 1; solute carrier family 34 (sodium phosphate), member 1; solute carrier family 34 (sodium phosphate), member 2; solute carrier family 34 (sodium phosphate), member 3; solute carrier family 35 (CMP-sialic acid transporter), member A1; solute carrier family 35 (UDP-galactose transporter), member A2; solute carrier family 35 (UDP-glucuronic acid/UDP-N-acetylgalactosamine dual transporter), member D1; solute carrier family 35 (UDP-N-acetylglucosamine (UDP-GlcNAc) transporter), member A3; solute carrier family 35, member A4, A5, B1, B2, B3, B4, C1, C2, D2, D3, E1, E2, E2B, E3, E4, F1, F2, F3, F4, F5, G1, G3, G4, G5, and G6; solute carrier family 36 (proton/amino acid symporter), member 1-4; solute carrier family 37 (glycerol-3-phosphate transporter), member 1-3; solute carrier family 38, member 1, 2, and 4-11; solute carrier family 39 (metal ion transporter), member 11; solute carrier family 39 (metal ion transporter), member 5; solute carrier family 39 (zinc transporter), member 1-4, and 6-14; solute carrier family 4 (anion exchanger), member 1, adaptor protein; solute carrier family 4, anion exchanger, member 1 (erythrocyte membrane protein band 3, Diego blood group); solute carrier family 4, anion exchanger, member 2 (erythrocyte membrane protein band 3-like 1); solute carrier family 4, anion exchanger, member 3; solute carrier family 4, sodium bicarbonate cotransporter, member 4-9; solute carrier family 4, sodium bicarbonate transporter, member 10; solute carrier family 4, sodium borate transporter, member 11; solute carrier family 40 (iron-regulated transporter), member 1; solute carrier family 41, member 1-3; solute carrier family 43, member 1-3; solute carrier family 44, member 1-5; solute carrier family 45, member 1-4; solute carrier family 46 (folate transporter), member 1; solute carrier family 46, member 2; solute carrier family 46, member 3; solute carrier family 47, member 1; solute carrier family 47, member 2; solute carrier family 48 (heme transporter), member 1; solute carrier family 5 (choline transporter), member 7; solute carrier family 5 (iodide transporter), member 8; solute carrier family 5 (low affinity glucose cotransporter), member 4; solute carrier family 5 (sodium iodide symporter), member 5; solute carrier family 5 (sodium/glucose cotransporter), member 1; solute carrier family 5 (sodium/glucose cotransporter), member 10; solute carrier family 5 (sodium/glucose cotransporter), member 11; solute carrier family 5 (sodium/glucose cotransporter), member 12; solute carrier family 5 (sodium/glucose cotransporter), member 2; solute carrier family 5 (sodium/glucose cotransporter), member 9; solute carrier family 5 (sodium/myo-inositol cotransporter), member 3; solute carrier family 5 (sodium-dependent vitamin transporter), member 6; solute carrier family 50 (sugar transporter), member 1; solute carrier family 6 (amino acid transporter), member 14; solute carrier family 6 (neurotransmitter transporter, betaine/GABA), member 12; solute carrier family 6 (neurotransmitter transporter, creatine), member 8; solute carrier family 6 (neurotransmitter transporter, dopamine), member 3; solute carrier family 6 (neurotransmitter transporter, GABA), member 1; solute carrier family 6 (neurotransmitter transporter, GABA), member 11; solute carrier family 6 (neurotransmitter transporter, GABA), member 13; solute carrier family 6 (neurotransmitter transporter, glycine), member 5; solute carrier family 6 (neurotransmitter transporter, glycine), member 9; solute carrier family 6 (neurotransmitter transporter, L-proline), member 7; solute carrier family 6 (neurotransmitter transporter, noradrenalin), member 2; solute carrier family 6 (neurotransmitter transporter, serotonin), member 4; solute carrier family 6 (neurotransmitter transporter, taurine), member 6; solute carrier family 6 (neutral amino acid transporter), member 15; solute carrier family 6 (neutral amino acid transporter), member 19; solute carrier family 6 (proline IMINO transporter), member 20; solute carrier family 6, member 16; solute carrier family 6, member 17; solute carrier family 6, member 18; solute carrier family 7 (amino acid transporter light chain, L system), member 5; solute carrier family 7 (amino acid transporter light chain, L system), member 8; solute carrier family 7 (amino acid transporter light chain, y+L system), member 6; solute carrier family 7 (amino acid transporter light chain, y+L system), member 7; solute carrier family 7 (anionic amino acid transporter light chain, xc-system), member 11; solute carrier family 7 (anionic amino acid transporter), member 13; solute carrier family 7 (cationic amino acid transporter, y+ system), member 1-3; solute carrier family 7 (glycoprotein-associated amino acid transporter light chain, bo, + system), member 9; solute carrier family 7 (neutral amino acid transporter light chain, asc system), member 10; solute carrier family 7 (orphan transporter), member 14; solute carrier family 7 (orphan transporter), member 4; solute carrier family 7, member 6 opposite strand; solute carrier family 8 (sodium/calcium exchanger), member 1; solute carrier family 8 (sodium/calcium exchanger), member 2; solute carrier family 8 (sodium/calcium exchanger), member 3; solute carrier family 9 (sodium/hydrogen exchanger), member 1; solute carrier family 9 (sodium/hydrogen exchanger), member 2; solute carrier family 9 (sodium/hydrogen exchanger), member 3; solute carrier family 9 (sodium/hydrogen exchanger), member 3 regulator 1; solute carrier family 9 (sodium/hydrogen exchanger), member 3 regulator 2; solute carrier family 9 (sodium/hydrogen exchanger), member 4; solute carrier family 9 (sodium/hydrogen exchanger), member 5; solute carrier family 9 (sodium/hydrogen exchanger), member 6; solute carrier family 9 (sodium/hydrogen exchanger), member 7; solute carrier family 9 (sodium/hydrogen exchanger), member 8; solute carrier family 9 (sodium/hydrogen exchanger), member 9; solute carrier family 9, member 10; solute carrier family 9, member 11; solute carrier family 9, subfamily B (cation proton antiporter 2), member 1; solute carrier family 9, subfamily B (cation proton antiporter 2), member 2; solute carrier organic anion transporter family, member 1A2; solute carrier organic anion transporter family, member 1B1; solute carrier organic anion transporter family, member 1B3; solute carrier organic anion transporter family, member 1B7 (non-functional); solute carrier organic anion transporter family, member 1C1; solute carrier organic anion transporter family, member 2A1; solute carrier organic anion transporter family, member 2B1; solute carrier organic anion transporter family, member 3A1; solute carrier organic anion transporter family, member 4A1; solute carrier organic anion transporter family, member 4C1; solute carrier organic anion transporter family, member 5A1; solute carrier organic anion transporter family, member 6A1; somatostatin; somatostatin receptor 1; somatostatin receptor 2; somatostatin receptor 3; somatostatin receptor 4; somatostatin receptor 5; SON DNA binding protein; sonic hedgehog; sorbitol dehydrogenase; sorcin; sortilin 1; sortilin-related receptor, L(DLR class) A repeats containing; sorting nexin 1-20, 22, 24, 25, 29, and 31-33; sorting nexin family member 21; sorting nexin family member 27; sorting nexin family member 30; Sp1 transcription factor; SP100 nuclear antigen; SP110 nuclear body protein; SP140 nuclear body protein; SP140 nuclear body protein-like; Sp2 transcription factor; Sp3 transcription factor; Sp4 transcription factor; Sp5 transcription factor; Sp6 transcription factor; Sp7 transcription factor; Sp8 transcription factor; SPANX family, member A2, B1, B2, C, D, and SPARC related modular calcium binding 1; SPARC related modular calcium binding 2; sparc/osteonectin, cwcv and kazal-like domains proteoglycan (testican) 1; sparc/osteonectin, cwcv and kazal-like domains proteoglycan (testican) 2; sparc/osteonectin, cwcv and kazal-like domains proteoglycan (testican) 3; SPARC-like 1 (hevin); spastic ataxia of Charlevoix-Saguenay (sacsin); spastic paraplegia 11 (autosomal recessive); spastic paraplegia 20 (Troyer syndrome); spastic paraplegia 21 (autosomal recessive, Mast syndrome); spastic paraplegia 7 (pure and complicated autosomal recessive); spastin; speckle-type POZ protein; speckle-type POZ protein-like; spectrin repeat containing, nuclear envelope 1; spectrin repeat containing, nuclear envelope 2; spectrin, alpha, erythrocytic 1 (elliptocytosis 2); spectrin, alpha, non-erythrocytic 1 (alpha-fodrin); spectrin, beta, erythrocytic; spectrin, beta, non-erythrocytic 1-5; SPEG complex locus; sperm acrosome associated 1; sperm acrosome associated 3; sperm acrosome associated 4; sperm acrosome associated 5; sperm acrosome associated 5B; sperm acrosome associated 7; sperm associated antigen 1; sperm associated antigen 11A; sperm associated antigen 11B; sperm associated antigen 16; sperm associated antigen 17; sperm associated antigen 4; sperm associated antigen 5; sperm associated antigen 6; sperm associated antigen 7; sperm associated antigen 8; sperm associated antigen 9; sperm autoantigenic protein 17; sperm equatorial segment protein 1; sperm flagellar 1; sperm flagellar 2; sperm mitochondria-associated cysteine-rich protein; sperm protein associated with the nucleus, X-linked, family member A1; sperm specific antigen 2; spermatid associated; spermatid maturation 1; spermatid perinuclear RNA binding protein; spermatogenesis and centriole associated 1; spermatogenesis and oogenesis specific basic helix-loop-helix 1; spermatogenesis and oogenesis specific basic helix-loop-helix 2; spermatogenesis associated 2-like, 3, 4, 5, 5-like 1, 6, 7, 8, 9, 12, 13, 16, 17, 19, 2, 20, 21, 22, 24, and 25; spermatogenesis associated, serine-rich 1; spermatogenesis associated, serine-rich 2; spermatogenesis associated, serine-rich 2-like; spermatogenic leucine zipper 1; spermidine synthase; spermidine/spermine N1-acetyl transferase-like 1; spermidine/spermine N1-acetyltransferase 1; spermidine/spermine N1-acetyltransferase family member 2; spermine oxidase; spermine synthase; S-phase cyclin A-associated protein in the ER; S-phase kinase-associated protein 1; S-phase kinase-associated protein 2 (p45); S-phase response (cyclin related); sphingomyelin phosphodiesterase 1, acid lysosomal; sphingomyelin phosphodiesterase 2, neutral membrane (neutral sphingomyelinase); sphingomyelin phosphodiesterase 3, neutral membrane (neutral sphingomyelinase II); sphingomyelin phosphodiesterase 4, neutral membrane (neutral sphingomyelinase-3); sphingomyelin phosphodiesterase, acid-like 3A; sphingomyelin phosphodiesterase, acid-like 3B; sphingomyelin synthase 1; sphingomyelin synthase 2; sphingosine kinase 1; sphingosine kinase 2; sphingosine-1-phosphate lyase 1; sphingosine-1-phosphate phosphatase 1; sphingosine-1-phosphate phosphatase 2; sphingosine-1-phosphate receptor 1; sphingosine-1-phosphate receptor 2; sphingosine-1-phosphate receptor 3; sphingosine-1-phosphate receptor 4; sphingosine-1-phosphate receptor 5; SPHK1 interactor, AKAP domain containing; Spi-B transcription factor (Spi-1/PU.1 related); Spi-C transcription factor (Spi-1/PU.1 related); spindle and centriole associated protein 1; spindle and kinetochore associated complex subunit 1; spindle and kinetochore associated complex subunit 2; spindle and kinetochore associated complex subunit 2-like; spindle and kinetochore associated complex subunit 3; spindlin 1; spindlin family, member 2A; spindlin family, member 2B; spindlin family, member 3; spindlin family, member 4; SPINLW1-WFDC6 readthrough; spleen focus forming virus (SFFV) proviral integration oncogene spil; spleen tyrosine kinase; splicing factor 1; splicing factor 3a, subunit 1, 120 kDa; splicing factor 3a, subunit 2, 66 kDa; splicing factor 3a, subunit 3, 60 kDa; splicing factor 3b, subunit 1, 155 kDa; splicing factor 3b, subunit 2, 145 kDa; splicing factor 3b, subunit 3, 130 kDa; splicing factor 3b, subunit 4, 49 kDa; splicing factor 3b, subunit 5, 10 kDa; splicing factor proline/glutamine-rich; splicing regulatory glutamine/lysine-rich protein 1; split hand/foot malformation (ectrodactyly) type 1; spondin 1, extracellular matrix protein; spondin 2, extracellular matrix protein; squalene epoxidase; squamous cell carcinoma antigen recognized by T cells; squamous cell carcinoma antigen recognized by T cells 3; SRA stem-loop interacting RNA binding protein; src kinase associated phosphoprotein 1; src kinase associated phosphoprotein 2; SRC kinase signaling inhibitor 1; Src-like-adaptor; Src-like-adaptor 2; src-related kinase lacking C-terminal regulatory tyrosine and N-terminal myristylation sites; SREBF chaperone; SREK1-interacting protein 1; SRR1 domain containing; SR-related CTD-associated factor 1; SR-related CTD-associated factor 11; SR-related CTD-associated factor 4; SR-related CTD-associated factor 8; SRSF protein kinase 1; SRSF protein kinase 2; SRSF protein kinase 3; SRY (sex determining region Y)-box 1-15, 17, 18, 21, and 30; ST3 beta-galactoside alpha-2,3-sialyltransferase 1-6; ST6 (alpha-N-acetyl-neuraminyl-2,3-beta-galactosyl-1,3)-N-acetylgalactosaminide alpha-2,6-sialyltransferase 1-6; ST6 beta-galactosamide alpha-2,6-sialyltranferase 1; ST6 beta-galactosamide alpha-2, 6-sialyltranferase 2; ST8 alpha-N-acetyl-neuraminide alpha-2,8-sialyltransferase 1-6; stabilin 1; stabilin 2; STAM binding protein; STAM binding protein-like 1; stannin; stanniocalcin 1; stanniocalcin 2; starch binding domain 1; STARD3 N-terminal like; statherin; stathmin 1; stathmin-like 2; stathmin-like 3; stathmin-like 4; STE20-like kinase; STE20-related kinase adaptor alpha; STE20-related kinase adaptor beta; STEAP family member 1B; STEAP family member 2, metalloreductase; STEAP family member 3, metalloreductase; STEAP family member 4; stearoyl-CoA desaturase (delta-9-desaturase); stearoyl-CoA desaturase 5; stem-loop binding protein; stereocilin; steroid 5 alpha-reductase 3; steroid receptor RNA activator 1; steroid sulfatase (microsomal), isozyme S; steroid-5-alpha-reductase, alpha polypeptide 1 (3-oxo-5 alpha-steroid delta 4-dehydrogenase alpha 1); steroidogenic acute regulatory protein; sterol carrier protein 2; sterol O-acyltransferase 1; sterol O-acyltransferase 2; sterol regulatory element binding transcription factor 1; sterol regulatory element binding transcription factor 2; stimulator of chondrogenesis 1; stomatin; stomatin (EPB72)-like 1; stomatin (EPB72)-like 2; stomatin (EPB72)-like 3; STON1-GTF2A1L readthrough; stonin 1; stonin 2; storkhead box 1; storkhead box 2; stratifin; stress responsive DNAJB4 interacting membrane protein 1; stress-associated endoplasmic reticulum protein 1; stress-associated endoplasmic reticulum protein family member 2; stress-induced-phosphoprotein 1; striatin, calmodulin binding protein; striatin, calmodulin binding protein 3; striatin, calmodulin binding protein 4; stromal antigen 1; stromal antigen 2; stromal antigen 3; stromal antigen 3-like 1; stromal antigen 3-like 2; stromal antigen 3-like 3; stromal antigen 3-like 4; stromal cell derived factor 4; stromal cell-derived factor 2; stromal cell-derived factor 2-like 1; stromal interaction molecule 1; stromal interaction molecule 2; structural maintenance of chromosomes 1A, 1B, and 2-6; structure specific recognition protein 1; submaxillary gland androgen regulated protein 3A; submaxillary gland androgen regulated protein 3B; succinate dehydrogenase complex assembly factor 1; succinate dehydrogenase complex assembly factor 2; succinate dehydrogenase complex, subunit A, flavoprotein (Fp); succinate dehydrogenase complex, subunit B, iron sulfur (Ip); succinate dehydrogenase complex, subunit C, integral membrane protein, 15 kDa; succinate dehydrogenase complex, subunit D, integral membrane protein; succinate receptor 1; succinate-CoA ligase, ADP-forming, beta subunit; succinate-CoA ligase, alpha subunit; succinate-CoA ligase, GDP-forming, beta subunit; sucrase-isomaltase (alpha-glucosidase); sulfatase 1; sulfatase 2; sulfatase modifying factor 1; sulfatase modifying factor 2; sulfide quinone reductase-like (yeast); sulfiredoxin 1; sulfite oxidase; Sulfotransferase 1A3/1A4; sulfotransferase family 1E, estrogen-preferring, member 1; sulfotransferase family 4A, member 1; sulfotransferase family, cytosolic, 1A, phenol-preferring, member 1; sulfotransferase family, cytosolic, 1A, phenol-preferring, member 2; sulfotransferase family, cytosolic, 1A, phenol-preferring, member 4; sulfotransferase family, cytosolic, 1B, member 1; sulfotransferase family, cytosolic, 1C, member 2; sulfotransferase family, cytosolic, 1C, member 3; sulfotransferase family, cytosolic, 1C, member 4; sulfotransferase family, cytosolic, 2A, dehydroepiandrosterone (DHEA)-preferring, member 1; sulfotransferase family, cytosolic, 2B, member 1; sulfotransferase family, cytosolic, 6B, member 1; SUMO/sentrin specific peptidase family member 8; SUMO1 activating enzyme subunit 1; SUMO1/sentrin specific peptidase 1; SUMO1/sentrin specific peptidase 5; SUMO1/sentrin specific peptidase 6; SUMO1/sentrin specific peptidase 7; SUMO1/sentrin/SMT3 specific peptidase 2; SUMO1/sentrin/SMT3 specific peptidase 3superoxide dismutase 1, soluble; superoxide dismutase 2, mitochondrial; superoxide dismutase 3, extracellular; supervillin; suppression of tumorigenicity 13 (colon carcinoma) (Hsp70 interacting protein); suppression of tumorigenicity 14 (colon carcinoma); suppression of tumorigenicity 18 (breast carcinoma) (zinc finger protein); suppression of tumorigenicity 5; suppression of tumorigenicity 7; suppression of tumorigenicity 7 like; suppressor of cancer cell invasion; suppressor of cytokine signaling 1-7; suppressor of IKBKE 1; suppressor of tumorigenicity 20; suprabasin; surfactant associated 2; surfactant associated 3; surfactant protein A1; surfactant protein A2; surfactant protein B; surfactant protein C; surfactant protein D; surfeit 1; surfeit 2; surfeit 4; surfeit 6; survival of motor neuron 1, telomeric; survival of motor neuron 2, centromeric; sushi, nidogen and EGF-like domains 1; SVOP-like; SWAP switching B-cell complex 70 kDa subunit; SWI/SNF related, matrix associated, actin dependent regulator of chromatin, subfamily a, member 1, 2, 4 and 5; SWI/SNF related, matrix associated, actin dependent regulator of chromatin, subfamily a-like 1; SWI/SNF related, matrix associated, actin dependent regulator of chromatin, subfamily b, member 1; SWI/SNF related, matrix associated, actin dependent regulator of chromatin, subfamily c, member 1; SWI/SNF related, matrix associated, actin dependent regulator of chromatin, subfamily c, member 2; SWI/SNF related, matrix associated, actin dependent regulator of chromatin, subfamily d, member 1; SWI/SNF related, matrix associated, actin dependent regulator of chromatin, subfamily d, member 2; SWI/SNF related, matrix associated, actin dependent regulator of chromatin, subfamily d, member 3; SWI/SNF related, matrix associated, actin dependent regulator of chromatin, subfamily e, member 1; SWI5-dependent recombination repair 1; SWIM-type zinc finger 7 associated protein 1; symplekin; synapse associated protein 1; synapse differentiation inducing 1; synapse differentiation inducing 1-like; synapsin I; synapsin II; synapsin III; synaptic Ras GTPase activating protein 1; synaptic vesicle glycoprotein 2A; synaptic vesicle glycoprotein 2B; synaptic vesicle glycoprotein 2C; synaptogyrin 1; synaptogyrin 2; synaptogyrin 3; synaptogyrin 4; synaptoj anin 1; synaptoj anin 2; synaptoj anin 2 binding protein; synaptonemal complex central element protein 1; synaptonemal complex central element protein 1-like; synaptonemal complex central element protein 2; synaptonemal complex protein 1; synaptonemal complex protein 2; synaptonemal complex protein 2-like; synaptonemal complex protein 3; synaptophysin; synaptophysin-like 1; synaptophysin-like 2; synaptopodin; synaptopodin 2; synaptopodin 2-like; synaptoporin; synaptosomal-associated protein, 23 kDa; synaptosomal-associated protein, 25 kDa; synaptosomal-associated protein, 29 kDa; synaptosomal-associated protein, 47 kDa; synaptotagmin binding, cytoplasmic RNA interacting protein; synaptotagmin I-XVII; synaptotagmin-like 1-like 5; syncoilin, intermediate filament protein; syncollin; syndecan 1; syndecan 2; syndecan 3; syndecan 4; syndecan binding protein (syntenin); syndecan binding protein (syntenin) 2; synemin, intermediate filament protein; synergin, gamma; synovial apoptosis inhibitor 1, synoviolin; synovial sarcoma translocation gene on chromosome 18-like 1; synovial sarcoma translocation gene on chromosome 18-like 2; synovial sarcoma translocation, chromosome 18; synovial sarcoma, X breakpoint 1; synovial sarcoma, X breakpoint 2; synovial sarcoma, X breakpoint 2 interacting protein; synovial sarcoma, X breakpoint 2B; synovial sarcoma, X breakpoint 3; synovial sarcoma, X breakpoint 4; synovial sarcoma, X breakpoint 4B; synovial sarcoma, X breakpoint 5; synovial sarcoma, X breakpoint 7; synovial sarcoma, X breakpoint 9; syntabulin (syntaxin-interacting); syntaphilin; syntaxin 1A (brain), 1B, 2-8, 10-12 and 16-19; syntaxin binding protein 1; syntaxin binding protein 2; syntaxin binding protein 3; syntaxin binding protein 4; syntaxin binding protein 5 (tomosyn); syntaxin binding protein 5-like; syntaxin binding protein 6 (amisyn); syntrophin, alpha 1 (dystrophin-associated protein A1, 59 kDa, acidic component); syntrophin, beta 1 (dystrophin-associated protein A1, 59 kDa, basic component 1); syntrophin, beta 2 (dystrophin-associated protein A1, 59 kDa, basic component 2); syntrophin, gamma 1; syntrophin, gamma 2; synuclein, alpha (non A4 component of amyloid precursor); synuclein, alpha interacting protein; synuclein, beta; synuclein, gamma (breast cancer-specific protein 1); T cell immunoreceptor with Ig and ITIM domains; T cell receptor associated transmembrane adaptor 1; T cell receptor beta constant 2; T cell receptor beta variable 20/0R9-2; T cell receptor beta variable 23/0R9-2; T cell receptor delta constant; T cell receptor gamma variable 3; T cell-interacting, activating receptor on myeloid cells 1; tachykinin 3; tachykinin 4 (hemokinin); tachykinin receptor 1; tachykinin receptor 2; tachykinin receptor 3; tachykinin, precursor 1; TAF1 RNA polymerase II, TATA box binding protein (TBP)-associated factor, 210 kDa-like; TAF1 RNA polymerase II, TATA box binding protein (TBP)-associated factor, 250 kDa; TAF10 RNA polymerase II, TATA box binding protein (TBP)-associated factor, 30 kDa; TAF11 RNA polymerase II, TATA box binding protein (TBP)-associated factor, 28 kDa; TAF12 RNA polymerase II, TATA box binding protein (TBP)-associated factor, 20 kDa; TAF13 RNA polymerase II, TATA box binding protein (TBP)-associated factor, 18 kDa; TAF15 RNA polymerase II, TATA box binding protein (TBP)-associated factor, 68 kDa; TAF2 RNA polymerase II, TATA box binding protein (TBP)-associated factor, 150 kDa; TAF3 RNA polymerase II, TATA box binding protein (TBP)-associated factor, 140 kDa; TAF4 RNA polymerase II, TATA box binding protein (TBP)-associated factor, 135 kDa; TAF4b RNA polymerase II, TATA box binding protein (TBP)-associated factor, 105 kDa; TAF5 RNA polymerase II, TATA box binding protein (TBP)-associated factor, 100 kDa; TAF5-like RNA polymerase II, p300/CBP-associated factor (PCAF)-associated factor, 65 kDa; TAF6 RNA polymerase II, TATA box binding protein (TBP)-associated factor, 80 kDa; TAF6-like RNA polymerase II, p300/CBP-associated factor (PCAF)-associated factor, 65 kDa; TAF7 RNA polymerase II, TATA box binding protein (TBP)-associated factor, 55 kDa; TAF7-like RNA polymerase II, TATA box binding protein (TBP)-associated factor, 50 kDa; TAF8 RNA polymerase II, TATA box binding protein (TBP)-associated factor, 43 kDa; TAF9 RNA polymerase II, TATA box binding protein (TBP)-associated factor, 32 kDa; TAF9B RNA polymerase II, TATA box binding protein (TBP)-associated factor, 31 kDa; tafazzin; talin 1; talin 2; tandem C2 domains, nuclear; TANK-binding kinase 1; tankyrase 1 binding protein 1, 182 kDa; tankyrase, TRF1-interacting ankyrin-related ADP-ribose polymerase; tankyrase, TRF1-interacting ankyrin-related ADP-ribose polymerase 2; TAO kinase 1; TAO kinase 2; TAO kinase 3; TAP binding protein (tapasin); TAP binding protein-like; taperin; TAR (HIV-1) RNA binding protein 1; TAR (HIV-1) RNA binding protein 2; TAR DNA binding protein; target of EGR1, member 1 (nuclear); target of myb 1 (chicken); target of myb 1 (chicken)-like 1; target of myb 1-like 2 (chicken); taspase, threonine aspartase, 1; taste receptor, type 1, member 1-3; taste receptor, type 2, member 1, 3, 4, 5, 7-10, 13, 14, 16, 19, 20, 30, 31, 38-43, 46, 50 and 60; TATA box binding protein; TATA box binding protein (TBP)-associated factor, RNA polymerase I, A, 48 kDa; TATA box binding protein (TBP)-associated factor, RNA polymerase I, B, 63 kDa; TATA box binding protein (TBP)-associated factor, RNA polymerase I, C, 110 kDa; TATA box binding protein (TBP)-associated factor, RNA polymerase I, D, 41 kDa; TATA box binding protein like 2; TATA element modulatory factor 1; tau tubulin kinase 1; tau tubulin kinase 2; Tax1 (human T-cell leukemia virus type I) binding protein 1; Taxl (human T-cell leukemia virus type I) binding protein 3; taxilin alpha; taxilin beta; taxilin gamma; TBC1 (tre-2/USP6, BUB2, cdc16) domain family, member 1; TBC1 domain family, member 2, 2B, 3, 3B, 3C, 3F, 3G, 3H, 4, 5, 7, 8 (with GRAM domain), 8B (with GRAM domain), 9 (with GRAM domain), 9B (with GRAM domain), 10A, 10B, 10C, 12, 13, 14, 15, 16, 17, 19, 20, 21, 22A, 22B, 23, 24, 25, 26, 28, 29, and 30; TBK1 binding protein 1; T-box 1; T-box 10; T-box 15; T-box 18; T-box 19; T-box 2; T-box 20; T-box 21; T-box 22; T-box 3; T-box 4; T-box 5; T-box 6; T-box, brain, 1; TBP-like 1; TCDD-inducible poly(ADP-ribose) polymerase; T-cell activation RhoGTPase activating protein; T-cell acute lymphocytic leukemia 1; T-cell acute lymphocytic leukemia 2; T-cell leukemia homeobox 1; T-cell leukemia homeobox 2; T-cell leukemia homeobox 3; T-cell leukemia translocation altered gene; T-cell leukemia/lymphoma 1A; T-cell leukemia/lymphoma 1B; T-cell lymphoma invasion and metastasis 1; T-cell lymphoma invasion and metastasis 2; T-cell, immune regulator 1, ATPase, H+ transporting, lysosomal V0 subunit A3; TCF3 (E2A) fusion partner (in childhood Leukemia); t-complex 1; t-complex 10 (mouse)-like; t-complex 11 (mouse)-like 1; t-complex 11 (mouse)-like 2; t-complex-associated-testis-expressed 1; t-complex-associated-testis-expressed 3; TDP-glucose 4,6-dehydratase; TEA domain family member 1 (SV40 transcriptional enhancer factor); TEA domain family member 2; TEA domain family member 3; TEA domain family member 4; teashirt zinc finger homeobox 1; teashirt zinc finger homeobox 2; teashirt zinc finger homeobox 3; tec protein tyrosine kinase; tectonic family member 1; tectonic family member 2; tectonic family member 3; tectorin alpha; tectorin beta; TEK tyrosine kinase, endothelial; tektin 1; tektin 2 (testicular); tektin 3; tektin 4; tektin 5; TELO2 interacting protein 1; TELO2 interacting protein 2; telomerase reverse transcriptase; telomerase-associated protein 1; telomeric repeat binding factor (NIMA-interacting) 1; telomeric repeat binding factor 2; telomeric repeat binding factor 2, interacting protein; tenascin C; tenascin N; tenascin R (restrictin, janusin); tenascin XB; tenomodulin; tensin 1; tensin 3; tensin 4; teratocarcinoma-derived growth factor 1; TERF1 (TRF1)-interacting nuclear factor 2; terminal uridylyl transferase 1, U6 snRNA-specific; tescalcin; testis derived transcript (3 LIM domains); testis expressed 10; testis expressed 101; testis expressed 11; testis expressed 12; testis expressed 13A; testis expressed 13B; testis expressed 14; testis expressed 15; testis expressed 19; testis expressed 2; testis expressed 261; testis expressed 264; testis expressed 28; testis expressed 9; testis specific protein, Y-linked 1; testis specific protein, Y-linked 10; testis specific protein, Y-linked 2; testis specific protein, Y-linked 3; testis specific protein, Y-linked 4; testis specific protein, Y-linked 8; testis specific, 10; testis specific, 13; testis, prostate and placenta expressed; testis-specific kinase 1; testis-specific kinase 2; testis-specific serine kinase 1B; testis-specific serine kinase 2; testis-specific serine kinase 3; testis-specific serine kinase 4; testis-specific serine kinase 6; testis-specific serine kinase substrate; tet methylcytosine dioxygenase 1; tet methylcytosine dioxygenase 2; tet methylcytosine dioxygenase 3; tetra-peptide repeat homeobox 1; tetra-peptide repeat homeobox-like; tetraspanin 1-19 and 31-33; tetratricopeptide repeat domain 1, 3, 4, 5, 6, 7A, 7B, 8, 9, 9B, 9C, 12, 13, 14, 16, 17, 18, 19, 21A, 21B, 22, 23, 23-like, 24, 26, 27, 29, 30A, 30B, 31, 32, 33, 34, 35, 36, 37, 38, 39A-C, and 40; TGFB1-induced anti-apoptotic factor 1; TGF-beta activated kinase 1/MAP3K7 binding protein 1; TGF-beta activated kinase 1/MAP3K7 binding protein 2; TGF-beta activated kinase 1/MAP3K7 binding protein 3; TGFB-induced factor homeobox 1; TGFB-induced factor homeobox 2; TGFB-induced factor homeobox 2-like, X-linked; TGFB-induced factor homeobox 2-like, Y-linked; THAP domain containing, apoptosis associated protein 1; THAP domain containing, apoptosis associated protein 2; THAP domain containing, apoptosis associated protein 3; thiamin pyrophosphokinase 1; thiamine triphosphatase; thimet oligopeptidase 1; thioesterase superfamily member 4; thioesterase superfamily member 5; thiopurine S-methyltransferase; thioredoxin; thioredoxin 2; thioredoxin interacting protein; thioredoxin reductase 1; thioredoxin reductase 2; thioredoxin reductase 3; thioredoxin reductase 3 neighbor; thioredoxin-like 1; thioredoxin-like 4A; thioredoxin-like 4B; thioredoxin-related transmembrane protein 1; thioredoxin-related transmembrane protein 2; thioredoxin-related transmembrane protein 3; thioredoxin-related transmembrane protein 4; thiosulfate sulfurtransferase (rhodanese); THO complex 1; THO complex 2; THO complex 3; THO complex 5; three prime repair exonuclease 1; three prime repair exonuclease 2; threonyl-tRNA synthetase; threonyl-tRNA synthetase-like 2; thrombomodulin; thrombopoietin; thrombospondin 1; thrombospondin 2; thrombospondin 3; thrombospondin 4; thrombospondin-type laminin G domain and EAR repeats; thromboxane A synthase 1 (platelet); thromboxane A2 receptor; Thy-1 cell surface antigen; thymic stromal lymphopoietin; thymidine kinase 1, soluble; thymidine kinase 2, mitochondrial; thymidine phosphorylase; thymidylate synthetase; thymine-DNA glycosylase; thymocyte nuclear protein 1; thymocyte selection associated; thymocyte selection-associated high mobility group box; thymopoietin; thymosin beta 10; thymosin beta 15a; thymosin beta 4, X-linked; thymosin beta 4, Y-linked; thyroglobulin; thyroid adenoma associated; thyroid hormone receptor associated protein 3; thyroid hormone receptor interactor 10; thyroid hormone receptor interactor 11; thyroid hormone receptor interactor 12; thyroid hormone receptor interactor 13; thyroid hormone receptor interactor 4; thyroid hormone receptor interactor 6; thyroid hormone receptor, alpha; thyroid hormone receptor, beta; thyroid hormone responsive; thyroid peroxidase; thyroid stimulating hormone receptor; thyroid stimulating hormone, beta; thyrotrophic embryonic factor; thyrotropin-releasing hormone; thyrotropin-releasing hormone degrading enzyme; thyrotropin-releasing hormone receptor; TIA1 cytotoxic granule-associated RNA binding protein; TIA1 cytotoxic granule-associated RNA binding protein-like 1; tigger transposable element derived 1; tigger transposable element derived 1-like 2; tigger transposable element derived 2; tigger transposable element derived 3; tigger transposable element derived 4; tigger transposable element derived 5; tigger transposable element derived 6; tigger transposable element derived 7; tight junction associated protein 1 (peripheral); tight junction protein 1 (zona occludens 1); tight junction protein 2 (zona occludens 2); tight junction protein 3 (zona occludens 3); TIMELESS interacting protein; TIMP metallopeptidase inhibitor 1; TIMP metallopeptidase inhibitor 2; TIMP metallopeptidase inhibitor 3; TIMP metallopeptidase inhibitor 4; tissue factor pathway inhibitor (lipoprotein-associated coagulation inhibitor); tissue factor pathway inhibitor 2; tissue specific transplantation antigen P35B; titin; titin-cap (telethonin); TLR9; TMED7-TICAM2 readthrough; TMEM189-UBE2V1 readthrough; TMEM9 domain family, member B; TMF1-regulated nuclear protein 1; TMPRSS11B N terminal-like; TNF receptor-associated factor 1; TNF receptor-associated factor 2; TNF receptor-associated factor 3; TNF receptor-associated factor 3 interacting protein 1; TNF receptor-associated factor 4; TNF receptor-associated factor 5; TNF receptor-associated factor 6; TNF receptor-associated factor 7; TNF receptor-associated protein 1; TNFAIP3 interacting protein 1; TNFAIP3 interacting protein 2; TNFAIP3 interacting protein 3; TNFRSF1A-associated via death domain; TNFSF12-TNFSF13 readthrough; tocopherol (alpha) transfer protein; tocopherol (alpha) transfer protein-like; toll interacting protein; toll-like receptor 1-10; toll-like receptor adaptor molecule 1; toll-like receptor adaptor molecule 2; tolloid-like 1; tolloid-like 2; tonsoku-like, DNA repair protein; topoisomerase (DNA) I; topoisomerase (DNA) I, mitochondrial; topoisomerase (DNA) II alpha 170 kDa; topoisomerase (DNA) II beta 180 kDa; topoisomerase (DNA) II binding protein 1; topoisomerase (DNA) III alpha; topoisomerase (DNA) III beta; topoisomerase I binding, arginine/serine-rich, E3 ubiquitin protein ligase; torsin A interacting protein 1; torsin A interacting protein 2; torsin family 1, member A (torsin A); torsin family 1, member B (torsin B); torsin family 2, member A; torsin family 3, member A; tousled-like kinase 1; tousled-like kinase 2; TOX high mobility group box family member 2; TOX high mobility group box family member 3; TOX high mobility group box family member 4; TP53 regulated inhibitor of apoptosis 1; TP53 regulating kinase; TP53 target 3; TP53 target 3B; TP53 target 5; TP53RK binding protein; TP53-target gene 3 protein; TraB domain containing; trace amine associated receptor 1; trace amine associated receptor 2; trace amine associated receptor 5; trace amine associated receptor 6; trace amine associated receptor 8; TRAF family member-associated NFKB activator; TRAF interacting protein; TRAF2 and NCK interacting kinase; TRAF3 interacting protein 2; TRAF3 interacting protein 3; trafficking protein particle complex 1; trafficking protein particle complex 10; trafficking protein particle complex 11; trafficking protein particle complex 12; trafficking protein particle complex 2; trafficking protein particle complex 2-like; trafficking protein particle complex 3; trafficking protein particle complex 4; trafficking protein particle complex 5; trafficking protein particle complex 6A; trafficking protein particle complex 6B; trafficking protein particle complex 8; trafficking protein particle complex 9; trafficking protein, kinesin binding 1; trafficking protein, kinesin binding 2; TRAF-interacting protein with forkhead-associated domain; TRAF-interacting protein with forkhead-associated domain, family member B; trans-2,3-enoyl-CoA reductase; trans-2,3-enoyl-CoA reductase-like; transaldolase 1; transcobalamin I (vitamin B12 binding protein, R binder family); transcobalamin II; transcription elongation factor A (SII)N-terminal and central domain containing; transcription elongation factor A (SII), 1; transcription elongation factor A (SII), 2; transcription elongation factor A (SII), 3; transcription elongation factor A (SII)-like 1; transcription elongation factor A (SII)-like 2; transcription elongation factor A (SII)-like 3; transcription elongation factor A (SII)-like 4; transcription elongation factor A (SII)-like 5; transcription elongation factor A (SII)-like 6; transcription elongation factor A (SII)-like 7; transcription elongation factor A (SII)-like 8; transcription elongation factor B (SIII), polypeptide 1 (15 kDa, elongin C); transcription elongation factor B (SIII), polypeptide 2 (18 kDa, elongin B); transcription elongation factor B (SIII), polypeptide 3 (110 kDa, elongin A); transcription elongation factor B polypeptide 3B (elongin A2); transcription elongation factor B polypeptide 3C (elongin A3); transcription elongation factor B polypeptide 3C-like; transcription elongation factor, mitochondrial; transcription elongation regulator 1; transcription elongation regulator 1-like; transcription factor 12; transcription factor 15 (basic helix-loop-helix); transcription factor 19; transcription factor 20 (AR1); transcription factor 21; transcription factor 23; transcription factor 25 (basic helix-loop-helix); transcription factor 3 (E2A immunoglobulin enhancer binding factors E12/E47); transcription factor 4; transcription factor 7 (T-cell specific, HMG-box); transcription factor 7-like 1 (T-cell specific, HMG-box); transcription factor 7-like 2 (T-cell specific, HMG-box); transcription factor A, mitochondrial; transcription factor AP-2 alpha (activating enhancer binding protein 2 alpha); transcription factor AP-2 beta (activating enhancer binding protein 2 beta); transcription factor AP-2 delta (activating enhancer binding protein 2 delta); transcription factor AP-2 epsilon (activating enhancer binding protein 2 epsilon); transcription factor AP-2 gamma (activating enhancer binding protein 2 gamma); transcription factor AP-4 (activating enhancer binding protein 4); transcription factor B1, mitochondrial; transcription factor B2, mitochondrial; transcription factor binding to IGHM enhancer 3; transcription factor CP2; transcription factor CP2-like 1; transcription factor Dp family, member 3; transcription factor Dp-1; transcription factor Dp-2 (E2F dimerization partner 2); transcription factor EB; transcription factor EC; transcription factor-like 5 (basic helix-loop-helix); transcription termination factor, RNA polymerase I; transcription termination factor, RNA polymerase II; transcriptional adaptor 1; transcriptional adaptor 2A; transcriptional adaptor 2B; transcriptional adaptor 3; transcriptional regulating factor 1; transducer of ERBB2, 1; transducer of ERBB2, 2; transducin (beta)-like 1 X-linked receptor 1; transducin (beta)-like 1, Y-linked; transducin (beta)-like 1X-linked; transducin (beta)-like 2; transducin (beta)-like 3; transferrin; transferrin receptor (p90, CD71); transferrin receptor 2; transformation/transcription domain-associated protein; transforming growth factor beta 1 induced transcript 1; transforming growth factor beta regulator 1; transforming growth factor beta regulator 4; transforming growth factor, alpha; transforming growth factor, beta 1; transforming growth factor, beta 2; transforming growth factor, beta 3; transforming growth factor, beta receptor 1; transforming growth factor, beta receptor associated protein 1; transforming growth factor, beta receptor II (70/80 kDa); transforming growth factor, beta receptor III; transforming growth factor, beta-induced, 68 kDa; transgelin; transgelin 2; transgelin 3; transglutaminase 1 (K polypeptide epidermal type I, protein-glutamine-gamma-glutamyltransferase); transglutaminase 2 (C polypeptide, protein-glutamine-gamma-glutamyltransferase); transglutaminase 3 (E polypeptide, protein-glutamine-gamma-glutamyltransferase); transglutaminase 4 (prostate); transglutaminase 5; transglutaminase 6; transglutaminase 7; trans-golgi network protein 2; transient receptor potential cation channel, subfamily A, member 1; transient receptor potential cation channel, subfamily C, member 1, member 3-7 and member 4 associated protein; transient receptor potential cation channel, subfamily M, member 1-8; transient receptor potential cation channel, subfamily V, member 1-6; transition protein 1 (during histone to protamine replacement); transition protein 2 (during histone to protamine replacement); transketolase; transketolase-like 1; transketolase-like 2; translational activator of mitochondrially encoded cytochrome c oxidase I; translin; translin-associated factor X; translin-associated factor X interacting protein 1; translocase of outer mitochondrial membrane 34; translocated promoter region (to activated MET oncogene); translocation associated membrane protein 1; translocation associated membrane protein 1-like 1; translocation associated membrane protein 2; translocator protein (18 kDa); translocator protein 2; transmembrane (C-terminal) protease, serine 12; transmembrane 4 L six family member 1; transmembrane 4 L six family member 18; transmembrane 4 L six family member 19; transmembrane 4 L six family member 20; transmembrane 4 L six family member 4; transmembrane 4 L six family member 5; transmembrane 6 superfamily member 1; transmembrane 6 superfamily member 2; transmembrane 7 superfamily member 2; transmembrane 7 superfamily member 3; transmembrane 7 superfamily member 4; transmembrane 9 superfamily member 1; transmembrane 9 superfamily member 1 isoform a precursor; transmembrane 9 superfamily member 2; transmembrane 9 superfamily member 3; transmembrane 9 superfamily protein member 4; transmembrane and coiled-coil domain family 1; transmembrane and coiled-coil domain family 2; transmembrane and coiled-coil domain family 3; transmembrane and coiled-coil domains 1; transmembrane and coiled-coil domains 2; transmembrane and coiled-coil domains 3; transmembrane and coiled-coil domains 4; transmembrane and coiled-coil domains 5A; transmembrane and coiled-coil domains 6; transmembrane and coiled-coil domains 7; transmembrane anterior posterior transformation 1; transmembrane channel-like 1-8; transmembrane emp24 domain trafficking protein 2; transmembrane emp24-like trafficking protein 10 (yeast); transmembrane epididymal protein 1; transmembrane inner ear; transmembrane protease, serine 2-7, 9 11A, 11B, 11D-F, 13 and 15; transmembrane protein 2, 5, 8A, 8B, 8C, 9, 11, 14A, 14B, 14C, 14E, 25, 26, 27, 30A, 30B, 30C, 31, 33, 35, 37, 38A, 38B, 39A, 39B, 40, 41A, 41B, 42, 43, 44, 45A, 45B, 47, 48, 50A, 50B, 51, 52, 53, 54, 55A, 55B, 56, 57, 59, 59-like, 60, 61, 62, 63A, 63B, 63C, 64, 65, 66, 67, 68, 69, 70, 71, 72, 74, 74B, 75, 78, 79, 80, 81, 82, 85, 86A, 86B, 87A, 87B, 88, 89, 91, 92, 93, 95, 97, 98, 99, 100, 101, 102, 104, 105, 106A, 106B, 106C, 107, 108, 109, 110, 111, 114, 115, 116, 117, 119, 120B, 121, 123, 125, 126A, 126B, 127, 128, 129, 130, 131, 132A, 132B, 132C, 132D, 132E, 133, 134, 135, 136, 138, 139, 140, 141, 143, 144, 145, 146, 147, 150A, 150B, 150C, 151A, 151B, 154, 155, 156, 159, 160, 161A, 161B, 163, 164, 165, 167A, 167B, 168, 169, 17, 170A, 170B, 171, 173, 174, 175, 176A, 176B, 177, 178, 179, 179B, 18, 180, 181, 182, 183A, 184A, 184B, 184C, 185A, 185B, 186, 187, 188, 189, 19, 190, 192, 194A, 194B, 196, 198, 199, 200A, 200B, 200C, 201, 202, 203, 204, 205, 206, 207, 208, 209, 211, 212, 213, 214, 215, 216, 217, 218, 219, 22, 220, 221, 222, 223, 225, 229A, 229B, 231, 232, 233, 234, 235, 236, 237, 238, 239, 240, 241, 242, ENSP00000343375 and FLJ78588; transmembrane protein with EGF-like and two follistatin-like domains 1; transmembrane protein with EGF-like and two follistatin-like domains 2; transmembrane protein with metallophosphoesterase domain; transmembrane protein, adipocyte asscociated 1; transporter 1, ATP-binding cassette, sub-family B (MDR/TAP); transporter 2, ATP-binding cassette, sub-family B (MDR/TAP); transportin 1; transportin 2; transportin 3; transthyretin; Treacher Collins-Franceschetti syndrome 1; trefoil factor 1; trefoil factor 2; trefoil factor 3 (intestinal); trehalase (brush-border membrane glycoprotein); triadin; trichohyalin; trichohyalin-like 1; trichoplein, keratin filament binding; trichorhinophalangeal syndrome I; triggering receptor expressed on myeloid cells 1; triggering receptor expressed on myeloid cells 2; triggering receptor expressed on myeloid cells-like 1; triggering receptor expressed on myeloid cells-like 2; triggering receptor expressed on myeloid cells-like 4; TRIM39-RPP21 readthrough; TRIM6-TRIM34 readthrough; trimethylguanosine synthase 1; trimethyllysine hydroxylase, epsilon; TRIO and F-actin binding protein; triosephosphate isomerase 1; tripartite motif family-like 1; tripartite motif family-like 2; tripeptidyl peptidase I; tripeptidyl peptidase II; triple functional domain (PTPRF interacting); TRK-fused gene; TRM1 tRNA methyltransferase 1-like; tRNA 5-methylaminomethyl-2-thiouridylate methyltransferase; tRNA aspartic acid methyltransferase 1; tRNA isopentenyltransferase 1; tRNA nucleotidyl transferase, CCA-adding, 1; tRNA phosphotransferase 1; tRNA selenocysteine 1 associated protein 1; tRNA-yW synthesizing protein 5; trophinin; trophinin associated protein (tastin); trophoblast glycoprotein; tropomodulin 1; tropomodulin 2 (neuronal); tropomodulin 3 (ubiquitous); tropomodulin 4 (muscle); tropomyosin 1 (alpha); tropomyosin 2 (beta); tropomyosin 3; tropomyosin 4; troponin C type 1 (slow); troponin C type 2 (fast); troponin I type 1 (skeletal, slow); troponin I type 2 (skeletal, fast); troponin I type 3 (cardiac); troponin T type 1 (skeletal, slow); troponin T type 2 (cardiac); troponin T type 3 (skeletal, fast); TROVE domain family, member 2; tryptase alpha/beta 1; tryptase delta 1; tryptase gamma 1; tryptophan 2,3-dioxygenase; tryptophan hydroxylase 1; tryptophan hydroxylase 2; tryptophan rich basic protein; tryptophanyl tRNA synthetase 2, mitochondrial; tryptophanyl-tRNA synthetase; Ts translation elongation factor, mitochondrial; TSC22 domain family, member 1; TSC22 domain family, member 2; TSC22 domain family, member 3; TSC22 domain family, member 4; TSPY-like 1; TSPY-like 2; TSPY-like 4; TSPY-like 5; TSPY-like 6; TTK protein kinase; Tu translation elongation factor, mitochondrial; tubby like protein 1; tubby like protein 2; tubby like protein 3; tubby like protein 4; tuberous sclerosis 1; tuberous sclerosis 2; tubulin folding cofactor A; tubulin folding cofactor B; tubulin folding cofactor C; tubulin folding cofactor D; tubulin folding cofactor E; tubulin folding cofactor E-like; tubulin polyglutamylase complex subunit 1; tubulin polyglutamylase complex subunit 2; tubulin polymerization promoting protein; tubulin polymerization-promoting protein family member 2; tubulin polymerization-promoting protein family member 3; tubulin tyrosine ligase; tubulin tyrosine ligase-like family, member 1-13; tubulin, alpha 1a; tubulin, alpha 1b; tubulin, alpha 1c; tubulin, alpha 3c; tubulin, alpha 3d; tubulin, alpha 3e; tubulin, alpha 4a; tubulin, alpha 8; tubulin, alpha-like 3; tubulin, beta 1 class VI; tubulin, beta 2A class IIa; tubulin, beta 2B class Hb; tubulin, beta 3 class III; tubulin, beta 4A class IVa; tubulin, beta 4B class IVb; tubulin, beta 6 class V; tubulin, beta 8 class VIII; tubulin, beta class I; tubulin, delta 1; tubulin, epsilon 1; tubulin, gamma 1; tubulin, gamma 2; tubulin, gamma complex associated protein 2; tubulin, gamma complex associated protein 3; tubulin, gamma complex associated protein 4; tubulin, gamma complex associated protein 5; tubulin, gamma complex associated protein 6; tubulointerstitial nephritis antigen; tubulointerstitial nephritis antigen-like 1; tudor and KH domain containing; tuftelin 1; tuftelin interacting protein 11; tumor necrosis factor; tumor necrosis factor (ligand) superfamily, member 10; tumor necrosis factor (ligand) superfamily, member 11; tumor necrosis factor (ligand) superfamily, member 12; tumor necrosis factor (ligand) superfamily, member 13; tumor necrosis factor (ligand) superfamily, member 13b; tumor necrosis factor (ligand) superfamily, member 14; tumor necrosis factor (ligand) superfamily, member 15; tumor necrosis factor (ligand) superfamily, member 18; tumor necrosis factor (ligand) superfamily, member 4; tumor necrosis factor (ligand) superfamily, member 8; tumor necrosis factor (ligand) superfamily, member 9; tumor necrosis factor receptor superfamily, member 10a; tumor necrosis factor receptor superfamily, member 10b; tumor necrosis factor receptor superfamily, member 10c, decoy without an intracellular domain; tumor necrosis factor receptor superfamily, member 10d, decoy with truncated death domain; tumor necrosis factor receptor superfamily, member 11a, NFKB activator; tumor necrosis factor receptor superfamily, member 11b; tumor necrosis factor receptor superfamily, member 12A; tumor necrosis factor receptor superfamily, member 13B; tumor necrosis factor receptor superfamily, member 13C; tumor necrosis factor receptor superfamily, member 14; tumor necrosis factor receptor superfamily, member 17; tumor necrosis factor receptor superfamily, member 18; tumor necrosis factor receptor superfamily, member 19; tumor necrosis factor receptor superfamily, member 1A; tumor necrosis factor receptor superfamily, member 1B; tumor necrosis factor receptor superfamily, member 21; tumor necrosis factor receptor superfamily, member 25; tumor necrosis factor receptor superfamily, member 4; tumor necrosis factor receptor superfamily, member 6b, decoy; tumor necrosis factor receptor superfamily, member 8; tumor necrosis factor receptor superfamily, member 9; tumor necrosis factor, alpha-induced protein 1 (endothelial); tumor necrosis factor, alpha-induced protein 2; tumor necrosis factor, alpha-induced protein 3; tumor necrosis factor, alpha-induced protein 6; tumor necrosis factor, alpha-induced protein 8; tumor necrosis factor, alpha-induced protein 8-like 1; tumor necrosis factor, alpha-induced protein 8-like 2; tumor necrosis factor, alpha-induced protein 8-like 3; tumor protein D52; tumor protein D52-like 1; tumor protein D52-like 2; tumor protein D52-like 3; tumor protein p53; tumor protein p53 binding protein 1; tumor protein p53 binding protein, 2; tumor protein p53 inducible nuclear protein 1; tumor protein p53 inducible nuclear protein 2; tumor protein p53 inducible protein 11; tumor protein p53 inducible protein 13; tumor protein p53 inducible protein 3; tumor protein p53 regulated apoptosis inducing protein 1; tumor protein p63; tumor protein p63 regulated 1; tumor protein p63 regulated 1-like; tumor protein p73; tumor protein, translationally-controlled 1; tumor suppressing subtransferable candidate 1; tumor suppressing subtransferable candidate 4; tumor suppressor candidate 1; tumor suppressor candidate 2; tumor suppressor candidate 3; tumor suppressor candidate 5; tumor susceptibility gene 101; tumor-associated calcium signal transducer 2; TWIST neighbor; two pore segment channel 1; two pore segment channel 2; TXK tyrosine kinase; Type III iodothyronine deiodinase; TYRO protein tyrosine kinase binding protein; TYRO3 protein tyrosine kinase; tyrosinase (oculocutaneous albinism IA); tyrosinase-related protein 1; tyrosine 3-monooxygenase/tryptophan 5-monooxygenase activation protein, beta polypeptide; tyrosine 3-monooxygenase/tryptophan 5-monooxygenase activation protein, epsilon polypeptide; tyrosine 3-monooxygenase/tryptophan 5-monooxygenase activation protein, eta polypeptide; tyrosine 3-monooxygenase/tryptophan 5-monooxygenase activation protein, gamma polypeptide; tyrosine 3-monooxygenase/tryptophan 5-monooxygenase activation protein, theta polypeptide; tyrosine 3-monooxygenase/tryptophan 5-monooxygenase activation protein, zeta polypeptide; tyrosine aminotransferase; tyrosine hydroxylase; tyrosine kinase 2; tyrosine kinase with immunoglobulin-like and EGF-like domains 1; tyrosine kinase, non-receptor, 1; tyrosine kinase, non-receptor, 2; Tyrosine-protein kinase SgK223; tyrosyl-DNA phosphodiesterase 1; tyrosyl-DNA phosphodiesterase 2; tyrosylprotein sulfotransferase 1; tyrosylprotein sulfotransferase 2; tyrosyl-tRNA synthetase; tyrosyl-tRNA synthetase 2, mitochondrial; U2 small nuclear RNA auxiliary factor 1; U2 small nuclear RNA auxiliary factor 1-like 4; U2 small nuclear RNA auxiliary factor 2; U2 snRNP-associated SURP domain containing; ubinuclein 1; ubinuclein 2; ubiquilin 1; ubiquilin 2; ubiquilin 3; ubiquilin 4; ubiquilin-like; ubiquinol-cytochrome c reductase binding protein; ubiquinol-cytochrome c reductase complex chaperone; ubiquinol-cytochrome c reductase core protein I; ubiquinol-cytochrome c reductase core protein II; ubiquinol-cytochrome c reductase hinge protein; ubiquinol-cytochrome c reductase, complex III subunit VII, 9.5 kDa; ubiquinol-cytochrome c reductase, complex III subunit X; ubiquinol-cytochrome c reductase, complex III subunit XI; ubiquinol-cytochrome c reductase, Rieske iron-sulfur polypeptide 1; ubiquitin A-52 residue ribosomal protein fusion product 1; ubiquitin associated protein 1; ubiquitin associated protein 1-like; ubiquitin associated protein 2; ubiquitin associated protein 2-like; ubiquitin B; ubiquitin C; ubiquitin carboxyl-terminal esterase L1 (ubiquitin thiolesterase); ubiquitin carboxyl-terminal esterase L3 (ubiquitin thiolesterase); Ubiquitin carboxyl-terminal hydrolase 17; ubiquitin carboxyl-terminal hydrolase 17-like; ubiquitin carboxyl-terminal hydrolase L5; ubiquitin D; ubiquitin fusion degradation 1 like (yeast); ubiquitin protein ligase E3 component n-recognin 1; ubiquitin protein ligase E3 component n-recognin 2; ubiquitin protein ligase E3 component n-recognin 4; ubiquitin protein ligase E3 component n-recognin 5; ubiquitin protein ligase E3A; ubiquitin protein ligase E3B; ubiquitin protein ligase E3C; ubiquitin related modifier 1; ubiquitin specific peptidase 1, 2, 3, 4 (proto-oncogene), 5 (isopeptidase T), 6 (Tre-2 oncogene), 7 (herpes virus-associated), 8, 9 (X-linked), 9 (Y-linked), 10, 11, 12, 13 (isopeptidase T-3), 14 (tRNA-guanine transglycosylase), 15, 16, 17-like 2, 18, 19, 20, 21, 22, 24, 25, 26, 28, 29, 30, 31, 32, 33, 34, 35, 36, 37, 38, 39, 40, 41, 42, 43, 44, 45, 46, 47, 48, 49, 50, 51, 53, 54 and like 1; ubiquitination factor E4A; ubiquitination factor E4B; ubiquitin-conjugating enzyme E2 variant 1, E2 variant 2, E2 J1 U, E2 J2, E2A, E2B, E2C, E2C binding protein, E2D 1, E2D 2, E2D 3, E2E 1, E2E 2, E2E 3, E2G 1, E2G 2, E2H, E2I, E2K, E2L 3, E2L 6, E2M, E2N, E2N-like, E20, E2Q family member 1, E2Q family member 2, E2Q family-like 1, E2R 2, E2S and E2Z; ubiquitin-fold modifier 1; ubiquitin-fold modifier conjugating enzyme 1; ubiquitin-like 3; ubiquitin-like 4A; ubiquitin-like 4B; ubiquitin-like 5; ubiquitin-like 7 (bone marrow stromal cell-derived); ubiquitin-like modifier activating enzyme 1; ubiquitin-like modifier activating enzyme 2; ubiquitin-like modifier activating enzyme 3; ubiquitin-like modifier activating enzyme 5; ubiquitin-like modifier activating enzyme 6; ubiquitin-like modifier activating enzyme 7; ubiquitin-like with PHD and ring finger domains 1; ubiquitin-like with PHD and ring finger domains 2; ubiquitously transcribed tetratricopeptide repeat gene, Y-linked; ubiquitously-expressed, prefoldin-like chaperone; UBX domain protein 1; UBX domain protein 10; UBX domain protein 11; UBX domain protein 2A; UBX domain protein 2B; UBX domain protein 4; UBX domain protein 6; UBX domain protein 7; UDP glucuronosyltransferase 1 family, polypeptide A1; UDP glucuronosyltransferase 1 family, polypeptide A10; UDP glucuronosyltransferase 1 family, polypeptide A3; UDP glucuronosyltransferase 1 family, polypeptide A4; UDP glucuronosyltransferase 1 family, polypeptide A5; UDP glucuronosyltransferase 1 family, polypeptide A6; UDP glucuronosyltransferase 1 family, polypeptide A7; UDP glucuronosyltransferase 1 family, polypeptide A8; UDP glucuronosyltransferase 1 family, polypeptide A9; UDP glucuronosyltransferase 2 family, polypeptide A1, complex locus; UDP glucuronosyltransferase 2 family, polypeptide A3; UDP glucuronosyltransferase 2 family, polypeptide B10; UDP glucuronosyltransferase 2 family, polypeptide B11; UDP glucuronosyltransferase 2 family, polypeptide B15; UDP glucuronosyltransferase 2 family, polypeptide B17; UDP glucuronosyltransferase 2 family, polypeptide B28; UDP glucuronosyltransferase 2 family, polypeptide B4; UDP glucuronosyltransferase 2 family, polypeptide B7; UDP glycosyltransferase 3 family, polypeptide A1; UDP glycosyltransferase 3 family, polypeptide A2; UDP glycosyltransferase 8; UDP-Gal:betaGal beta 1,3-galactosyltransferase polypeptide 6; UDP-Gal:betaGlcNAc beta 1,3-galactosyltransferase, polypeptide 1; UDP-Gal:betaGlcNAc beta 1,3-galactosyltransferase, polypeptide 2; UDP-Gal:betaGlcNAc beta 1,3-galactosyltransferase, polypeptide 4; UDP-Gal:betaGlcNAc beta 1,3-galactosyltransferase, polypeptide 5; UDP-Gal:betaGlcNAc beta 1,4-galactosyltransferase, polypeptide 1-6; UDP-galactose-4-epimerase; UDP-GlcNAc:betaGal beta-1,3-N-acetylglucosaminyltransferase 1; UDP-GlcNAc:betaGal beta-1,3-N-acetylglucosaminyltransferase 2; UDP-GlcNAc:betaGal beta-1,3-N-acetylglucosaminyltransferase 3; UDP-GlcNAc:betaGal beta-1,3-N-acetylglucosaminyltransferase 4; UDP-GlcNAc:betaGal beta-1,3-N-acetylglucosaminyltransferase 5; UDP-GlcNAc:betaGal beta-1,3-N-acetylglucosaminyltransferase 6 (core 3 synthase); UDP-GlcNAc:betaGal beta-1,3-N-acetylglucosaminyltransferase 7; UDP-GlcNAc:betaGal beta-1,3-N-acetylglucosaminyltransferase 8; UDP-GlcNAc:betaGal beta-1,3-N-acetylglucosaminyltransferase 9; UDP-GlcNAc:betaGal beta-1,3-N-acetylglucosaminyltransferase-like 1; UDP-glucose 6-dehydrogenase; UDP-glucose ceramide glucosyltransferase; UDP-glucose glycoprotein glucosyltransferase 1; UDP-glucose glycoprotein glucosyltransferase 2; UDP-glucose pyrophosphorylase 2; UDP-glucuronate decarboxylase 1; UDP-glucuronosyltransferase 2A3 precursor; UDP-glucuronosyltransferase 2B10 isoform 1 precursor; UDP-N-acetyl-alpha-D-galactosamine:polypeptide N-acetylgalactosaminyltransferase 1 (GalNAc-T1); UDP-N-acetyl-alpha-D-galactosamine:polypeptide N-acetylgalactosaminyltransferase 10 (GalNAc-T10); UDP-N-acetyl-alpha-D-galactosamine:polypeptide N-acetylgalactosaminyltransferase 11 (GalNAc-T11); UDP-N-acetyl-alpha-D-galactosamine:polypeptide N-acetylgalactosaminyltransferase 12 (GalNAc-T12); UDP-N-acetyl-alpha-D-galactosamine:polypeptide N-acetylgalactosaminyltransferase 13 (GalNAc-T13); UDP-N-acetyl-alpha-D-galactosamine:polypeptide N-acetylgalactosaminyltransferase 14 (GalNAc-T14); UDP-N-acetyl-alpha-D-galactosamine:polypeptide N-acetylgalactosaminyltransferase 2 (GalNAc-T2); UDP-N-acetyl-alpha-D-galactosamine:polypeptide N-acetylgalactosaminyltransferase 3 (GalNAc-T3); UDP-N-acetyl-alpha-D-galactosamine:polypeptide N-acetylgalactosaminyltransferase 4 (GalNAc-T4); UDP-N-acetyl-alpha-D-galactosamine:polypeptide N-acetylgalactosaminyltransferase 5 (GalNAc-T5); UDP-N-acetyl-alpha-D-galactosamine:polypeptide N-acetylgalactosaminyltransferase 6 (GalNAc-T6); UDP-N-acetyl-alpha-D-galactosamine:polypeptide N-acetylgalactosaminyltransferase 7 (GalNAc-T7); UDP-N-acetyl-alpha-D-galactosamine:polypeptide N-acetylgalactosaminyltransferase 8 (GalNAc-T8); UDP-N-acetyl-alpha-D-galactosamine:polypeptide N-acetylgalactosaminyltransferase 9 (GalNAc-T9); UDP-N-acetyl-alpha-D-galactosamine:polypeptide N-acetylgalactosaminyltransferase-like 1; UDP-N-acetyl-alpha-D-galactosamine:polypeptide N-acetylgalactosaminyltransferase-like 2; UDP-N-acetyl-alpha-D-galactosamine:polypeptide N-acetylgalactosaminyltransferase-like 4; UDP-N-acetyl-alpha-D-galactosamine:polypeptide N-acetylgalactosaminyltransferase-like 5; UDP-N-acetyl-alpha-D-galactosamine:polypeptide N-acetylgalactosaminyltransferase-like 6; UDP-N-acteylglucosamine pyrophosphorylase 1; UDP-N-acteylglucosamine pyrophosphorylase 1-like 1; UEV and lactate/malate dehyrogenase domains; UFM1-specific ligase 1; UFM1-specific peptidase 1 (non-functional); UFM1-specific peptidase 2; UHRF1 binding protein 1; UHRF1 binding protein 1-like; UL16 binding protein 1; UL16 binding protein 2; UL16 binding protein 3; d membrane protein C19orf24; uncoupling protein 1 (mitochondrial, proton carrier); uncoupling protein 2 (mitochondrial, proton carrier); uncoupling protein 3 (mitochondrial, proton carrier); undifferentiated embryonic cell transcription factor 1; UPF0627 protein ENSP00000341061/ENSP00000339743; UPF0627 protein ENSP00000358171; UPF0638 protein B; upper zone of growth plate and cartilage matrix associated; upregulator of cell proliferation; upstream binding protein 1 (LBP-1a); upstream binding transcription factor, RNA polymerase I; upstream transcription factor 1; upstream transcription factor 2, c-fos interacting; uracil-DNA glycosylase; ureidopropionase, beta; prefoldin-like chaperone; uridine monophosphate synthetase; uridine phosphorylase 1; uridine phosphorylase 2; uridine-cytidine kinase 1; uridine-cytidine kinase 1-like 1; uridine-cytidine kinase 2; urocortin; urocortin 2; urocortin 3 (stresscopin); uromodulin; uromodulin-like 1; uronyl-2-sulfotransferase; uroplakin 1A; uroplakin 1B; uroplakin 2; uroplakin 3A; uroplakin 3B; uroplakin 3B-like; uroporphyrinogen decarboxylase; uroporphyrinogen III synthase; urotensin 2; urotensin 2 domain containing; urotensin 2 receptor; Usher syndrome 1C (autosomal recessive, severe); Usher syndrome 1C binding protein 1; Usher syndrome 1G (autosomal recessive); Usher syndrome 2A (autosomal recessive, mild); USP6 N-terminal like; UTP11-like, U3 small nucleolar ribonucleoprotein, (yeast); utrophin; UV radiation resistance associated gene; uveal autoantigen with coiled-coil domains and ankyrin repeats; vaccinia related kinase 1; vaccinia related kinase 2; vaccinia related kinase 3; vacuole membrane protein 1; valyl-tRNA synthetase; Valyl-tRNA synthetase, mitochondrial; valyl-tRNA synthetase, mitochondrial isoform 2 precursor; VAMP (vesicle-associated membrane protein)-associated protein A, 33 kDa; VAMP (vesicle-associated membrane protein)-associated protein B and C; vanin 1; vanin 2; vanin 3; variable charge, X-linked; variable charge, X-linked 2; variable charge, X-linked 3A; variable charge, X-linked 3B; variable charge, Y-linked; variable charge, Y-linked 1B; vascular cell adhesion molecule 1; vascular endothelial growth factor A; vascular endothelial growth factor B; vascular endothelial growth factor C; vascular endothelial zinc finger 1; vasoactive intestinal peptide; vasoactive intestinal peptide receptor 1; vasoactive intestinal peptide receptor 2; vasodilator-stimulated phosphoprotein; vasohibin 1; vasohibin 2; vasorin; vav 1 guanine nucleotide exchange factor; vav 2 guanine nucleotide exchange factor; vav 3 guanine nucleotide exchange factor; VENT homeobox; ventral anterior homeobox 1; ventral anterior homeobox 2; versican; very low density lipoprotein receptor; vesicle-associated membrane protein 1 (synaptobrevin 1); vesicle-associated membrane protein 2 (synaptobrevin 2); vesicle-associated membrane protein 3 (cellubrevin); vesicle-associated membrane protein 4; vesicle-associated membrane protein 5 (myobrevin); vesicle-associated membrane protein 7; vesicle-associated membrane protein 8 (endobrevin); vesicular, overexpressed in cancer, prosurvival protein 1; vezatin, adherens junctions transmembrane protein; VGF nerve growth factor inducible; villin 1; villin-like; vimentin; vimentin-type intermediate filament associated coiled-coil protein; vinculin; visinin-like 1; visual system homeobox 1; visual system homeobox 2; vitamin D (1,25-dihydroxyvitamin D3) receptor; vitamin K epoxide reductase complex, subunit 1; vitamin K epoxide reductase complex, subunit 1-like 1; vitrin; vitronectin; v-myc myelocytomatosis viral related oncogene, neuroblastoma derived (avian); voltage-dependent anion channel 1; voltage-dependent anion channel 2; voltage-dependent anion channel 3; vomeronasal 1 receptor 1; vomeronasal 1 receptor 2; vomeronasal 1 receptor 4; von Hippel-Lindau binding protein 1; von Hippel-Lindau tumor suppressor; von Willebrand factor; von Willebrand factor C and EGF domains; von Willebrand factor D and EGF domains; Vpr (HIV-1) binding protein; WAP four-disulfide core domain 1; WAP four-disulfide core domain 10A; WAP four-disulfide core domain 10B; WAP four-disulfide core domain 11; WAP four-disulfide core domain 12; WAP four-disulfide core domain 13; WAP four-disulfide core domain 2; WAP four-disulfide core domain 3; WAP four-disulfide core domain 5; WAP four-disulfide core domain 6; WAP four-disulfide core domain 8; WAP four-disulfide core domain 9; WAS protein family, member 1; WAS protein family, member 2; WAS protein family, member 3; WAS/WASL interacting protein family, member 1; WAS/WASL interacting protein family, member 2; WAS/WASL interacting protein family, member 3; WBP2 N-terminal like; WDFY family member 4; WDR45-like; Werner helicase interacting protein 1; Werner syndrome, RecQ helicase-like; widely interspaced zinc finger motifs; Williams Beuren syndrome chromosome region 22; Williams Beuren syndrome chromosome region 27; Williams-Beuren syndrome chromosome region 16; Williams-Beuren syndrome chromosome region 17; Williams-Beuren syndrome chromosome region 28; Wilms tumor 1; Wilms tumor 1 associated protein; Wilms tumor 1 interacting protein; wingless-type MMTV integration site family member 1, 2, 2B, 3, 3A, 4, 5A, 5B, 6, 7A, 7B, 8A, 8B, 9A, 9B, 10A, 10B, 11, and 16; Wiskott-Aldrich syndrome (eczema-thrombocytopenia); Wiskott-Aldrich syndrome-like; WNK lysine deficient protein kinase 1; WNK lysine deficient protein kinase 2; WNK lysine deficient protein kinase 3; WNK lysine deficient protein kinase 4; WNT inhibitory factor 1; WNT1 inducible signaling pathway protein 1; WNT1 inducible signaling pathway protein 2; WNT1 inducible signaling pathway protein 3; Wolf-Hirschhorn syndrome candidate 1; Wolf-Hirschhorn syndrome candidate 1-like 1; Wolf-Hirschhorn syndrome candidate 2; Wolfram syndrome 1 (wolframin); WW domain binding protein 1; WW domain binding protein 11; WW domain binding protein 2; WW domain binding protein 4 (formin binding protein 21); WW domain binding protein 5; WWC family member 3; X antigen family, member 1A; X antigen family, member 1B; X antigen family, member 1C; X antigen family, member 1D; X antigen family, member 1E; X antigen family, member 2; X antigen family, member 2B; X antigen family, member 3; X antigen family, member 5; xanthine dehydrogenase; X-box binding protein 1; xenotropic and polytropic retrovirus receptor 1; xeroderma pigmentosum, complementation group A; xeroderma pigmentosum, complementation group C; Xg blood group; XIAP associated factor 1; XK, Kell blood group complex subunit-related family, member 3; XK, Kell blood group complex subunit-related family, member 4; XK, Kell blood group complex subunit-related family, member 6; XK, Kell blood group complex subunit-related family, member 7; XK, Kell blood group complex subunit-related family, member 8; XK, Kell blood group complex subunit-related family, member 9; XK, Kell blood group complex subunit-related, X-linked; X-linked inhibitor of apoptosis; X-linked Kx blood group (McLeod syndrome); XPA binding protein 2; X-prolyl aminopeptidase (aminopeptidase P) 1, soluble; X-prolyl aminopeptidase (aminopeptidase P) 2, membrane-bound; X-ray radiation resistance associated 1; X-ray repair complementing defective repair in Chinese hamster cells 1; X-ray repair complementing defective repair in Chinese hamster cells 2; X-ray repair complementing defective repair in Chinese hamster cells 3; X-ray repair complementing defective repair in Chinese hamster cells 4; X-ray repair complementing defective repair in Chinese hamster cells 5 (double-strand-break rejoining); X-ray repair complementing defective repair in Chinese hamster cells 6; XRCC6 binding protein 1; xyloside xylosyltransferase 1; xylosylprotein beta 1,4-galactosyltransferase, polypeptide 7 (galactosyltransferase I); xylosyltransferase I; xylosyltransferase II; Y box binding protein 1; Y box binding protein 2; Yes-associated protein 1; Yipl domain family, member 1-7; YTH domain family, member 1; YTH domain family, member 2; YY1 associated factor 2; YY1 associated protein 1; YY1 transcription factor; YY2 transcription factor; Z-DNA binding protein 1; zeta-chain (TCR) associated protein kinase 70 kDa; Zic family member 1; Zic family member 2; Zic family member 3; Zic family member 4; Zic family member 5; zinc activated ligand-gated ion channel; zinc and ring finger 1; zinc and ring finger 2; zinc and ring finger 3; zinc and ring finger 4; zinc finger (CCCH type), RNA-binding motif and serine/arginine rich 1; zinc finger (CCCH type), RNA-binding motif and serine/arginine rich 2; zinc finger and AT hook domain containing; zinc finger CCCH-type, antiviral 1; zinc finger CCCH-type, antiviral 1-like; zinc finger CCHC-type and RNA binding motif 1; Zinc finger domain-related protein TSRM; zinc finger E-box binding homeobox 1; zinc finger E-box binding homeobox 2; zinc finger family member 673; zinc finger family member 783; zinc finger family member 788; zinc finger homeobox 2; zinc finger homeobox 3; zinc finger homeobox 4; Zinc finger imprinted 2; zinc finger protein 2, 3, 7, 8, 12, 14, 16, 17, 18, 19, 20, 22 (KOX 15), 23 (KOX 16), 24, 25, 26, 28, 30, 32, 33A, 33B, 34, 35, 36 C3H type-like 1, 36 C3H type-like 2, 37A, 41, 43, 44, 45, 48, 70, 74, 75a, 75D, 76, 80, 81, 83, 84, 85, 90, 91, 92, 93, 98, 99, 100, 101, 107, 114, 117, 121, 124, 131, 132, 133, 134, 135, 136, 138, 140, 141, 142, 143, 146, 148, 154, 155, 157, 160, 165, 167, 169, 174, 175, 177, 180, 181, 182, 184, 185 (LIM domain), 189, 192, 193, 195, 197, 200, 202, 205, 207, 208, 211, 212, 213, 214, 215, 217, 219, 221, 222, 223, 224, 225, 226, 227, 229, 230, 232, 233, 235, 236, 238, 239, 248, 250, 251, 252, 253, 254, 256, 257, 259, 260, 263, 264, 266, 267, 268, 273, 274, 275, 276, 277, 280A, 280B, 280C, 280D, 281, 282, 283, 284, 285, 286A, 287, 292, 295, 296, 300, 302, 304, 311, 317, 318, 319, 320, 322, 323, 324, 324B, 326, 329, 330, 331, 333, 334, 335, 337, 341, 343, 345, 346, 347, 350, 354A, 354B, 354C, 358, 362, 365, 366, 367, 382, 383, 384, 385A, 385B, 385D, 391, 394, 395, 396, 397, 398, 404, 407, 408, 410, 414, 415, 416, 417, 418, 419, 420, 423, 425, 426, 428, 429, 430, 431, 432, 433, 434, 436, 438, 439, 440, 441, 442, 443, 444, 445, 446, 449, 451, 454, 460, 461, 462, 467, 468, 469, 470, 471, 473, 474, 479, 480, 483, 484, 485, 486, 488, 490, 491, 492, 493, 496, 497, 498, 500, 501, 502, 503, 506, 507, 510, 511, 512, 512B, 513, 514, 516, 517, 518B, 519, 521, 524, 525, 526, 527, 528, 529, 530, 532, 534, 536, 540, 541, 543, 544, 546, 547, 548, 549, 550, 551, 552, 554, 555, 556, 557, 558, 559, 560, 561, 562, 563, 564, 565, 566, 567, 568, 569, 57, 570, 571, 572, 573, 574, 575, 576, 577, 578, 579, 580, 581, 582, 583, 584, 585A, 585B, 586, 587, 589, 592, 593, 594, 595, 596, 597, 598, 599, 600, 605, 606, 607, 608, 609, 610, 611, 613, 614, 615, 616, 618, 619, 620, 621, 622, 623, 624, 625, 626, 627, 628, 629, 630, 638, 639, 641, 642, 643, 644, 645, 646, 648, 649, 652, 653, 654, 655, 658, 660, 662, 664, 665, 667, 668, 669, 670, 671, 672, 674, 675, 676, 677, 678, 679, 680, 681, 682, 683, 684, 687, 688, 689, 69, 691, 692, 695, 696, 697, 699, 700, 701, 703, 704, 705A, 705B, 705D, 705G, 706, 707, 708, 709, 71, 710, 711, 713, 714, 716, 717, 720, 721, 726, 727, 729, 730, 732, 736, 737, 738, 740, 746, 747, 749, 750, 763, 764, 765, 766, 768, 77, 770, 771, 772, 773, 774, 775, 776, 777, 778, 780A, 780B, 781, 782, 784, 785, 786, 787, 788, 789, 79, 790, 791, 792, 793, 799, 800, 804A, 804B, 805, 808, 812, 813, 814, 816, 821, 823, 827, 829, 830, 831, 835, 836, 837, 839, 841, 843, 844, 845, 846, 852, 860, 862, 879, 880 and 891; Zinc finger protein ENSP00000375192; zinc finger protein, multitype 1; zinc finger protein, multitype 2; zinc finger protein, X-linked; zinc finger protein, P-linked; zinc finger protein-like 1; zinc finger RNA binding protein; zinc finger RNA binding protein 2; zinc finger with KRAB and SCAN domains 1-5; zinc finger with UFM1-specific peptidase domain; zinc finger, AN1-type domain 1, 2A, 2B and 3-6; zinc finger, B-box domain containing; zinc finger, C3H1-type containing; zinc finger, C4H2 domain containing; zinc finger, CCCH-type with G patch domain; zinc finger, CW type with PWWP domain 1; zinc finger, CW type with PWWP domain 2; zinc finger, GATA-like protein 1; zinc finger, imprinted 2; zinc finger, imprinted 3; zinc finger, matrin-type 1; zinc finger, matrin-type 2; zinc finger, matrin-type 3; zinc finger, matrin-type 4; zinc finger, matrin-type 5; zinc finger, MYM-type 1-6; zinc finger, X-linked, duplicated A; zinc finger, X-linked, duplicated B; zinc finger, ZZ-type with EF-hand domain 1; zinc fingers and homeoboxes 1-3; zinzona pellucida binding protein; zona pellucida binding protein 2; zona pellucida glycoprotein 1 (sperm receptor); zona pellucida glycoprotein 2 (sperm receptor); zona pellucida glycoprotein 3 (sperm receptor); zona pellucida glycoprotein 4; zonadhesin; ZW10 interactor; ZXD family zinc finger C; zygote arrest 1; zygote arrest 1-like; and zyxin.

In certain embodiments, the targeting moiety or moieties of the conjugate are present at a predetermined molar weight percentage from about 1% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the molar weight percentages of the components of the conjugate is 100%. The amount of targeting moieties of the conjugate may also be expressed in terms of proportion to the active agent(s), for example, in a ratio of ligand to active agent of about 10:1, 9:1, 8:1, 7:1, 6:1, 5:1, 4:1, 3:1, 2:1, 1:1, 1:2, 1:3, 1:4; 1:5, 1:6, 1:7, 1:8, 1:9, or 1:10.

C. Linkers

The conjugates contain one or more linkers attaching the active agents and targeting moieties. The linker, Y, is bound to one or more active agents and one or more targeting ligands to form a conjugate. The linker Y is attached to the targeting moiety X and the active agent Z by functional groups independently selected from an ester bond, disulfide, amide, acylhydrazone, ether, carbamate, carbonate, and urea. Alternatively the linker can be attached to either the targeting ligand or the active drug by a non-cleavable group such as provided by the conjugation between a thiol and a maleimide, an azide and an alkyne. The linker is independently selected from the group consisting alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl, ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl.

In some embodiments, the linker comprises a cleavable functionality that is cleavable. The cleavable functionality may be hydrolyzed in vivo or may be designed to be hydrolyzed enzymatically, for example by Cathepsin B. A “cleavable” linker, as used herein, refers to any linker which can be cleaved physically or chemically. Examples for physical cleavage may be cleavage by light, radioactive emission or heat, while examples for chemical cleavage include cleavage by re-dox-reactions, hydrolysis, pH-dependent cleavage or cleavage by enzymes.

In some embodiments the alkyl chain of the linker may optionally be interrupted by one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid. In some embodiments, the linker Y may be X′—R¹—Y′—R²—Z′ and the conjugate can be a compound according to Formula Ia:

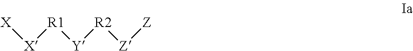

wherein X is a targeting moiety defined above; Z is an active agent; X′, R¹, Y′, R² and Z′ are as defined herein.

X′ is either absent or independently selected from carbonyl, amide, urea, amino, ester, aryl, arylcarbonyl, aryloxy, arylamino, one or more natural or unnatural amino acids, thio or succinimido; R¹ and R² are either absent or comprised of alkyl, substituted alkyl, aryl, substituted aryl, polyethylene glycol (2-30 units); Y′ is absent, substituted or unsubstituted 1,2-diaminoethane, polyethylene glycol (2-30 units) or an amide; Z′ is either absent or independently selected from carbonyl, amide, urea, amino, ester, aryl, arylcarbonyl, aryloxy, arylamino, thio or succinimido. In some embodiments, the linker can allow one active agent molecule to be linked to two or more ligands, or one ligand to be linked to two or more active agent molecule.

In some embodiments, the linker Y may be Aₘ and the conjugate can be a compound according to Formula Ib:

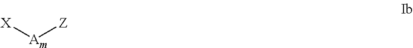

wherein A is defined herein, m=0-20.

A in Formula Ia is a spacer unit, either absent or independently selected from the following substituents. For each substituent, the dashed lines represent substitution sites with X, Z or another independently selected unit of A wherein the X, Z, or A can be attached on either side of the substituent:

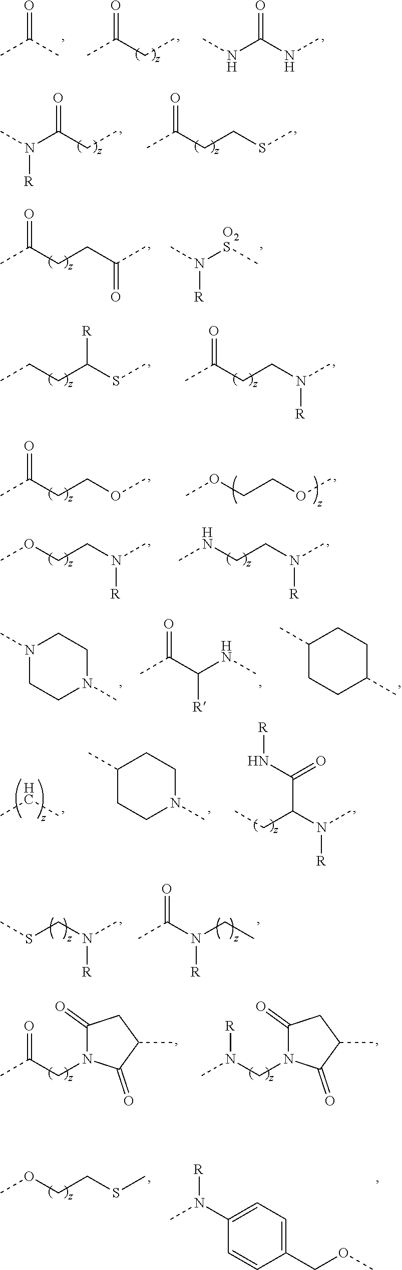

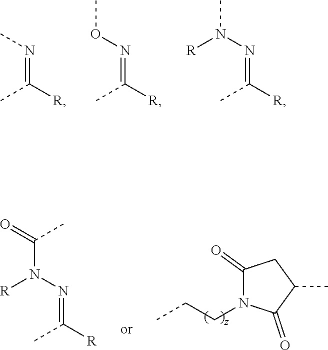

wherein z=0-40, R is H or an optionally substituted alkyl group, and R′ is any side chain found in either natural or unnatural amino acids.

In some embodiments, the conjugate may be a compound according to Formula Ic:

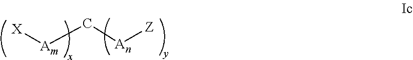

wherein A is defined above, m=0-40, n=0-40, x=1-5, y=1-5, and C is a branching element defined herein.

C in Formula Ic is a branched unit containing three to six functionalities for covalently attaching spacer units, ligands, or active drugs, selected from amines, carboxylic acids, thiols, or succinimides, including amino acids such as lysine, 2,3-diaminopropanoic acid, 2,4-diaminobutyric acid, glutamic acid, aspartic acid, and cysteine.

In some embodiments, the linker may be cleavable and is cleaved to release the active agent. In one embodiment, the linker may be cleaved by an enzyme. As a non-limiting example, the linker may be a polypeptide moiety, e.g. AA in WO2010093395 to Govindan, the contents of which are incorporated herein by reference in their entirety, that is cleavable by intracellular peptidase. Govindan teaches AA in the linker may be a di, tri, or tetrapeptide such as Ala-Leu, Leu-Ala-Leu, and Ala-Leu-Ala-Leu. In another example, the cleavable linker may be a branched peptide. The branched peptide linker may comprise two or more amino acid moieties that provide an enzyme cleavage site. Any branched peptide linker disclosed in WO1998019705 to Dubowchik, the contents of which are incorporated herein by reference in their entirety, may be used as a linker in the conjugate of the present invention. As another example, the linker may comprise a lysosomally cleavable polypeptide disclosed in U.S. Pat. No. 8,877,901 to Govindan et al., the conents of which are incorporated herein by reference in their entirety. As another example, the linker may comprise a protein peptide sequence which is selectively enzymatically cleavable by tumor associated proteases, such as any Y and Z structures disclosed in U.S. Pat. No. 6,214,345 to Firestone et al., the contents of which are incorporated herein by reference in their entirety.

In one embodiment, the cleaving of the linker is non-enzymatic. Any linker disclosed in US 20110053848 to Cleemann et al., the contents of which are incorporated herein by reference in their entirety, may be used. For example, the linker may be a non-biologically active linker represented by formula (I).

In one embodiment, the linker may be a beta-glucuronide linker disclosed in US 20140031535 to Jeffrey, the contents of which are incorporated herein by reference in their entirety. In another embodiment, the linker may be a self-stabilizing linker such as a succinimide ring, a maleimide ring, a hydrolyzed succinimide ring or a hydrolyzed maleimide ring, disclosed in US20130309256 to Lyon et al., the contents of which are incorporated herein by reference in their entirety. In another embodiment, the linker may be a human serum albumin (HAS) linker disclosed in US 20120003221 to McDonagh et al., the contents of which are incorporated herein by reference in their entirety. In another embodiment, the linker may comprise a fullerene, e.g., C60, as disclosed in US 20040241173 to Wilson et al., the contents of which are incorporated herein by reference in their entirety. In another embodiment, the linker may be a recombinant albumin fused with polycysteine peptide as disclosed in U.S. Pat. No. 8,541,378 to Ahn et al., the contents of which are incorporated herein by reference in their entirety. In another embodiment, the linker comprises a heterocycle ring. For example, the linker may be any heterocyclic 1,3-substituted five- or six-member ring, such as thiazolidine, disclosed in US 20130309257 to Giulio, the contents of which are incorporated herein by reference in their entirety.

In some embodiments, the linker Y may be a Linker Unit (LU) as described in US2011/0070248, the contents of which are incorporated herein by reference in their entirety. In formula (I) where the Ligand Drug Conjugate has formula L-(LU-D)ₚ the targeting moiety X corresponds to L (the Ligand unit) and the active agent Z corresponds to D (the drug unit).

The conjugate X—Y—Z can be a conjugate as described in WO2014/134486, the contents of which are incorporated herein by reference in their entirety. The targeting moiety X, corresponds to the cell binding agent, CBA in formula (I′) or (I) as reproduced here, wherin the linker Y and the active agent Z together correspond to the remainder of the formula (in parentheses).

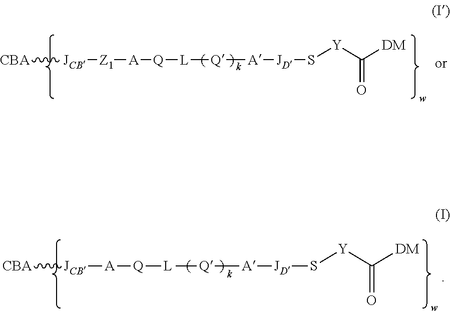

The conjugate X—Y—Z can be a conjugate as described in U.S. Pat. No. 7,601,332, the contents of which are incorporated herein by reference in their entirety, wherein conjugates are described as follows, and the targeting moiety X corresponds to V (the vitamin receptor binding moiety), the active agent Z corresponds to D (drugs and includes analogs or derivatives thereof), and the linker Y corresponds to the bivalent linker (L) which can comprise one or more components selected from spacer linkers (ls), releasable linkers (lr), and heteroatom linkers (lH), and combinations thereof, in any order:

V-L-D

V-(lᵣ)_{c}-D

V-(lₛ)ₐ-D

V-(lₛ)ₐ-(lᵣ)_{c}-D

V-(lᵣ)_{c}-(lₛ)ₐ-D

V-(l_{H})_{b}-(lᵣ)_{c}-D

V-(lᵣ)_{c}-(l_{H})_{b}-D

V-(l_{H})_{d}-(lᵣ)_{c}-(l_{H})ₑ-D

V-(lₛ)ₐ-(l_{H})_{b}-(lᵣ)_{c}-D

V-(lᵣ)_{c}-(l_{H})_{b}-(lₛ)ₐ-D

V-(l_{H})_{d}-(lₛ)ₐ-(lᵣ)_{c}-(l_{H})ₑ-D

V-(l_{H})_{d}-(lᵣ)_{c}-(lₛ)ₐ-(l_{H})ₑ-D

V-(l_{H})_{d}-(lₛ)ₐ-(l_{H})_{b}-(lᵣ)_{c}-(l_{H})ₑ-D

V-(l_{H})_{d}-(lᵣ)_{c}-(l_{H})_{b}-(lₛ)ₐ-(l_{H})ₑ-D

V-(lₛ)ₐ-(lᵣ)_{c}-(l_{H})_{b}-D

V-[(lₛ)ₐ-(l_{H})_{b}]_{d}-(lᵣ)_{c}-(l_{H})ₑ-D

In some embodiments, the conjugate is a small molecule drug conjugates (SMDC). In some embodiments, the conjugate comprises a targeting moiety that binds to the folate receptor. In some embodiments, the conjugate comprises folic acid as a targeting moiety. As a non-lmiting example, the conjugate is vintafolide (EC145) as disclosed in WO2012142281 to Ritter et al., the contents of which are incorporated herein by reference in their entirety. Vintafolide comprises a highly potent vinca alkaloid cytotoxic compound, desacetylvinblastine hydrazide (DAVLBH), conjugated to folate. As shown in the structure below, it comprises a hydrophobic payload (vinblastine), hydrophilic peptide linker (4 acids, one arginine) and folic acid targeting the folate receptor. The conjugates comprising a targeting moiety that binds to the folate receptor may also comprise a folate-targeting agent as an active agent.

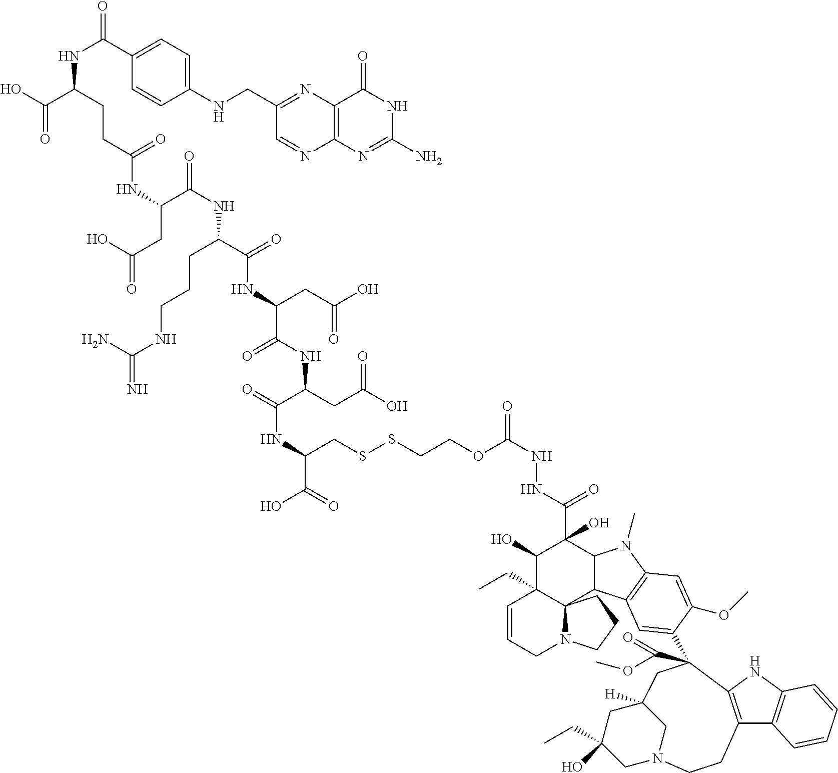

In some embodiments, the conjugate comprises tubulysin. As a non-limiting example, the conjugate is EC1456 as disclosed in US20140107316 to Vlahov et al., the contents of which are incorporated herein by reference in their entirety. As shown in the structure below, EC1456 comprises a hydrophobic peptide payload (tubulysin), hydrophilic peptide linker (3 acids, three polyols) and folic acid targeting the folate receptor.

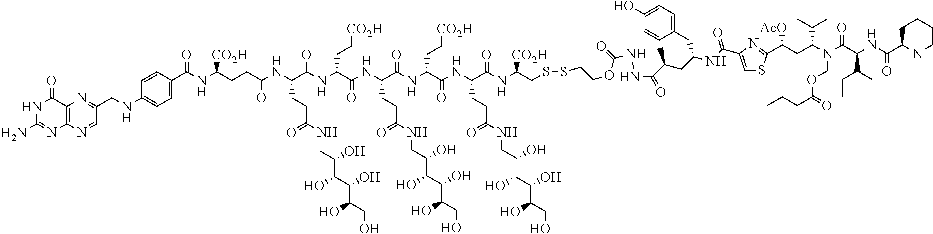

As another non-limiting example, the conjugate is EC1169 as disclosed in WO 2014078484 to Radoslavov et al., the contents of which are incorporated herein by reference in their entirety. As shown in the structure below, EC1169 comprises a hydrophobic peptide payload (tubulysin), hydrophilic peptide linker (3 acids) and a moiety targeting PSMA.

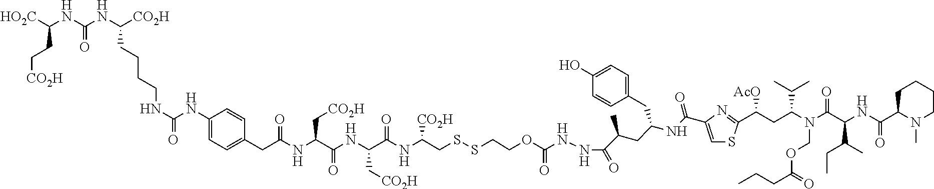 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(NC(=O)NCCCC[C@H](NC(=O)N[C@@H](CCC(=O)O)C(=O)O)C(=O)O)cc2)C(=O)O)cs1)C(C)C]

In some embodiments, the targeting moiety of the conjugate binds to LHRHR. Non-limiting examples of the conjugate include:

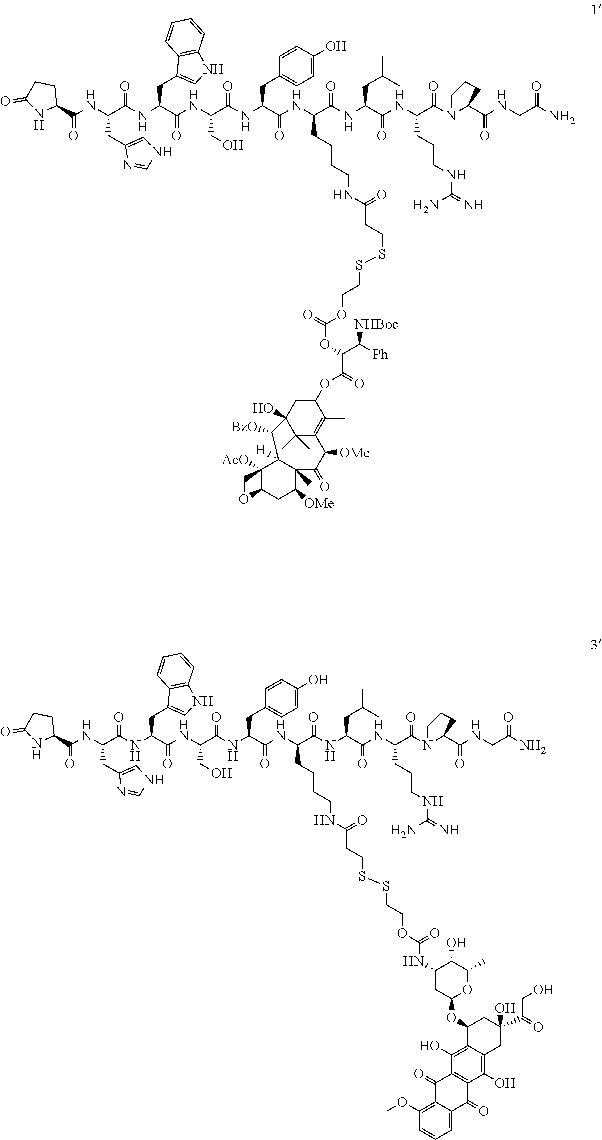

In some embodiments, the targeting moiety binds to a somatostatin receptor. Non-limiting examples of the conjugate include:

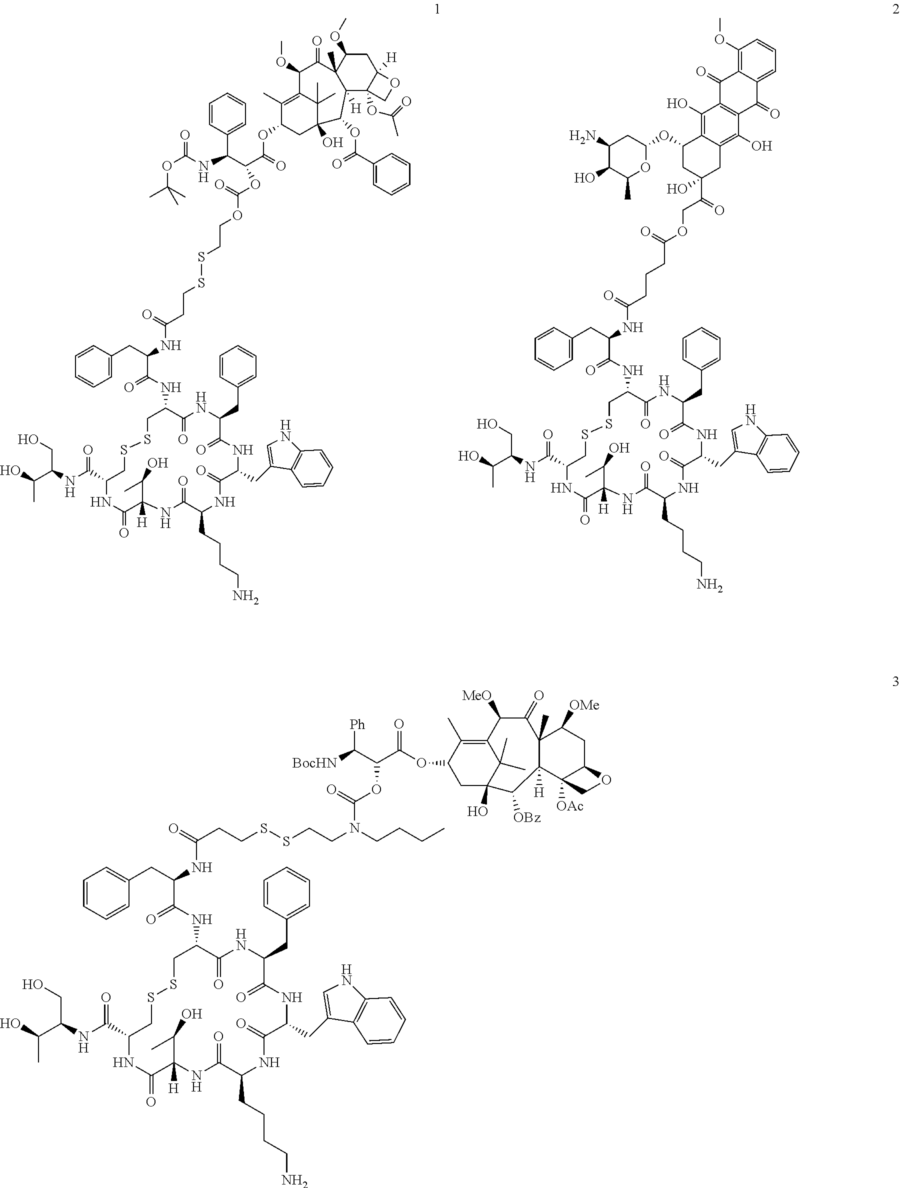

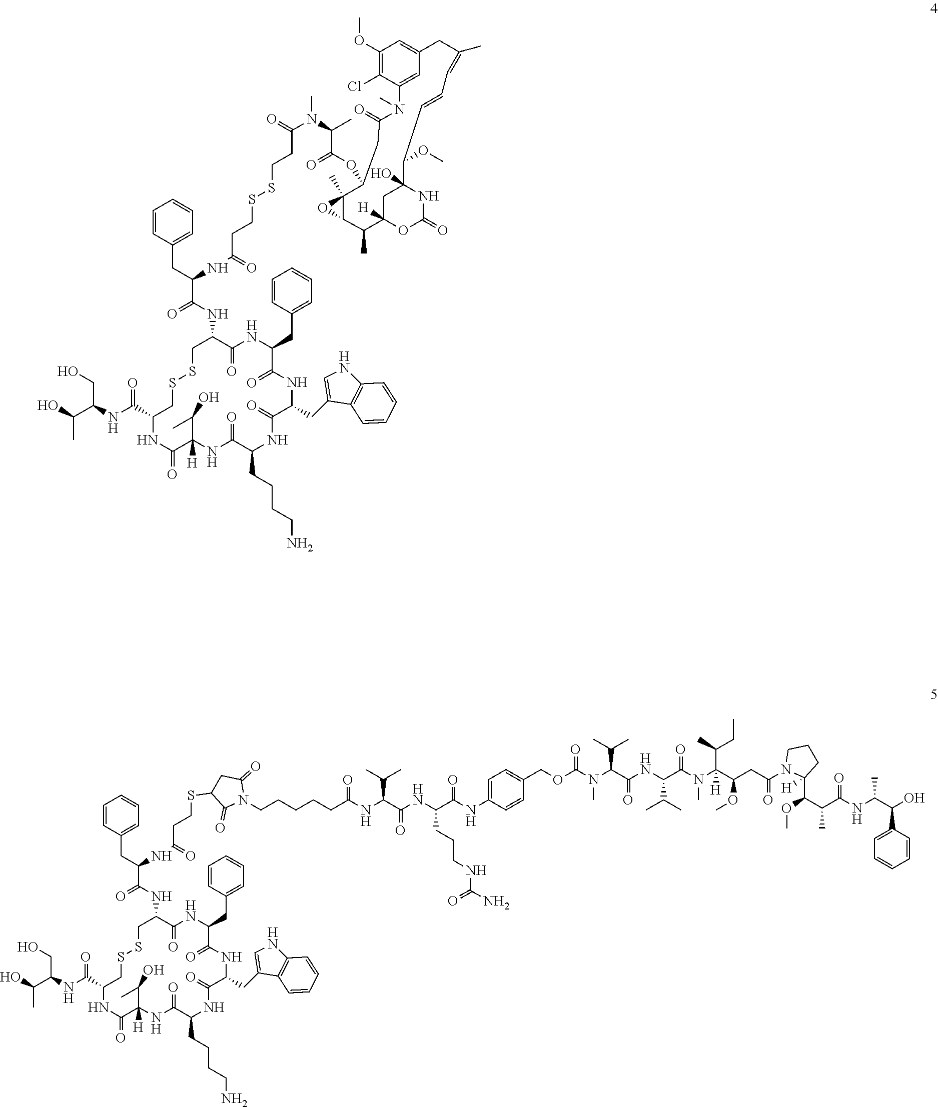

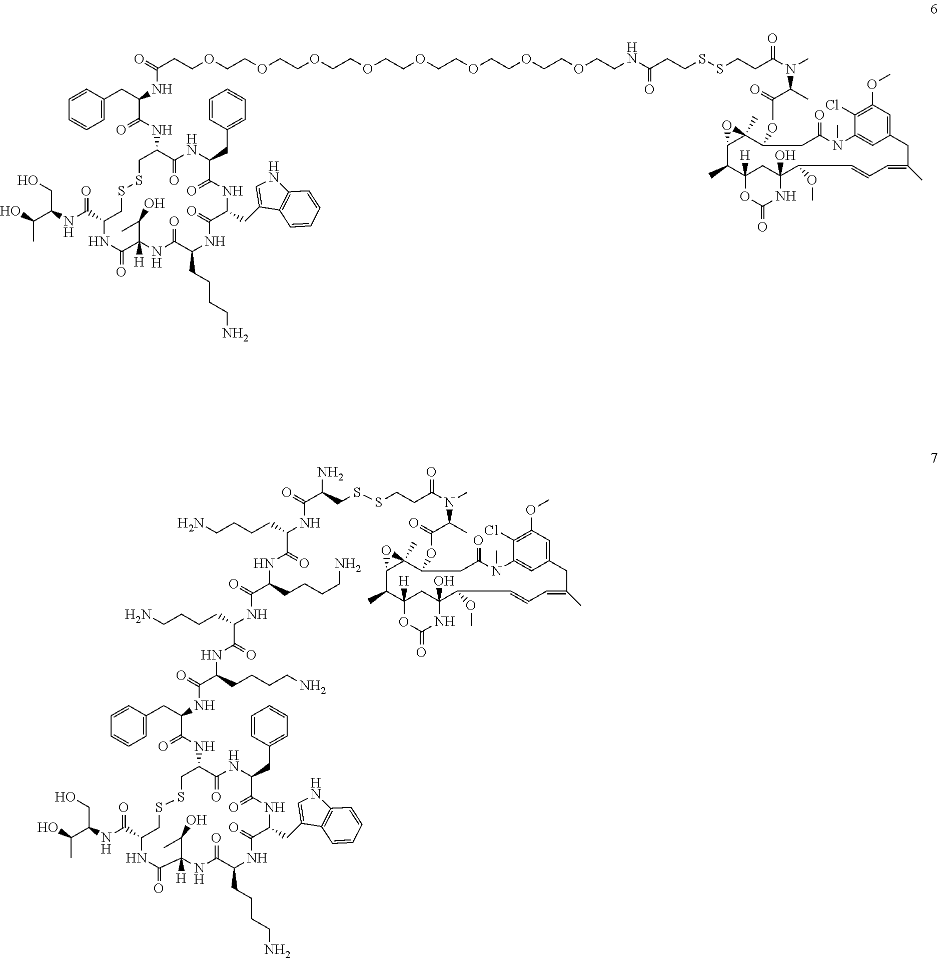

### II. Particles

Particles comprising one or more conjugates can be polymeric particles, lipid particles, solid lipid particles, self assembled particles, composite nanoparticles of conjugate phospholipids, surfactants, proteins, polyaminoacids, inorganic particles, or combinations thereof (e.g., lipid stabilized polymeric particles). In some embodiments, the conjugates are substantially encapsulated or particially encapsulated in the particles. In some embodiments, the conjugates are deposited and/or absorbed on the surface of the partciles. In some embodiments, the conjutaes are incorporated in the particles. In some embodiments, the conjugates are part of or a component of the particle. The conjugates may be attached to the surface of the particles with covalent bonds, or non-covalent interactions. In some embodiments, the conjugates of the present invention self-assemble into a particle.

As used herein, the term “encapsulate” means to enclose, surround or encase. As it relates to the formulation of the conjugates of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of conjugate of the invention may be enclosed, surrounded or encased within the particle. “Partially encapsulation” means that less than 10, 10, 20, 30, 40 50 or less of the conjugate of the invention may be enclosed, surrounded or encased within the particle. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the particle. Encapsulation may be determined by any known method.

In some embodiments, the particles are polymeric particles or contain a polymeric matrix. The particles can contain any of the polymers described herein or derivatives or copolymers thereof. The particles will generally contain one or more biocompatible polymers. The polymers can be biodegradable polymers. The polymers can be hydrophobic polymers, hydrophilic polymers, or amphiphilic polymers. In some embodiments, the particles contain one or more polymers having an additional targeting moiety attached thereto. In some embodiments, the particles are inorganic particles, such as but not limited to, gold nanoparticles and iron oxide nanoparticles.

The size of the particles can be adjusted for the intended application. The particles can be nanoparticles or microparticles. The particle can have a diameter of about 10 nm to about 10 microns, about 10 nm to about 1 micron, about 10 nm to about 500 nm, about 20 nm to about 500 nm, or about 25 nm to about 250 nm. In some embodiments the particle is a nanoparticle having a diameter from about 25 nm to about 250 nm. In some embodiments, the particle is a nanoparticle having a diameter from about 50 nm to about 150 nm. In some embodiments, the particle is a nanoparticle having a diameter from about 70 nm to about 130 nm. In some embodiments, the particle is a nanoparticle having a diameter of about 100 nm. It is understood by those in the art that a plurality of particles will have a range of sizes and the diameter is understood to be the median diameter of the particle size distribution. Polydispersity index (PDI) of the particles may be ≤about 0.5, ≤about 0.2, or ≤about 0.1. Drug loading may be ≥about 0.1%, ≥about 1%, ≥about 5%, ≥about 10%, or ≥out 20%. Drug loading, as used herein, refers to the weight ratio of the conjugates, where the conjugate is the drug and the weight ratio refers to the weight of the conjugate relative to the weight of the nanoparticle. Drug loading may depend on delivery system composition, drug concentration, a lyophilized weight, and reconstituted drug concentration. The weight of the dried composition can be measured, the drug concentration could be measured, and a weight by weight % of the drug can be subsequently calculated. Particle ζ-potential (in 1/10^{th} PBS) may be ≤0 mV or from about −30 to 0 mV. Drug released in vitro from the particle at 2h may be less than about 60%, less than about 40%, or less than about 20%. Regarding pharmacokinetics, plasma area under the curve (AUC) in a plot of concentration of drug in blood plasma against time may be at least 2 fold greater than free drug conjugate, at least 4 fold greater than free drug conjugate, at least 5 fold greater than free drug conjugate, at least 8 fold greater than free drug conjugate, or at least 10 fold greater than free drug conjugate. Tumor PK/PD of the particle may be at least 5 fold greater than free drug conjugate, at least 8 fold greater than free drug conjugate, at least 10 fold greater than free drug conjugate, or at least 15 fold greater than free drug conjugate. The ratio of Cₘₐₓ of the particle to Cₘₐₓ of free drug conjugate may be at least about 2, at least about 4, at least about 5, or at least about 10. Cₘₐₓ, as used herein, refers to the maximum or peak serum concentration that a drug achieves in a specified compartment or test area of the body after the drug has been administrated and prior to the administration of a second dose. The ratio of MTD of a particle to MTD of free drug conjugate may be at least about 0.5, at least about 1, at least about 2, or at least about 5. Efficacy in tumor models, e.g., TGI %, of a particle is better than free drug conjugate. Toxicity of a particle is lower than free drug conjugate.

In various embodiments, a particle may be a nanoparticle, i.e., the particle has a characteristic dimension of less than about 1 micrometer, where the characteristic dimension of a particle is the diameter of a perfect sphere having the same volume as the particle. The size distribution of the particles can be characterized by an average diameter (e.g., the average diameter for the plurality of particles). In some embodiments, the diameter of the particles may have a Gaussian-type distribution. In some embodiments, the size distribution of the particles have an average diameter of less than about 300 nm, less than about 250 nm, less than about 200 nm, less than about 150 nm, less than about 100 nm, less than about 50 nm, less than about 30 nm, less than about 10 nm, less than about 3 nm, or less than about 1 nm. In some embodiments, the particles have an average diameter of at least about 5 nm, at least about 10 nm, at least about 30 nm, at least about 50 nm, at least about 100 nm, at least about 150 nm, or greater. In certain embodiments, the plurality of the particles have an average diameter of about 10 nm, about 25 nm, about 50 nm, about 100 nm, about 150 nm, about 200 nm, about 250 nm, about 300 nm, about 500 nm, or the like. In some embodiments, the plurality of particles have an average diameter between about 10 nm and about 500 nm, between about 50 nm and about 400 nm, between about 100 nm and about 300 nm, between about 150 nm and about 250 nm, between about 175 nm and about 225 nm, or the like. In some embodiments, the plurality of particles have an average diameter between about 10 nm and about 500 nm, between about 20 nm and about 400 nm, between about 30 nm and about 300 nm, between about 40 nm and about 200 nm, between about 50 nm and about 175 nm, between about 60 nm and about 150 nm, between about 70 nm and about 130 nm, or the like. For example, the average diameter can be between about 70 nm and 130 nm. In some embodiments, the plurality of particles have an average diameter between about 20 nm and about 220 nm, between about 30 nm and about 200 nm, between about 40 nm and about 180 nm, between about 50 nm and about 170 nm, between about 60 nm and about 150 nm, or between about 70 nm and about 130 nm. In one embodiment, the particles have a size of 40 to 120 nm with a zeta potential close to 0 mV at low to zero ionic strengths (1 to 10 mM), with zeta potential values between +5 to −5 mV, and a zero/neutral or a small −ve surface charge.

A. Conjugates

The particles contain one or more conjugates as described above. The conjugates can be present in the interior of the particle, on the surface of the particle, or both. In some embodiments, the conjutaes are incorporated in the particles. In some embodiments, the conjugates are part of or a component of the particle.

In some embodiments, the conjugate is a small molecule drug conjugate (SMDC). As a non-lmiting example, the conjugate is vintafolide (EC145) as disclosed in WO2012142281 to Ritter et al., the contents of which are incorporated herein by reference in their entirety. Vintafolide comprises a highly potent vinca alkaloid cytotoxic compound, desacetylvinblastine hydrazide (DAVLBH), conjugated to folate. As shown in the structure below, it comprises a hydrophobic payload (vinblastine), hydrophilic peptide linker (4 acids, one arginine) and folic acid targeting the folate receptor.

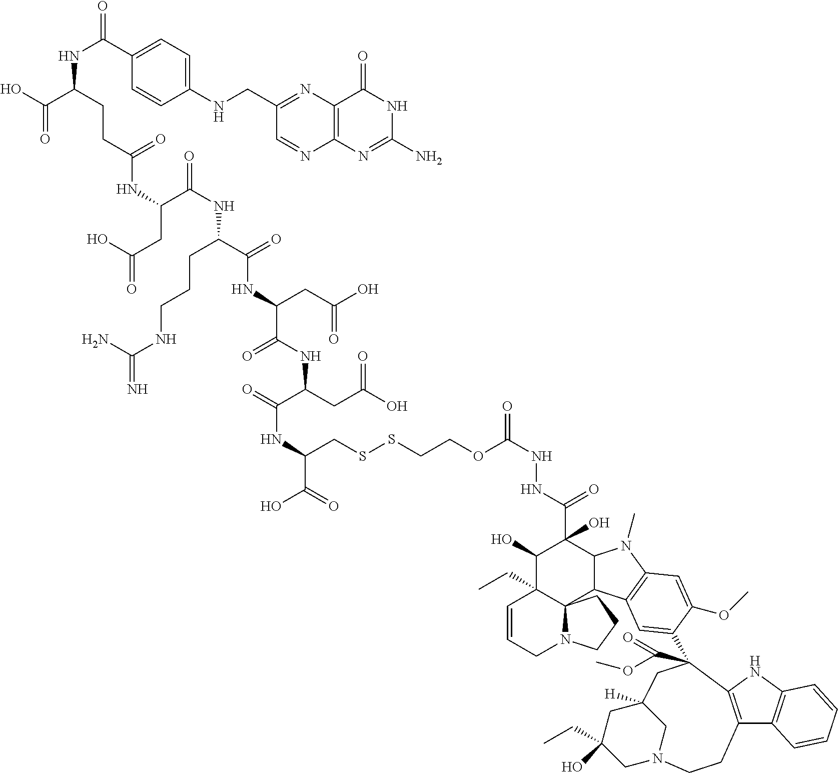

As another non-limiting example, the conjugate is EC1456 as disclosed in US20140107316 to Vlahov et al., the contents of which are incorporated herein by reference in their entirety. As shown in the structure below, EC1456 comprises a hydrophobic peptide payload, hydrophilic peptide linker (3 acids, three polyols) and folic acid targeting the folate receptor.

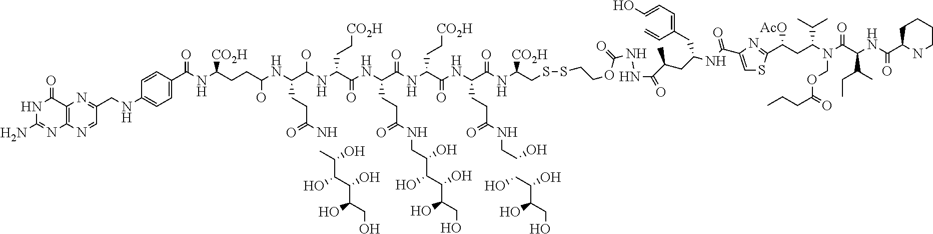

As another non-limiting example, the conjugate is EC1169 as disclosed in WO 2014078484 to Radoslavov et al., the contents of which are incorporated herein by reference in their entirety. As shown in the structure below, EC1169 comprises a hydrophobic peptide payload, hydrophilic peptide linker (3 acids) and a moiety targeting PSMA.

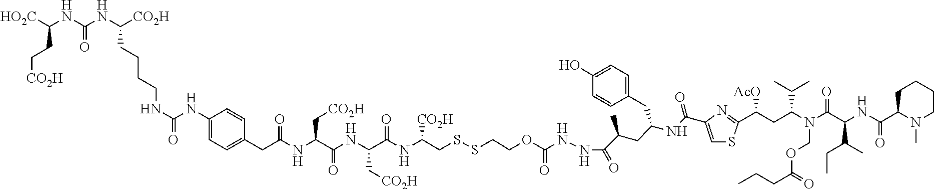 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(NC(=O)NCCCC[C@H](NC(=O)N[C@@H](CCC(=O)O)C(=O)O)C(=O)O)cc2)C(=O)O)cs1)C(C)C]

In some embodiments, the targeting moiety of the conjugate binds to LHRHR. Non-limiting examples of the conjugate include:

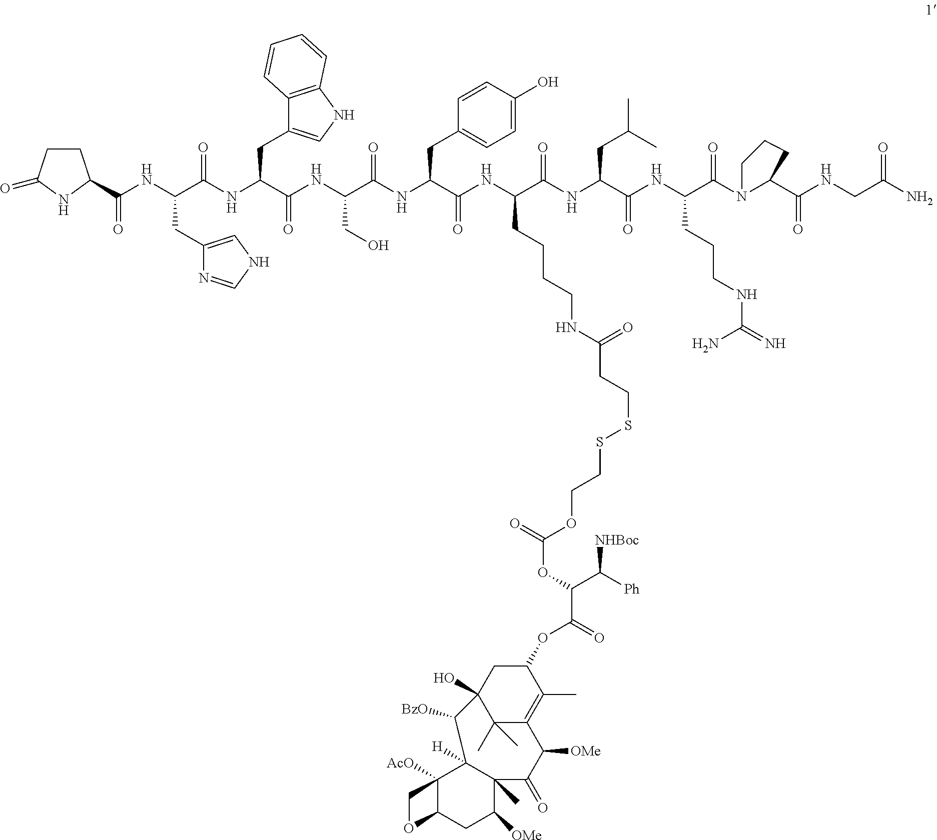

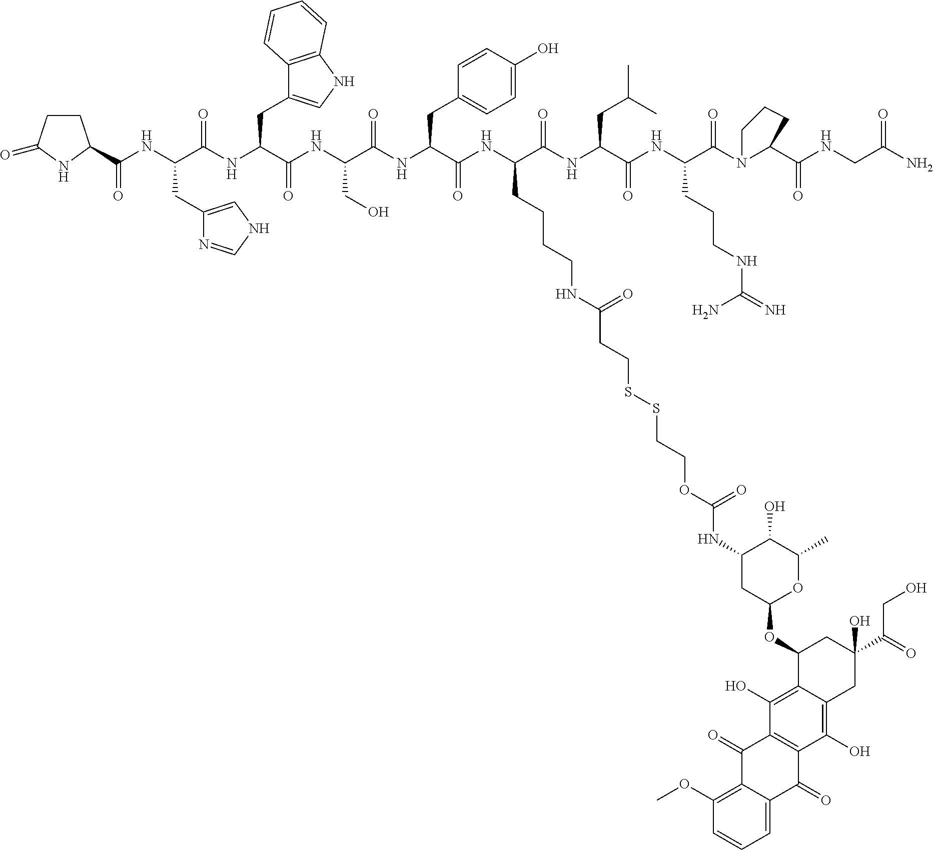 [SMILES: COc1cccc2c1C(=O)c1c(O)c3c(c(O)c1C2=O)C[C@@](O)(C(=O)CO)C[C@@H]3O[C@H]1C[C@H](NC(=O)OCCSSCCC(=O)NCCCC[C@@H](NC(=O)[C@H](Cc2ccc(O)cc2)NC(=O)[C@H](CO)NC(=O)[C@H](Cc2c[nH]c3ccccc23)NC(=O)[C@H](Cc2c[nH]cn2)NC(=O)[C@@H]2CCC(=O)N2)C(=O)N[C@@H](CC(C)C)C(=O)N[C@@H](CCCNC(=N)N)C(=O)N2CCC[C@H]2C(=O)NCC(N)=O)[C@H](O)[C@H](C)O1]

In some embodiments, the targeting moiety binds to a somatostatin receptor. Non-limiting examples of the conjugate include:

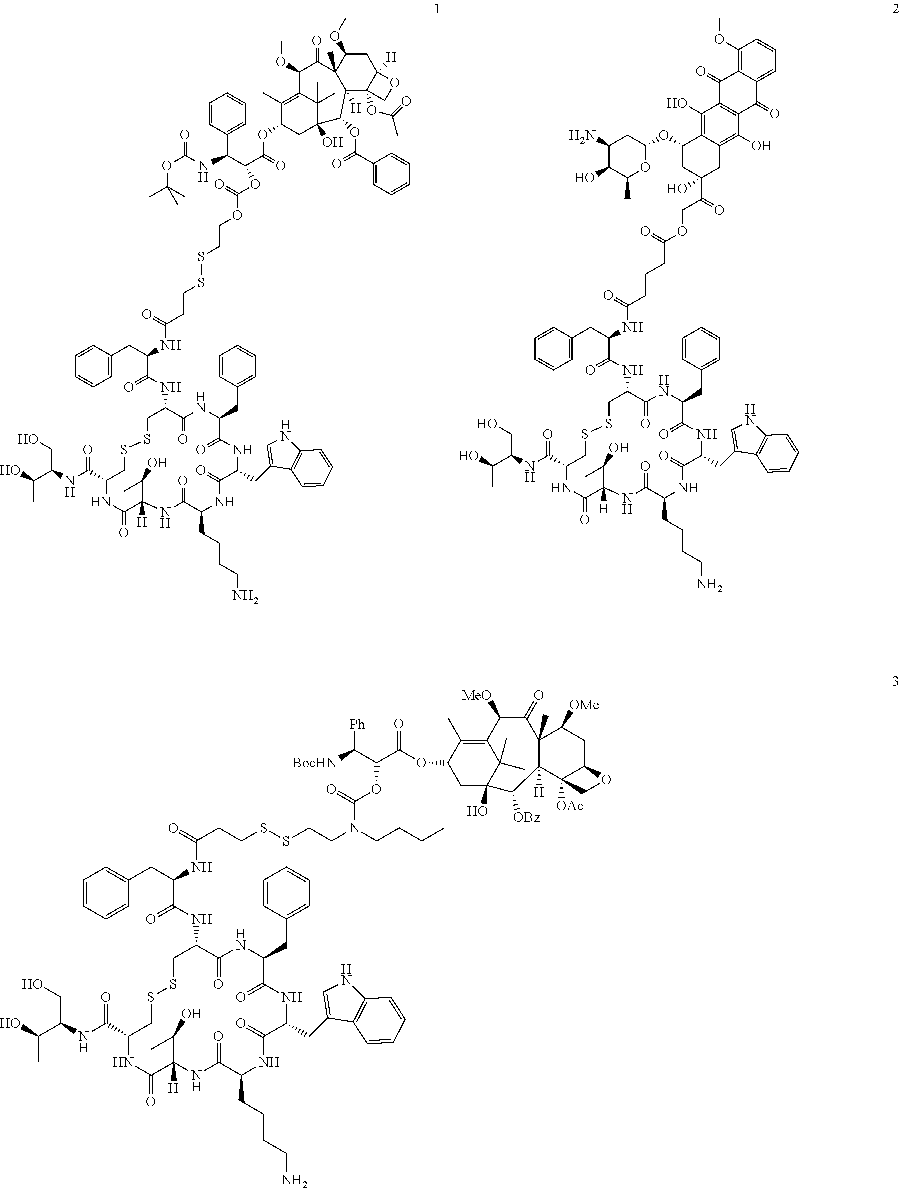

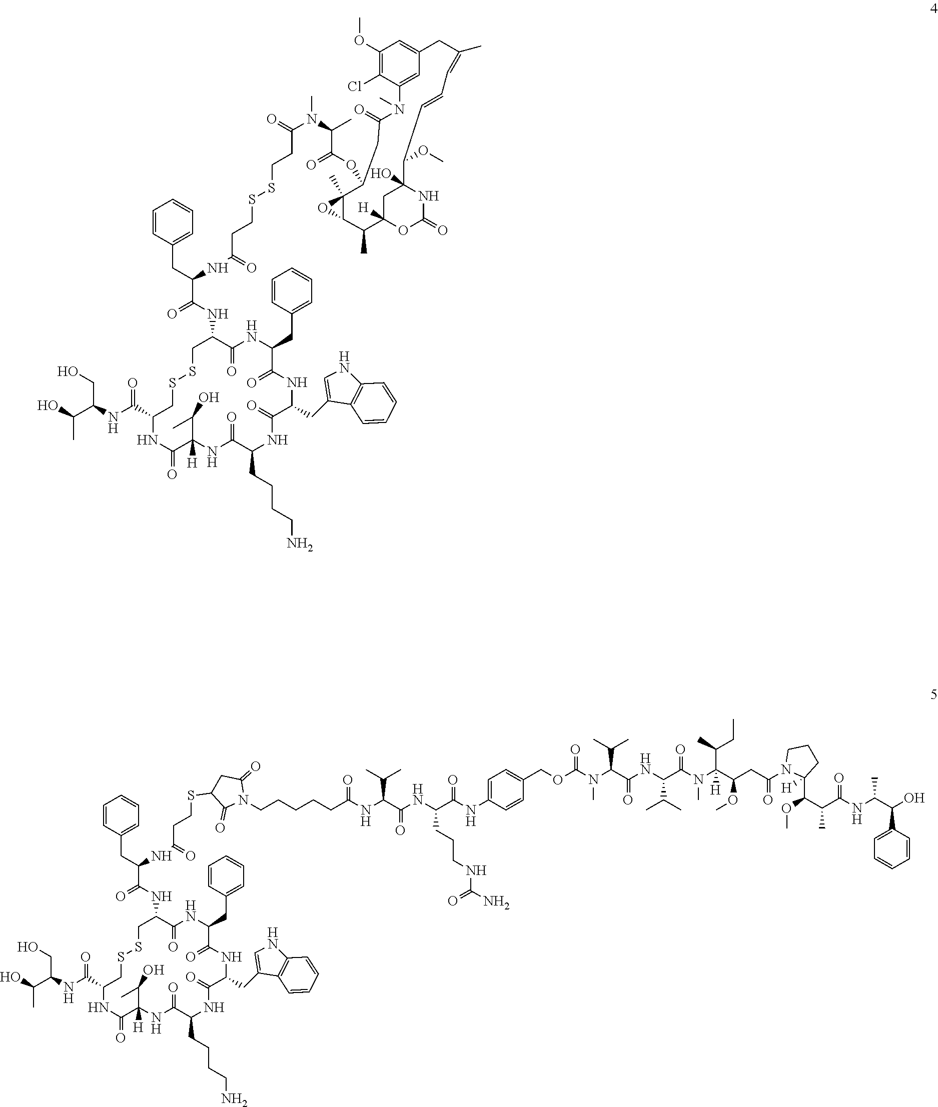

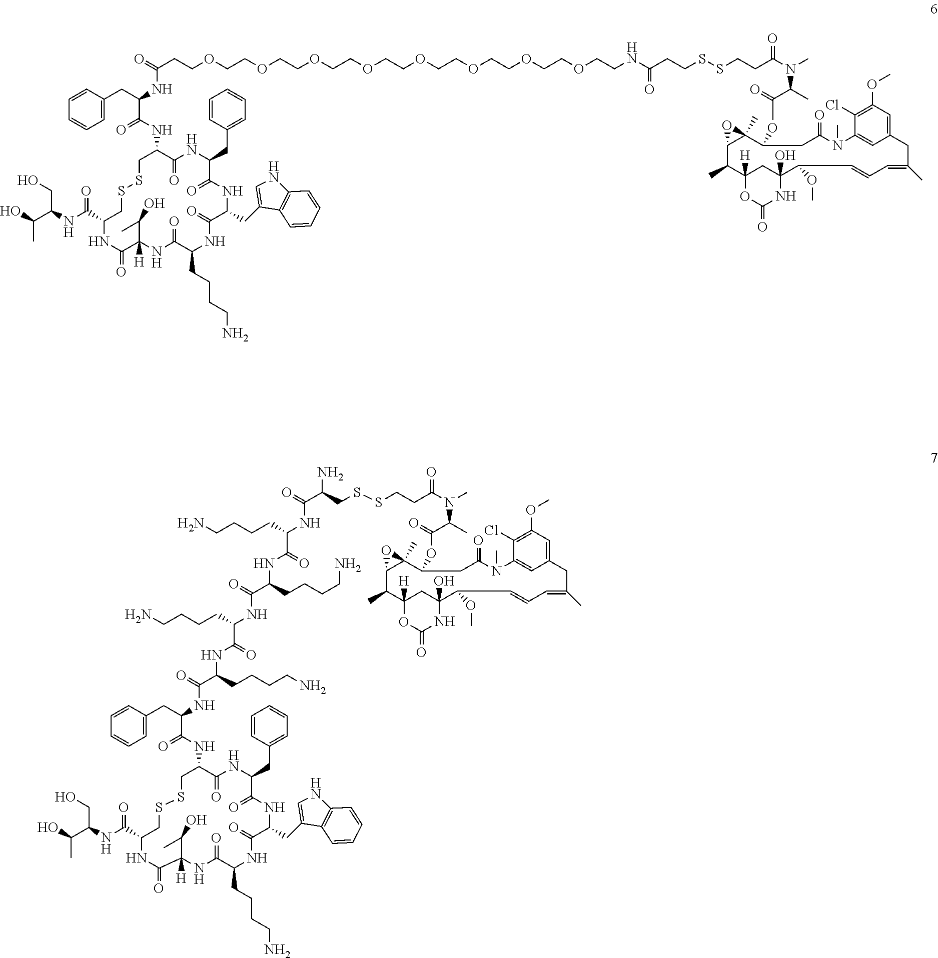

The particles may comprise hydrophobic ion-pairing complexes or hydrophobic ioin-pairs formed by one or more conjugates described above and counterions.

Hydrophobic ion-pairing (HIP) is the interaction between a pair of oppositely charged ions held together by Coulombic attraction. HIP, as used here in, refers to the interaction between the conjugate of the present invention and its counterions, wherein the counterion is not H⁺ or HO⁻ ions. Hydrophobic ion-pairing complex or hydrophobic ion-pair, as used herein, refers to the complex formed by the conjugate of the present invention and its counterions. In some embodiments, the counterions are hydrophobic. In some embodiments, the counterions are provided by a hydrophobic acid or a salt of a hydrophobic acid. In some embodiments, the counterions are provided by bile acids or salts, fatty acids or salts, lipids, phospholipids, amino acids, polyaminoacids or proteins. In some embodiments, the counterions are negatively charged (anionic). In some embodiments, the counterions are or positively charged (cataionic). Non-limited examples of negative charged counterions include the counterions sodium sulfosuccinate (AOT), sodium oleate, sodium dodecyl sulfate (SDS), human serum albumin (HSA), dextran sulphate, sodium deoxycholate, sodium cholate, sodium stearate, anionic lipids, phospholipids, amino acids, or any combination thereof. Non-limited examples of positively charged counterions include 1,2-dioleoyl-3-trimethylammonium-propane (chloride salt) (DOTAP), cetrimonium bromide (CTAB), quaternary ammonium salt didodecyl dimethylammonium bromide (DMAB) or Didodecyldimethylammonium bromide (DDAB). Without wishing to be bound by any theory, in some embodiments, HIP may increase the hydrophobicity and/or lipophilicity of the conjugate of the present invention. In some embodiments, increasing the hydrophobicity and/or lipophilicity of the conjugate of the present invention may be beneficial for particle formulations and may provide higher solubility of the conjugate of the present invention in organic solvents and lower solubility in an aqueous medium. Without wishing to be bound by any theory, it is believed that particle formulations that include HIP pairs have improved formulation properties, such as encapsulation efficiency, drug loading and/or release profile. Without wishing to be bound by any theory, in some embodiments, slow release of the conjugate of the invention from the particles may occur, due to a decrease in the conjugate's solubility in aqueous solution. In addition, without wishing to be bound by any theory, complexing the conjugate with large hydrophobic counterions may slow diffusion of the conjugate within a polymeric matrix. In some emobodiments, HIP occurs without covalent conjuatation of the counterion to the conjugate of the present invention.

Without wishing to be bound by any theory, the strength of HIP may impact the encapsulation efficiency, drug load and release rate of the particles of the invention. In some embodiments, the strength of the HIP may be increased by increasing the magnitude of the difference between the pKa of the conjugate of the present invention and the pKa of the agent providing the counterion. Also without wishing to be bound by any theory, the conditions for ion pair formation may impact the drug load and release rate of the particles of the invention.

In some embodiments, any suitable hydrophobic acid or a combination thereof may form a HIP pair with the conjugate of the present invention. In some embodiments, the hydrophobic acid may be a carboxylic acid (such as but not limited to a monocarboxylic acid, dicarboxylic acid, tricarboxylic acid), a sulfinic acid, a sulfenic acid, or a sulfonic acid. In some embodiments, a salt of a suitable hydrophobic acid or a combination thereof may be used to form a HIP pair with the conjugate of the present invention. Examples of hydrophobic acids, saturated fatty acids, unsaturated fatty acids, aromatic acids, bile acid, polyelectrolyte, their dissociation constant in water (pKa) and log P values were disclosed in WO2014/043,625, the contents of which are incorporated herein by reference in their entirety. The strength of the hydrophobic acid, the difference between the pKa of the hydrophobic acid and the pKa of the conjugate of the present invention, log P of the hydrophobic acid, the phase transition temperature of the hydrophobic acid, the molar ratio of the hydrophobic acid to the conjugate of the present invention, and the concentration of the hydrophobic acid were also disclosed in WO2014/043,625, the contents of which are incorporated herein by reference in their entirety.

In some embodiments, particles of the present invention including a HIP complex and/or prepared by a process that provides a counterion to form HIP complex with the conjugate may have a higher encapsulation efficiency and/or drug loading than particles without a HIP complex or prepared by a process that does not provide any counterion to form HIP complex with the conjugate. In some embodiments, encapsulation efficiency or drug loading may increase 50%, 100%, 2 times, 3 times, 4 times, 5 times, 6 times, 7 times, 8 times, 9 times, or 10 times.

In some embodiments, the particles of the invention may retain the total amount of conjugate for at least about 1 minute, at least about 15 minutes, at least about 1 hour, or at least about 2 hour when placed in a phosphate buffer solution at 37° C.

In some embodiments, the weight percentage of the conjugate in the particles is at least about 0.05%, 0.1%, 0.5%, 1%, 5%, 10%, 15%, 20%, 25%, 30%, 35%, 40%, 45%, or 50% such that the sum of the weight percentages of the components of the particles is 100%. In some embodiments, the weight percentage of the conjugate in the particles is from about 0.5% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the weight percentages of the components of the particles is 100%.

In some instances, a conjugate may have a molecular weight of less than about 50,000 Da, less than about 40,000 Da, less than about 30,000 Da, less than about 20,000 Da, less than about 15,000 Da, less than about 10,000 Da, less than about 8,000 Da, less than about 5,000 Da, or less than about 3,000 Da. In some cases, the conjugate may have a molecular weight of between about 1,000 Da and about 50,000 Da, in some embodiments between about 1,000 Da and about 40,000 Da, in some embodiments between about 1,000 Da and about 30,000 Da, in some embodiments bout 1,000 Da and about 50,000 Da, between about 1,000 Da and about 20,000 Da, in some embodiments between about 1,000 Da and about 15,000 Da, in some embodiments between about 1,000 Da and about 10,000 Da, in some embodiments between about 1,000 Da and about 8,000 Da, in some embodiments between about 1,000 Da and about 5,000 Da, and in some embodiments between about 1,000 Da and about 3,000 Da. The molecular weight of the conjugate may be calculated as the sum of the atomic weight of each atom in the formula of the conjugate multiplied by the number of each atom. It may also be measured by mass spectrometry, NMR, chromatography, light scattering, viscosity, and/or any other methods known in the art. It is known in the art that the unit of molecular weight may be g/mol, Dalton (Da), or atomic mass unit (amu), wherein 1 g/mol=1 Da=1 amu.

B. Polymers

The particles may contain one or more polymers. Polymers may contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(c-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker.

The particles may contain one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol); polyoxazoline; and copolymers thereof.

The particles may contain one or more hydrophobic polymers. Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In some embodiments, the hydrophobic polymer is poly(lactic acid), poly(glycolic acid), or poly(lactic acid-co-glycolic acid).

The particles can contain one or more biodegradable polymers. Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water.

Biodegradable polymers in the particle can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose such as methyl cellulose and ethyl cellulose, hydroxyalkyl celluloses such as hydroxypropyl cellulose, hydroxy-propyl methyl cellulose, and hydroxybutyl methyl cellulose, cellulose ethers, cellulose esters, nitro celluloses, cellulose acetate, cellulose propionate, cellulose acetate butyrate, cellulose acetate phthalate, carboxylethyl cellulose, cellulose triacetate, cellulose sulphate sodium salt, polymers of acrylic and methacrylic esters such as poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxyalkanoates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In some embodiments the particle contains biodegradable polyesters or polyanhydrides such as poly(lactic acid), poly(glycolic acid), and poly(lactic-co-glycolic acid).

The particles can contain one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In some embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end, or both. The particle can contain two or more amphiphilic polymers.

In one embodiment, the conjugate comprising the active agent of the invention may be delivered with a block copolymer drug delivery system for coordination of cisplatin and gemcitabine into liposomes as disclosed in U.S. RE45471 to Harada, et al., (Nanocarrier), the contents of which are incorporated herein by reference in their entirety. The block copolymers are comprised of PEG- and polyamino acids.

In one embodiment, the conjugate comprising the active agent of the invention may be delivered with a polymer micelle and having a pH values of 3.0 to 7.0 and comprises a coordination compound having a block copolymer of polyethylene glycol and polyglutamic acid and cisplatin that is coordinate-bonded to the block copolymer as disclosed in U.S. Pat. No. 8,895,076 to Kataoka, et al., (Nanocarrier), the contents of which are incorporated herein by reference in their entirety. The block copolymers are comprised of PEG- and polyamino acids.

In one embodiment, the conjugate comprising the active agent of the invention may be a lyophilized preparation, comprising a drug-encapsulating polymer micelle and saccharides and/or polyethylene glycol as a stabilizing agent as disclosed in US 20140141072 to Ogawa, et al., (Nanocarrier), the contents of which are incorporated herein by reference in their entirety. The drug-encapsulating polymer micelle is formed from a block copolymer having in the molecule, a hydrophilic polymer segment and a polymer segment which is hydrophobic or chargeable or which comprises the repetitive units of both of them, and it is a substantially spherical core-shell type micelle in which the drug is carried principally in a core part and in which a shell part is constituted by the above hydrophilic polymer segment. The block copolymers are comprised of PEG- and polyamino acids. The stabilizing agent is selected from the group consisting of saccharides which are maltose, trehalose, xylitol, glucose, sucrose, fructose, lactose, mannitol and dextrin and polyethylene glycol.

In one embodiment, the conjugate comprising the active agent of the invention may be a micellar preparation comprising a novel block copolymer and a sparingly water-soluble anticancer agent, as disclosed in US 20140142167 to Shimizu, et al., (Nanocarrier), the contents of which are incorporated herein by reference in their entirety. The block copolymers are comprised of PEG- and polyamino acids.

In one embodiment, the conjugate comprising the active agent of the invention may be a preparation containing drug-encapsulating polymer micelles with a controlled size, which comprises forming a solution by dispersing and dissolving a block copolymer with hydrophilic and hydrophobic segments, and a sparingly water-soluble drug, as disclosed in US 20060057219 to Nagasaki, et al., (Nanocarrier), the contents of which are incorporated herein by reference in their entirety. The block copolymers are comprised of PEG- and polyamino acids.

In one embodiment, the conjugate comprising the active agent of the invention may comprise a water-scarcely soluble (or oil-soluble) drug and be charged into a polymeric micelle block copolymer having a hydrophilic segment and a hydrophobic segment and further to provide a polymeric micelle charged therein with a stable drug which can significantly raise a drug concentration in water or a buffered or isotonic aqueous solution as described in EP 1127570 to Honzawa, et al., (Nanocarrier), the contents of which are incorporated herein by reference in their entirety. The “block copolymer having a hydrophilic segment and a hydrophobic segment” means a copolymer which can be present in an aqueous medium in the form of a core (mainly comprising hydrophobic segments)-shell (mainly comprising hydrophilic segments) type polymeric micelle. The “hydrophilic segment” constituting such block copolymer includes segments originating in poly-(ethylene oxide), poly(malic acid), poly(saccharide), poly(acrylic acid), poly(vinyl alcohol) and poly(vinylpyrrolidone). The “hydrophobic segment” includes segments originating in poly(β-benzyl aspartate), poly(γ-benzyl glutamate), poly-(β-alkyl aspartate), poly(lactide), poly(ε-caprolactone), poly(δ-valerolactone), poly(γ-butyrolactone), poly(α-amino acid) and two or more kinds thereof.

In one embodiment, the conjugate comprising the active agent of the invention may be a stable liquid composition of a cisplatin coordination compound as described in EP 2305275 to Kataoka, et al., (Nanocarrier), the contents of which are incorporated herein by reference in their entirety. The stabilized liquid composition comprises a coordination compound in which cisplatin is coordinate-bonded to a block copolymer consisting of polyethylene glycol and polyglutamic acid.

In one embodiment, the conjugate of the invention may be encapsulated in polymer micelles formed from a block copolymer having a hydrophilic segment and hydrophobic segment, and has been subjected to high-pressure treatment as described in EP 1815869 to Yamamoto, et al., (Nanocarrier), the contents of which are incorporated herein by reference in their entirety. The block copolymer used for the invention having a hydrophilic segment and a hydrophobic segment. The polymer composed of the hydrophilic segment is not limited, and there may be mentioned segments of polyethylene glycol, polyphosphoric acid, polyoxyethylene, polysaccharides, polyacrylamide, polyacrylic acid, polymethacrylamide, polymethacrylic acid, polyvinylpyrrolidone, polyvinyl alcohol, polymethacrylic acid ester, polyacrylic acid ester, polyamino acid, and derivatives thereof. Preferred among these are segments composed of polyethylene glycol. The hydrophilic segment may have a low molecular functional group on the opposite side of the end bonding with the hydrophobic segment, so long as it does not adversely affect formation of the polymer micelles. The hydrophobic segment is also not limited, and there may be mentioned polypeptides, particularly polypeptides of polyhomoamino acids, and for example, L- or D-amino acids or their racemic mixtures, and especially L-amino acids such as poly(aspartic acid), poly(glutamic acid), polyaspartic acid esters, polyglutamic acid esters or their partial hydrolysates, polylysine, polyacrylic acid, polymethacrylic acid, polymalic acid, polylactic acid, polyalkylene oxides, long-chain alcohols, and other known biocompatible polymers, biodegradable polymers and the like. The hydrophobic segment may have a low molecular functional group on the opposite side of the end bonding with the hydrophilic segment, similar to that explained for the hydrophilic segment, so long as it does not adversely affect interaction between the drug and the hydrophobic segment during formation of the polymer micelles. The hydrophilic segment and hydrophobic segment are not restricted in size so long as they can form polymer micelles in an aqueous solution (or aqueous medium) in the presence of a water-insoluble drug, but generally the hydrophilic segment has preferably 30-1000 and more preferably 50-600 repeating units, while the hydrophobic segment preferably has 10-100 and more preferably 15-80 repeating units

In some embodiments, the conjugates of the invention are formulated into polymeric nanoparticles containing at least one polymer and any therapeutic agent or imaging agent as described in U.S. Pat. No. 8,618,240 to Podobinski, et al., (Cerulean), the contents of which are incorporated herein by reference in their entirety. The polymer can be any of poly(lactide-co-glycolide), poly(lactide), poly(epsilon-caprolactone), poly(isobutylcyanoacrylate), poly(isohexylcyanoacrylate), poly(n-butylcyanoacrylate), poly(acrylate), poly(methacrylate), poly(lactide)-poly(ethylene glycol), poly(lactide-co-glycolide)-poly(ethylene glycol), poly(epsilon-caprolactone)-poly(ethylene glycol), and poly(hexadecylcyanoacrylate-co-poly(ethylene glycol) cyanoacrylate). In some embodiments, the conjugates of the invention are formulated into polymeric nanoparticles through systems and methods that allow concurrent generation of a nanoparticle-containing fluid and its filtration to increase the concentration of the nanoparticles therein as described in U.S. Pat. No. 8,546,521 to Ramstack et al., (Cerulean), the contents of which are incorporated herein by reference in their entirety. The preparation of polymeric nanoparticles, which include any of polylactic acid (PLA) and polyglycolic acid (PGA), comprise a therapeutic agent such as a taxane, or such as docetaxel attached to a polymer component.

In some embodiments, the conjugates of the invention are formulated into nanoparticles comprising a cyclodextrin polymer delivery system and docetaxel (CRLX-301) or camptothecin (CRLX-101) as described in U.S. Pat. No. 8,618,240, US 20140099263, and WO2013025337 to Crawford et al., (Cerulean), the contents of each of which are incorporated herein by reference in their entirety. The cyclodextrin containing polymer (CDP) comprises various combinations of cyclodextrins (e.g., beta-cyclodextrin), comonomers (e.g., PEG containing comonomers), linkers linking the cyclodextrins and comonomers, and/or linkers tethering the docetaxel or campththecin to the CDP, and the PEG has a molecular weight less than 3.4 kDa.

In some embodiments, the conjugates of the invention are formulated into liquid polymeric compositions forming a peptide or protein drug-containing implant in a living body as described in EP 2359860 to Kang, et al., (Samyang), the contents of which are incorporated herein by reference in their entirety. The formulation comprises a water-soluble biocompatible liquid polyethylene glycol derivative, a biodegradable block copolymer which is insoluble in water but soluble in said water-soluble biocompatible liquid polyethylene glycol derivative and a peptide or protein drug, wherein when injected into a living body, the composition forms a polymeric implant containing the physiologically active substance that gradually release the physiologically active substance and then decomposes into materials harmless to the human body.

In some embodiments, the conjugates of the invention are formulated into polymeric micellar nanoparticle compositions as described in EP 2376062 to Seo, et al., (Samyang), the contents of which are incorporated herein by reference in their entirety. The formulation comprises dissolving a poorly water-soluble drug, a salt of polylactic acid or polylactic acid derivative, whose carboxylic acid end is bound to an alkali metal ion, and an amphiphilic block copolymer into an organic solvent; and adding an aqueous solution to the resultant mixture in the organic solvent to form micelles. The copolymer is a diblock copolymer polymerized from a hydrophilic segment and a hydrophobic segment. In the block copolymer, polyethylene oxide is used as a hydrophilic segment and polyaminoacid or hydrophobic group-bound polyaminoacid is used as a hydrophobic segment. The poorly water-soluble drug may be selected from taxane anticancer agents. Particular examples of the taxane anticancer agents may include paclitaxel, docetaxel, 7-epipaclitaxel, t-acetyl paclitaxel, 10-desacetyl-paclitaxel, 10-desacetyl-7-epipaclitaxel, 7-xylosylpaclitaxel, 10-desacetyl-7-glutarylpaclitaxel, 7-N,N-dimethylglycylpaclitaxel, 7-L-alanylpaclitaxel or a mixture thereof. More particularly, the taxane anticancer agent may be paclitaxel or docetaxel.

In some embodiments, the conjugates of the invention are formulated into polymeric micellar nanoparticle compositions as described in EP 2376062 to Seo, et al., (Samyang), the contents of which are incorporated herein by reference in their entirety. The formulation comprises polylactic acid or its derivative as the hydrophobic block and may be one or more selected from a group consisting of polylactic acid, polylactide, polyglycolide, polymandelic acid, polycaprolactone, polydioxan-2-one, polyamino acid, polyorthoester, polyanhydride and a copolymer thereof. Specifically, it may be polylactic acid, polylactide, polyglycolide, polymandelic acid, polycaprolactone or polydioxan-2-one. More specifically, the polylactic acid or its derivative may be one or more selected from a group consisting of polylactic acid, polylactide, polycaprolactone, a copolymer of lactic acid and mandelic acid, a copolymer of lactic acid and glycolic acid, a copolymer of lactic acid and caprolactone, and a copolymer of lactic acid and 1,4-dioxan-2-one. In an embodiment, the hydrophilic block may have a number average molecular weight of 500-20,000 daltons. The hydrophobic block may have a number average molecular weight of 500-10,000 daltons. In another embodiment, the content of the hydrophilic block may be 40-70 wt % based on the total weight of the diblock copolymer. Within this range, the micelle of the amphiphilic diblock copolymer can be maintained stably. The amount of the amphiphilic diblock copolymer may be 80-99.9 wt % based on the total weight of the composition. In an embodiment, the composition may comprise: 0.01-10 wt % of taxane; 0.01-10 wt % of cyclosporin; and 80-99.8 wt % of an amphiphilic diblock copolymer, based on the total weight of the composition. In another embodiment, the composition may comprise: 0.01-10 wt % of taxane; 0.01-10 wt % of cyclosporin; 40-90 wt % of an amphiphilic diblock copolymer; and 10-50 wt % of a polylactic acid alkali metal salt having a terminal carboxyl group. The complex amphiphilic diblock copolymer micelle composition in which taxane and cyclosporin are encapsulated together may have a particle size of 10-200 nm in an aqueous solution, and may be in solid state when freeze dried.

In some embodiments, the cojugates may be incorporated into particles comprising block copolymers with amphilic polymer complexes. For example, the particles may comprise a polyoxyethylene polyoxypropylene copolymer mixture, wherein the copolymer mixture contains two block copolymers, one of which is a hydrophobic copolymer having an ethylene oxide content of from about 10% to about 50% by weight of the copolymer mixture and the other block copolymer being a hydrophilic copolymer having an ethylene oxide content of from about 50% by weight to about 90% by weight of the copolymer mixture as disclosed in U.S. Pat. No. 8,148,338 to Klinski et al. (Supratek Pharma), the contents of which are incorporated herein by reference in their entirety.

In some embodiments, the conjugates may be incorporated into particles that are responsive to temperature, pH, and ionic conditions. For example, the particles may comprise an ionizable network of covalently cross-linked homopolymeric ionizable monomers wherein the ionizable network is covalently attached to a single terminal region of an amphiphilic copolymer to form a plurality of ‘dangling chains’ and wherein the ‘dangling chains’ of amphiphilic copolymer form immobile intra-network aggregates in aqueous solution, as disclosed in U.S. Pat. No. 7,204,997 to Bromberg et al., the contents of which are incorporated herein by reference in their entirety.

In some embodiments, the conjugates may be incorporated into cyclodextrin polymers. The cyclodextrin polymers may target transferrin. For example, the particles may comprise polyconjugates for delivering the RNA interference polynucleotide to a mammalian cell in vivo comprising a membrane inactive reversibly modified amphipathic membrane active random copolymer as disclosed in U.S. Pat. No. 8,658,211 or 8,137,695 to Rozema et al. (Calandro), the contents of which are incorporated herein by reference in their entirety.

In some embodiments, the conjugates may be incorporated into nanoparticles with cyclic oligosaccharide molecules localized on the surface. Any nanparticle comprising a polymer and having cyclic oligosaccharide molecules on the surface disclosed in U.S. Pat. No. 6,881,421 to da Silveira et al. (Bioalliance Pharma), the contents of which are incorporated herein by reference in their entirety. For example, the nanoparticles may comprise polymers such as poly(alkylcyanoacrylate) and the cyclic oligosaccharide is a neutral or charged, native, branched or polymerized or chemically modified cyclodextrin. Any nanoparticle comprising at least one poly(alkylcyanoacrylate) and at least one cyclodextrin disclosed in WO2012131018 to Pisani et al. may be used.

### C. Lipids

The particles may contain one or more lipids or amphiphilic compounds. For example, the particles can be liposomes, lipid micelles, solid lipid particles, or lipid-stabilized polymeric particles. The lipid particle can be made from one or a mixture of different lipids. Lipid particles are formed from one or more lipids, which can be neutral, anionic, or cationic at physiologic pH. The lipid particle is preferably made from one or more biocompatible lipids. The lipid particles may be formed from a combination of more than one lipid, for example, a charged lipid may be combined with a lipid that is non-ionic or uncharged at physiological pH.

The particle can be a lipid micelle. Lipid micelles for drug delivery are known in the art. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

The particle can be a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate agents, by trapping hydrophilic agents in the aqueous interior or between bilayers, or by trapping hydrophobic agents within the bilayer.

The liposomes typically have an aqueous core. The aqueous core can contain water or a mixture of water and alcohol. Suitable alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

The particle can be a solid lipid particle. Solid lipid particles present an alternative to the colloidal micelles and liposomes. Solid lipid particles are typically submicron in size, i.e. from about 5 nm to about 1 micron, from 5 nm to about 500 nm, or from 5 nm to about 250 nm. Solid lipid particles are formed of lipids that are solids at room temperature. They are derived from oil-in-water emulsions, by removing the liquid oil with a solid lipid particle.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-di stearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-a-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

Suitable cationic lipids include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N′-dimethylamino-ethane)carbamoyl] cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC14-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Suitable solid lipids can include cetyl palmitate, beeswax, or cyclodextrin.

Amphiphilic compounds include, but are not limited to, phospholipids, such as 1,2 distearoyl-sn-glycero-3-phosphoethanolamine (DSPE), dipalmitoylphosphatidylcholine (DPPC), di stearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), for example, between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and 3-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

In one embodiment, the conjugate comprising the active agent of the invention may be delivered with a drug delivery system for encapsulating cisplatin and other positively charged drugs into liposomes as disclosed in US 20090280164 to Boulikas (Regulon), the contents of which are incorporated herein by reference in their entirety. PEG coated liposomes comprising neutral and anionic lipids comprising DPPG to help the particles fuse with cellular membranes. The active agents may be combinations of cisplatin with anticancer genes including but not limited to p53, IL-2, IL-12, angiostatin, and oncostatin, as well as combinations of cisplatin with HSV-tk plus ganciclovir.

In one embodiment, the conjugate comprising the active agent of the invention may be delivered with a targeted drug delivery system for encapsulating plasmids, oligonucleotides or negatively-charged drugs in to liposomes as disclosed in US 20030072794 to Boulikas (Regulon), the contents of which are incorporated herein by reference in their entirety. The formulation includes complex formation between DNA with cationic lipid molecules and fusogenic/NLS peptide conjugates composed of a hydrophobic chain of about 10-20 amino acids and also containing four or more histidine residues or NLS at their one end. The encapsulated molecules display therapeutic efficacy in eradicating a variety of solid human tumors including but not limited to breast carcinoma and prostate carcinoma.

In one embodiment, the conjugate comprising the active agent of the invention may be delivered with a drug delivery system for encapsulating Lipoplatin into liposomes as disclosed in WO 2014027994 to Boulikas, et al., (Regulon), the contents of which are incorporated herein by reference in their entirety. Lipoplatin can be prepared by mixing cisplatin with DPPG (dipalmitoyl phosphatidyl glycerol) or other negatively-charged lipid molecules at a 1:1 to 1:2, variations in the molar ratio between cisplatin and DPPG are also of therapeutic value targeting different tissues. The cisplatin-DPPG micelle complex is converted into liposomes encapsulating the cisplatin-DPPG-monolayer or to other type of complexes by direct addition of premade liposomes followed by dialysis against saline and extrusion through membranes to downsize these to 100-160 nm in diameter. Encapsulation of doxorubicin and other positively charged antineoplastic compounds by variations in the process. Addition of positively charged groups to neutral or negatively-charged compounds allows their encapsulation similarly into liposomes.

In some embodiments, the conjugates of the invention are loaded into targeted liposomes encapsulating drug for the treatment of cancer and other diseases as described in U.S. Pat. No. 8,758,810 to Okada, et al., (Mebiopharm), the contents of which are incorporated herein by reference in their entirety. In some embodiments, the conjugates of the invention are formulated with liposomes comprising one or more phosphatidylcholines selected from the group consisting of DMPC, DPPC, POPC, and DSPC, an N-(ω)-dicarboxylic acid-derivatized phosphatidyl ethanolamine, a targeting factor-modified N-(ω)-dicarboxylic acid-derivatized phosphatidyl ethanolamine, an encapsulated drug, and cholesterol. The targeting moiety may comprise transferrin-modified N-(ω)-dicarboxylic acid-derivatized phosphatidyl ethanolamines, folic acid, folate, hyaluronic acid, sugar chains (e.g., galactose, mannose, etc.), fragments of monoclonal antibodies, asialoglycoprotein, etc. In particular embodiments, the targeting factor is a protein or peptide directed to a cell surface receptor (e.g., transferrin, folate, folic acid, asialoglycoprotein, etc.). In other embodiments, the targeting factor is directed to an antigen (e.g., fragments of monoclonal antibodies (e.g., Fab, Fab′, F(ab′)₂, Fc, etc. In a certain embodiments, the targeting factor is transferrin.

In some embodiments, the conjugates of the invention are loaded into a liposome preparation containing oxaliplatin and derivatized with a hydrophilic polymer and a ligand, as described in US 20040022842 to Eriguchi, et al., (Mebiopharm), the contents of which are incorporated herein by reference in their entirety. In one embodiment the hydrophilic polymer is polyethylene glycol, polymethylethylene glycol, polyhydroxypropylene glycol, polypropylene glycol, polymethylpropylene glycol and polyhydroxypropylene oxide, and the ligand is transferrin, folic acid, hyaluronic acid, a sugar chain, a monoclonal antibody and a Fab′ fragment of a monoclonal antibody.

In some embodiments, the conjugates of the invention are formulated into liposomal irinotecan nanoparticles, such as MM-398, as described in WO 2013188586 to Bayever, et al., (Merrimack), the contents of which are incorporated herein by reference in their entirety. The liposome is a unilamellar lipid bilayer vesicle of approximately 80-140 nm in diameter that encapsulates an aqueous space which contains irinotecan complexed in a gelated or precipitated state as a salt with sucrose octasulfate. The lipid membrane of the liposome is composed of phosphatidylcholine, cholesterol, and a polyethyleneglycol-derivatized phosphatidyl-ethanolamine in the amount of approximately one polyethyleneglycol (PEG) molecule for 200 phospholipid molecules.

In some embodiments, the conjugates of the invention are formulated into an immunoliposome loaded with anthracycline and a targeting moiety that is a first anti-HER2 antibody and an anti-cancer therapeutic comprising a second anti-HER2 antibody, such as MM-302, as described in WO 2014089127 to Moyo, et al., (Merrimack), the contents of which are incorporated herein by reference in their entirety. Imunoliposomes are antibody (typically antibody fragment) targeted liposomes that provide advantages over non-immunoliposomal preparations because they are selectively internalized by cells bearing cell surface antigens targeted by the antibody. Such antibodies and immunoliposomes are described, for example, in the following US patents and patent applications: U.S. Pat. Nos. 7,871,620, 6,214,388, 7,135,177, and 7,507,407 (“Immunoliposomes that optimize internalization into target cells”); U.S. Pat. No. 6,210,707 (“Methods of forming protein-linked lipidic microparticles and compositions thereof); U.S. Pat. No. 7,022,336 (“Methods for attaching protein to lipidic microparticles with high efficiency”); and U.S. Pat. Nos. 7,892,554 and 7,244,826 (“Internalizing ErbB2 antibodies.”).

Immunoliposomes targeting HER2 can be prepared in accordance with the foregoing patent disclosures.

In some embodiments, the conjugates of the invention are encapsulated into a liposomal carrier with an anthracycline agent and a cytidine analog as described in U.S. Pat. No. 8,431,806 to Mayer, et al., (Celator), the contents of which are incorporated herein by reference in their entirety. In some embodiments, the conjugates of the invention are encapsulated into a liposomal carrier with cytarabine and daunorubicin at a fixed, molar ratio of cytarabine to daunorubicin of about 5:1 ratio as described in U.S. Pat. No. 8,092,828 to Louie et al., (Celator), the contents of which are incorporated herein by reference in their entirety. A method to treat a leukemia in a human patient, said method comprising administering intravenously to said patient wherein the liposomes comprise DSPC:DSPG:cholesterol at 7:2:1 molar ratio.

In some embodiments, the conjugates of the invention are encapsulated into a liposomal carrier with a fixed, non-antagonistic molar ratio of irinotecan and floxuridine as described in U.S. Pat. No. 8,431,806 to Janoff, et al., (Celator), the contents of which are incorporated herein by reference in their entirety. Any suitable delivery vehicle can be employed that permits the sustained delivery of irinotecan:floxuridine combination in the fixed non-antagonistic molar ratio. In some embodiments, a liposomal formulation may be employed. The liposomes are designed for sustained delivery of the encapsulated drugs at a fixed ratio to a tumor site. In one embodiment, irinotecan and floxuridine are stably associated with the liposomes. Typically, the liposomes have a diameter of less than 300 nm, sometimes less than 200 nm. In one example, the nominal size of these liposomes is approximately 110 nm and sterilization is achieved by filtration through a 0.2 μm filter. In a specific embodiment, the liposome membrane is composed of di stearoylphosphatidylcholine (DSPC), di stearoylphosphatidylglycerol (DSPG) and cholesterol (CHOL) in a 7:2:1:molar ratio. In one instance, the liposomes are prepared by an water in oil derived liposome method and extruded liposomes are suspended in phosphate-buffered sucrose at pH 7.0. Any suitable means of encapsulating the drug combination in the liposomes can be employed. In a specific embodiment, irinotecan and floxuridine are encapsulated in the liposome using a copper gluconate/triethanolamine-based active loading procedure whereby irinotecan accumulates due to complexation inside pre-formed liposomes and floxuridine is passively encapsulated.

In some embodiments, the conjugates of the invention comprise liposomes having controlled release of campththecens/plantiums as described in U.S. Pat. No. 8,431,806 to Tardi, et al., (Celator), the contents of which are incorporated herein by reference in their entirety. The platinum-based liposomes comprise a mixture of at least two phosphatidyl choline lipids of varying acyl chain length including 5-55% of a phosphatidyl choline lipid containing acyl groups of chain length of 14-17 carbon atoms, and at least 5-55% of a second phosphatidyl choline lipid containing acyl groups of chain length of at least 18 carbon atoms. The liposomes comprise DSPC and either DMPC or DPPC at a ratio in the range of about 13:1 to 1:13, with the platinum-based drug is cisplatin, carboplatin or oxaliplatin. The liposomes further comprise cholesterol, phosphatidylglycerol, and an additional therapeutic agent is is irinotecan (CPT-Il), topotecan, 9-aminocamptothecin or lurtotecan, or is a hydrophilic salt of a water-insoluble camptothecin. Additionally, the platinum-based drug and said additional therapeutic agent are present in a mole ratio that has a non-antagonistic cytotoxic or cytostatic effect to relevant cells or tumor cell homogenates, and wherein said platinum-based drug and additional therapeutic agent are stably associated with delivery vehicles such that a non-antagonistic mole ratio is maintained in the blood of a subject for at least one hour after administration. The water-soluble camptothecin is irinotecan (CPT-II), topotecan, 9-aminocamptothecin or lurtotecan, or is a hydrophilic salt of a water-insoluble camptothecin and the platinum-based drug is cisplatin, carboplatin or oxaliplatin. The liposomes comprise a mixture of DSPC and a second phosphatidylcholine lipid that is not DSPC at a ratio in the range of about 13:1 to 1:13, the phosphatidyl choline lipids are DSPC and either DPPC or DMPC. The liposomes further comprise phosphatidylglycerol or a phosphatidylinositol, such as DSPG or DMPG. The liposome may comprise of cholesterol or a third agent.

In some embodiments, the conjugates of the invention comprise pharmaceutical capsules which comprises a suspension of microparticles suspended in an oil as described in EP 2501365 to Duena, et al., (GP Pharm), the contents of which are incorporated herein by reference in their entirety. The pharmaceutical capsule comprises a suspension of polymeric microcapsules which comprise at least one polymer and an active pharmaceutical ingredient selected from the group formed by the angiotensin-converting enzyme inhibitors and the angiotensin receptor blockers, these microcapsules being suspended in an oil which contains polyunsaturated fatty acid alkyl esters. The polyunsaturated fatty acids of these alkyl esters belong to the omega-3 series and include eicosapentaenoic acid, docosahexaenoic acid, and/or mixtures thereof. The alkyl radical of these alkyl esters is selected from the group formed by short chain alkyl radicals, with from 1 to 8 carbon atoms, and may comprise more than 50% of polyunsaturated fatty acid alkyl esters. The angiotensin-converting enzyme inhibitor is selected from the group formed by captopril, enalapril, enalaprilat, ramipril, quinapril, perindopril, lisinopril, benazepril, fosinopril, spirapril, trandolapril, moexipril, cilazapril, imidapril, rentiapril, temocapril, alacepril, delapril, moveltipril, zofenopril, pentopril, libenzapril, pivopril, ceronapril, indolapril, teprotide, their pharmaceutically acceptable salts and their acids. The angiotensin II receptor blocker is selected from the group formed by candesartan, eprosartan, irbesartan, losartan, olmesartan, telmisartan, valsartan, tasosartan, pratosartan, azilsartan, saralasin, ripisartan, elisartan, milfasartan, embusartan, fonsartan, saprisartan, zolasartan, forasartan, pomisartan, abitesartan, fimasartan, N-benzyl-losartan, enoltasosartan, glycyl-losartan, opomisartan, trityl-losartan, sarmesin, isoteolin and their pharmaceutically acceptable salts. The polymer of these microcapsules is selected from the group formed by proteins, polyesters, polyacrylates, polycyanoacrylates, polysaccharides, polyethylene glycol and/or mixtures thereof, and include the group formed by gelatin, albumin, alginates, carrageenans, pectins, gum arabic, chitosan, carboxymethyl cellulose, ethyl cellulose, hydroxypropyl methylcellulose, nitrocellulose, cellulose acetate butyrate, cellulose acetate phthalate, hydroxypropyl methylcellulose phthalate, hydroxypropyl methylcellulose acetate-succinate, polyvinyl acetate phthalate, poly(e-caprolactone), poly(p-dioxanone), poly(6-valerolactone), poly(p-hydroxybutyrate), poly(p-hydroxybutyrate) and β-hydroxyvalerate copolymers, poly(p-hydroxypropionate), methacrylic acid copolymers, dimethylaminoethyl methacrylate copolymers, trimethylammonium ethyl methacrylate copolymers, polymers and copolymers of lactic and glycolic acids, polymers and copolymers of lactic and glycolic acids and polyethylene glycol and/or mixtures thereof. The microcapsules represent between 0.001% and 80% of the total weight of the capsule, and contain at least one plasticizer, a fluidifying agent and/or an antioxidant. The capsule comprises an enteric coating.

In some embodiments, the conjugates of the invention comprise nebulized liposomal amikacin formulation as described in US 20130089598 to Gupta (Insmed Corp.), the contents of which are incorporated herein by reference in their entirety. The nebulized liposomal amikacin formulation comprises a lipid to amikacin ratio of about 0.3 to about 1.0 by weight comprising a lipid selected from the group consisting of egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidyl choline (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), di stearoylphosphatidylcholine (DSPC), di stearoylphosphatidylglycerol (DSPG), dioleylphosphatidylethanolamine (DOPE), palmitoylstearoylphosphatidylcholine (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholipids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), phosphatidylglycerols (PGs), phosphatidic acids (PAs), phosphatidylinositols (PIs), phosphatidyl serines (PSs), di stearoylphosphatidylglycerol (DSPG), dimyristoylphosphatidylacid (DMPA), dipalmitoylphosphatidylacid (DPPA), di stearoylphosphatidylacid (DSPA), dimyristoylphosphatidylinositol (DMPI), dipalmitoylphosphatidylinositol (DPPI), di stearoylphospatidylinositol (DSPI), dimyristoylphosphatidylserine (DMPS), dipalmitoylphosphatidylserine (DPPS), distearoylphosphatidylserine (DSPS), and mixtures thereof.

In some embodiments, the conjugates of the invention comprise sublingual formulations comprising fentanyl as described in U.S. Pat. No. 8,486,972 to Kottayil, et al., (Insys Therapeutics), the contents of which are incorporated herein by reference in their entirety. The non-propellant sublingual fentanyl formulation comprising of discrete liquid droplets of about 0.1% to about 0.8% by weight of fentanyl or a pharmaceutically acceptable salt, about 20% to 60% by weight of ethanol, about 4% to 6% by weight of propylene glycol, and the discrete liquid droplets have a size distribution of from about 10 μm to about 200

In some embodiments, the conjugates of the invention comprise oral cannabinoid formulations, including an aqueous-based oral dronabinol solution as described in U.S. Pat. No. 8,222,292 to Goskonda, et al., (Insys Therapeutics), the contents of which are incorporated herein by reference in their entirety. The oral cannabinoid formulations comprising essentially of dronabinol, 30-33% w/w water, about 50% w/w ethanol, 0.01% w/w butylated hydroxylanisole (BHA) or 0.1% w/w ethylenediaminetetraacetic acid (EDTA) and 5-21% w/w co-solvent, having a combined total of 100%, where the co-solvent is selected from the group consisting of propylene glycol, polyethylene glycol and combinations thereof.

In some embodiments, the conjugates of the invention comprise a thermosensitive liposome for the delivery of active agents as described in EP 2217209 to Mei, et al., (Celison), the contents of which are incorporated herein by reference in their entirety. The thermosensitive liposome comprises at least one phosphatidylcholine, at least one phosphatidylglycerol and at least one lysolipid, and the gel to liquid phase transition temperature of the liposome is from 39 0° C. to 45° C. The formulation may comprise of PEGylated phospholipid phosphatidylcholine, dipalmitoylphosphatidylcholine (DPPC), di stearoylphosphatidylglycerol (DSPG), and the lysolipid is monostearoylphosphatidylcholine (MSPC), lipid is PEG-2000 modified di stearoylphosphatidylethanolamine (DSPE-PEG2000). The liposome may comprising DPPC:DSPG:MSPC DSPE-PEG2000:active agent in the ratio of 60-80:6-12:6-12:4-15:1-30 on a weight basis. The active agent may comprise of alkylating agents, antimetabolites, spindle poison plant alkaloids, cytotoxic antitumor antibiotics, topoisomerase inhibitors, monoclonal antibodies or fragments thereof, photosensitizers, kinase inhibitors, antitumor enzymes and inhibitors of enzymes, apoptosis-inducers, biological response modifiers, anti-hormones, retinoids and platinum containing compounds.

In some embodiments, the conjugates may be incorporated into lipid-based systems. The lipid-based systems may comprise a lipid or lysolipid derivative, e.g., liposomes (and micelles) including lipid derivatives having an aliphatic group and a hydrophilic moiety as described in U.S. Pat. Nos. 7,368,254, 7,166,297 or WO2007107161 to Jorgensen et al. (Liplasome Pharma), the contents of which are incorporated herein by reference in their entirety. In another example, the lipid-based system may be a liposome comprising between 25% and 45% (mol/mol) of an anionic lipid, less than 1% cholesterol (mol/mol) wherein the liposome has been exposed to a divalent cation at a concentration between 0.1 mM and 1 mM as described in US 20120009243 to Vikbjerg et al., the contents of which are incorporated herein by reference in their entirety.

D. Inorganic Nanoparticles

Inorganic nanoparticles exhibit a combination of physical, chemical, optical and electronic properties and provide a highly multifunctional platform to image and diagnose diseases, to selectively deliver therapeutic agens, and to sensitive cells and tissues to treatment regiments. Not wishing to be bound to any theory, enhanced permeability and retention (EPR) effect provides a basis for the selective accumulation of many high-molecular-weight drugs. Circulating inorganic nanoparticles preferentially accumulate at tumor sites and in inflamed tissues (Yuan et al., Cancer Res., vo1.55(17):3752-6, 1995, the contents of which are incorporated herein by reference in their entirety) and remain lodged due to their low diffusivity (Pluen et al., PNAS, vo1.98(8):4628-4633, 2001, the contents of which are incorporated herein by reference in their entirety). The size of the inorganic nanoparticles may be 10 nm-500 nm, 10 nm-100 nm or 100 nm-500 nm. The inorganic nanoparticles may comprise metal (gold, iron, silver, copper, nickel, etc.), oxides (ZnO, TiO₂, Al₂O₃, SiO₂, iron oxide, copper oxide, nickel oxide, etc.), or semiconductor (CdS, CdSe, etc.). The inorganic nanoparticles may also be perfluorocarbon or FeCo.

Inorganic nanoparticles have high surface area per unit volume. Therefore, they may be loaded with therapeutic drugs and imaging agents at high densitives. A variety of methods may be used to load therapeutic drugs into/onto the inorganic nanoparticles, including but not limited to, colvalent bonds, electrostatic interactions, entrapment, and encapsulation. In addition to therapeutic agent drug loads, the inorganic nanoparticles may be funcationalized with targeting moieties, such as tumor-targeting ligands, on the surface. Formulating therapeutic agents with inorganic nanoparticles allows imaging, detection and monitoring of the therapeutic agents.

In some embodiments, conjugates of the invention are formulated with gold nanoparticles. Gold nanoparticles may be in the forms of nanospheres, nanorods, nanoshells (e.g., a particle with silica core and gold shell), nanocages, etc and may be synthesized with any known method, such as colloidal methods, seeded growth methods, etc. The conjugates of the invention may be attached to the surface of the gold nanoparticles with covalent bonds, linkers, or non-covalent bonds with any known method. Once synthesized, the surface of gold nanoparticles is usually surrounded by a stabilizing agent, which creates an overall surface charge. A variety of molecules may be attached to the surface of gold nanoparticles through electrostatic interactions. McIntosh et al. utilized mixed monolayer protected Au clusters coated with a cationic stabilizing agent, 11-trimethylammoniumundecanethiol, to non-covalently attach the negatively charged phosphate backbone of DNA to the surface of the nanoparticle (McIntosh et al., JACS, vol. 123(31):7626-7629, 2001, the contents of which are incorporated herein by reference in their entirety). Huo et al. coupled prostate-specific antigen antibodies to the surface of anionic, citrate-stabilized gold nanospheres through electrostatic interactions (Huo et al., JACS, vol. 130(9):2780-2782, 2008, the contents of which are incorporated herein by reference in their entirety).

In one embodiment, the conjugate of the invention is hydrophobic and may be form a kinetically stable complex with gold nanoparticles funcationalized with water-soluble zwitterionic ligands disclosed by Kim et al. (Kim et al., JACS, vol. 131(4):1360-1361, 2009, the contents of which are incorporated herein by reference in their entirety). Kim et al. demonstrated that hydrophobic drugs carried by the gold nanoparticles are efficiently released into cells with little or no cellular uptake of the gold nanoparticles.

In one embodiment, the conjugates of the invention may be formulated with gold nanoshells. As a non-limiting example, the conjugates may be delivered with a temperature sensitive system comprising polymers and gold nanoshells and may be released photothermally. Sershen et al. designed a delivery vehicle comprising hydrogel and gold nanoshells, wherein the hydrogels are made of copolymers of N-isopropylacrylamide (NIPAAm) and acrylamide (AAm) and the gold nanoshells are made of gold and gold sulfide (Sershen et al., J Biomed Mater, vol. 51:293-8, 2000, the contents of which are incorporated herein by reference in their entirety). Irradiation at 1064 nm was absorbed by the nanoshells and converted to heat, which led to the collapose of the hydrogen and release of the drug. The conjugate of the invention may also be encapsulated inside hollow gold nanoshells.

In some embodiments, the conjugates of the invention may be attached to gold nanoparticles via covalent bonds. Covalent attachment to gold nanoparticles may be achieved through a linker, such as a free thiol, amine or carboxylate funcational group. In some embodiments, the linkers are located on the surface of the gold nanoparticles. In some embodiments, the conjugates of the invention may be modified to comprise the linkers. The linkers may comprise a PEG or oligoethylene glycol moiety with varying length to increase the particles' stability in biological environment and to control the density of the drug loads. PEG or oligoethylene glycol moieties also minimize nonspecific adsorption of undesired biomolecules. PEG or oligoethylene gycol moieties may be branched or linear. Tong et al. disclosed that branched PEG moieties on the surface of gold nanoparticles increase circulatory half-life of the gold nanoparticles and reduced serum protein binding (Tong et al., Langmuir, vol. 25(21):12454-9, 2009, the contents of which are incorporated herein by reference in their entirety).

In one embodiment, the conjugate of the invention may comprise PEG-thiol groups and may attach to gold nanoparticles via the thiol group. The synthesis of thiol-PEGylated conjugates and the attachment to gold nanoparticles may follow the method disclosed by E1-Sayed et al. (E1-Sayed et al., Bioconjug. Chem., vol. 20(12):2247-2253, 2010, the contents of which are incorporated herein by reference in their entirety).

In another embodiment, the conjugate of the invention may be tethered to an amine-funcationalized gold nanoparticles. Lippard et al. disclosed that Pt(IV) prodrugs may be delivered with amine-functionalized polyvalent oligonucleotide gold nanoparticles and are only activated into their active Pt(II) forms after crossing the cell membrane and undergoing intracellular reduction (Lippard et al., JACS, vol. 131(41):14652-14653, 2009, the contents of which are incorporated herein by reference in their entirety). The cytotoxic effects for the Pt(IV)-gold nanoparticle complex are higher than the free Pt(IV) drugs and free cisplatin.

In some embodiments, conjugates of the invention are formulated with magnetic nanoparticle such as iron, cobalt, nickel and oxides thereof, or iron hydroxide nanoparticles. Localized magnetic field gradients may be used to attract magnetic nanoparticles to a chosen site, to hold them until the therapy is complete, and then to remove them. Magnetic nanoparticles may also be heated by magnetic fields. Alexiou et al. prepared an injection of magnetic particle, ferrofluids (FFs), bound to anticancer agents and then concentrated the particles in the desired tumor area by an external magnetic field (Alexiou et al., Cancer Res. vol. 60(23):6641-6648, 2000, the contents of which are incorporated herein by reference in their entirety). The desorption of the anticancer agent took place within 60 min to make sure that the drug can act freely once localized to the tumor by the magnetic field.

In some embodiments, the conjugates of the invention are loaded onto iron oxide nanoparticles. In some embodiments, the conjugates of the invention are formulated with superparamagnetic nanoparticles based on a core consisting of iron oxides (SPION). SPION are coated with inorganic materials (silica, gold, etc.) or organic materials (phospholipids, fatty acids, polysaccharides, peptides or other surfactants and polymers) and can be further functionalized with drugs, proteins or plasmids.

In one embodiment, water-dispersible oleic acid (OA)-poloxamer-coated iron oxide magnetic nanoparticles disclosed by Jain et al. (Jain, Mol. Pharm., vol. 2(3):194-205, 2005, the contents of which are incorporated herein by reference in their entirety) may be used to deliver the conjugates of the invention. Therapeutic drugs partition into the OA shell surrounding the iron oxide nanoparticles and the poloxamer copolymers (i.e., Pluronics) confers aqueous dispersity to the formulation. According to Jain et al., neither the formulation components nor the drug loading affected the magnetic properties of the core iron oxide nanoparticles. Sustained release of the therapeutic drugs was achieved.

In one embodiment, the conjugates of the invention are bonded to magnetic nanoparticles with a linker. The linker may be a linker capable of undergoing an intramolecular cyclization to release the conjugates of the invention. Any linker and nanoparticles disclosed in WO2014124329 to Knipp et al., the contents of which are incorporated herein by reference in their entirety, may be used. The cyclization may be induced by heating the magnetic nanoparticle or by application of an alternating electromagnetic field to the magnetic nanoparticle.

In one embodiment, the conjugates of the invention may be delivered with a drug delivery system disclosed in U.S. Pat. No. 7,329,638 to Yang et al., the contents of which are incorporated herein by reference in their entirety. The drug delivery system comprises a magnetic nanoparticle associated with a positively charged cationic molecule, at least one therapeutic agent and a molecular recognition element.

In one embodiment, nanoparticles having a phosphate moiety are used to deliver the conjuates of the invention. The phosphate-containing nanparticle disclosed in U.S. Pat. No. 8,828,975 to Hwu et al., the contents of which are incorporated herein by reference in their entirety, may be used. The nanoparticles may comprise gold, iron oxide, titanium dioxide, zinc oxide, tin dioxide, copper, aluminum, cadmium selenide, silicon dioxide or diamond. The nanoparticles may contain a PEG moiety on the surface.

In some embodiments, conjuates may be bound delivered with metal vehicles. The colloidal metal vehicles may be any metal particle disclosed in U.S. Pat. No. 8,137,989 to Tarmakin et al., the contents of which are incorporated herein by reference in their entirety. The colloidal metal vehicles may also be PEGylated metal particles disclosed in U.S. Pat. Nos. 8,785,202, 7,229,841, or U.S. Pat. No. 7,387,900 to Tamarkin et al. (Cytimmune), the contents of which are incorporated herein by reference in their entirety, such as colloidal gold particles with PEG thiol derivatives covalently bound to the gold particles. For another example, the colloidal metal vehicles may be gold nanoparticles, silver nanoparticles, silica nanoparticles, iron nanoparticles, metal hybrid nanoparticles such as gold/iron nanoparticles, nanoshells, gold nanoshells, silver nanoshells, gold nanorods, silver nanorods, metal hybrid nanorods, quantum dots, nanoclusters, liposomes, dendrimers, metal/lipsome particles, metal/dendrimer nanohybrids or carbon nanotubes as disclosed in WO2009039502 to Tamarkin et al., the contents of which are incorporated herein by reference in their entirety. A stealth agent may be employed such as PEG, PolyPEG, polyoxypropylene polymers, polyvinylpyrrolidone polymers, rPEG, or hydroxyethyl starch, hydrophilic agents and polymers.

In some embodiments, conjugates may be delivered with nanoparticles that partially transduce an external energy into heat energy for increasing the temperature of a target area and allow for focused hyperthermia, including nanoshells, nanorods, carbon nanotubes, fullerenes, carbon fullerenes, paramagnetic particles, metallic nanoparticles, metal colloids, carbon particles, buckyballs, nanocubes, nanostars, indocyanine green encapsulated in nanoparticles, acoustic particles, and any combination thereof as disclosed in US20130197295 to Krishnan et al., the contents of which are incorporated herein by reference in their entirety. For example, conjugates may be delivered with gold nanoshells with silica cores or gold-gold sulfide nanoshells disclosed by Krishnan et al.

### E. Additional Targeting Moieties

The particles can contain one or more targeting moieties targeting the particle to a specific organ, tissue, cell type, or subcellular compartment in addition to the targeting moieties of the conjugate. The additional targeting moieties can be present on the surface of the particle, on the interior of the particle, or both. The additional targeting moieties can be immobilized on the surface of the particle, e.g., can be covalently attached to polymer or lipid in the particle. In preferred embodiments, the additional targeting moieties are covalently attached to an amphiphilic polymer or a lipid such that the targeting moieties are oriented on the surface of the particle.

### F. Additional Active Agents

The particles can contain one or more additional active agents in addition to those in the conjugates. The additional active agents can be therapeutic, prophylactic, diagnostic, or nutritional agents as listed above.

The additional active agents can be present in any amount, e.g. from about 0.05% to about 90%, from about 1% to about 50%, from about 0.05% to about 25%, from about 0.05% to about 20%, from about 0.05% to about 10%, from about 1% to about 90%, from about 1% to about 50%, from about 1% to about 25%, from about 1% to about 20%, from about 1% to about 10%, or from about 5% to about 10% (w/w) based upon the weight of the particle. In one embodiment, the agents are incorporated in a about 1% to about 10% loading w/w.

### III. Parmaceutical Compositions and Formulations

In some embodiments, compositions are administered to humans, human patients, healthy volunteers, or any other subjects. For the purposes of the present disclosure, the phrase “active ingredient” generally refers to the conjugate or particles containing the conjugates to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to animals, e.g. mammals, rodents, or avians. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with one or more excipients and/or one or more other accessory ingredients including solvents and aqueous solutions, and then, if necessary and/or desirable, dissolving, dividing, sterilizing, filling or shaping and/or packaging the product into a desired single- or multi-use units.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.05% and 100%, e.g., between 0.1 and 75%, between 0.5 and 50%, between 1-30%, between 5-80%, at least 80% (w/w) active ingredient.

The conjugates or particles of the present invention can be formulated using one or more excipients to: (1) increase stability; (2) permit the sustained or delayed release (e.g., from a depot formulation of the monomaleimide); (3) alter the biodistribution (e.g., target the monomaleimide compounds to specific tissues or cell types); (4) alter the release profile of the monomaleimide compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present invention may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the invention may include one or more excipients, each in an amount that together increases the stability of the monomaleimide compounds.

In some embodiments, the conjugates and/or particles comprising such conjugates of the present invention are formulated in aqueous formulations such as pH 7.4 phosphate-buffered formulation, or pH 6.2 citrate-buffered formulation; formulations for lyophilization such as pH 6.2 citrate-buffered formulation with 3% mannitol, pH 6.2 citrate-buffered formulation with 4% mannitol/1% sucrose; or a formulation prepared by the process disclosed in U.S. Pat. No. 8,883,737 to Reddy et al. (Endocyte), the contents of which are incorporated herein by reference in their entirety.

In some embodiments, the conjugates and/or particles comprising such conjugates of the present invention targets folate receoptors and are formulated in liposomes prepared following methods by Leamon et al. in Bioconjugate Chemistry, vol. 14 738-747 (2003), the contents of which are incorporated herein by reference in their entirety. Briefly, folate-targeted liposomes will consist of 40 mole % cholesterol, either 4 mole % or 6 mole % polyethyleneglycol (Mr^{˜}2000)-derivatized phosphatidylethanolamine (PEG2000-PE, Nektar, Ala., Huntsville, Ala.), either 0.03 mole % or 0.1 mole % folate-cysteine-PEG3400-PE and the remaining mole % will be composed of egg phosphatidylcholine, as disclosed in U.S. Pat. No. 8,765,096 to Leamon et al. (Endocyte), the contents of which are incorporated herein by reference in their entirety. Lipids in chloroform will be dried to a thin film by rotary evaporation and then rehydrated in PBS containing the drug. Rehydration will be accomplished by vigorous vortexing followed by 10 cycles of freezing and thawing. Liposomes will be extruded 10 times through a 50 nm pore size polycarbonate membrane using a high-pressure extruder. Similarly liposomes not targeting folate receports may be prepared identically with the absence offolate-cysteine-PEG3400-PE.

In some embodiments, the conjugates and/or particles comprising such conjugates of the present invention are formulated in parenteral dosage forms including but limited to aqueous solutions of the conjugates and/or particles comprising such conjugates, in an isotonic saline, 5% glucose or other pharmaceutically acceptable liquid carriers such as liquid alcohols, glycols, esters, and amides, as disclosed in U.S. Pat. No. 7,910,594 to Vlahov et al. (Endocyte), the contents of which are incorporated herein by reference in their entirety. The parenteral dosage form may be in the form of a reconstitutable lyophilizate comprising the dose of the conjugates and/or particles comprising such conjugates. Any prolonged release dosage forms known in the art can be utilized such as, for example, the biodegradable carbohydrate matrices described in U.S. Pat. Nos. 4,713,249; 5,266,333; and 5,417,982, the disclosures of which are incorporated herein by reference, or, alternatively, a slow pump (e.g., an osmotic pump) can be used.

In some embodiments, the parenteral formulations are aqueous solutions containing carriers or excipients such as salts, carbohydrates and buffering agents (e.g., at a pH of from 3 to 9). In some embodiments, the conjugates and/or particles comprising such conjugates of the present invention may be formulated as a sterile non-aqueous solution or as a dried form and may be used in conjunction with a suitable vehicle such as sterile, pyrogen-free water. The preparation of parenteral formulations under sterile conditions, for example, by lyophilization under sterile conditions, may readily be accomplished using standard pharmaceutical techniques well-known to those skilled in the art. The solubility of a conjugates and/or particles comprising such conjugates used in the preparation of a parenteral formulation may be increased by the use of appropriate formulation techniques, such as the incorporation of solubility-enhancing agents.

In some embodiments, the conjugates and/or particles comprising such conjugates of the present invention may be prepared in an aqueous sterile liquid formulation comprisimg monobasic sodium phosphate monohydrate, dibasic disodium phosphate dihydrate, sodium chloride, potassium chloride and water for injection, as disclosed in US 20140140925 to Leamon et al., the contents of which are incorporated herein by reference in their entirety. For example, the conjugates and/or particles comprising such conjugates of the present invention may be formulated in an aqueous liquid of pH 7.4, phosphate buffered formulation for intravenous administration as disclosed in Example 23 of WO2011014821 to Leamon et al. (Endocyte), the contents of which are incorporated herein by reference in their entirety. According to Leamon, the aqueous formulation needs to be stored in the frozen state to ensure its stability.

In some embodiments, the conjugates and/or particles comprising such conjugates of the present invention are formulated for intravenous (IV) administration. Any formulation or any formulation prepared according to the process disclosed in US 20140030321 to Ritter et al. (Endocyte), the contents of which are incorporated herein by reference in their entirety, may be used. For example, the conjugates and/or particles comprising such conjugates may be formulated in an aequous sterile liquid formulation of pH 7.4 phosphate buffered composition comprising sodium phosphate, monobasic monohydrate, disodium phosphate, dibasic dehydrate, sodium chloride, and water for injection. As another example, the conjugates and/or particles comprising such conjugates may be formulated in pH 6.2 citrated-buffered formulation comprising trisodium citrate, dehydrate, citric acid and water for injection. As another example, the conjugates and/or particles comprising such conjugates may be formulated with 3% mannitol in a pH 6.2 citrate-buffered formulation for lyophilization comprising trisodium citrate, dehydrate, citric acid and mannitol. 3% mannitol may be replaced with 4% mannitol and 1% sucrose.

In some embodiments, the particles comprise biocompatible polymers. In some embodiments, the particles comprise about 0.2 to about 35 weight percent of a therapeutic agent; and about 10 to about 99 weight percent of a biocompatible polymer such as a diblock poly(lactic) acid-poly(ethylene)glycol as disclosed in US 20140356444 to Troiano et al. (BIND Therapeutics), the contents of which are incorporated herein by reference in their entirety. Any therapeutical particle composition in U.S. Pat. Nos. 8,663,700, 8,652,528, 8,609,142, 8,293,276 and 8,420,123, the contents of each of which are incorporated herein by reference in their entirety, may also be used.

In some embodiments, the particles comprise a hydrophobic acid. In some embodiments, the particles comprise about 0.05 to about 30 weight percent of a substantially hydrophobic acid; about 0.2 to about 20 weight percent of a basic therapeutic agent having a protonatable nitrogen; wherein the pKa of the basic therapeutic agent is at least about 1.0 pKa units greater than the pKa of the hydrophobic acid; and about 50 to about 99.75 weight percent of a diblock poly(lactic) acid-poly(ethylene)glycol copolymer or a diblock poly(lactic acid-co-glycolic acid)-poly(ethylene)glycol copolymer, wherein the therapeutic nanoparticle comprises about 10 to about 30 weight percent poly(ethylene)glycol as disclosed in WO2014043625 to Figueiredo et al. (BIND Therapeutics), the contents of which are incorporated herein by reference in their entirety. Any therapeutical particle composition in US 20140149158, 20140248358, 20140178475 to Figueiredo et al., the contents of each of which are incorporated herein by reference in their entirety, may also be used.

In some embodiments, the particles comprise a chemotherapeutic agent; a diblock copolymer of poly(ethylene)glycol and polylactic acid; and a ligand conjugate, as disclosed in US 20140235706 to Zale et al. (BIND Therapeutics), the contents of which are incorporated herein by reference in their entirety. Any of the particle compositions in U.S. Pat. Nos. 8,603,501, 8,603,500, 8,603,499, 8,273,363, 8,246,968, 20130172406 to Zale et al., may also be used.

In some embodiments, the particles comprise a targeting moiety. As a non-limiting example, the particles may comprise about 1 to about 20 mole percent PLA-PEG-basement vascular membrane targeting peptide, wherein the targeting peptide comprises PLA having a number average molecular weight of about 15 to about 20 kDa and PEG having a number average molecular weight of about 4 to about 6 kDa; about 10 to about 25 weight percent anti-neointimal hyperplasia (NIH) agent; and about 50 to about 90 weight percent non-targeted poly-lactic acid-PEG, wherein the therapeutic particle is capable of releasing the anti-NIH agent to a basement vascular membrane of a blood vessel for at least about 8 hours when the therapeutic particle is placed in the blood vessel as disclosed in U.S. Pat. No. 8,563,041 to Grayson et al. (BIND Therapeutics), the contents of which are incorporated herein by reference in their entirety.

In some embodiments, the particles comprise about 4 to about 25% by weight of an anti-cancer agent; about 40 to about 99% by weight of poly(D,L-lactic)acid-poly(ethylene)glycol copolymer; and about 0.2 to about 10 mole percent PLA-PEG-ligand; wherein the pharmaceutical aqueous suspension have a glass transition temperature between about 39 and 41° C., as disclosed in U.S. Pat. No. 8,518,963 to Ali et al. (BIND Therapeutics), the contents of which are incorporated herein by reference in theire entirety.

In some embodiments, the particles comprise about 0.2 to about 35 weight percent of a therapeutic agent; about 10 to about 99 weight percent of a diblock poly(lactic) acid-poly(ethylene)glycol copolymer or a diblock poly(lactic)-co-poly (glycolic) acid-poly(ethylene)glycol copolymer; and about 0 to about 75 weight percent poly(lactic) acid or poly(lactic) acid-co-poly (glycolic) acid as disclosed in WO2012166923 to Zale et al. (BIND Therapeutics), the contents of which are incorporated herein by reference in their entirety.

In some embodiments, the particles are long circulating and may be formulated in a biocompatible and injectable formulation. For example, the particles may be a sterile, biocompatible and injectable nanoparticle composition comprising a plurality of long circulating nanoparticles having a diameter of about 70 to about 130 nm, each of the plurality of the long circulating nanoparticles comprising about 70 to about 90 weight percent poly(lactic) acid-co-poly(ethylene) glycol, wherein the weight ratio of poly(lactic) acid to poly(ethylene) glycol is about 15 kDa/2 kDa to about 20 kDa/10 kDa, and a therapeutic agent encapsulated in the nanoparticles as disclosed in US 20140093579 to Zale et al. (BIND Therapeutics), the contents of which are incorporated herein by reference in their entirety.

In some embodiments, provided is a reconstituted lyophilized pharmaceutical composition suitable for parenteral administration comprising the particles of the present invention and an appropriate lyoprotectant (bulking agent). For example, the reconstituted lyophilized pharmaceutical composition may comprise a 0.1-100 mg/mL concentration of polymeric nanoparticles in an aqueous medium; wherein the polymeric nanoparticles comprise: a poly(lactic) acid-block-poly(ethylene)glycol copolymer or poly(lactic)-co-poly(glycolic) acid-block-poly(ethylene)glycol copolymer, and a taxane agent; 4 to 6 weight percent sucrose or trehalose; and 7 to 12 weight percent hydroxypropyl β-cyclodextrin, as disclosed in U.S. Pat. No. 8,637,083 to Troiano et al. (BIND Therapeutics), the contents of which are incorporated herein by reference in their entirety. Any pharmaceutical composition in U.S. Pat. Nos. 8,603,535, 8,357,401, 20130230568, 20130243863 to Troiano et al. may also be used.

In some embodiments, the conjugates of the inventnion may be delivered with a bacteriophage. For example, a bacteriophage may be conjugated through a labile/non labile linker or directly to at least 1,000 therapeutic drug molecules such that the drug molecules are conjugated to the outer surface of the bacteriophage as disclosed in US 20110286971 to Yacoby et al., the contents of which are incorporated herein by reference in their entirety. According to Yacoby et al., the bacteriophage may comprise an exogenous targeting moiety that binds a cell surface molecule on a target cell.

In some embodiments, the conjugates of the invention may be delivered with a dendrimer. The conjugates may be encapsulated in a dendrimer, or disposed on the surface of a dendrimer. For example, the conjugates may bind to a scaffold for dendritic encapsulation, wherein the scaffold is covalently or non-covalently attached to a polysaccharide, as disclosed in US 20090036553 to Piccariello et al., the contents of which are incorporated herein by reference in their entirety. The scaffold may be any peptide or oligonucleotide scaffold disclosed by Piccariello et al.

In some embodiments, the conjugates of the invention may be delivered by a cyclodextrin. In one embodiment, the conjugates may be formulated with a polymer comprising a cyclodexrin moiety and a linker moiety as disclosed in US 20130288986 to Davis et al., the contents of which are incorporated herein by reference in their entirety. Davis et al. also teaches that the conjugate may be covalently attached to a polymer through a tether, wherein the tether comprises a self-cyclizing moiety.

In some embodiments, the conjugates of the invention may be delivered with an aliphatic polymer. For example, the aliphatic polymer may comprise polyesters with grafted zwitterions, such as polyester-graft-phosphorylcholine polymers prepared by ring-opening polymerization and click chemistry as disclosed in U.S. Pat. No. 8,802,738 to Emrick, the contents of which are incorporated herein by reference in their entirety.

Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfate, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, retinoid-like excipient (e.g. excipients that resemble vitamin A), coloring agents, coating agents, sweetening, flavoring, and/or perfuming agents can be present in the composition, according to the judgment of the formulator.

Lipidoids

The synthesis of lipidoids has been extensively described. Provided herein is lipidoids formulated and uses in delivering conjugates of the present invention. Complexes, micelles, liposomes or particles can be prepared containing these lipidoids and therefore, can result in an effective delivery of the conjugates of the present invention, as judged by the production of an encoded protein, following the injection of a lipidoid formulation via localized and/or systemic routes of administration. Lipidoid complexes of conjugates of the present invention can be administered by various means including, but not limited to, intravenous, intramuscular, or subcutaneous routes.

In vivo delivery of therapeutica agents may be affected by many parameters, including, but not limited to, the formulation composition, nature of particle PEGylation, degree of loading, drug to lipid ratio, and biophysical parameters such as, but not limited to, particle size (Akinc et al., Mol Ther. 2009 17:872-879; herein incorporated by reference in its entirety). As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 401:61 (2010); herein incorporated by reference in its entirety), C12-200 (including derivatives and variants), and MD1, can be tested for in vivo activity.

The lipidoid referred to herein as “98N12-5” is disclosed by Akinc et al., Mol Ther. 2009 17:872-879 and is incorporated by reference in its entirety.

The lipidoid referred to herein as “C12-200” is disclosed by Love et al., Proc Natl Acad Sci USA. 2010 107:1864-1869 and Liu and Huang, Molecular Therapy. 2010 669-670 (see FIG. 1); both of which are herein incorporated by reference in their entirety. The lipidoid formulations can include particles comprising either 3 or 4 or more components in addition to conjugates of the present invention. As an example, formulations with certain lipidoids, include, but are not limited to, 98N12-5 and may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In one embodiment, conjugates of the present invention formulated with a lipidoid for systemic intravenous administration can target the liver. For example, a final optimized intravenous formulation using conjugates of the present invention, and comprising a lipid molar composition of 42% 98N12-5, 48% cholesterol, and 10% PEG-lipid with a final weight ratio of about 7.5 to 1 total lipid to conjugates, and a C14 alkyl chain length on the PEG lipid, with a mean particle size of roughly 50-60 nm, can result in the distribution of the formulation to be greater than 90% to the liver (see, Akine et al., Mol Ther. 2009 17:872-879; herein incorporated by reference in its entirety). In another example, an intravenous formulation using a C12-200 (see U.S. provisional application 61/175,770 and published international application WO2010129709, each of which is herein incorporated by reference in their entirety) lipidoid may have a molar ratio of 50/10/38.5/1.5 of C12-200/disteroylphosphatidyl choline/cholesterol/PEG-DMG, with a weight ratio of 7 to 1 total lipid to conjugates, and a mean particle size of 80 nm may be effective to deliver conjugates of the present invention to hepatocytes (see, Love et al., Proc Natl Acad Sci USA. 2010 107:1864-1869 herein incorporated by reference in its entirety). In another embodiment, an MD1 lipidoid-containing formulation may be used to effectively deliver conjugates of the present invention to hepatocytes in vivo. The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see, Akinc et al., Mol Ther. 2009 17:872-879 herein incorporated by reference in its entirety), use of a lipidoid-formulated conjugates to deliver the formulation to other cells types including, but not limited to, endothelial cells, myeloid cells, and muscle cells may not be similarly size-limited. Use of lipidoid formulations to deliver therapeutic agents in vivo to other non-hepatocyte cells such as myeloid cells and endothelium has been reported (see Akinc et al., Nat Biotechnol. 2008 26:561-569; Leuschner et al., Nat Biotechnol. 2011 29:1005-1010; Cho et al. Adv. Funct. Mater. 2009 19:3112-3118; 8^{th} International Judah Folkman Conference, Cambridge, Mass. Oct. 8-9, 2010; each of which is herein incorporated by reference in its entirety). Effective delivery to myeloid cells, such as monocytes, lipidoid formulations may have a similar component molar ratio. Different ratios of lipidoids and other components including, but not limited to, disteroylphosphatidyl choline, cholesterol and PEG-DMG, may be used to optimize the formulation of the conjugates for delivery to different cell types including, but not limited to, hepatocytes, myeloid cells, muscle cells, etc. For example, the component molar ratio may include, but is not limited to, 50% C12-200, 10% disteroylphosphatidyl choline, 38.5% cholesterol, and %1.5 PEG-DMG (see Leuschner et al., Nat Biotechnol 2011 29:1005-1010; herein incorporated by reference in its entirety). The use of lipidoid formulations for the localized delivery of conjugates to cells (such as, but not limited to, adipose cells and muscle cells) via either subcutaneous or intramuscular delivery, may not require all of the formulation components desired for systemic delivery, and as such may comprise only the lipidoid and the conjugates.

Liposomes, Lipoplexes, and Lipid Nanoparticles

The conjugates of the invention can be formulated using one or more liposomes, lipoplexes, or lipid nanoparticles. In one embodiment, pharmaceutical compositions of the conjugates of the invention include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.). The original manufacture method by Wheeler et al. was a detergent dialysis method, which was later improved by Jeffs et al. and is referred to as the spontaneous vesicle formation method. The liposome formulations are composed of 3 to 4 lipid components in addition to the conjugates of the invention. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In one embodiment, the conjugates of the invention may be formulated in a lipid vesicle which may have crosslinks between functionalized lipid bilayers.

In one embodiment, the the conjugates of the invention may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326; herein incorporated by reference in its entirety. In another embodiment, the conjugates of the invention may be formulated in a lipid-polycation complex which may further include a neutral lipid such as, but not limited to, cholesterol or dioleoyl phosphatidylethanolamine (DOPE).

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain 1-5% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol) or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871; each of which is herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365 and WO2012044638; each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. 0520100036115; each of which is herein incorporated by reference in their entirety. As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-1 6, 19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl} dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoylo ctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In one embodiment, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724 and WO201021865; each of which is herein incorporated by reference in their entirety.

In one embodiment, the LNP formulation may contain PEG-c-DOMG at 3% lipid molar ratio. In another embodiment, the LNP formulation may contain PEG-c-DOMG at 1.5% lipid molar ratio.

In one embodiment, the LNP formulation may contain PEG-DMG 2000 (1,2-dimyristoyl-sn-glycero-3-phophoethanolamine-N-[methoxy(polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g. Geall et al., Nonviral delivery of self-amplifying RNA vaccines, PNAS 2012; PMID: 22908294; herein incorporated by reference in its entirety).

In one embodiment, the LNP formulation may be formulated by the methods described in International Publication Nos. WO2011127255 or WO2008103276, each of which is herein incorporated by reference in their entirety. As a non-limiting example, modified RNA described herein may be encapsulated in LNP formulations as described in WO2011127255 and/or WO2008103276; each of which is herein incorporated by reference in their entirety. As another non-limiting example, modified RNA described herein may be formulated in a nanoparticle to be delivered by a parenteral route as described in U.S. Pub. No. 20120207845; herein incorporated by reference in its entirety.

In one embodiment, LNP formulations described herein may comprise a polycationic composition. As a non-limiting example, the polycationic composition may be selected from formula 1-60 of US Patent Publication No. US20050222064; herein incorporated by reference in its entirety. In another embodiment, the LNP formulations comprising a polycationic composition may be used for the delivery of the modified RNA described herein in vivo and/or in vitro.

In one embodiment, the LNP formulations described herein may additionally comprise a permeability enhancer molecule. Non-limiting permeability enhancer molecules are described in US Patent Publication No. US20050222064; herein incorporated by reference in its entirety.

In one embodiment, the pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

The nanoparticle formulations may be a carbohydrate nanoparticle comprising a carbohydrate carrier and a modified nucleic acid molecule (e.g., mmRNA). As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; herein incorporated by reference in its entirety).

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In one embodiment, the internal ester linkage may be located on either side of the saturated carbon. Non-limiting examples of reLNPs include,

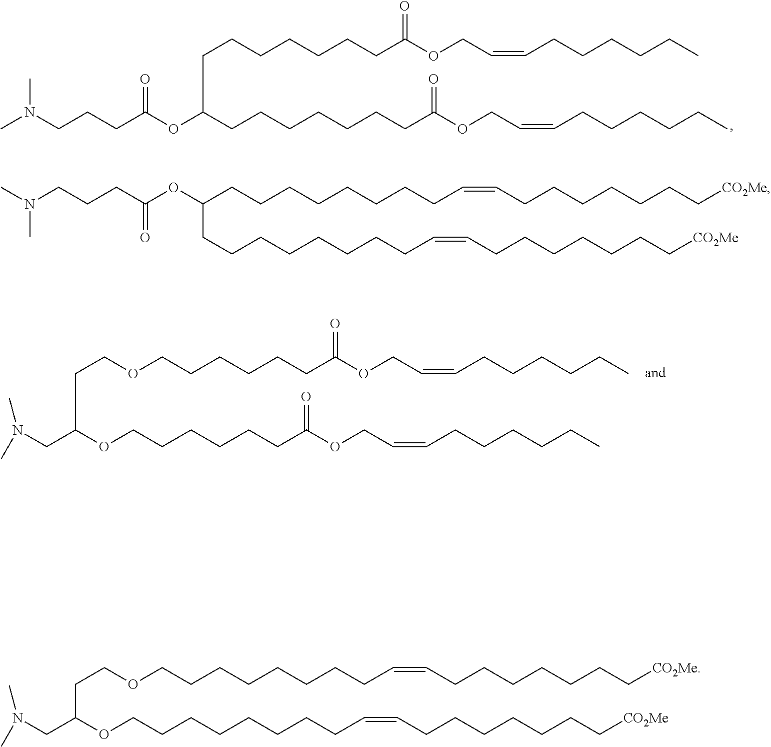

Lipid nanoparticles may be engineered to alter the surface properties of particles so the lipid nanoparticles may penetrate the mucosal barrier. Mucus is located on mucosal tissue such as, but not limted to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosla tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670, herein incorporated by reference in its entirety.

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. The polymeric material may additionally be irradiated. As a non-limiting example, the polymeric material may be gamma irradiated (See e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), and trimethylene carbonate, polyvinylpyrrolidone. The lipid nanoparticle may be coated or associated with a co-polymer such as, but not limited to, a block co-polymer, and (poly(ethylene glycol))-(poly(propylene oxide))-(poly(ethylene glycol)) triblock copolymer (see e.g., US Publication 20120121718 and US Publication 20100003337 and U.S. Pat. No. 8,263,665; each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, mmRNA, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., US Publication 20100215580 and US Publication 20080166414; each of which is herein incorporated by reference in their entirety).

The mucus penetrating lipid nanoparticles may comprise at least one conjugate described herein. The conjugate may be encapsulated in the lipid nanoparticle and/or disposed on the surface of the paricle. The conjugate may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion which may increase the delivery of the mucus penetrating lipid nanoparticles to the mucosal tissue.

In one embodiment, the conjugate of the invention is formulated as a lipoplex, such as, without limitation, the ATUPLEX′ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of therapeutic agents (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; all of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364; herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; all of which are incorporated herein by reference in its entirety).

In one embodiment, the conjugates of the invention are formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; herein incorporated by reference in its entirety).

In one embodiment, the conjugates of the invention can be formulated for controlled release and/or targeted delivery. As used herein, “controlled release” refers to a pharmaceutical composition or compound release profile that conforms to a particular pattern of release to effect a therapeutic outcome. In one embodiment, the conjugates of the invention may be encapsulated into a delivery agent described herein and/or known in the art for controlled release and/or targeted delivery. As used herein, the term “encapsulate” means to enclose, surround or encase. As it relates to the formulation of the conjugates of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of conjugate of the invention may be enclosed, surrounded or encased within the particle. “Partially encapsulation” means that less than 10, 10, 20, 30, 40 50 or less of the conjugate of the invention may be enclosed, surrounded or encased within the particle. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the particle.

In one embodiment, the controlled release formulation may include, but is not limited to, tri-block co-polymers. As a non-limiting example, the formulation may include two different types of tri-block co-polymers (International Pub. No. WO2012131104 and WO2012131106; each of which is herein incorporated by reference in its entirety).

In another embodiment, the conjugates of the invention may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In another embodiment, the lipid nanoparticle may be encapsulated into any polymer known in the art which may form a gel when injected into a subject. As a non-limiting example, the lipid nanoparticle may be encapsulated into a polymer matrix which may be biodegradable.

In one embodiment, the conjugate formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the conjugate of the present invention may be encapsulated in a therapeutic nanoparticle. Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286 and US20120288541, and U.S. Pat. Nos. 8,206,747, 8,293,276 8,318,208 and 8,318,211; each of which is herein incorporated by reference in their entirety. In another embodiment, therapeutic polymer nanoparticles may be identified by the methods described in US Pub No. US20120140790, herein incorporated by reference in its entirety.

In one embodiment, the therapeutic nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the conjugate of the present invention (see International Pub No. 2010075072 and US Pub No. US20100216804, US20110217377 and US20120201859, each of which is herein incorporated by reference in their entirety).

In one embodiment, the therapeutic nanoparticles may be formulated to be target specific. As a non-limiting example, the thereapeutic nanoparticles may include a corticosteroid (see International Pub. No. WO2011084518 herein incorporated by reference in its entirety). In one embodiment, the therapeutic nanoparticles of the present invention may be formulated to be cancer specific. As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, each of which is herein incorporated by reference in their entirety.

In one embodiment, the nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one embodiment, the therapeutic nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

As a non-limiting example the therapeutic nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968, herein incorporated by reference in its entirety).

In one embodiment, the therapeutic nanoparticle may comprise a multiblock copolymer (See e.g., U.S. Pat. Nos. 8,263,665 and 8,287,910; each of which is herein incorporated by reference in its entirety).

In one embodiment, the block copolymers described herein may be included in a polyion complex comprising a non-polymeric micelle and the block copolymer. (See e.g., U.S. Pub. No. 20120076836; herein incorporated by reference in its entirety).

In one embodiment, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the therapeutic nanoparticles may comprise at least one cationic polymer described herein and/or known in the art.

In one embodiment, the therapeutic nanoparticles may comprise at least one amine-containing polymer such as, but not limited to polylysine, polyethylene imine, poly(amidoamine) dendrimers, poly(beta-amino esters) (See e.g., U.S. Pat. No. 8,287,849; herein incorporated by reference in its entirety) and combinations thereof.

In one embodiment, the therapeutic nanoparticles may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In another embodiment, the therapeutic nanoparticle may include a conjugation of at least one targeting ligand. The targeting ligand may be any ligand known in the art such as, but not limited to, a monoclonal antibody. (Kirpotin et al, Cancer Res. 2006 66:6732-6740; herein incorporated by reference in its entirety).

In one embodiment, the therapeutic nanoparticle may be formulated in an aqueous solution which may be used to target cancer (see International Pub No. WO2011084513 and US Pub No. US20110294717, each of which is herein incorporated by reference in their entirety).

In one embodiment, the conjugates of the invention may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411 and WO2012149454 and US Pub. Nos. US20110262491, US20100104645, US20100087337 and US20120244222, each of which is herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US20120244222, each of which is herein incorporated by reference in their entirety. In another embodiment, the synthetic nanocarrier formulations may be lyophilized by methods described in International Pub. No. WO2011072218 and U.S. Pat. No. 8,211,473; each of which is herein incorporated by reference in their entirety.

In one embodiment, the synthetic nanocarriers may contain reactive groups to release the conjugates described herein (see International Pub. No. WO20120952552 and US Pub No. US20120171229, each of which is herein incorporated by reference in their entirety).

In one embodiment, the synthetic nanocarriers may be formulated for targeted release. In one embodiment, the synthetic nanocarrier is formulated to release the conjugates at a specified pH and/or after a desired time interval. As a non-limiting example, the synthetic nanoparticle may be formulated to release the conjugates after 24 hours and/or at a pH of 4.5 (see International Pub. Nos. WO2010138193 and WO2010138194 and US Pub Nos. US20110020388 and US20110027217, each of which is herein incorporated by reference in their entirety).

In one embodiment, the synthetic nanocarriers may be formulated for controlled and/or sustained release of conjugates described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In one embodiment, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343; herein incorporated by reference in its entirety.

Polymers, Biodegradable Nanoparticles, and Core-Shell Nanoparticles

The conjugates of the invention can be formulated using natural and/or synthetic polymers. Non-limiting examples of polymers which may be used for delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY™ (Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers, RONDEL™ (RNAi/Oligonucleotide Nanoparticle Delivery) polymers (Arrowhead Research Corporation, Pasadena, Calif.) and pH responsive co-block polymers such as, but not limited to, PHASERX™ (Seattle, Wash.).

A non-limiting example of chitosan formulation includes a core of positively charged chitosan and an outer portion of negatively charged substrate (U.S. Pub. No. 20120258176; herein incorporated by reference in its entirety). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

In one embodiment, the polymers used in the present invention have undergone processing to reduce and/or inhibit the attachement of unwanted substances such as, but not limited to, bacteria, to the surface of the polymer. The polymer may be processed by methods known and/or described in the art and/or described in International Pub. No. WO2012150467, herein incorporated by reference in its entirety.

A non-limiting example of PLGA formulations include, but are not limited to, PLGA injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space).

Many of these polymer approaches have demonstrated efficacy in delivering therapeutic agents in vivo into the cell cytoplasm (reviewed in deFougerolles Hum Gene Ther. 2008 19:125-132; herein incorporated by reference in its entirety). Two polymer approaches that have yielded robust in vivo delivery of nucleic acids, in this case with small interfering RNA (siRNA), are dynamic polyconjugates and cyclodextrin-based nanoparticles. The first of these delivery approaches uses dynamic polyconjugates and has been shown in vivo in mice to effectively deliver siRNA and silence endogenous target mRNA in hepatocytes (Rozema et al., Proc Natl Acad Sci USA. 2007 104:12982-12887; herein incorporated by reference in its entirety). This particular approach is a multicomponent polymer system whose key features include a membrane-active polymer to which nucleic acid, in this case siRNA, is covalently coupled via a disulfide bond and where both PEG (for charge masking) and N-acetylgalactosamine (for hepatocyte targeting) groups are linked via pH-sensitive bonds (Rozema et al., Proc Natl Acad Sci USA. 2007 104:12982-12887; herein incorporated by reference in its entirety). On binding to the hepatocyte and entry into the endosome, the polymer complex disassembles in the low-pH environment, with the polymer exposing its positive charge, leading to endosomal escape and cytoplasmic release of the siRNA from the polymer. Through replacement of the N-acetylgalactosamine group with a mannose group, it was shown one could alter targeting from asialoglycoprotein receptor-expressing hepatocytes to sinusoidal endothelium and Kupffer cells. Another polymer approach involves using transferrin-targeted cyclodextrin-containing polycation nanoparticles. These nanoparticles have demonstrated targeted silencing of the EWS-FLI1 gene product in transferrin receptor-expressing Ewing's sarcoma tumor cells (Hu-Lieskovan et al., Cancer Res. 2005 65: 8984-8982; herein incorporated by reference in its entirety) and siRNA formulated in these nanoparticles was well tolerated in non-human primates (Heidel et al., Proc Natl Acad Sci USA 2007 104:5715-21; herein incorporated by reference in its entirety). Both of these delivery strategies incorporate rational approaches using both targeted delivery and endosomal escape mechanisms.

The polymer formulation can permit the sustained or delayed release of the conjugates of the invention (e.g., following intramuscular or subcutaneous injection). The polymer formulation may also be used to increase the stability of the conjugate. Biodegradable polymers have been previously used to protect conjugates from degradation and been shown to result in sustained release of payloads in vivo (Rozema et al., Proc Natl Acad Sci USA. 2007 104:12982-12887; Sullivan et al., Expert Opin Drug Deliv. 2010 7:1433-1446; Convertine et al., Biomacromolecules. 2010 Oct. 1; Chu et al., Acc Chem Res. 2012 Jan. 13; Manganiello et al., Biomaterials. 2012 33:2301-2309; Benoit et al., Biomacromolecules. 2011 12:2708-2714; Singha et al., Nucleic Acid Ther. 2011 2:133-147; deFougerolles Hum Gene Ther. 2008 19:125-132; Schaffert and Wagner, Gene Ther. 2008 16:1131-1138; Chaturvedi et al., Expert Opin Drug Deliv. 2011 8:1455-1468; Davis, Mol Pharm. 2009 6:659-668; Davis, Nature 2010 464:1067-1070; each of which is herein incorporated by reference in its entirety).

In one embodiment, the pharmaceutical compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

As a non-limiting example modified mRNA may be formulated in PLGA microspheres by preparing the PLGA microspheres with tunable release rates (e.g., days and weeks) and encapsulating the modified mRNA in the PLGA microspheres while maintaining the integrity of the modified mRNA during the encapsulation process. EVAc are non-biodegradeable, biocompatible polymers which are used extensively in pre-clinical sustained release implant applications (e.g., extended release products Ocusert a pilocarpine ophthalmic insert for glaucoma or progestasert a sustained release progesterone intrauterine deivce; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic ineraction to provide a stabilizing effect.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009 6:659-668; Davis, Nature 2010 464:1067-1070; each of which is herein incorporated by reference in its entirety).

The conjugates of the invention may be formulated with or in a polymeric compound. The polymer may include at least one polymer such as, but not limited to, polyethenes, polyethylene glycol (PEG), poly(l-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

As a non-limiting example, the conjugate of the invention may be formulated with the polymeric compound of PEG grafted with PLL as described in U.S. Pat. No. 6,177,274; herein incorporated by reference in its entirety. In another example, the conjugate may be suspended in a solution or medium with a cationic polymer, in a dry pharmaceutical composition or in a solution that is capable of being dried as described in U.S. Pub. Nos. 20090042829 and 20090042825; each of which are herein incorporated by reference in their entireties.

As another non-limiting example the conjugate of the invention may be formulated with a PLGA-PEG block copolymer (see US Pub. No. US20120004293 and U.S. Pat. No. 8,236,330, each of which are herein incorporated by reference in their entireties) or PLGA-PEG-PLGA block copolymers (See U.S. Pat. No. 6,004,573, herein incorporated by reference in its entirety). As a non-limiting example, the conjugate of the invention may be formulated with a diblock copolymer of PEG and PLA or PEG and PLGA (see U.S. Pat. No. 8,246,968, herein incorporated by reference in its entirety).

A polyamine derivative may be used to deliver conjugates of the invention or to treat and/or prevent a disease or to be included in an implantable or injectable device (U.S. Pub. No. 20100260817 herein incorporated by reference in its entirety). As a non-limiting example, a pharmaceutical composition may include the conjugates of the invention and the polyamine derivative described in U.S. Pub. No. 20100260817 (the contents of which are incorporated herein by reference in its entirety). As a non-limiting example the conjugates of the invention may be delivered using a polyaminde polymer such as, but not limited to, a polymer comprising a 1,3-dipolar addition polymer prepared by combining a carbohydrate diazide monomer with a dilkyne unite comprising oligoamines (U.S. Pat. No. 8,236,280; herein incorporated by reference in its entirety).

The conjugate of the invention may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the conjugates of the invention may be formulated with at least one polymer and/or derivatives thereof described in International Publication Nos. WO2011115862, WO2012082574 and WO2012068187 and U.S. Pub. No. 20120283427, each of which are herein incorporated by reference in their entireties. In another embodiment, the conjugates of the invention may be formulated with a polymer of formula Z as described in WO2011115862, herein incorporated by reference in its entirety. In yet another embodiment, the conjugates of the invention may be formulated with a polymer of formula Z, Z′ or Z″ as described in International Pub. Nos. WO2012082574 or WO2012068187, each of which are herein incorporated by reference in their entireties. The polymers formulated with the conjugates of the present invention may be synthesized by the methods described in International Pub. Nos. WO2012082574 or WO2012068187, each of which are herein incorporated by reference in their entireties.

Formulations of conjugates of the invention may include at least one amine-containing polymer such as, but not limited to polylysine, polyethylene imine, poly(amidoamine) dendrimers or combinations thereof.

For example, the conjugate of the invention may be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof. The biodegradable cationic lipopolymer may be made by methods known in the art and/or described in U.S. Pat. No. 6,696,038, U.S. App. Nos. 20030073619 and 20040142474 each of which is herein incorporated by reference in their entireties. The poly(alkylene imine) may be made using methods known in the art and/or as described in U.S. Pub. No. 20100004315, herein incorporated by reference in its entirety. The biodegradabale polymer, biodegradable block copolymer, the biodegradable random copolymer, biodegradable polyester block copolymer, biodegradable polyester polymer, or biodegradable polyester random copolymer may be made using methods known in the art and/or as described in U.S. Pat. Nos. 6,517,869 and 6,267,987, the contents of which are each incorporated herein by reference in their entirety. The linear biodegradable copolymer may be made using methods known in the art and/or as described in U.S. Pat. No. 6,652,886. The PAGA polymer may be made using methods known in the art and/or as described in U.S. Pat. No. 6,217,912 herein incorporated by reference in its entirety. The PAGA polymer may be copolymerized to form a copolymer or block copolymer with polymers such as but not limited to, poly-L-lysine, polyargine, polyornithine, histones, avidin, protamines, polylactides and poly(lactide-co-glycolides). The biodegradable cross-linked cationic multi-block copolymers may be made my methods known in the art and/or as described in U.S. Pat. No. 8,057,821 or U.S. Pub. No. 2012009145 each of which are herein incorporated by reference in their entireties. For example, the multi-block copolymers may be synthesized using linear polyethyleneimine (LPEI) blocks which have distinct patterns as compared to branched polyethyleneimines. Further, the composition or pharmaceutical composition may be made by the methods known in the art, described herein, or as described in U.S. Pub. No. 20100004315 or U.S. Pat. Nos. 6,267,987 and 6,217,912 each of which are herein incorporated by reference in their entireties.

The conjugates of the invention may be formulated with at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

The conjugate of the invention may be formulated with at least one crosslinkable polyester. Crosslinkable polyesters include those known in the art and described in US Pub. No. 20120269761, herein incorporated by reference in its entirety.

In one embodiment, the polymers described herein may be conjugated to a lipid-terminating PEG. As a non-limiting example, PLGA may be conjugated to a lipid-terminating PEG forming PLGA-DSPE-PEG. As another non-limiting example, PEG conjugates for use with the present invention are described in International Publication No. WO2008103276, herein incorporated by reference in its entirety. The polymers may be conjugated using a ligand conjugate such as, but not limited to, the conjugates described in U.S. Pat. No. 8,273,363, herein incorporated by reference in its entirety.

In one embodiment, the conjugates of the invention may be conjugated with another compound. Non-limiting examples of conjugates are described in U.S. Pat. Nos. 7,964,578 and 7,833,992, each of which are herein incorporated by reference in their entireties. In another embodiment, the conjugates of the invention may be conjugated with conjugates of formula 1-122 as described in U.S. Pat. Nos. 7,964,578 and 7,833,992, each of which are herein incorporated by reference in their entireties. The modified RNA described herein may be conjugated with a metal such as, but not limited to, gold. (See e.g., Giljohann et al. Journ. Amer. Chem. Soc. 2009 131(6): 2072-2073; herein incorporated by reference in its entirety). In another embodiment, the conjugates of the invention may be conjugated and/or encapsulated in gold-nanoparticles. (Interantional Pub. No. WO201216269 and U.S. Pub. No. 20120302940; each of which is herein incorporated by reference in its entirety).

In one embodiment, the polymer formulation of the present invention may be stabilized by contacting the polymer formulation, which may include a cationic carrier, with a cationic lipopolymer which may be covalently linked to cholesterol and polyethylene glycol groups. The polymer formulation may be contacted with a cationic lipopolymer using the methods described in U.S. Pub. No. 20090042829 herein incorporated by reference in its entirety. The cationic carrier may include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B—[N—(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof.

The conjugates of the invention may be formulated in a polyplex of one or more polymers (U.S. Pub. No. 20120237565 and 20120270927; each of which is herein incorporated by reference in its entirety). In one embodiment, the polyplex comprises two or more cationic polymers. The catioinic polymer may comprise a poly(ethylene imine) (PEI) such as linear PEI.

The conjugates of the invention can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so that delivery of the conjugates of the invention may be enhanced (Wang et al., Nat Mater. 2006 5:791-796; Fuller et al., Biomaterials. 2008 29:1526-1532; DeKoker et al., Adv Drug Deliv Rev. 2011 63:748-761; Endres et al., Biomaterials. 2011 32:7721-7731; Su et al., Mol Pharm. 2011 Jun. 6; 8(3):774-87; each of which is herein incorporated by reference in its entirety). As a non-limiting example, the nanoparticle may comprise a plurality of polymers such as, but not limited to hydrophilic-hydrophobic polymers (e.g., PEG-PLGA), hydrophobic polymers (e.g., PEG) and/or hydrophilic polymers (International Pub. No. WO20120225129; herein incorporated by reference in its entirety).

Biodegradable calcium phosphate nanoparticles in combination with lipids and/or polymers have been shown to deliver therapeutic agents in vivo. In one embodiment, a lipid coated calcium phosphate nanoparticle, which may also contain a targeting ligand such as anisamide, may be used to deliver the conjugate of the present invention. For example, to effectively deliver a therapeutic agent in a mouse metastatic lung model a lipid coated calcium phosphate nanoparticle was used (Li et al., J Contr Rel. 2010 142: 416-421; Li et al., J Contr Rel. 2012 158:108-114; Yang et al., Mol Ther. 2012 20:609-615; herein incorporated by refereince in its entirety). This delivery system combines both a targeted nanoparticle and a component to enhance the endosomal escape, calcium phosphate, in order to improve delivery of the therapeutic agent.

In one embodiment, calcium phosphate with a PEG-polyanion block copolymer may be used to deliver modified nucleic acid molecules and mmRNA (Kazikawa et al., J Contr Rel. 2004 97:345-356; Kazikawa et al., J Contr Rel. 2006 111:368-370; herein incorporated by reference in its entirety).

In one embodiment, a PEG-charge-conversional polymer (Pitella et al., Biomaterials. 2011 32:3106-3114) may be used to form a nanoparticle to deliver the conjugate of the present invention. The PEG-charge-conversional polymer may improve upon the PEG-polyanion block copolymers by being cleaved into a polycation at acidic pH, thus enhancing endosomal escape.

The use of core-shell nanoparticles has additionally focused on a high-throughput approach to synthesize cationic cross-linked nanogel cores and various shells (Siegwart et al., Proc Natl Acad Sci USA. 2011 108:12996-13001). The complexation, delivery, and internalization of the polymeric nanoparticles can be precisely controlled by altering the chemical composition in both the core and shell components of the nanoparticle. For example, the core-shell nanoparticles may efficiently deliver a therapeutic agent to mouse hepatocytes after they covalently attach cholesterol to the nanoparticle.

In one embodiment, a hollow lipid core comprising a middle PLGA layer and an outer neutral lipid layer containg PEG may be used to delivery of the conjguate of the present invention. As a non-limiting example, in mice bearing a luciferease-expressing tumor, it was determined that the lipid-polymer-lipid hybrid nanoparticle significantly suppressed luciferase expression, as compared to a conventional lipoplex (Shi et al, Angew Chem Int Ed. 2011 50:7027-7031; herein incorporated by reference in its entirety).

In one embodiment, the lipid nanoparticles may comprise a core of the conjugates disclosed herein and a polymer shell. The polymer shell may be any of the polymers described herein and are known in the art. In an additional embodiment, the polymer shell may be used to protect the modified nucleic acids in the core.

Core-shell nanoparticles for use with the conjugates of the present invention are described and may be formed by the methods described in U.S. Pat. No. 8,313,777 herein incorporated by reference in its entirety.

In one embodiment, the core-shell nanoparticles may comprise a core of the conjugates disclosed herein and a polymer shell. The polymer shell may be any of the polymers described herein and are known in the art. In an additional embodiment, the polymer shell may be used to protect the modified nucleic acid molecules in the core.

Peptides and Proteins

The conjugate of the invention can be formulated with peptides and/or proteins in order to increase peneration of cells by the conjugates of the invention. In one embodiment, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention include a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3(3):310-8 (2001); Langel, Cell-Penetrating Peptides: Processes and Applications (CRC Press, Boca Raton Fla., 2002); E1-Andaloussi et al., Curr. Pharm. Des. 11(28):3597-611 (2003); and Deshayes et al., Cell. Mol. Life Sci. 62(16):1839-49 (2005), all of which are incorporated herein by reference). The compositions can also be formulated to include a cell penetrating agent, e.g., liposomes, which enhance delivery of the compositions to the intracellular space. The conjugates of the invention may be complexed to peptides and/or proteins such as, but not limited to, peptides and/or proteins from Aileron Therapeutics (Cambridge, Mass.) and Permeon Biologics (Cambridge, Mass.) in order to enable intracellular delivery (Cronican et al., ACS Chem. Biol. 2010 5:747-752; McNaughton et al., Proc. Natl. Acad. Sci. USA 2009 106:6111-6116; Sawyer, Chem Biol Drug Des. 2009 73:3-6; Verdine and Hilinski, Methods Enzymol. 2012; 503:3-33; all of which are herein incorporated by reference in its entirety).

In one embodiment, the cell-penetrating polypeptide may comprise a first domain and a second domain. The first domain may comprise a supercharged polypeptide. The second domain may comprise a protein-binding partner. As used herein, “protein-binding partner” includes, but are not limited to, antibodies and functional fragments thereof, scaffold proteins, or peptides. The cell-penetrating polypeptide may further comprise an intracellular binding partner for the protein-binding partner. The cell-penetrating polypeptide may be capable of being secreted from a cell where conjugates of the invention may be introduced.

Administration

The conjugates and/or particles comprising such conjugates of the present invention may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier.

The formulations described herein contain an effective amount of conjugates and/or particles comprising such conjugates in a pharmaceutical carrier appropriate for administration to an individual in need thereof. The may be administered parenterally (e.g., by injection or infusion). The formulations or variations thereof may be administered in any manner including enterally, topically (e.g., to the eye), or via pulmonary administration. In some embodiments the formulations are administered topically.

A. Parenteral Formulations

The conjugates and/or particles comprising such conjugates can be formulated for parenteral delivery, such as injection or infusion, in the form of a solution, suspension or emulsion. The formulation can be administered systemically, regionally or directly to the organ or tissue to be treated.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the conjugates and/or particles comprising such conjugates can be prepared in water or another solvent or dispersing medium suitably mixed with one or more pharmaceutically acceptable excipients including, but not limited to, surfactants, dispersants, emulsifiers, pH modifying agents, and combinations thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-1501aurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s), conjugates and/or particles comprising such conjugates.

The formulation is typically buffered to a pH of 3-8 for parenteral administration upon reconstitution. Suitable buffers include, but are not limited to, phosphate buffers, acetate buffers, and citrate buffers. If using 10% sucrose or 5% dextrose, a buffer may not be required.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the conjugates and/or particles comprising such conjugates in the required amount in the appropriate solvent or dispersion medium with one or more of the excipients listed above, as required, followed by filtered sterilization. Generally, dispersions are prepared by incorporating the various sterilized conjugates and/or particles comprising such conjugates into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the conjugates and/or particle comprising such conjugates plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration can be in the form of a sterile aqueous solution or suspension of conjugates and/or particles comprising such conjugates formed from one or more polymer-drug conjugates. Acceptable solvents include, for example, water, Ringer's solution, phosphate buffered saline (PBS), and isotonic sucrose, dextrose or sodium chloride solution. The formulation may also be a sterile solution, suspension, or emulsion in a nontoxic, parenterally acceptable diluent or solvent such as 1,3-butanediol.

In some instances, the formulation is distributed or packaged in a liquid form. Alternatively, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for parenteral administration may also contain one or more tonicity agents to adjust the isotonic range of the formulation. Suitable tonicity agents are well known in the art and some examples include glycerin, sucrose, dextrose, mannitol, sorbitol, sodium chloride, and other electrolytes.

Solutions, suspensions, or emulsions for parenteral administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for parenteral administration may also contain one or more excipients known art, such as dispersing agents, wetting agents, and suspending agents.

B. Mucosal Topical Formulations

The conjugates and/or particles comprising such conjugates can be formulated for topical administration to a mucosal surface Suitable dosage forms for topical administration include creams, ointments, salves, sprays, gels, lotions, emulsions, liquids, and transdermal patches. The formulation may be formulated for transmucosal transepithelial, or transendothelial administration. The compositions contain one or more chemical penetration enhancers, membrane permeability agents, membrane transport agents, emollients, surfactants, stabilizers, and combination thereof. In some embodiments, the conjugates and/or particles comprising such conjugates can be administered as a liquid formulation, such as a solution or suspension, a semi-solid formulation, such as a lotion or ointment, or a solid formulation. In some embodiments, the conjugates and/or particles comprising such conjugates are formulated as liquids, including solutions and suspensions, such as eye drops or as a semi-solid formulation, to the mucosa, such as the eye or vaginally or rectally.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the dispersion of one liquid in another and promote the formation of a stable mixture, or emulsion, of oil and water or water in oil. Common emulsifiers are: anaionic, cataionic and nonionic surfactants or micttures of surfactants, certain animal and vegetable oils, and various polar surface active compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

Dosing

The present invention provides methods comprising administering conjugates and/or particles containing the conjugate as described herein to a subject in need thereof. Conjugates and/or particles containing the conjugates as described herein may be administered to a subject using any amount and any route of administration effective for preventing or treating or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like.

Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

As used herein, a “split dose” is the division of single unit dose or total daily dose into two or more doses, e.g, two or more administrations of the single unit dose. As used herein, a “single unit dose” is a dose of any therapeutic administed in one dose/at one time/single route/single point of contact, i.e., single administration event. As used herein, a “total daily dose” is an amount given or prescribed in 24 hr period. It may be administered as a single unit dose. In one embodiment, the monomaleimide compounds of the present invention are administed to a subject in split doses. The monomaleimide compounds may be formulated in buffer only or in a formulation described herein.

Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, subcutaneous).

### Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

### Injectable

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the monomaleimide compounds then depends upon its rate of dissolution which, in turn, may depend upon crystal size and crystalline form. Alternatively, delayed absorption of a parenterally administered monomaleimide compound may be accomplished by dissolving or suspending the monomalimide in an oil vehicle. Injectable depot forms are made by forming microencapsule matrices of the monomaleimide compunds in biodegradable polymers such as polylactide-polyglycolide. Depending upon the ratio of monomaleimide compounds to polymer and the nature of the particular polymer employed, the rate of monomaleimide compound release can be controlled. Examples of other biodegradable polymers include, but are not limited to, poly(orthoesters) and poly(anhydrides). Depot injectable formulations may be prepared by entrapping the monomaleimide compounds in liposomes or microemulsions which are compatible with body tissues.

### Pulmonary

Formulations described herein as being useful for pulmonary delivery may also be used for intranasal delivery of a pharmaceutical composition. Another formulation suitable for intranasal administration may be a coarse powder comprising the active ingredient and having an average particle from about 0.2 um to 500 um. Such a formulation may be administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### IV. Methods of Making Conjugates

The conjugates can be made by many different synthetic procedures. The conjugates can be prepared from linkers having one or more reactive coupling groups or from one or more linker precursors capable of reacting with a reactive coupling group on an active agent or targeting moiety to form a covalent bond.

The conjugates can be prepared from a linker precursor capable of reacting with a reactive coupling group on an active agent or targeting moiety to form the linker covalently bonded to the active agent or targeting moiety.

The linker precursor can be a diacid or substituted diacid. Diacids, as used herein, can refer to substituted or unsubstituted alkyl, heteroalkyl, aryl, or heteroaryl compounds having two or more carboxylic acid groups, preferably having between 2 and 50, between 2 and 30, between 2 and 12, or between 2 and 8 carbon atoms. Suitable diacids can include oxalic acid, malonic acid, succinic acid, glutaric acid, adipic acid, pimelic acid, suberic acid, azelaic acid, sebacic acid, phthalic acid, iso-phthalic acid, terepthalic acid, and derivatives thereof.

The linker precursor can be an activated diacid derivative such as a diacid anhydride, diacid ester, or diacid halide. The diacid anhydride can be a cyclic anhydride obtained from the intramolecular dehydration of a diacid or diacid derivative such as those described above. The diacid anhydride can be malonic anhydride, succinic anhydride, glutaric anhydride, adipic anhydride, pimelic anhydride, phthalic anhydride, diglycolic anhydride, or a derivative thereof; preferably succinic anhydride, diglycolic anhydride, or a derivative thereof. The diacid ester can be an activated ester of any of the diacids described above, including methyl and butyl diesters or bis-(p-nitrophenyl) diesters of oxalic acid, malonic acid, succinic acid, glutaric acid, adipic acid, pimelic acid, suberic acid, azelaic acid, sebacic acid, phthalic acid, iso-phthalic acid, terepthalic acid, and derivatives thereof. The diacid halide can include the corresponding acid fluorides, acid chlorides, acid bromides, or acid iodides of the diacids described above. In preferred embodiments the diacid halide is succinyl chloride or diglycolyl chloride. For example, a therapeutic agent having a reactive (—OH) coupling group and a targeting moiety having a reactive (—NH2) coupling group can be used to prepare a conjugate having a disuccinate linker according to the following general scheme.

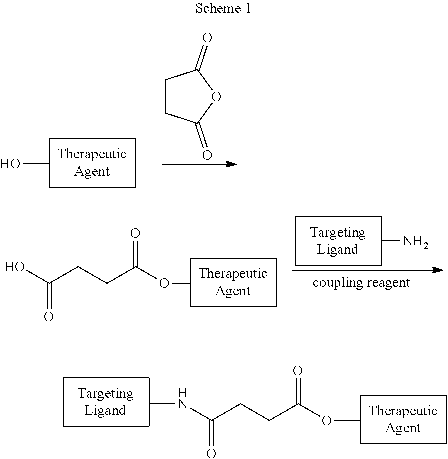

Referring to Scheme I above, the conjugates can be prepared by providing an active agent having a hydroxyl group and reacting it with a succinic anhydride linker precursor to form the conjugate of active agent-succinate-SSPy. A targeting moiety with an available —NH₂ group is reacted with a coupling reagent and the active agent-succinate-SSPy to form the targeting moiety-linker-active agent conjugate.

Other functional groups that can be linked to include, but are not limited to, —SH, —COOH, alkenyl, phosphate, sulfate, heterocyclic NH, alkyne and ketone.

The coupling reaction can be carried out under esterification conditions known to those of ordinary skill in the art such as in the presence of activating agents, e.g., carbodiimides (such as diisopropoylcarbodiimide (DIPC)), with or without catalyst such as dimethylaminopyridine (DMAP). This reaction can be carried out in an appropriate solvent, such as dichloromethane, chloroform or ethyl acetate, at a temperature or between about 0° C. and the reflux temperature of the solvent (e.g., ambient temperature). The coupling reaction is generally performed in a solvent such as pyridine or in a chlorinated solvent in the presence of a catalyst such as DMAP or pyridine at a temperature between about 0° C. and the reflux temperature of the solvent (e.g., ambient temperature). In preferred embodiments, the coupling reagent is selected from the group consisting of 4-(2-pyridyldithio)-butanoic acid, and a carbodiimide coupling reagent such as DCC in a chlorinated, ethereal or amidic solvent (such as N,N-dimethylformamide) in the presence of a catalyst such as DMAP at a temperature between about 0° C. and the reflux temperature of the solvent (e.g., ambient temperature).

The conjugates can be prepared by coupling an active agent and/or targeting moiety having one or more reactive coupling groups to a linker having complimentary reactive groups capable of reacting with the reactive coupling groups on the active agent or targeting moiety to form a covalent bond. For example, an active agent or targeting moiety having a primary amine group can be coupled to a linker having an isothiocyonate group or another amine-reactive coupling group. In some embodiments the linker contains a first reactive coupling group capable of reacting with a complimentary functional group on the active agent and a second reactive coupling group different from the first and capable of reacting with a complimentary group on the targeting moiety. In some embodiments one or both of the reactive coupling groups on the linker can be protected with a suitable protecting group during part of the synthesis.

In some embodiments, the conjguates of the invention may be synthesized with ‘click chemistry’ of the copper ion-catalyzed acetylene-azide cycloaddition reaction. For example, WO2010093395 to Govindan, the contents of which are incorporated herein by reference in their entirety, teaches that the targeting moiety comprises L2, wherein L2 comprises a targeting moiety-coupling end and one or more acetylene or azide groups at the other end. The active agent moiety comprises L1, wherein L1 comprises a defined PEG with azide or acetylene at one end, complementary to the acetylene or azide moiety in L2, and a reactive group such as carboxylic acid or hydroxyl group at the other end. ‘Click chemistry’ between L2 and L1 yields a conjugate comprising the targeting moiety and the active agent.

In some embodiments, the conjugates of the invention may be syntheized with thiol-ene ‘click chemistry’. For example, US 20130323169 to Xu et al., the contents of which are incorporated herein by reference in their entirety, teaches prepareing a drug conjugate by reacting a sulfhydryl or thiol group (—SH) on the targeting moiety with a double bond on the linker moiety.

### V. Methods of Making Particles

In various embodiments, a method of making the particles includes providing a conjugate; providing a base component such as PLA-PEG or PLGA-PEG, optionally mixed with PLA or PLGA, for forming a particle; combining the conjugate and the base component in an organic solution to form a first organic phase; and combining the first organic phase with a first aqueous solution to form a second phase; emulsifying the second phase to form an emulsion phase; and recovering particles. In various embodiments, the emulsion phase is further homogenized.

In some embodiments, the first phase includes about 5 to about 50% weight, e.g., about 1 to about 40% solids, or about 5 to about 30% solids, e.g. about 5%, 10%, 15%, and 20%, of the conjugate and the base component. In certain embodiments, the first phase includes about 5% weight of the conjugate and the base component. In various embodiments, the organic phase comprises acetonitrile, tetrahydrofuran, ethyl acetate, isopropyl alcohol, isopropyl acetate, dimethylformamide, methylene chloride, dichloromethane, chloroform, acetone, benzyl alcohol, TWEEN® 80, SPAN® 80, or a combination thereof. In some embodiments, the organic phase includes benzyl alcohol, ethyl acetate, or a combination thereof.

In various embodiments, the aqueous solution includes water, sodium cholate, ethyl acetate, and/or benzyl alcohol. In various embodiments, a surfactant or a surfactant mixture is added into the first phase, the second phase, or both. A surfactant, in some instances, can act as an emulsifier or a stabilizer for a composition disclosed herein. A suitable surfactant can be a cationic surfactant, an anionic surfactant, or a nonionic surfactant. In some embodiments, a surfactant suitable for making a composition described herein includes sorbitan fatty acid esters, polyoxyethylene sorbitan fatty acid esters and polyoxyethylene stearates. Examples of such fatty acid ester nonionic surfactants are the TWEEN® 80, SPAN® 80- and MYJ® surfactants from ICI. SPAN® surfactants include C₁₂-Cis sorbitan monoesters. TWEEN® surfactants include poly(ethylene oxide) C₁₂-Cis sorbitan monoesters. MYJ® surfactants include poly(ethylene oxide) stearates. In certain embodiments, the aqueous solution also comprises a surfactant (e.g., an emulsifier), including a polysorbate. For example, the aqueous solution can include polysorbate 80. In some embodiments, a suitable surfactant includes a lipid-based surfactant. For example, the composition can include 1,2-dihexanoyl-sn-glycero-3-phosphocholine, 1,2-diheptanoyl-sn-glycero-3-phosphocholine, PEGlyated 1,2-distearoyl-sn-glycero-3-phosphoethanolamine (including PEG5000-DSPE), PEGlyated 1,2-dioleoyl-sn-glycero-3-phosphoethanolamine (including 1,2-dioleoyl-sn-glycero-3-phosphoethanolamine-N-[methoxy(polyethylene glycol)-5000] (ammonium salt)).

Emulsifying the second phase to form an emulsion phase may be performed in one or two emulsification steps. For example, a primary emulsion may be prepared, and then emulsified to form a fine emulsion. The primary emulsion can be formed, for example, using simple mixing, a high pressure homogenizer, probe sonicator, stir bar, or a rotor stator homogenizer. The primary emulsion may be formed into a fine emulsion through the use of e.g. a probe sonicator or a high pressure homogenizer, e.g. by pass(es) through a homogenizer. For example, when a high pressure homogenizer (microfluidizer) is used, the pressure used may be about 1,000 to about 30,000 psi, about 4000 to about 10,000 psi, or 4000 or 5000 psi.

Either solvent evaporation or dilution may be needed to complete the extraction of the solvent and solidify the particles. For better control over the kinetics of extraction and a more scalable process, a solvent dilution via aqueous quench may be used. For example, the emulsion can be diluted into cold water to a concentration sufficient to dissolve all of the organic solvent to form a quenched phase. Quenching may be performed at least partially at a temperature of about 5° C. or less. For example, water used in the quenching may be at a temperature that is less that room temperature (e.g. about 0 to about 10° C., or about 0 to about 5° C.).

In various embodiments, the particles are purified and recovered by filtration. For example, ultrafiltration membranes can be used. Exemplary filtration may be performed using a tangential flow filtration system. For example, by using a membrane with a pore size suitable to retain particles while allowing solutes, micelles, and organic solvent to pass, particles can be selectively separated. Exemplary membranes with molecular weight cut-offs of about 100-500 kDa (−3-25 nm) may be used.

In various embodiments, the particles are freeze-dried or lyophilized, in some instances, to extend their shelf life. In some embodiments, the composition also includes a lyoprotectant. In certain embodiments, a lyoprotectant is selected from a sugar, a polyalcohol, or a derivative thereof. In some embodiments, a lyoprotectant is selected from a monosaccharide, a disaccharide, or a mixture thereof. For example, a lyoprotectant can be sucrose, lactulose, trehalose, lactose, glucose, maltose, mannitol, cellobiose, or a mixture thereof.

Methods of making particles containing one or more conjugates are provided. The particles can be polymeric particles, lipid particles, self-assembled particles, mixed michelles, or combinations thereof. The various methods described herein can be adjusted to control the size and composition of the particles, e.g. some methods are best suited for preparing microparticles while others are better suited for preparing particles. The selection of a method for preparing particles having the descried characteristics can be performed by the skilled artisan without undue experimentation.

i. Polymeric Particles

Methods of making polymeric particles are known in the art. Polymeric particles can be prepared using any suitable method known in the art. Common microencapsulation techniques include, but are not limited to, spray drying, interfacial polymerization, hot melt encapsulation, phase separation encapsulation (spontaneous emulsion microencapsulation, solvent evaporation microencapsulation, and solvent removal microencapsulation), coacervation, low temperature microsphere formation, and phase inversion nanoencapsulation (PIN). A brief summary of these methods is presented below.

1. Spray Drying

Methods for forming polymeric particles using spray drying techniques are described in U.S. Pat. No. 6,620,617. In this method, the polymer is dissolved in an organic solvent such as methylene chloride or in water. A known amount of one or more conjugates or additional active agents to be incorporated in the particles is suspended (in the case of an insoluble active agent) or co-dissolved (in the case of a soluble active agent) in the polymer solution. The solution or dispersion is pumped through a micronizing nozzle driven by a flow of compressed gas, and the resulting aerosol is suspended in a heated cyclone of air, allowing the solvent to evaporate from the microdroplets, forming particles. Microspheres/nanospheres ranging between 0.1 10 microns can be obtained using this method.

2. Interfacial Polymerization

Interfacial polymerization can also be used to encapsulate one or more conjugates and/or active agents. Using this method, a monomer and the conjugates or active agent(s) are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

3. Hot Melt Microencapsulation

Microspheres can be formed from polymers such as polyesters and polyanhydrides using hot melt microencapsulation methods as described in Mathiowitz et al., Reactive Polymers, 6:275 (1987). In this method, the use of polymers with molecular weights between 3,000-75,000 daltons is typical. In this method, the polymer first is melted and then mixed with the solid particles of one or more active agents to be incorporated that have been sieved to less than 50 microns. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microspheres are washed by decanting with petroleum ether to produce a free flowing powder.

4. Phase Separation Microencapsulation

In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

a. Spontaneous Emulsion Microencapsulation

Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

b. Solvent Evaporation Microencapsulation

Methods for forming microspheres using solvent evaporation techniques are described in Mathiowitz et al., J. Scanning Microscopy, 4:329 (1990); Beck et al., Fertil. Steril., 31:545 (1979); Beck et al., Am. J. Obstet. Gynecol. 135(3) (1979); Benita et al., J. Pharm. Sci., 73:1721 (1984); and U.S. Pat. No. 3,960,757. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microparticles/nanoparticles. This method is useful for relatively stable polymers like polyesters and polystyrene.

c. Solvent Removal Microencapsulation

The solvent removal microencapsulation technique is primarily designed for polyanhydrides and is described, for example, in WO 93/21906. In this method, the substance to be incorporated is dispersed or dissolved in a solution of the selected polymer in a volatile organic solvent, such as methylene chloride. This mixture is suspended by stirring in an organic oil, such as silicon oil, to form an emulsion. Microspheres that range between 1-300 microns can be obtained by this procedure. Substances which can be incorporated in the microspheres include pharmaceuticals, pesticides, nutrients, imaging agents, and metal compounds.

5. Coacervation

Encapsulation procedures for various substances using coacervation techniques are known in the art, for example, in GB-B-929 406; GB-B-929 40 1; and U.S. Pat. Nos. 3,266,987, 4,794,000, and 4,460,563. Coacervation involves the separation of a macromolecular solution into two immiscible liquid phases. One phase is a dense coacervate phase, which contains a high concentration of the polymer encapsulant (and optionally one or more active agents), while the second phase contains a low concentration of the polymer. Within the dense coacervate phase, the polymer encapsulant forms nanoscale or microscale droplets. Coacervation may be induced by a temperature change, addition of a non-solvent or addition of a micro-salt (simple coacervation), or by the addition of another polymer thereby forming an interpolymer complex (complex coacervation).

6. Low Temperature Casting of Microspheres

Methods for very low temperature casting of controlled release particles are described in U.S. Pat. No. 5,019,400. In this method, a polymer is dissolved in a solvent optionally with one or more dissolved or dispersed active agents. The mixture is then atomized into a vessel containing a liquid non solvent at a temperature below the freezing point of the polymer substance solution which freezes the polymer droplets. As the droplets and non solvent for the polymer are warmed, the solvent in the droplets thaws and is extracted into the non solvent, resulting in the hardening of the microspheres.

7. Phase Inversion Nanoencapsulation (PIN)

Particles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non solvent for the polymer, to spontaneously produce, under favorable conditions, polymeric microspheres, wherein the polymer is either coated with the particles or the particles are dispersed in the polymer. For example, see, U.S. Pat. No. 6,143,211. The method can be used to produce monodisperse populations of particles and microparticles in a wide range of sizes, including, for example, about 100 nanometers to about 10 microns.

Advantageously, an emulsion need not be formed prior to precipitation. The process can be used to form microspheres from thermoplastic polymers.

8. Emulsion Methods

In some embodiments, a particle is prepared using an emulsion solvent evaporation method. For example, a polymeric material is dissolved in a water immiscible organic solvent and mixed with a drug solution or a combination of drug solutions. In some embodiments a solution of a therapeutic, prophylactic, or diagnostic agent to be encapsulated is mixed with the polymer solution. The polymer can be, but is not limited to, one or more of the following: PLA, PGA, PCL, their copolymers, polyacrylates, the aforementioned PEGylated polymers. The drug molecules can include one or more conjugates as described above and one or more additional active agents. The water immiscible organic solvent, can be, but is not limited to, one or more of the following: chloroform, dichloromethane, and acyl acetate. The drug can be dissolved in, but is not limited to, one or more of the following: acetone, ethanol, methanol, isopropyl alcohol, acetonitrile and Dimethyl sulfoxide (DMSO).

An aqueous solution is added into the resulting polymer solution to yield emulsion solution by emulsification. The emulsification technique can be, but not limited to, probe sonication or homogenization through a homogenizer.

9. Nanoprecipitation

In another embodiment, a conjugate containing particle is prepared using nanoprecipitation methods or microfluidic devices. The conjugate containing polymeric material is mixed with a drug or drug combinations in a water miscible organic solvent, optionally containing additional polymers. The additional polymer can be, but is not limited to, one or more of the following: PLA, PGA, PCL, their copolymers, polyacrylates, the aforementioned PEGylated polymers. The water miscible organic solvent, can be, but is not limited to, one or more of the following: acetone, ethanol, methanol, isopropyl alcohol, acetonitrile and dimethyl sulfoxide (DMSO). The resulting mixture solution is then added to a polymer non-solvent, such as an aqueous solution, to yield particle solution.

10. Microfluidics

Methods of making particles using microfluidics are known in the art. Suitable methods include those described in U.S. Patent Application Publication No. 2010/0022680 A1. In general, the microfluidic device comprises at least two channels that converge into a mixing apparatus. The channels are typically formed by lithography, etching, embossing, or molding of a polymeric surface. A source of fluid is attached to each channel, and the application of pressure to the source causes the flow of the fluid in the channel. The pressure may be applied by a syringe, a pump, and/or gravity. The inlet streams of solutions with polymer, targeting moieties, lipids, drug, payload, etc. converge and mix, and the resulting mixture is combined with a polymer non-solvent solution to form the particles having the desired size and density of moieties on the surface. By varying the pressure and flow rate in the inlet channels and the nature and composition of the fluid sources particles can be produced having reproducible size and structure.

ii. Lipid Particles

Methods of making lipid particles are known in the art. Lipid particles can be lipid micelles, liposomes, or solid lipid particles prepared using any suitable method known in the art. Common techniques for created lipid particles encapsulating an active agent include, but are not limited to high pressure homogenization techniques, supercritical fluid methods, emulsion methods, solvent diffusion methods, and spray drying. A brief summary of these methods is presented below.

1. High Pressure Homogenization (HPH) Methods

High pressure homogenization is a reliable and powerful technique, which is used for the production of smaller lipid particles with narrow size distributions, including lipid micelles, liposomes, and solid lipid particles. High pressure homogenizers push a liquid with high pressure (100-2000 bar) through a narrow gap (in the range of a few microns). The fluid can contain lipids that are liquid at room temperature or a melt of lipids that are solid at room temperature. The fluid accelerates on a very short distance to very high velocity (over 1000 Km/h). This creates high shear stress and cavitation forces that disrupt the particles, generally down to the submicron range. Generally 5-10% lipid content is used but up to 40% lipid content has also been investigated.

Two approaches of HPH are hot homogenization and cold homogenization, work on the same concept of mixing the drug in bulk of lipid solution or melt.

a. Hot Homogenization:

Hot homogenization is carried out at temperatures above the melting point of the lipid and can therefore be regarded as the homogenization of an emulsion. A pre-emulsion of the drug loaded lipid melt and the aqueous emulsifier phase is obtained by a high-shear mixing. HPH of the pre-emulsion is carried out at temperatures above the melting point of the lipid. A number of parameters, including the temperature, pressure, and number of cycles, can be adjusted to produce lipid particles with the desired size. In general, higher temperatures result in lower particle sizes due to the decreased viscosity of the inner phase. However, high temperatures increase the degradation rate of the drug and the carrier. Increasing the homogenization pressure or the number of cycles often results in an increase of the particle size due to high kinetic energy of the particles.

b. Cold Homogenization

Cold homogenization has been developed as an alternative to hot homogenization. Cold homogenization does not suffer from problems such as temperature-induced drug degradation or drug distribution into the aqueous phase during homogenization. The cold homogenization is particularly useful for solid lipid particles, but can be applied with slight modifications to produce liposomes and lipid micelles. In this technique the drug containing lipid melt is cooled, the solid lipid ground to lipid microparticles and these lipid microparticles are dispersed in a cold surfactant solution yielding a pre-suspension. The pre-suspension is homogenized at or below room temperature, where the gravitation force is strong enough to break the lipid microparticles directly to solid lipid nanoparticles.

2. Ultrasonication/High Speed Homogenization Methods

Lipid particles, including lipid micelles, liposomes, and solid lipid particles, can be prepared by ultrasonication/high speed homogenization. The combination of both ultrasonication and high speed homogenization is particularly useful for the production of smaller lipid particles. Liposomes are formed in the size range from 10 nm to 200 nm, preferably 50 nm to 100 nm, by this process.

3. Solvent Evaporation Methods

Lipid particles can be prepared by solvent evaporation approaches. The lipophilic material is dissolved in a water-immiscible organic solvent (e.g., cyclohexane) that is emulsified in an aqueous phase. Upon evaporation of the solvent, particles dispersion is formed by precipitation of the lipid in the aqueous medium. Parameters such as temperature, pressure, choices of solvents can be used to control particle size and distribution. Solvent evaporation rate can be adjusted through increased/reduced pressure or increased/reduced temperature.

4. Solvent Emulsification-Diffusion Methods

Lipid particles can be prepared by solvent emulsification-diffusion methods. The lipid is first dissolved in an organic phase, such as ethanol and acetone. An acidic aqueous phase is used to adjust the zeta potential to induce lipid coacervation. The continuous flow mode allows the continuous diffusion of water and alcohol, reducing lipid solubility, which causes thermodynamic instability and generates liposomes

5. Supercritical Fluid Methods

Lipid particles, including liposomes and solid lipid particles, can be prepared from supercritical fluid methods. Supercritical fluid approaches have the advantage of replacing or reducing the amount of the organic solvents used in other preparation methods. The lipids, active agents to be encapsulated, and excipients can be solvated at high pressure in a supercritical solvent. The supercritical solvent is most commonly CO₂, although other supercritical solvents are known in the art. To increase solubility of the lipid, a small amount of co-solvent can be used. Ethanol is a common co-solvent, although other small organic solvents that are generally regarded as safe for formulations can be used. The lipid particles, lipid micelles, liposomes, or solid lipid particles can be obtained by expansion of the supercritical solution or by injection into a non-solvent aqueous phase. The particle formation and size distribution can be controlled by adjusting the supercritical solvent, co-solvent, non-solvent, temperatures, pressures, etc.

6. Microemulsion Based Methods

Microemulsion based methods for making lipid particles are known in the art. These methods are based upon the dilution of a multiphase, usually two-phase, system. Emulsion methods for the production of lipid particles generally involve the formation of a water-in-oil emulsion through the addition of a small amount of aqueous media to a larger volume of immiscible organic solution containing the lipid. The mixture is agitated to disperse the aqueous media as tiny droplets throughout the organic solvent and the lipid aligns itself into a monolayer at the boundary between the organic and aqueous phases. The size of the droplets is controlled by pressure, temperature, the agitation applied and the amount of lipid present.

The water-in-oil emulsion can be transformed into a liposomal suspension through the formation of a double emulsion. In a double emulsion, the organic solution containing the water droplets is added to a large volume of aqueous media and agitated, producing a water-in-oil-in-water emulsion. The size and type of lipid particle formed can be controlled by the choice of and amount of lipid, temperature, pressure, co-surfactants, solvents, etc.

7. Spray Drying Methods

Spray drying methods similar to those described above for making polymeric particle can be employed to create solid lipid particles. This works best for lipid with a melting point above 70° C.

In some embodiments, conjugates of the present invention may be encapsulated in polymeric particles using a single oil in water emulsion method. As a non-limiting example, the conjugate and a suitable polymer or block copolymer or a mixture of polymers/block copolymers, are dissolved in organic solvents such as, but not limited to, dichloromethane (DCM), ethyl acetate (EtAc) or choloform to form the oil phase. Co-solvents such as, but not limited to, dimethyl formamide (DMF), acetonitrile (CAN) or benzyl alcohol (BA) may be used to control the size of the particles and/or to solubilize the conjugate. Polymers used in the formulation may include, but not limited to, PLA97-b-PEG5, PLA35-b-PEG5 and PLA16-b-PEG5 copolymers.

In some embodiments, the particle may be prepared by combining a therapeutic agent, a first polymer, and an organic acid with an organic solvent to form a first organic phase having about 1 to about 50% solids; combining the first organic phase with a first aqueous solution to form the plurality of therapeutic nanoparticles; and recovering the therapeutic nanoparticles by filtration as disclosed in WO2014043618 to Figueiredo et al. (BIND), the contents of which are incorporated herein by reference in their entirety.

Particle formulations may be prepared by varying the lipophilicity of conjugates of the present invention. The lipophilicity may be varied by using hydrophobic ion-pairs or hydrophobic ion-paring (HIP) of the conjugates with different counterions. HIP alters the solubility of the conjugates of the present invention. The aqueous solubility may drop and the solubility in organic phases may increase.

Any suitable agent may be used to provide counterions to form HIP complex with the conjugate of the present invention. In some embodiments, the HIP complex may be formed prior to formulation of the particles.

### VI. Methods of Using the Conjugates and Particles

The conjugates and/or particles comprising such conjugates as described herein or formulations containing the conjugates or particles as described herein can be administered to treat any hyperproliferative disease, metabolic disease, infectious disease, inflammatory disease, cancer, or any other disease, as appropriate. The formulations can be used for immunization. The formulations may be delivered to various body parts, such as but not limited to, brain and central nervous system, eyes, ears, lungs, bone, heart, kidney, liver, spleen, breast, ovary, colon, pancreas, muscles, gastrointestinal tract, mouth, skin, to treat disesase associated with such body parts. Formulations may be administered by injection, orally, or topically, typically to a mucosal surface (lung, nasal, oral, buccal, sublingual, vaginally, rectally) or to the eye (intraocularly or transocularly).

In some embodiments, the conjugates and/or particles comprising such conjugates of the present invention may be combined with at least one other active agent to form a composition. The at least one active agent may be a therapeutic, prophylactic, diagnostic, or nutritional agent. It may be a small molecule, protein, peptide, lipid, glycolipid, glycoprotein, lipoprotein, carbohydrate, sugar, or nucleic acid. The conjugates and/or particles comprising such conjugates of the present invention and the at least one other active agent may have the same target and/or treat the same disease.

The conjugates and/or particles comprising such conjugates of the present invention and the at least one other active agent may be administered semitaneously or sequentially. They may be present as a mixture for simultaneous administration, or may each be present in separate containers for sequential administration.

The term “simultaneous administration”, as used herein, is not specifically restricted and means that the conjugates and/or particles comprising such conjugates and the at least one other active agent are substantially administered at the same time, e.g. as a mixture or in immediate subsequent sequence.

The term “sequential administration”, as used herein, is not specifically restricted and means that the conjugates and/or particles comprising such conjugates and the at least one other active agent are not administered at the same time but one after the other, or in groups, with a specific time interval between administrations. The time interval may be the same or different between the respective administrations of the conjugates and/or particles comprising such conjugates and the at least one other active agent and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

In some embodiments, more than one conjugate and/or particle comprising such conjugates may be combined to form a composition. The particles may comprise different conjugates, wherein the conjugates may have different active agents, different linkers, and/or different targeting moieties. The particles may have different particle compostions, different drug loadings, and/or different sizes. The conjugates and/or particles comprising such conjugates in the compostion may be administered semitaneously or sequentially. They may be present as a mixture for simultaneous administration, or may each be present in separate containers for sequential administration.

In some embodiments, conjugates and/or particles comprising such conjugates may be combined to form a composition. Pharmcokinetic properties of the composition, such as Cmax, may be modulated by adjusting the weight percent ratio of the conjugates and/or the particles comprising such conjugates.

In various embodiments, methods for treating a subject having a cancer are provided, wherein the method comprises administering a therapeutically-effective amount of the conjugates and/or particles comprising such conjugates, as described herein, to a subject having a cancer, suspected of having cancer, or having a predisposition to a cancer. According to the present invention, cancer embraces any disease or malady characterized by uncontrolled cell proliferation, e.g., hyperproliferation. Cancers may be characterized by tumors, e.g., solid tumors or any neoplasm.

In some embodiments, provided is a method for treating a subjection having inflammation, comprising administering a therapeutically-effective amount of the conjugates and/or particles comprising such conjugates, as described herein, to the subject. In some embodiments, the conjugates and/or particles comprising such conjugates may comprise a folate-targeting active agent, or a targeting moiety that binds to the folate receptor.

In some embodiments, the subject may be otherwise free of indications for treatment with the conjugates and/or particles comprising such conjugates. In some embodiments, methods include use of cancer cells, including but not limited to mammalian cancer cells. In some instances, the mammalian cancer cells are human cancer cells.

In some embodiments, the conjugates and/or particles comprising such conjugates of the present teachings have been found to inhibit cancer and/or tumor growth. They may also reduce, including cell proliferation, invasiveness, and/or metastasis, thereby rendering them useful for the treatment of a cancer.

In some embodiments, the conjugates and/or particles comprising such conjugates of the present teachings may be used to prevent the growth of a tumor or cancer, and/or to prevent the metastasis of a tumor or cancer. In some embodiments, compositions of the present teachings may be used to shrink or destroy a cancer.

In some embodiments, the conjugates and/or particles comprising such conjugates provided herein are useful for inhibiting proliferation of a cancer cell. In some embodiments, the conjugates and/or particles comprising such conjugates provided herein are useful for inhibiting cellular proliferation, e.g., inhibiting the rate of cellular proliferation, preventing cellular proliferation, and/or inducing cell death. In general, the conjugates and/or particles comprising such conjugates as described herein can inhibit cellular proliferation of a cancer cell or both inhibiting proliferation and/or inducing cell death of a cancer cell.

The cancers treatable by methods of the present teachings generally occur in mammals. Mammals include, for example, humans, non-human primates, dogs, cats, rats, mice, rabbits, ferrets, guinea pigs horses, pigs, sheep, goats, and cattle. In various embodiments, the cancer is lung cancer, breast cancer, e.g., mutant BRCA1 and/or mutant BRCA2 breast cancer, non-BRCA-associated breast cancer, colorectal cancer, neuroendodrine cancer, ovarian cancer, pancreatic cancer, colorectal cancer, bladder cancer, prostate cancer, cervical cancer, renal cancer, leukemia, central nervous system cancers, myeloma, and melanoma. In some embodiments, the cancer is lung cancer. In certain embodiments, the cancer is human lung carcinoma, ovarian cancer, pancreatic cancer or colorectal cancer.

The conjugates and/or particles comprising such conjugates as described herein or formulations containing the conjugates and/or particles comprising such conjugates as described herein can be used for the selective tissue delivery of a therapeutic, prophylactic, or diagnostic agent to an individual or patient in need thereof. Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. Dosage unit form as used herein refers to physically discrete units suited as unitary dosages for the mammalian subjects to be treated; each unit containing a predetermined quantity of active compound calculated to produce the desired therapeutic.

In various embodiments, a conjugate contained within a particle is released in a controlled manner. The release can be in vitro or in vivo. For example, particles can be subject to a release test under certain conditions, including those specified in the U.S. Pharmacopeia and variations thereof.

In various embodiments, less than about 90%, less than about 80%, less than about 70%, less than about 60%, less than about 50%, less than about 40%, less than about 30%, less than about 20% of the conjugate contained within particles is released in the first hour after the particles are exposed to the conditions of a release test. In some embodiments, less that about 90%, less than about 80%, less than about 70%, less than about 60%, or less than about 50% of the conjugate contained within particles is released in the first hour after the particles are exposed to the conditions of a release test. In certain embodiments, less than about 50% of the conjugate contained within particles is released in the first hour after the particles are exposed to the conditions of a release test.

With respect to a conjugate being released in vivo, for example, the conjugate contained within a particle administered to a subject may be protected from a subject's body, and the body may also be isolated from the conjugate until the conjugate is released from the particle.

Thus, in some embodiments, the conjugate may be substantially contained within the particle until the particle is delivered into the body of a subject. For example, less than about 90%, less than about 80%, less than about 70%, less than about 60%, less than about 50%, less than about 40%, less than about 30%, less than about 20%, less than about 15%, less than about 10%, less than about 5%, or less than about 1% of the total conjugate is released from the particle prior to the particle being delivered into the body, for example, a treatment site, of a subject. In some embodiments, the conjugate may be released over an extended period of time or by bursts (e.g., amounts of the conjugate are released in a short period of time, followed by a periods of time where substantially no conjugate is released). For example, the conjugate can be released over 6 hours, 12 hours, 24 hours, or 48 hours. In certain embodiments, the conjugate is released over one week or one month.

In some embodiments, the conjugates and/or particles comprising such conjugates of the present teachings may be administered to tumors with a high level of enhanced permeability and retention (EPR) effect. In some embodiments, tumors with a high level of enhanced permeability and retention effect may be identified with imaging techniques. As a non-limited example, iron oxide nanoparticle magnetic resonance imaging may be administered to a patient and EPR effects are measured.

In some embodiments, compounds and/or composition of the present teachings may be administered to a subject selected with the method disclosed in WO2015017506, the contents of which are incorporated herein by reference in their entirety, the method comprising:

(a) administering a contrast agent to the subject;

(b) measuring the level of accumulation of the contrast agent at at least one intended site of treatment; and
(c) selecting the subject based on the level of the accumulation of the contrast agent;

wherein the intended site of treatment is a tumor.

### VII. Kits and Devices

The invention provides a variety of kits and devices for conveniently and/or effectively carrying out methods of the present invention. Typically kits will comprise sufficient amounts and/or numbers of components to allow a user to perform multiple treatments of a subject(s) and/or to perform multiple experiments.

In one embodiment, the present invention provides kits for inhibiting tumor cell growth in vitro or in vivo, comprising a conjugate and/or particle comprising such conjugates of the present invention or a combination of conjugates and/or particles comprising such conjugates of the present invention, optionally in combination with any other active agents.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of the conjugates and/or particles comprising such conjugates in the buffer solution over a period of time and/or under a variety of conditions.

The present invention provides for devices which may incorporate conjugates and/or particles comprising such conjugates of the present invention. These devices contain in a stable formulation available to be immediately delivered to a subject in need thereof, such as a human patient. In some embodiments, the subject has cancer.

Non-limiting examples of the devices include a pump, a catheter, a needle, a transdermal patch, a pressurized olfactory delivery device, iontophoresis devices, multi-layered microfluidic devices. The devices may be employed to deliver conjugates and/or particles comprising such conjugates of the present invention according to single, multi- or split-dosing regiments. The devices may be employed to deliver conjugates and/or particles comprising such conjugates of the present invention across biological tissue, intradermal, subcutaneously, or intramuscularly.

### VIII. Definitions

The term “compound”, as used herein, is meant to include all stereoisomers, geometric isomers, tautomers, and isotopes of the structures depicted. In the present application, compound is used interechangably with conjugate. Therefore, conjugate, as used herein, is also meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

The terms “subject” or “patient”, as used herein, refer to any organism to which the particles may be administered, e.g., for experimental, therapeutic, diagnostic, and/or prophylactic purposes. Typical subjects include animals (e.g., mammals such as mice, rats, rabbits, guinea pigs, cattle, pigs, sheep, horses, dogs, cats, hamsters, lamas, non-human primates, and humans).

The terms “treating” or “preventing”, as used herein, can include preventing a disease, disorder or condition from occurring in an animal that may be predisposed to the disease, disorder and/or condition but has not yet been diagnosed as having the disease, disorder or condition; inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

A “target”, as used herein, shall mean a site to which targeted constructs bind. A target may be either in vivo or in vitro. In certain embodiments, a target may be cancer cells found in leukemias or tumors (e.g., tumors of the brain, lung (small cell and non-small cell), ovary, prostate, breast and colon as well as other carcinomas and sarcomas). In still other embodiments, a target may refer to a molecular structure to which a targeting moiety or ligand binds, such as a hapten, epitope, receptor, dsDNA fragment, carbohydrate or enzyme. A target may be a type of tissue, e.g., neuronal tissue, intestinal tissue, pancreatic tissue, liver, kidney, prostate, ovary, lung, bone marrow, or breast tissue

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human.

The term “modulation” is art-recognized and refers to up regulation (i.e., activation or stimulation), down regulation (i.e., inhibition or suppression) of a response, or the two in combination or apart.

“Parenteral administration”, as used herein, means administration by any method other than through the digestive tract (enteral) or non-invasive topical routes. For example, parenteral administration may include administration to a patient intravenously, intradermally, intraperitoneally, intrapleurally, intratracheally, intraossiously, intracerebrally, intrathecally, intramuscularly, subcutaneously, subjunctivally, by injection, and by infusion.

“Topical administration”, as used herein, means the non-invasive administration to the skin, orifices, or mucosa. Topical administrations can be administered locally, i.e., they are capable of providing a local effect in the region of application without systemic exposure. Topical formulations can provide systemic effect via adsorption into the blood stream of the individual. Topical administration can include, but is not limited to, cutaneous and transdermal administration, buccal administration, intranasal administration, intravaginal administration, intravesical administration, ophthalmic administration, and rectal administration.

“Enteral administration”, as used herein, means administration via absorption through the gastrointestinal tract. Enteral administration can include oral and sublingual administration, gastric administration, or rectal administration.

“Pulmonary administration”, as used herein, means administration into the lungs by inhalation or endotracheal administration. As used herein, the term “inhalation” refers to intake of air to the alveoli. The intake of air can occur through the mouth or nose.

The terms “sufficient” and “effective”, as used interchangeably herein, refer to an amount (e.g., mass, volume, dosage, concentration, and/or time period) needed to achieve one or more desired result(s). A “therapeutically effective amount” is at least the minimum concentration required to effect a measurable improvement or prevention of at least one symptom or a particular condition or disorder, to effect a measurable enhancement of life expectancy, or to generally improve patient quality of life. The therapeutically effective amount is thus dependent upon the specific biologically active molecule and the specific condition or disorder to be treated. Therapeutically effective amounts of many active agents, such as antibodies, are known in the art. The therapeutically effective amounts of compounds and compositions described herein, e.g., for treating specific disorders may be determined by techniques that are well within the craft of a skilled artisan, such as a physician.

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The term “prodrug” refers to an agent, including a nucleic acid or protein that is converted into a biologically active form in vitro and/or in vivo. Prodrugs can be useful because, in some situations, they may be easier to administer than the parent compound. For example, a prodrug may be bioavailable by oral administration whereas the parent compound is not. The prodrug may also have improved solubility in pharmaceutical compositions compared to the parent drug. A prodrug may be converted into the parent drug by various mechanisms, including enzymatic processes and metabolic hydrolysis. Harper, N.J. (1962) Drug Latentiation in Jucker, ed. Progress in Drug Research, 4:221-294; Morozowich et al. (1977) Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997) Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996) Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogs, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs-principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996) Improved oral drug delivery: solubility limitations overcome by the use of prodrugs, Adv. Drug Delivery Rev. 19(2): 115-130; Fleisher et al. (1985) Design of prodrugs for improved gastrointestinal absorption by intestinal enzyme targeting, Methods Enzymol. 112: 360-81; Farquhar D, et al. (1983) Biologically Reversible Phosphate-Protective Groups, J. Pharm. Sci., 72(3): 324-325; Han, H. K. et al. (2000) Targeted prodrug design to optimize drug delivery, AAPS PharmSci., 2(1): E6; Sadzuka Y. (2000) Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl. 2:S15-27; Wang, W. et al. (1999) Prodrug approaches to the improved delivery of peptide drugs. Curr. Pharm. Des., 5(4):265-87.

The term “biocompatible”, as used herein, refers to a material that along with any metabolites or degradation products thereof that are generally non-toxic to the recipient and do not cause any significant adverse effects to the recipient. In general, biocompatible materials are materials that do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks or even longer.

The term “pharmaceutically acceptable”, as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier”, as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

The term “small molecule”, as used herein, generally refers to an organic molecule that is less than 2000 g/mol in molecular weight, less than 1500 g/mol, less than 1000 g/mol, less than 800 g/mol, or less than 500 g/mol. Small molecules are non-polymeric and/or non-oligomeric.

The term “hydrophilic”, as used herein, refers to substances that have strongly polar groups that readily interact with water.

The term “hydrophobic”, as used herein, refers to substances that lack an affinity for water; tending to repel and not absorb water as well as not dissolve in or mix with water.

The term “lipophilic”, as used herein, refers to compounds having an affinity for lipids.

The term “amphiphilic”, as used herein, refers to a molecule combining hydrophilic and lipophilic (hydrophobic) properties. “Amphiphilic material” as used herein refers to a material containing a hydrophobic or more hydrophobic oligomer or polymer (e.g., biodegradable oligomer or polymer) and a hydrophilic or more hydrophilic oligomer or polymer.

The term “targeting moiety”, as used herein, refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. In some embodiments, a targeting moiety can specifically bind to a selected molecule.

The term “reactive coupling group”, as used herein, refers to any chemical functional group capable of reacting with a second functional group to form a covalent bond. The selection of reactive coupling groups is within the ability of the skilled artisan. Examples of reactive coupling groups can include primary amines (—NH₂) and amine-reactive linking groups such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these conjugate to amines by either acylation or alkylation. Examples of reactive coupling groups can include aldehydes (—COH) and aldehyde reactive linking groups such as hydrazides, alkoxyamines, and primary amines. Examples of reactive coupling groups can include thiol groups (—SH) and sulfhydryl reactive groups such as maleimides, haloacetyls, and pyridyl disulfides. Examples of reactive coupling groups can include photoreactive coupling groups such as aryl azides or diazirines. The coupling reaction may include the use of a catalyst, heat, pH buffers, light, or a combination thereof.

The term “protective group”, as used herein, refers to a functional group that can be added to and/or substituted for another desired functional group to protect the desired functional group from certain reaction conditions and selectively removed and/or replaced to deprotect or expose the desired functional group. Protective groups are known to the skilled artisan. Suitable protective groups may include those described in Greene and Wuts., Protective Groups in Organic Synthesis, (1991). Acid sensitive protective groups include dimethoxytrityl (DMT), tert-butylcarbamate (tBoc) and trifluoroacetyl (tFA). Base sensitive protective groups include 9-fluorenylmethoxycarbonyl (Fmoc), isobutyrl (iBu), benzoyl (Bz) and phenoxyacetyl (pac). Other protective groups include acetamidomethyl, acetyl, tert-amyloxycarbonyl, benzyl, benzyloxycarbonyl, 2-(4-biphenylyl)-2-propyloxycarbonyl, 2-bromobenzyloxycarbonyl, tert-butyl₇ tert-butyloxycarbonyl, 1-carbobenzoxamido-2,2.2-trifluoroethyl, 2,6-dichlorobenzyl, 2-(3,5-dimethoxyphenyl)-2-propyloxycarbonyl, 2,4-dinitrophenyl, dithiasuccinyl, formyl, 4-methoxybenzenesulfonyl, 4-methoxybenzyl, 4-methylbenzyl, o-nitrophenylsulfenyl, 2-phenyl-2-propyloxycarbonyl, a-2,4,5-tetramethylbenzyloxycarbonyl, p-toluenesulfonyl, xanthenyl, benzyl ester, N-hydroxysuccinimide ester, p-nitrobenzyl ester, p-nitrophenyl ester, phenyl ester, p-nitrocarbonate, p-nitrobenzylcarbonate, trimethylsilyl and pentachlorophenyl ester.

The term “activated ester”, as used herein, refers to alkyl esters of carboxylic acids where the alkyl is a good leaving group rendering the carbonyl susceptible to nucleophilic attack by molecules bearing amino groups. Activated esters are therefore susceptible to aminolysis and react with amines to form amides. Activated esters contain a carboxylic acid ester group —CO₂R where R is the leaving group.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths. Throughout the application, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

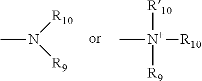

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkly, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

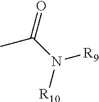

wherein R₉ and R₁₀ are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF3, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or non-aromatic ring in which each atom of the ring is carbon.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, and —CN.

The term “carbonyl” is art-recognized and includes such moieties as can be represented by the general formula:

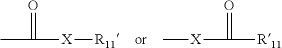

wherein X is a bond or represents an oxygen or a sulfur, and Ru represents a hydrogen, an alkyl, a cycloalkyl, an alkenyl, an cycloalkenyl, or an alkynyl, R′₁₁ represents a hydrogen, an alkyl, a cycloalkyl, an alkenyl, an cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′ii is hydrogen, the formula represents a “formate”. In general, where the oxygen atom of the above formula is replaced by sulfur, the formula represents a “thiocarbonyl” group. Where X is a sulfur and Ru or is not hydrogen, the formula represents a “thioester.” Where X is a sulfur and Ru is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “monoester” as used herein refers to an analog of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “heteroatom” as used herein means an atom of any element other than carbon or hydrogen. Examples of heteroatoms are boron, nitrogen, oxygen, phosphorus, sulfur and selenium. Other heteroatoms include silicon and arsenic.

As used herein, the term “nitro” means —NO2; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative sub stituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, or elimination.

In a broad aspect, the permissible substituents include acyclic and cyclic, branched and unbranched, carbocyclic and heterocyclic, aromatic and nonaromatic sub stituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkyl sulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The term “copolymer” as used herein, generally refers to a single polymeric material that is comprised of two or more different monomers. The copolymer can be of any form, such as random, block, graft, etc. The copolymers can have any end-group, including capped or acid end groups.

The term “mean particle size”, as used herein, generally refers to the statistical mean particle size (diameter) of the particles in the composition. The diameter of an essentially spherical particle may be referred to as the physical or hydrodynamic diameter of a spherical particle with an equivalent volume. The diameter of a non-spherical particle may refer to the hydrodynamic diameter. As used herein, the diameter of a non-spherical particle may refer to the largest linear distance between two points on the surface of the particle. Mean particle size can be measured using methods known in the art such as dynamic light scattering (DLS), electron microscopy, laser diffraction, MALDI-TDF, zeta potential measurement, AFM, TEM, SEM X-Ray microanalysis, or nanoparticle tracking analysis. Two populations can be said to have a “substantially equivalent mean particle size” when the statistical mean particle size of the first population of particles is within 20% of the statistical mean particle size of the second population of particles; for example, within 15%, or within 10%.

The terms “monodisperse” and “homogeneous size distribution”, as used interchangeably herein, describe a population of particles, microparticles, or nanoparticles all having the same or nearly the same size. As used herein, a monodisperse distribution refers to particle distributions in which 90% of the distribution lies within 5% of the mean particle size.

The term “polydispersity index” is used herein as a measure of the size distribution of an ensemble of particles, e.g., nanoparticles. The polydispersity index can be calculated based on dynamic light scattering measurements.

The terms “polypeptide,” “peptide” and “protein” generally refer to a polymer of amino acid residues. As used herein, the term also applies to amino acid polymers in which one or more amino acids are chemical analogs or modified derivatives of corresponding naturally-occurring amino acids. The term “protein”, as generally used herein, refers to a polymer of amino acids linked to each other by peptide bonds to form a polypeptide for which the chain length is sufficient to produce tertiary and/or quaternary structure. The term “protein” excludes small peptides by definition, the small peptides lacking the requisite higher-order structure necessary to be considered a protein.

A “functional fragment” of a protein, polypeptide or nucleic acid is a protein, polypeptide or nucleic acid whose sequence is not identical to the full-length protein, polypeptide or nucleic acid, yet retains at least one function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA binding function of a polypeptide can be determined, for example, by filter-binding, electrophoretic mobility shift, or immunoprecipitation assays. DNA cleavage can be assayed by gel electrophoresis. The ability of a protein to interact with another protein can be determined, for example, by co-immunoprecipitation, two-hybrid assays or complementation, e.g., genetic or biochemical. See, for example, Fields et al. (1989) Nature 340:245-246; U.S. Pat. No. 5,585,245 and PCT WO 98/44350.

As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15,16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. Those of skill in the art will recognize that each of these groups may in turn be substituted. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers.

The term “pharmaceutically acceptable counter ion” refers to a pharmaceutically acceptable anion or cation. In various embodiments, the pharmaceutically acceptable counter ion is a pharmaceutically acceptable ion. For example, the pharmaceutically acceptable counter ion is selected from citrate, malate, acetate, oxalate, chloride, bromide, iodide, nitrate, sulfate, bisulfate, phosphate, acid phosphate, isonicotinate, acetate, lactate, salicylate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)). In some embodiments, the pharmaceutically acceptable counter ion is selected from chloride, bromide, iodide, nitrate, sulfate, bisulfate, phosphate, acid phosphate, citrate, malate, acetate, oxalate, acetate, and lactate. In particular embodiments, the pharmaceutically acceptable counter ion is selected from chloride, bromide, iodide, nitrate, sulfate, bisulfate, and phosphate.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

If the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

A pharmaceutically acceptable salt can be derived from an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, hydrochloric acid, isethionic, isobutyric acid, lactic acid, lactobionic acid, lauric acid, maleic acid, malic acid, malonic acid, mandelic acid, methanesulfonic acid, mucic, naphthalene-1,5-disulfonic acid, naphthalene-2-sulfonic acid, nicotinic acid, nitric acid, oleic acid, oxalic acid, palmitic acid, pamoic acid, pantothenic, phosphoric acid, proprionic acid, pyroglutamic acid, salicylic acid, sebacic acid, stearic acid, succinic acid, sulfuric acid, tartaric acid, thiocyanic acid, toluenesulfonic acid, trifluoroacetic, and undecylenic acid.

The term “bioavailable” is art-recognized and refers to a form of the subject invention that allows for it, or a portion of the amount administered, to be absorbed by, incorporated to, or otherwise physiologically available to a subject or patient to whom it is administered.

It will be appreciated that the following examples are intended to illustrate but not to limit the present invention. Various other examples and modifications of the foregoing description and examples will be apparent to a person skilled in the art after reading the disclosure without departing from the spirit and scope of the invention, and it is intended that all such examples or modifications be included within the scope of the appended claims. All publications and patents referenced herein are hereby incorporated by reference in their entirety.

It will be appreciated that the following examples are intended to illustrate but not to limit the present invention. Various other examples and modifications of the foregoing description and examples will be apparent to a person skilled in the art after reading the disclosure without departing from the spirit and scope of the invention, and it is intended that all such examples or modifications be included within the scope of the appended claims. All publications and patents referenced herein are hereby incorporated by reference in their entirety.

### EXAMPLES

### Example 1: Analysis of Products Using C18 Reverse Phase HPLC (Method 1)

HPLC analysis of drug conjugates, e.g., RGD-SS-cabazitaxel drug conjugate, was carried out on Zorbax Eclipse XDB-C18 reverse phase column (4.6×100 mm, 3.5 μm, Agilent PN: 961967-902) with a mobile phase consisting of water+0.1% TFA (solvent A) and acetonitrile+0.1% TFA (solvent B at a flow rate of the 1.5 mL/minute and column temperature of 35° C. The injection volume was 104, and the analyte was detected using UV at 220 and 254 nm. The gradient is shown in Table 1.

TABLE 1
Gradient

| Time (mins) | % A | % B |
|---|---|---|
| 0 | 95 | 5 |
| 6 | 5 | 95 |
| 8 | 5 | 95 |
| 8.01 | 95 | 5 |
| 10 | 95 | 5 |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

1. A conjugate comprising an active agent coupled to a targeting moiety by a linker, wherein the targeting moiety is a stapled peptide.

2. The conjugate of claim 1, wherein the stapled peptide has a cross-linker between at least two amino acids.

3. The conjugate of claim 2, wherein the cross-linker comprises a disulfide bond, an amide bond, or a carbon-carbon bond.

4. The conjugate of claim 3, wherein the cross-linker comprises consecutive carbon-carbon bonds.

5. The conjugate of claim 4, wherein the cross-linker comprises at least 5 consecutive carbon-carbon bonds.

6. The conjugate of claim 5, wherein the cross-linker comprises at least 10 consecutive carbon-carbon bonds.

7. The conjugate of claim 1, wherein the targeting moiety binds to a luteinizing-hormone-releasing hormone (LHRH) receptor, a somatostatin receptor, a receptor tyrosine kinases (RTK), a serine or threonine kinase, G-protein coupled receptor, methyl CpG binding protein, cell surface glycoprotein, cancer stem cell antigen or marker, carbonic anhydrase, cytolytic T lymphocyte antigen, DNA methyltransferase, an ectoenzyme, a glycosylphosphatidylinositol-anchored co-receptor, a glypican-related integral membrane proteoglycan, a heat shock protein, a hypoxia induced protein, a multi drug resistant transporter, a Tumor-associated macrophage marker, a tumor associated carbohydrate antigen, a TNF receptor family member, a transmembrane protein, a tumor necrosis factor receptor superfamily member, a tumour differentiation antigen, a zinc dependent metallo-exopeptidase, a zinc transporter, a sodium-dependent transmembrane transport protein, a member of the SIGLEC family of lectins, a matrix metalloproteinase, a cell surface marker, CD19, CD70, CD56, PSMA, alpha integrin, CD22, CD138, EphA2, AGS-5, Nectin-4, HER2, GPMNB, CD74, Le, any protein in Category A, or any protein in Category B.

8. The conjugate of claim 7, wherein the RTK is selected from the group consisting of any member of RTK class I, RTK class II, RTK class III, RTK class IV, RTK class V, RTK class VI, RTK class VII, RTK class VIII, RTK class IX, RTK class X, RTK class XI, RTK class XII, RTK class XIII, RTK class XIV, RTK class XV, RTK class XVI, and RTK class XVII.

9. The conjugate of claim 8, wherein the RTK is selected from the group consisting of any member of the EGF receptor family, ErbB family, Insulin receptor family, PDGF receptor family, FGF receptor family, VEGF receptors family, HGF receptor family, Trk receptor family, Eph receptor family, AXL receptor family, LTK receptor family, TIE receptor family, ROR receptor family, DDR receptor family, RET receptor family, KLG receptor family, RYK receptor family, MuSK receptor family.

10. The conjugate of claim 7, wherein the cell surface marker is selected from the group consisting of HER-2, HER-3, EGFR, and folate receptor.

11. The conjugate of claim 1, wherein the conjugate comprises a formula selected from the group X—Y—Z, X—Y—Z—Y—X, X—(Y—Z)ₙ, (X—Y)ₙ—Z, X—Y—Zₙ, and (X—Y—Z—Y)ₙ—Z;
wherein X is the stapled peptide,
Y is the linker,
Z is the active agent, and
n is an integer between 2 and 1,000.

12. The conjugate of claim 11, wherein the conjugate comprises the formula X—Y—Z;
wherein X is the stapled peptide,
Y is the linker, and
Z is the active agent.

13.-15. (canceled)

16. The conjugate of claim 1, wherein the linker comprises alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl, ether, alkoxy, aryloxy, amino, amide, carbamate, alkyl, alkenyl, alkynyl, aryl, arylalkyl, cycloalkyl, heteroaryl, or heterocyclyl optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl.

17. The conjugate of claim 1, wherein the linker is not a cleavable linker.

18. The conjugate of claim 1, wherein the linker is a cleavable linker.

19. The conjugate of claim 1, wherein the linker comprises an ester bond, disulfide, amide, acylhydrazone, ether, carbamate, carbonate, or urea.

20. The conjugate of claim 1, wherein the linker is not polymeric.

21. The conjugate of claim 1, wherein the active agent is selected from the group consisting of therapeutic, prophylactic, nutraceutical, and diagnostic agents.

22. The conjugate of claim 21, wherein the active agent is selected from chemotherapeutic agents, anti-cancer agents, anti-infective agents, anti-inflammatory agents, antibiotics, and combinations thereof.

23. The conjugate of claim 22, wherein the active agent is a protein, peptide, lipid, carbohydrate, sugar, nucleic acid, small molecule, or combination thereof.

24. The conjugate of claim 21, wherein the active agent is an organometallic compound.

25. The conjugate of claim 24, wherein the active agent is a platinum compound.

26. (canceled)

27. The conjugate of claim 22, wherein the active agent is cabazitaxel.

28. The conjugate of claim 22, wherein the active agent is tubulysin or its analog or derivative.

29. The conjugate of claim 1, wherein the stapled peptide targets cancer cells.

30. The conjugate of claim 1, wherein the conjugate has a molecular weight of less than 50,000 Da.

31. (canceled)

32. A particle comprising a conjugate of claim 1, wherein the particle comprises at least one polymeric matrix.

33. The particle of 32, wherein the polymeric matrix comprises one or more polymers selected from the group consisting of hydrophobic polymers, hydrophilic polymers, and copolymers thereof.

34.-35. (canceled)

36. The particle of claim 32, wherein the polymeric matrix comprises one or more polymers selected from the group consisting of poly(lactic acid), poly(glycolic acid), poly(lactic-co-glycolic acid), poly(ethylene oxide), poly(ethylene glycol), poly(propylene glycol), and copolymers thereof.

37. The particle of claim 32, wherein the particle has a diameter between 10 nm and 5000 nm.

38.-42. (canceled)

43. The particle of claim 3242, wherein the particle has a diameter between about 10 nm and 500 nm.

44. The particle of claim 32, wherein the conjugate is present in an amount between 0.05% and 50% (w/w) based upon the weight of the particle.

45. A pharmaceutical formulation comprising the conjugate of claim 1 and at least one pharmaceutically acceptable excipient.

46. A method of treating a subject in need thereof comprising administering a therapeutically effective amount of the formulation of claim 45.

47. The method of claim 46, wherein the subject has cancer.

48. The method of claim 46, wherein the subject has inflammation.
